SEQUENCE LISTING (391 sequences)

SEQ ID NO: 1 - protein, 5 aa, synthetic construct
  REGION 1..5: note = Synthetic construct
KYTMH

SEQ ID NO: 2 - protein, 17 aa, synthetic construct
  REGION 1..17: note = Synthetic construct
HFNPNNDETKYNQKFTG

SEQ ID NO: 3 - protein, 10 aa, synthetic construct
  REGION 1..10: note = Synthetic construct
DGLGLRLFAD

SEQ ID NO: 4 - protein, 11 aa, synthetic construct
  REGION 1..11: note = Synthetic construct
GASENIYGALN

SEQ ID NO: 5 - protein, 7 aa, synthetic construct
  REGION 1..7: note = Synthetic construct
GATNLAD

SEQ ID NO: 6 - protein, 9 aa, synthetic construct
  REGION 1..9: note = Synthetic construct
QNILDTPWT

SEQ ID NO: 7 - protein, 5 aa, synthetic construct
  REGION 1..5: note = Synthetic construct
KYAIS

SEQ ID NO: 8 - protein, 17 aa, synthetic construct
  REGION 1..17: note = Synthetic construct
HFNPNNDETKYNQKFQG

SEQ ID NO: 9 - protein, 10 aa, synthetic construct
  REGION 1..10: note = Synthetic construct
DGLGLRLFAD

SEQ ID NO: 10 - protein, 11 aa, synthetic construct
  REGION 1..11: note = Synthetic construct
RASENIYGALN

SEQ ID NO: 11 - protein, 7 aa, synthetic construct
  REGION 1..7: note = Synthetic construct
GATNLAD

SEQ ID NO: 12 - protein, 9 aa, synthetic construct
  REGION 1..9: note = Synthetic construct
QNILDTPWT

SEQ ID NO: 13 - protein, 5 aa, synthetic construct
  REGION 1..5: note = Synthetic construct
NFAIS

SEQ ID NO: 14 - protein, 17 aa, synthetic construct
  REGION 1..17: note = Synthetic construct
GIIPGHAKANYAQKFQG

SEQ ID NO: 15 - protein, 10 aa, synthetic construct
  REGION 1..10: note = Synthetic construct
DGLGIRLFAD

SEQ ID NO: 16 - protein, 11 aa, synthetic construct
  REGION 1..11: note = Synthetic construct
RASQEIYGALN

SEQ ID NO: 17 - protein, 7 aa, synthetic construct
  REGION 1..7: note = Synthetic construct
GATNLQS

SEQ ID NO: 18 - protein, 9 aa, synthetic construct
  REGION 1..9: note = Synthetic construct
QDIYDAPWT

SEQ ID NO: 19 - protein, 5 aa, synthetic construct
  REGION 1..5: note = Synthetic construct
KFAIS

SEQ ID NO: 20 - protein, 17 aa, synthetic construct
  REGION 1..17: note = Synthetic construct
GIIPGHAKANYAQKFQG

SEQ ID NO: 21 - protein, 10 aa, synthetic construct
  REGION 1..10: note = Synthetic construct
DGLGIRLFAD

SEQ ID NO: 22 - protein, 11 aa, synthetic construct
  REGION 1..11: note = Synthetic construct
RASQEIYGALN

SEQ ID NO: 23 - protein, 7 aa, synthetic construct
  REGION 1..7: note = Synthetic construct
GATNLQS

SEQ ID NO: 24 - protein, 9 aa, synthetic construct
  REGION 1..9: note = Synthetic construct
QDIYDAPWT

SEQ ID NO: 25 - protein, 5 aa, synthetic construct
  REGION 1..5: note = Synthetic construct
KYAIS

SEQ ID NO: 26 - protein, 17 aa, synthetic construct
  REGION 1..17: note = Synthetic construct
GIIPGHAKANYAQKFQG

SEQ ID NO: 27 - protein, 10 aa, synthetic construct
  REGION 1..10: note = Synthetic construct
DGLGIRLFAD

SEQ ID NO: 28 - protein, 11 aa, synthetic construct
  REGION 1..11: note = Synthetic construct
RASQKIYGALN

SEQ ID NO: 29 - protein, 7 aa, synthetic construct
  REGION 1..7: note = Synthetic construct
GATNLQS

SEQ ID NO: 30 - protein, 9 aa, synthetic construct
  REGION 1..9: note = Synthetic construct
QNTYDTPWT

SEQ ID NO: 31 - protein, 5 aa, synthetic construct
  REGION 1..5: note = Synthetic construct
GHAIS

SEQ ID NO: 32 - protein, 17 aa, synthetic construct
  REGION 1..17: note = Synthetic construct
GIIPGHAKANYAQKFQG

SEQ ID NO: 33 - protein, 10 aa, synthetic construct
  REGION 1..10: note = Synthetic construct
DGLGIRLFAD

SEQ ID NO: 34 - protein, 11 aa, synthetic construct
  REGION 1..11: note = Synthetic construct
RASQKIYGALN

SEQ ID NO: 35 - protein, 7 aa, synthetic construct
  REGION 1..7: note = Synthetic construct
GATNLQS

SEQ ID NO: 36 - protein, 9 aa, synthetic construct
  REGION 1..9: note = Synthetic construct
QNTYDTPWT

SEQ ID NO: 37 - protein, 5 aa, synthetic construct
  REGION 1..5: note = Synthetic construct
DYGMS

SEQ ID NO: 38 - protein, 17 aa, synthetic construct
  REGION 1..17: note = Synthetic construct
DINWSGEITAYADSVKG

SEQ ID NO: 39 - protein, 12 aa, synthetic construct
  REGION 1..12: note = Synthetic construct
SNSYRWDDALDI

SEQ ID NO: 40 - protein, 11 aa, synthetic construct
  REGION 1..11: note = Synthetic construct
RASQSVSSNLA

SEQ ID NO: 41 - protein, 7 aa, synthetic construct
  REGION 1..7: note = Synthetic construct
GASSRAT

SEQ ID NO: 42 - protein, 10 aa, synthetic construct
  REGION 1..10: note = Synthetic construct
QQYGSSPPVT

SEQ ID NO: 43 - protein, 5 aa, synthetic construct
  REGION 1..5: note = Synthetic construct
DYAMH

SEQ ID NO: 44 - protein, 17 aa, synthetic construct
  REGION 1..17: note = Synthetic construct
VISYDGSNKYYADSVKG

SEQ ID NO: 45 - protein, 13 aa, synthetic construct
  REGION 1..13: note = Synthetic construct
DRIGWYDYDAFDI

SEQ ID NO: 46 - protein, 11 aa, synthetic construct
  REGION 1..11: note = Synthetic construct
RASHSVGSNLA

SEQ ID NO: 47 - protein, 7 aa, synthetic construct
  REGION 1..7: note = Synthetic construct
DASNRAT

SEQ ID NO: 48 - protein, 9 aa, synthetic construct
  REGION 1..9: note = Synthetic construct
QQRSNWPPT

SEQ ID NO: 49 - protein, 7 aa, synthetic construct
  REGION 1..7: note = Synthetic construct
GYTFTKY

SEQ ID NO: 50 - protein, 6 aa, synthetic construct
  REGION 1..6: note = Synthetic construct
NPNNDE

SEQ ID NO: 51 - protein, 7 aa, synthetic construct
  REGION 1..7: note = Synthetic construct
GYRFHNF

SEQ ID NO: 52 - protein, 6 aa, synthetic construct
  REGION 1..6: note = Synthetic construct
IPGHAK

SEQ ID NO: 53 - protein, 7 aa, synthetic construct
  REGION 1..7: note = Synthetic construct
GSRFYKF

SEQ ID NO: 54 - protein, 7 aa, synthetic construct
  REGION 1..7: note = Synthetic construct
GSGFRGH

SEQ ID NO: 55 - protein, 7 aa, synthetic construct
  REGION 1..7: note = Synthetic construct
GFTFDDY

SEQ ID NO: 56 - protein, 6 aa, synthetic construct
  REGION 1..6: note = Synthetic construct
NWSGEI

SEQ ID NO: 57 - protein, 6 aa, synthetic construct
  REGION 1..6: note = Synthetic construct
SYDGSN

SEQ ID NO: 58 - protein, 119 aa, synthetic construct
  REGION 1..119: note = Synthetic construct
QVHLQQSGPELVKPGASVKMSCKTSGYTFTKYTMHWVKQGHEESLEWIGHFNPNNDETKYNQKFTGKATLTVDKSSTTAYMELRSLTSDDSALYYCARDGLGLRLFADWGQGTLITVSA

SEQ ID NO: 59 - protein, 107 aa, synthetic construct
  REGION 1..107: note = Synthetic construct
DIQMTQSPASLSASVGETVTITCGASENIYGALNWYQRKQGKSPQLLIFGATNLADGVSSRFSGSGSDRQYSLKISSLHPDDVATYYCQNILDTPWTFGGGTKLEIK

SEQ ID NO: 60 - protein, 119 aa, synthetic construct
  REGION 1..119: note = Synthetic construct
QVQLVQSGAEVKKPGSSVKVSCKASGYTFTKYAISWVRQAPGQGLEWMGHFNPNNDETKYNQKFQGRVTITADESTSTAYMELSSLRSEDTAVYYCARDGLGLRLFADWGQGTLVTVSS

SEQ ID NO: 61 - protein, 107 aa, synthetic construct
  REGION 1..107: note = Synthetic construct
DIQMTQSPSSLSASVGDRVTITCRASENIYGALNWYQQKPGKAPKLLIYGATNLADGVPSRFSGSGSGTDFTLTISSLQPEDFATYYCQNILDTPWTFGGGTKLEIK

SEQ ID NO: 62 - protein, 119 aa, synthetic construct
  REGION 1..119: note = Synthetic construct
QVQLVQSGAEVKKPGSSVKVSCKASGYRFHNFAISWVRQAPGQGLEWMGGIIPGHAKANYAQKFQGRVTITADESTSTAYMELSSLRSEDTAVYYCARDGLGIRLFADWGQGTLVTVSS

SEQ ID NO: 63 - protein, 107 aa, synthetic construct
  REGION 1..107: note = Synthetic construct
DIQMTQSPSSLSASVGDRVTITCRASQEIYGALNWYQQKPGKAPKLLIYGATNLQSGVPSRFSGSGSGTDFTLTISSLQPEDFATYYCQDIYDAPWTFGGGTKVEIK

SEQ ID NO: 64 - protein, 119 aa, synthetic construct
  REGION 1..119: note = Synthetic construct
QVQLVQSGAEVKKPGSSVKVSCKASGSRFYKFAISWVRQAPGQGLEWMGGIIPGHAKANYAQKFQGRVTITADESTSTAYMELSSLRSEDTAVYYCARDGLGIRLFADWGQGTLVTVSS

SEQ ID NO: 65 - protein, 107 aa, synthetic construct; identical to SEQ ID NO: 63
  REGION 1..107: note = Synthetic construct

SEQ ID NO: 66 - protein, 119 aa, synthetic construct
  REGION 1..119: note = Synthetic construct
QVQLVQSGAEVKKPGSSVKVSCKASGYTFTKYAISWVRQAPGQGLEWMGGIIPGHAKANYAQKFQGRVTITADESTSTAYMELSSLRSEDTAVYYCARDGLGIRLFADWGQGTLVTVSS

SEQ ID NO: 67 - protein, 107 aa, synthetic construct
  REGION 1..107: note = Synthetic construct
DIQMTQSPSSLSASVGDRVTITCRASQKIYGALNWYQQKPGKAPKLLIYGATNLQSGVPSRFSGSGSGTDFTLTISSLQPEDFATYYCQNTYDTPWTFGGGTKVEIK

SEQ ID NO: 68 - protein, 119 aa, synthetic construct
  REGION 1..119: note = Synthetic construct
QVQLVQSGAEVKKPGSSVKVSCKASGSGFRGHAISWVRQAPGQGLEWMGGIIPGHAKANYAQKFQGRVTITADESTSTAYMELSSLRSEDTAVYYCARDGLGIRLFADWGQGTLVTVSS

SEQ ID NO: 69 - protein, 107 aa, synthetic construct; identical to SEQ ID NO: 67
  REGION 1..107: note = Synthetic construct

SEQ ID NO: 70 - protein, 121 aa, synthetic construct
  REGION 1..121: note = Synthetic construct
EVQLVESGGGAVRPGGSLRLSCAASGFTFDDYGMSWVRQAPGKGLEWVSDINWSGEITAYADSVKGRFTISRDNAKNSLYLQMNSLRAEDTAVYYCARSNSYRWDDALDIWGQGTMVTVSS

SEQ ID NO: 71 - protein, 108 aa, synthetic construct
  REGION 1..108: note = Synthetic construct
EIVLTQSPATLSVSPGERATLSCRASQSVSSNLAWYQQKPGQAPRLLIYGASSRATGIPDRFSGSGSGTDFTLTISRLEPEDFAVYYCQQYGSSPPVTFGQGTKVEIK

SEQ ID NO: 72 - protein, 122 aa, synthetic construct
  REGION 1..122: note = Synthetic construct
EVQLVESGGGLVQPGRSLRLSCAASGFTFDDYAMHWVRQAPGKGLEWVAVISYDGSNKYYADSVKGRFTISRDNSKNTLYLQMNSLRAEDTAVYYCAKDRIGWYDYDAFDIWGQGTMVTVSS

SEQ ID NO: 73 - protein, 107 aa, synthetic construct
  REGION 1..107: note = Synthetic construct
EIVLTQSPATLSVTPGEGATLSCRASHSVGSNLAWYQQKPGQAPRLLIYDASNRATGIPARFSGSGSGTDFTLTISSLEPEDLAVYYCQQRSNWPPTFGQGTRLEIK

SEQ ID NO: 74 - protein, 5 aa, synthetic construct
  REGION 1..5: note = Synthetic construct
  VARIANT 1..4: note = can be present in repeats of any integer from 1 to 12
  VARIANT 5: note = can be absent or present in repeats of any integer from 1 to 3
GGGSG

SEQ ID NO: 75 - protein, 6 aa, synthetic construct
  REGION 1..6: note = Synthetic construct
  VARIANT 1..5: note = can be present in repeats of any integer from 1 to 12
  VARIANT 6: note = can be absent or present in repeats of any integer from 1 to 3
GGGGSG

SEQ ID NO: 76 - protein, 7 aa, synthetic construct
  REGION 1..7: note = Synthetic construct
  VARIANT 1..6: note = can be present in repeats of any integer from 1 to 12
  VARIANT 7: note = can be absent or present in repeats of any integer from 1 to 3
GGGGGSG

SEQ ID NO: 77 - protein, 6 aa, synthetic construct
  REGION 1..6: note = Synthetic construct
  VARIANT 1..5: note = can be present in repeats of any integer from 2 to 4
  VARIANT 6: note = absent
GGGGSG

SEQ ID NO: 78 - protein, 6 aa, synthetic construct
  REGION 1..6: note = Synthetic construct
  VARIANT 1..5: note = present in repeats of 3
  VARIANT 6: note = absent
GGGGSG

SEQ ID NO: 79 - protein, 15 aa, synthetic construct
  REGION 1..15: note = Synthetic construct
GGGGSGGGGSGGGGS

SEQ ID NO: 80 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 81 - protein, 133 aa, Homo sapiens [sequence omitted]

SEQ ID NO: 82 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 83 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 84 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 85 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 86 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 87 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFCQSIISTLT

SEQ ID NO: 88 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 89 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISSINVIVLELKGSETTFMCEYADETATIVEFLNRWITFCQSIISTLT

SEQ ID NO: 90 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFCQSIISTLT

SEQ ID NO: 91 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISNINEIVLELKGSETTFMCEYADETATIVEFLNRWITFCQSIISTLT

SEQ ID NO: 92 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRHLISNINVIVLELKGSETTFMCEYADETATIVEFLNRWITFCQSIISTLT

SEQ ID NO: 93 - protein, 133 aa, synthetic construct
  REGION 1..133: note = Synthetic construct
APTSSSTKKTQLQLEDLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 94 - protein, 133 aa, synthetic construct
  REGION 1..133: note = Synthetic construct
APTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 95 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 96 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISSINVIVLELKGSETTFMCEYADETATIVEFLNRWITFCQSIISTLT

SEQ ID NO: 97 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISAINVIVLELKGSETTFMCEYADETATIVEFLNRWITFCQSIISTLT

SEQ ID NO: 98 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISNINEIVLELKGSETTFMCEYADETATIVEFLNRWITFCQSIISTLT

SEQ ID NO: 99 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRHLISNINVIVLELKGSETTFMCEYADETATIVEFLNRWITFCQSIISTLT

SEQ ID NO: 100 - protein, 133 aa, synthetic construct
  REGION 1..133: note = Synthetic construct
APTSSSTKKTQLQLEDLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 101 - protein, 133 aa, synthetic construct
  REGION 1..133: note = Synthetic construct
APTSSSTKKTQLQLEELLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 102 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 103 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISSINVIVLELKGSETTFMCEYADETATIVEFLNRWITFCQSIISTLT

SEQ ID NO: 104 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISAINVIVLELKGSETTFMCEYADETATIVEFLNRWITFCQSIISTLT

SEQ ID NO: 105 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISNINEIVLELKGSETTFMCEYADETATIVEFLNRWITFCQSIISTLT

SEQ ID NO: 106 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRHLISNINVIVLELKGSETTFMCEYADETATIVEFLNRWITFCQSIISTLT

SEQ ID NO: 107 - protein, 133 aa, synthetic construct
  REGION 1..133: note = Synthetic construct
APTSSSTKKTQLQLEDLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 108 - protein, 133 aa, synthetic construct
  REGION 1..133: note = Synthetic construct
APTSSSTKKTQLQLEELLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 109 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 110 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISSINVIVLELKGSETTFMCEYADETATIVEFLNRWITFCQSIISTLT

SEQ ID NO: 111 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISAINVIVLELKGSETTFMCEYADETATIVEFLNRWITFCQSIISTLT

SEQ ID NO: 112 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISNINEIVLELKGSETTFMCEYADETATIVEFLNRWITFCQSIISTLT

SEQ ID NO: 113 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRHLISNINVIVLELKGSETTFMCEYADETATIVEFLNRWITFCQSIISTLT

SEQ ID NO: 114 - protein, 133 aa, synthetic construct
  REGION 1..133: note = Synthetic construct
APTSSSTKKTQLQLEDLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFCQSIISTLT

SEQ ID NO: 115 - protein, 133 aa, synthetic construct
  REGION 1..133: note = Synthetic construct
APTSSSTKKTQLQLEELLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFCQSIISTLT

SEQ ID NO: 116 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFCSSIISTLT

SEQ ID NO: 117 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 118 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 119 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFAQSIISTLT

SEQ ID NO: 120 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 121 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISSINVIVLELKGSETTFMCEYADETATIVEFLNRWITFAQSIISTLT

SEQ ID NO: 122 - protein, 133 aa, synthetic construct; identical to SEQ ID NO: 80
  REGION 1..133: note = Synthetic construct

SEQ ID NO: 123 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISNINEIVLELKGSETTFMCEYADETATIVEFLNRWITFAQSIISTLT

SEQ ID NO: 124 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRHLISNINVIVLELKGSETTFMCEYADETATIVEFLNRWITFAQSIISTLT

SEQ ID NO: 125 - protein, 133 aa, synthetic construct
  REGION 1..133: note = Synthetic construct
APTSSSTKKTQLQLEDLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 126 - protein, 133 aa, synthetic construct
  REGION 1..133: note = Synthetic construct
APTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 127 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFASSIISTLT

SEQ ID NO: 128 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISSINVIVLELKGSETTFMCEYADETATIVEFLNRWITFAQSIISTLT

SEQ ID NO: 129 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 130 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISNINEIVLELKGSETTFMCEYADETATIVEFLNRWITFAQSIISTLT

SEQ ID NO: 131 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRHLISNINVIVLELKGSETTFMCEYADETATIVEFLNRWITFAQSIISTLT

SEQ ID NO: 132 - protein, 133 aa, synthetic construct
  REGION 1..133: note = Synthetic construct
APTSSSTKKTQLQLEDLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 133 - protein, 133 aa, synthetic construct
  REGION 1..133: note = Synthetic construct
APTSSSTKKTQLQLEELLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 134 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFASSIISTLT

SEQ ID NO: 135 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISSINVIVLELKGSETTFMCEYADETATIVEFLNRWITFAQSIISTLT

SEQ ID NO: 136 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 137 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISNINEIVLELKGSETTFMCEYADETATIVEFLNRWITFAQSIISTLT

SEQ ID NO: 138 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRHLISNINVIVLELKGSETTFMCEYADETATIVEFLNRWITFAQSIISTLT

SEQ ID NO: 139 - protein, 133 aa, synthetic construct
  REGION 1..133: note = Synthetic construct
APTSSSTKKTQLQLEDLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 140 - protein, 133 aa, synthetic construct
  REGION 1..133: note = Synthetic construct
APTSSSTKKTQLQLEELLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 141 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFASSIISTLT

SEQ ID NO: 142 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISSINVIVLELKGSETTFMCEYADETATIVEFLNRWITFAQSIISTLT

SEQ ID NO: 143 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISAINVIVLELKGSETTFMCEYADETATIVEFLNRWITFAQSIISTLT

SEQ ID NO: 144 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISNINEIVLELKGSETTFMCEYADETATIVEFLNRWITFAQSIISTLT

SEQ ID NO: 145 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRHLISNINVIVLELKGSETTFMCEYADETATIVEFLNRWITFAQSIISTLT

SEQ ID NO: 146 - protein, 133 aa, synthetic construct
  REGION 1..133: note = Synthetic construct
APTSSSTKKTQLQLEDLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFAQSIISTLT

SEQ ID NO: 147 - protein, 133 aa, synthetic construct
  REGION 1..133: note = Synthetic construct
APTSSSTKKTQLQLEELLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFAQSIISTLT

SEQ ID NO: 148 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFASSIISTLT

SEQ ID NO: 149 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEELKPLEEVLNLAQSKNFHLRPRDLISSINVIVLELKGSETTFMCEYADETATIVEFLNRWITFAQSIISTLT

SEQ ID NO: 150 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 151 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEELKPLEEVLNLAQSKNFHLRPRDLISNINEIVLELKGSETTFMCEYADETATIVEFLNRWITFAQSIISTLT

SEQ ID NO: 152 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEELKPLEEVLNLAQSKNFHLRPRHLISNINVIVLELKGSETTFMCEYADETATIVEFLNRWITFAQSIISTLT

SEQ ID NO: 153 - protein, 133 aa, synthetic construct
  REGION 1..133: note = Synthetic construct
APTSSSTKKTQLQLEDLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 154 - protein, 133 aa, synthetic construct
  REGION 1..133: note = Synthetic construct
APTSSSTKKTQLQLEELLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 155 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFASSIISTLT

SEQ ID NO: 156 - protein, 214 aa, synthetic construct
  REGION 1..214: note = Synthetic construct
DIQMTQSPASLSASVGETVTITCGASENIYGALNWYQRKQGKSPQLLIFGATNLADGVSSRFSGSGSDRQYSLKISSLHPDDVATYYCQNILDTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 597 aa, synthetic construct
  REGION 1..597: note = Synthetic construct
QVHLQQSGPELVKPGASVKMSCKTSGYTFTKYTMHWVKQGHEESLEWIGHFNPNNDETKYNQKFTGKATLTVDKSSTTAYMELRSLTSDDSALYYCARDGLGLRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 158 - protein, 448 aa, synthetic construct
  REGION 1..448: note = Synthetic construct
QVHLQQSGPELVKPGASVKMSCKTSGYTFTKYTMHWVKQGHEESLEWIGHFNPNNDETKYNQKFTGKATLTVDKSSTTAYMELRSLTSDDSALYYCARDGLGLRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 159 - protein, 214 aa, synthetic construct
  REGION 1..214: note = Synthetic construct
DIQMTQSPSSLSASVGDRVTITCRASQEIYGALNWYQQKPGKAPKLLIYGATNLQSGVPSRFSGSGSGTDFTLTISSLQPEDFATYYCQDIYDAPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 597 aa, synthetic construct
  REGION 1..597: note = Synthetic construct
QVQLVQSGAEVKKPGSSVKVSCKASGYRFHNFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 161 - protein, 448 aa, synthetic construct
  REGION 1..448: note = Synthetic construct
QVQLVQSGAEVKKPGSSVKVSCKASGYRFHNFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 162 - protein, 214 aa, synthetic construct; identical to SEQ ID NO: 159
  REGION 1..214: note = Synthetic construct

SEQ ID NO: 163 - protein, 597 aa, synthetic construct
  REGION 1..597: note = Synthetic construct
QVQLVQSGAEVKKPGSSVKVSCKASGSRFYKFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 164 - protein, 448 aa, synthetic construct
  REGION 1..448: note = Synthetic construct
QVQLVQSGAEVKKPGSSVKVSCKASGSRFYKFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 165 - protein, 214 aa, synthetic construct
  REGION 1..214: note = Synthetic construct
DIQMTQSPSSLSASVGDRVTITCRASQKIYGALNWYQQKPGKAPKLLIYGATNLQSGVPSRFSGSGSGTDFTLTISSLQPEDFATYYCQNTYDTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 597 aa, synthetic construct
  REGION 1..597: note = Synthetic construct
QVQLVQSGAEVKKPGSSVKVSCKASGYTFTKYAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 167 - protein, 448 aa, synthetic construct
  REGION 1..448: note = Synthetic construct
QVQLVQSGAEVKKPGSSVKVSCKASGYTFTKYAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - protein, 214 aa, synthetic construct; identical to SEQ ID NO: 165
  REGION 1..214: note = Synthetic construct

SEQ ID NO: 169 - protein, 597 aa, synthetic construct
  REGION 1..597: note = Synthetic construct
QVQLVQSGAEVKKPGSSVKVSCKASGSGFRGHAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 170 - protein, 448 aa, synthetic construct
  REGION 1..448: note = Synthetic construct
QVQLVQSGAEVKKPGSSVKVSCKASGSGFRGHAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 171 - protein, 215 aa, synthetic construct
  REGION 1..215: note = Synthetic construct
EIVLTQSPATLSVSPGERATLSCRASQSVSSNLAWYQQKPGQAPRLLIYGASSRATGIPDRFSGSGSGTDFTLTISRLEPEDFAVYYCQQYGSSPPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 599 aa, synthetic construct
  REGION 1..599: note = Synthetic construct
EVQLVESGGGAVRPGGSLRLSCAASGFTFDDYGMSWVRQAPGKGLEWVSDINWSGEITAYADSVKGRFTISRDNAKNSLYLQMNSLRAEDTAVYYCARSNSYRWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 173 - protein, 450 aa, synthetic construct
  REGION 1..450: note = Synthetic construct
EVQLVESGGGAVRPGGSLRLSCAASGFTFDDYGMSWVRQAPGKGLEWVSDINWSGEITAYADSVKGRFTISRDNAKNSLYLQMNSLRAEDTAVYYCARSNSYRWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 174 - protein, 214 aa, synthetic construct
  REGION 1..214: note = Synthetic construct
EIVLTQSPATLSVTPGEGATLSCRASHSVGSNLAWYQQKPGQAPRLLIYDASNRATGIPARFSGSGSGTDFTLTISSLEPEDLAVYYCQQRSNWPPTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 600 aa, synthetic construct
  REGION 1..600: note = Synthetic construct
EVQLVESGGGLVQPGRSLRLSCAASGFTFDDYAMHWVRQAPGKGLEWVAVISYDGSNKYYADSVKGRFTISRDNSKNTLYLQMNSLRAEDTAVYYCAKDRIGWYDYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 176 - protein, 451 aa, synthetic construct
  REGION 1..451: note = Synthetic construct
EVQLVESGGGLVQPGRSLRLSCAASGFTFDDYAMHWVRQAPGKGLEWVAVISYDGSNKYYADSVKGRFTISRDNSKNTLYLQMNSLRAEDTAVYYCAKDRIGWYDYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 177 - protein, 5 aa, synthetic construct
  REGION 1..5: note = Synthetic construct
SYWMN

SEQ ID NO: 178 - protein, 17 aa, synthetic construct
  REGION 1..17: note = Synthetic construct
QIYPGDGDTNYNGKFKG

SEQ ID NO: 179 - protein, 13 aa, synthetic construct
  REGION 1..13: note = Synthetic construct
SGAAFSSYYAMDY

SEQ ID NO: 180 - protein, 11 aa, synthetic construct
  REGION 1..11: note = Synthetic construct
RASENIYSNLA

SEQ ID NO: 181 - protein, 7 aa, synthetic construct
  REGION 1..7: note = Synthetic construct
AATNLAD

SEQ ID NO: 182 - protein, 9 aa, synthetic construct
  REGION 1..9: note = Synthetic construct
QHFWGTPWT

SEQ ID NO: 183 - protein, 7 aa, synthetic construct
  REGION 1..7: note = Synthetic construct
GYAFSSY

SEQ ID NO: 184 - protein, 6 aa, synthetic construct
  REGION 1..6: note = Synthetic construct
YPGDGD

SEQ ID NO: 185 - protein, 122 aa, synthetic construct
  REGION 1..122: note = Synthetic construct
QVQLQQSGAELVRPGSSVKISCKASGYAFSSYWMNWVKQRPGQGLEWIGQIYPGDGDTNYNGKFKGKATLTADKSSSTAYMQLSSLTSEDSAVYFCARSGAAFSSYYAMDYWGQGTSVTVSS

SEQ ID NO: 186 - protein, 107 aa, synthetic construct
  REGION 1..107: note = Synthetic construct
DIQMTQSPASLSVSVGETVTITCRASENIYSNLAWYQQKQGKSPQLLVYAATNLADGVPSRFSGSGSGTQYSLKINSLQSEDFGSYYCQHFWGTPWTFGGGTKLEIK

SEQ ID NO: 187 - protein, 214 aa, synthetic construct
  REGION 1..214: note = Synthetic construct
DIQMTQSPASLSVSVGETVTITCRASENIYSNLAWYQQKQGKSPQLLVYAATNLADGVPSRFSGSGSGTQYSLKINSLQSEDFGSYYCQHFWGTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 188 - protein, 600 aa, synthetic construct
  REGION 1..600: note = Synthetic construct
QVQLQQSGAELVRPGSSVKISCKASGYAFSSYWMNWVKQRPGQGLEWIGQIYPGDGDTNYNGKFKGKATLTADKSSSTAYMQLSSLTSEDSAVYFCARSGAAFSSYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 189 - protein, 451 aa, synthetic construct
  REGION 1..451: note = Synthetic construct
QVQLQQSGAELVRPGSSVKISCKASGYAFSSYWMNWVKQRPGQGLEWIGQIYPGDGDTNYNGKFKGKATLTADKSSSTAYMQLSSLTSEDSAVYFCARSGAAFSSYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 190 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISGINVIVLELKGSETTFMCEYADETATIVEFLNRWITFCQSIISTLT

SEQ ID NO: 191 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRKLISNINVIVLELKGSETTFMCEYADETATIVEFLNRWITFCQSIISTLT

SEQ ID NO: 192 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRRLISNINVIVLELKGSETTFMCEYADETATIVEFLNRWITFCQSIISTLT

SEQ ID NO: 193 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISGINVIVLELKGSETTFMCEYADETATIVEFLNRWITFCQSIISTLT

SEQ ID NO: 194 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRKLISNINVIVLELKGSETTFMCEYADETATIVEFLNRWITFCQSIISTLT

SEQ ID NO: 195 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRRLISNINVIVLELKGSETTFMCEYADETATIVEFLNRWITFCQSIISTLT

SEQ ID NO: 196 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISGINVIVLELKGSETTFMCEYADETATIVEFLNRWITFCQSIISTLT

SEQ ID NO: 197 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRKLISNINVIVLELKGSETTFMCEYADETATIVEFLNRWITFCQSIISTLT

SEQ ID NO: 198 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRRLISNINVIVLELKGSETTFMCEYADETATIVEFLNRWITFCQSIISTLT

SEQ ID NO: 199 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISGINVIVLELKGSETTFMCEYADETATIVEFLNRWITFCQSIISTLT

SEQ ID NO: 200 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRKLISNINVIVLELKGSETTFMCEYADETATIVEFLNRWITFCQSIISTLT

SEQ ID NO: 201 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRRLISNINVIVLELKGSETTFMCEYADETATIVEFLNRWITFCQSIISTLT

SEQ ID NO: 202 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISGINVIVLELKGSETTFMCEYADETATIVEFLNRWITFAQSIISTLT

SEQ ID NO: 203 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 204 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRRLISNINVIVLELKGSETTFMCEYADETATIVEFLNRWITFAQSIISTLT

SEQ ID NO: 205 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISGINVIVLELKGSETTFMCEYADETATIVEFLNRWITFAQSIISTLT

SEQ ID NO: 206 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 207 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRRLISNINVIVLELKGSETTFMCEYADETATIVEFLNRWITFAQSIISTLT

SEQ ID NO: 208 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISGINVIVLELKGSETTFMCEYADETATIVEFLNRWITFAQSIISTLT

SEQ ID NO: 209 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 210 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRRLISNINVIVLELKGSETTFMCEYADETATIVEFLNRWITFAQSIISTLT

SEQ ID NO: 211 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISGINVIVLELKGSETTFMCEYADETATIVEFLNRWITFAQSIISTLT

SEQ ID NO: 212 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRKLISNINVIVLELKGSETTFMCEYADETATIVEFLNRWITFAQSIISTLT

SEQ ID NO: 213 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRRLISNINVIVLELKGSETTFMCEYADETATIVEFLNRWITFAQSIISTLT

SEQ ID NO: 214 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEELKPLEEVLNLAQSKNFHLRPRDLISGINVIVLELKGSETTFMCEYADETATIVEFLNRWITFAQSIISTLT

SEQ ID NO: 215 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 216 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEELKPLEEVLNLAQSKNFHLRPRRLISNINVIVLELKGSETTFMCEYADETATIVEFLNRWITFAQSIISTLT

SEQ ID NO: 217 - protein, 449 aa, synthetic construct
  REGION 1..449: note = Synthetic construct
QVHLQQSGPELVKPGASVKMSCKTSGYTFTKYTMHWVKQGHEESLEWIGHFNPNNDETKYNQKFTGKATLTVDKSSTTAYMELRSLTSDDSALYYCARDGLGLRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 449 aa, synthetic construct
  REGION 1..449: note = Synthetic construct
QVQLVQSGAEVKKPGSSVKVSCKASGYRFHNFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 449 aa, synthetic construct
  REGION 1..449: note = Synthetic construct
QVQLVQSGAEVKKPGSSVKVSCKASGSRFYKFAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 449 aa, synthetic construct
  REGION 1..449: note = Synthetic construct
QVQLVQSGAEVKKPGSSVKVSCKASGYTFTKYAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 449 aa, synthetic construct
  REGION 1..449: note = Synthetic construct
QVQLVQSGAEVKKPGSSVKVSCKASGSGFRGHAISWVRQAPGQGLEWMGGIIPGHAKANYAQKFQGRVTITADESTSTAYMELSSLRSEDTAVYYCARDGLGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 451 aa, synthetic construct
  REGION 1..451: note = Synthetic construct
EVQLVESGGGAVRPGGSLRLSCAASGFTFDDYGMSWVRQAPGKGLEWVSDINWSGEITAYADSVKGRFTISRDNAKNSLYLQMNSLRAEDTAVYYCARSNSYRWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - protein, 452 aa, synthetic construct
  REGION 1..452: note = Synthetic construct
EVQLVESGGGLVQPGRSLRLSCAASGFTFDDYAMHWVRQAPGKGLEWVAVISYDGSNKYYADSVKGRFTISRDNSKNTLYLQMNSLRAEDTAVYYCAKDRIGWYDYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 452 aa, synthetic construct
  REGION 1..452: note = Synthetic construct
QVQLQQSGAELVRPGSSVKISCKASGYAFSSYWMNWVKQRPGQGLEWIGQIYPGDGDTNYNGKFKGKATLTADKSSSTAYMQLSSLTSEDSAVYFCARSGAAFSSYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 225 - protein, 5 aa, synthetic construct
  REGION 1..5: note = Synthetic construct
SYAIS

SEQ ID NO: 226 - protein, 17 aa, synthetic construct
  REGION 1..17: note = Synthetic construct
GIIPGAATANYAQKFQG

SEQ ID NO: 227 - protein, 10 aa, synthetic construct
  REGION 1..10: note = Synthetic construct
DAAGIRLFAD

SEQ ID NO: 228 - protein, 9 aa, synthetic construct
  REGION 1..9: note = Synthetic construct
QSTYDAPWT

SEQ ID NO: 229 - protein, 5 aa, synthetic construct
  REGION 1..5: note = Synthetic construct
SYAMS

SEQ ID NO: 230 - protein, 17 aa, synthetic construct
  REGION 1..17: note = Synthetic construct
DITYAGGSTAYADSVKG

SEQ ID NO: 231 - protein, 12 aa, synthetic construct
  REGION 1..12: note = Synthetic construct
SNAYAWDDALDI

SEQ ID NO: 232 - protein, 17 aa, synthetic construct
  REGION 1..17: note = Synthetic construct
GIIPGYATANYAQKFQG

SEQ ID NO: 233 - protein, 10 aa, synthetic construct
  REGION 1..10: note = Synthetic construct
DAAGIRLFAD

SEQ ID NO: 234 - protein, 11 aa, synthetic construct
  REGION 1..11: note = Synthetic construct
RASQSIYGALN

SEQ ID NO: 235 - protein, 7 aa, synthetic construct
  REGION 1..7: note = Synthetic construct
GASNLQS

SEQ ID NO: 236 - protein, 9 aa, synthetic construct
  REGION 1..9: note = Synthetic construct
QSTYTAPWT

SEQ ID NO: 237 - protein, 17 aa, synthetic construct
  REGION 1..17: note = Synthetic construct
DISYAGGSTAYADSVKG

SEQ ID NO: 238 - protein, 7 aa, synthetic construct
  REGION 1..7: note = Synthetic construct
GGTFSSY

SEQ ID NO: 239 - protein, 6 aa, synthetic construct
  REGION 1..6: note = Synthetic construct
IPGAAT

SEQ ID NO: 240 - protein, 7 aa, synthetic construct
  REGION 1..7: note = Synthetic construct
GFTFSSY

SEQ ID NO: 241 - protein, 6 aa, synthetic construct
  REGION 1..6: note = Synthetic construct
TYAGGS

SEQ ID NO: 242 - protein, 12 aa, synthetic construct
  REGION 1..12: note = Synthetic construct
SNAYAWDDALDI

SEQ ID NO: 243 - protein, 6 aa, synthetic construct
  REGION 1..6: note = Synthetic construct
IPGYAT

SEQ ID NO: 244 - protein, 6 aa, synthetic construct
  REGION 1..6: note = Synthetic construct
SYAGGS

SEQ ID NO: 245 - protein, 119 aa, synthetic construct
  REGION 1..119: note = Synthetic construct
QVQLVQSGAEVKKPGSSVKVSCKASGGTFSSYAISWVRQAPGQGLEWMGGIIPGAATANYAQKFQGRVTITADESTSTAYMELSSLRSEDTAVYYCARDAAGIRLFADWGQGTLVTVSS

SEQ ID NO: 246 - protein, 107 aa, synthetic construct
  REGION 1..107: note = Synthetic construct
DIQMTQSPSSLSASVGDRVTITCRASQEIYGALNWYQQKPGKAPKLLIYGATNLQSGVPSRFSGSGSGTDFTLTISSLQPEDFATYYCQSTYDAPWTFGGGTKVEIK

SEQ ID NO: 247 - protein, 121 aa, synthetic construct
  REGION 1..121: note = Synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTMVTVSS

SEQ ID NO: 248 - protein, 108 aa, synthetic construct
  REGION 1..108: note = Synthetic construct
EIVLTQSPGTLSLSPGERATLSCRASQSVSSNLAWYQQKPGQAPRLLIYGASSRATGIPDRFSGSGSGTDFTLTISRLEPEDFAVYYCQQYGSSPPVTFGQGTKVEIK

SEQ ID NO: 249 - protein, 121 aa, synthetic construct
  REGION 1..121: note = Synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSS

SEQ ID NO: 250 - protein, 108 aa, synthetic construct; identical to SEQ ID NO: 248
  REGION 1..108: note = Synthetic construct

SEQ ID NO: 251 - protein, 119 aa, synthetic construct
  REGION 1..119: note = Synthetic construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSS

SEQ ID NO: 252 - protein, 107 aa, synthetic construct
  REGION 1..107: note = Synthetic construct
DIQMTQSPSSLSASVGDRVTITCRASQSIYGALNWYQQKPGKAPKLLIYGASNLQSGVPSRFSGSGSGTDFTLTISSLQPEDFATYYCQSTYTAPWTFGGGTKVEIK

SEQ ID NO: 253 - protein, 119 aa, synthetic construct; identical to SEQ ID NO: 251
  REGION 1..119: note = Synthetic construct

SEQ ID NO: 254 - protein, 107 aa, synthetic construct; identical to SEQ ID NO: 246
  REGION 1..107: note = Synthetic construct

SEQ ID NO: 255 - protein, 121 aa, synthetic construct
  REGION 1..121: note = Synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTMVTVSS

SEQ ID NO: 256 - protein, 108 aa, synthetic construct; identical to SEQ ID NO: 248
  REGION 1..108: note = Synthetic construct

SEQ ID NO: 257 - protein, 121 aa, synthetic construct
  REGION 1..121: note = Synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTLVTVSS

SEQ ID NO: 258 - protein, 108 aa, synthetic construct; identical to SEQ ID NO: 248
  REGION 1..108: note = Synthetic construct

SEQ ID NO: 259 - skipped

SEQ ID NO: 260 - protein, 17 aa, synthetic construct
  REGION 1..17: note = Synthetic construct
  VARIANT 1: note = Xaa = G or H
  VARIANT 2: note = Xaa = I or F
  VARIANT 3: note = Xaa = I, N, or M
  VARIANT 5: note = Xaa = G, N, H, S, R, I, or A
  VARIANT 6: note = Xaa = A, N, H, S, T, F, or Y
  VARIANT 7: note = Xaa = A, D, or G
  VARIANT 8: note = Xaa = T, E, K, V, Q, or A
  VARIANT 9: note = Xaa = A or T
  VARIANT 10: note = Xaa = N or K
  VARIANT 12: note = Xaa = A or N
  VARIANT 16: note = Xaa = Q or T
XXXPXXXXXXYXQKFXG

SEQ ID NO: 261 - skipped

SEQ ID NO: 262 - protein, 11 aa, synthetic construct
  REGION 1..11: note = Synthetic construct
  VARIANT 1: note = Xaa = R or G
  VARIANT 2: note = Xaa = A or T
  VARIANT 4: note = Xaa = Q or E
  VARIANT 5: note = Xaa = E, N, T, S, A, K, D, G, R, or Q
  VARIANT 7: note = Xaa = Y or S
  VARIANT 9: note = Xaa = A or V
XXSXXIXGXLN

SEQ ID NO: 263 - skipped

SEQ ID NO: 264 - protein, 9 aa, synthetic construct
  REGION 1..9: note = Synthetic construct
  VARIANT 2: note = Xaa = S, N, D, Q, A, or E
  VARIANT 3: note = Xaa = T, I, or S
  VARIANT 4: note = Xaa = Y, L, or F
  VARIANT 5: note = Xaa = D, G, T, E, Q, A, or Y
  VARIANT 6: note = Xaa = A, T, R, S, K, or Y
QXXXXXPWT

SEQ ID NO: 265-268 - skipped (4 entries)

SEQ ID NO: 269 - protein, 17 aa, synthetic construct
  REGION 1..17: note = Synthetic construct
  VARIANT 3: note = Xaa = T, N, S, Q, E, H, R, or A
  VARIANT 4: note = Xaa = Y, W, F, or H
  VARIANT 5: note = Xaa = A, S, Q, E, or T
  VARIANT 7: note = Xaa = G or E
  VARIANT 8: note = Xaa = S or I
  VARIANT 10: note = Xaa = A or G
DIXXXGXXTXYADSVKG

SEQ ID NO: 270 - protein, 12 aa, synthetic construct
  REGION 1..12: note = Synthetic construct
  VARIANT 1: note = Xaa = S or A
  VARIANT 2: note = Xaa = N, H, A, D, L, Q, Y, or R
  VARIANT 3: note = Xaa = A, N, S, or G
  VARIANT 5: note = Xaa = A, V, R, E, or S
  VARIANT 7: note = Xaa = D or S
  VARIANT 8: note = Xaa = D, N, Q, E, S, T, or L
  VARIANT 10: note = Xaa = L, F, or M
  VARIANT 12: note = Xaa = I, Y, or V
XXXYXWXXAXDX

SEQ ID NO: 271 - protein, 7 aa, synthetic construct
  REGION 1..7: note = Synthetic construct
  VARIANT 5: note = Xaa = S, D, E, Q, S, or A
  VARIANT 6: note = Xaa = S, D, E, A, or Q
GFTFXXY

SEQ ID NO: 272 - skipped

SEQ ID NO: 273 - protein, 12 aa, synthetic construct
  REGION 1..12: note = Synthetic construct
  VARIANT 1: note = Xaa = S or A
  VARIANT 2: note = Xaa = N, H, A, D, L, Q, Y, or R
  VARIANT 3: note = Xaa = A, N, S, or G
  VARIANT 5: note = Xaa = A, V, R, E, or S
  VARIANT 7: note = Xaa = D or S
  VARIANT 8: note = Xaa = D, N, Q, E, S, T, or L
  VARIANT 10: note = Xaa = L, F, or M
  VARIANT 12: note = Xaa = I, Y, or V
XXXYXWXXAXDX

SEQ ID NO: 274 - protein, 30 aa, synthetic construct
  REGION 1..30: note = Synthetic construct
QVQLVQSGAEVKKPGSSVKVSCKASGGTFS

SEQ ID NO: 275 - protein, 14 aa, synthetic construct
  REGION 1..14: note = Synthetic construct
WVRQAPGQGLEWMG

SEQ ID NO: 276 - protein, 32 aa, synthetic construct
  REGION 1..32: note = Synthetic construct
RVTITADESTSTAYMELSSLRSEDTAVYYCAR

SEQ ID NO: 277 - protein, 11 aa, synthetic construct
  REGION 1..11: note = Synthetic construct
WGQGTLVTVSS

SEQ ID NO: 278 - protein, 25 aa, synthetic construct [sequence omitted]

SEQ ID NO: 279 - protein, 19 aa, synthetic construct
  REGION 1..19: note = Synthetic construct
AISWVRQAPGQGLEWMGGI

SEQ ID NO: 280 - protein, 41 aa, synthetic construct
  REGION 1..41: note = Synthetic construct
ANYAQKFQGRVTITADESTSTAYMELSSLRSEDTAVYYCAR

SEQ ID NO: 281 - protein, 30 aa, synthetic construct
  REGION 1..30: note = Synthetic construct
EVQLVESGGGLVQPGGSLRLSCAASGFTFS

SEQ ID NO: 282 - protein, 14 aa, synthetic construct
  REGION 1..14: note = Synthetic construct
WVRQAPGKGLEWVS

SEQ ID NO: 283 - protein, 32 aa, synthetic construct
  REGION 1..32: note = Synthetic construct
RFTISRDNAKNSLYLQMNSLRAEDTAVYYCAR

SEQ ID NO: 284 - protein, 11 aa, synthetic construct
  REGION 1..11: note = Synthetic construct
WGQGTMVTVSS

SEQ ID NO: 285 - protein, 11 aa, synthetic construct
  REGION 1..11: note = Synthetic construct
WGQGTLVTVSS

SEQ ID NO: 286 - protein, 25 aa, synthetic construct [sequence omitted]

SEQ ID NO: 287 - protein, 19 aa, synthetic construct
  REGION 1..19: note = Synthetic construct
AMSWVRQAPGKGLEWVSDI

SEQ ID NO: 288 - protein, 41 aa, synthetic construct
  REGION 1..41: note = Synthetic construct
TAYADSVKGRFTISRDNAKNSLYLQMNSLRAEDTAVYYCAR

SEQ ID NO: 289 - protein, 23 aa, synthetic construct
  REGION 1..23: note = Synthetic construct
DIQMTQSPSSLSASVGDRVTITC

SEQ ID NO: 290 - protein, 15 aa, synthetic construct
  REGION 1..15: note = Synthetic construct
WYQQKPGKAPKLLIY

SEQ ID NO: 291 - protein, 32 aa, synthetic construct
  REGION 1..32: note = Synthetic construct
GVPSRFSGSGSGTDFTLTISSLQPEDFATYYC

SEQ ID NO: 292 - protein, 10 aa, synthetic construct
  REGION 1..10: note = Synthetic construct
FGGGTKVEIK

SEQ ID NO: 293 - protein, 23 aa, synthetic construct
  REGION 1..23: note = Synthetic construct
EIVLTQSPGTLSLSPGERATLSC

SEQ ID NO: 294 - protein, 15 aa, synthetic construct
  REGION 1..15: note = Synthetic construct
WYQQKPGQAPRLLIY

SEQ ID NO: 295 - protein, 32 aa, synthetic construct
  REGION 1..32: note = Synthetic construct
GIPDRFSGSGSGTDFTLTISRLEPEDFAVYYC

SEQ ID NO: 296 - protein, 10 aa, synthetic construct
  REGION 1..10: note = Synthetic construct
FGQGTKVEIK

SEQ ID NO: 297 - protein, 133 aa, synthetic construct; identical to SEQ ID NO: 126
  REGION 1..133: note = Synthetic construct

SEQ ID NO: 298 - protein, 214 aa, synthetic construct
  REGION 1..214: note = Synthetic construct
DIQMTQSPSSLSASVGDRVTITCRASQEIYGALNWYQQKPGKAPKLLIYGATNLQSGVPSRFSGSGSGTDFTLTISSLQPEDFATYYCQSTYDAPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 299 - protein, 597 aa, synthetic construct
  REGION 1..597: note = Synthetic construct
QVQLVQSGAEVKKPGSSVKVSCKASGGTFSSYAISWVRQAPGQGLEWMGGIIPGAATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 300 - protein, 448 aa, synthetic construct
  REGION 1..448: note = Synthetic construct
QVQLVQSGAEVKKPGSSVKVSCKASGGTFSSYAISWVRQAPGQGLEWMGGIIPGAATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 301 - protein, 449 aa, synthetic construct
  REGION 1..449: note = Synthetic construct
QVQLVQSGAEVKKPGSSVKVSCKASGGTFSSYAISWVRQAPGQGLEWMGGIIPGAATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 302 - protein, 215 aa, synthetic construct
  REGION 1..215: note = Synthetic construct
EIVLTQSPGTLSLSPGERATLSCRASQSVSSNLAWYQQKPGQAPRLLIYGASSRATGIPDRFSGSGSGTDFTLTISRLEPEDFAVYYCQQYGSSPPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 303 - protein, 599 aa, synthetic construct
  REGION 1..599: note = Synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 304 - protein, 450 aa, synthetic construct
  REGION 1..450: note = Synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 305 - protein, 451 aa, synthetic construct
  REGION 1..451: note = Synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 306 - protein, 215 aa, synthetic construct; identical to SEQ ID NO: 302
  REGION 1..215: note = Synthetic construct

SEQ ID NO: 307 - protein, 599 aa, synthetic construct
  REGION 1..599: note = Synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 308 - protein, 450 aa, synthetic construct
  REGION 1..450: note = Synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 309 - protein, 451 aa, synthetic construct
  REGION 1..451: note = Synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 310 - protein, 214 aa, synthetic construct
  REGION 1..214: note = Synthetic construct
DIQMTQSPSSLSASVGDRVTITCRASQSIYGALNWYQQKPGKAPKLLIYGASNLQSGVPSRFSGSGSGTDFTLTISSLQPEDFATYYCQSTYTAPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 311 - protein, 597 aa, synthetic construct
  REGION 1..597: note = Synthetic construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 312 - protein, 448 aa, synthetic construct
  REGION 1..448: note = Synthetic construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 313 - protein, 449 aa, synthetic construct
  REGION 1..449: note = Synthetic construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 314 - protein, 214 aa, synthetic construct; identical to SEQ ID NO: 298
  REGION 1..214: note = Synthetic construct

SEQ ID NO: 315 - protein, 597 aa, synthetic construct; identical to SEQ ID NO: 311
  REGION 1..597: note = Synthetic construct

SEQ ID NO: 316 - protein, 448 aa, synthetic construct; identical to SEQ ID NO: 312
  REGION 1..448: note = Synthetic construct

SEQ ID NO: 317 - protein, 449 aa, synthetic construct; identical to SEQ ID NO: 313
  REGION 1..449: note = Synthetic construct

SEQ ID NO: 318 - protein, 215 aa, synthetic construct; identical to SEQ ID NO: 302
  REGION 1..215: note = Synthetic construct

SEQ ID NO: 319 - protein, 599 aa, synthetic construct
  REGION 1..599: note = Synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 320 - protein, 450 aa, synthetic construct
  REGION 1..450: note = Synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 321 - protein, 451 aa, synthetic construct
  REGION 1..451: note = Synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 215 aa, synthetic construct; identical to SEQ ID NO: 302
  REGION 1..215: note = Synthetic construct

SEQ ID NO: 323 - protein, 599 aa, synthetic construct
  REGION 1..599: note = Synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 324 - protein, 450 aa, synthetic construct
  REGION 1..450: note = Synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 325 - protein, 451 aa, synthetic construct
  REGION 1..451: note = Synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 214 aa, synthetic construct; identical to SEQ ID NO: 298
  REGION 1..214: note = Synthetic construct

SEQ ID NO: 327 - protein, 597 aa, synthetic construct
  REGION 1..597: note = Synthetic construct
QVQLVQSGAEVKKPGSSVKVSCKASGGTFSSYAISWVRQAPGQGLEWMGGIIPGAATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 328 - protein, 448 aa, synthetic construct; identical to SEQ ID NO: 300
  REGION 1..448: note = Synthetic construct

SEQ ID NO: 329 - protein, 449 aa, synthetic construct; identical to SEQ ID NO: 301
  REGION 1..449: note = Synthetic construct

SEQ ID NO: 330 - protein, 215 aa, synthetic construct; identical to SEQ ID NO: 302
  REGION 1..215: note = Synthetic construct

SEQ ID NO: 331 - protein, 599 aa, synthetic construct
  REGION 1..599: note = Synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 332 - protein, 450 aa, synthetic construct; identical to SEQ ID NO: 304
  REGION 1..450: note = Synthetic construct

SEQ ID NO: 333 - protein, 451 aa, synthetic construct; identical to SEQ ID NO: 305
  REGION 1..451: note = Synthetic construct

SEQ ID NO: 334 - protein, 215 aa, synthetic construct; identical to SEQ ID NO: 302
  REGION 1..215: note = Synthetic construct

SEQ ID NO: 335 - protein, 599 aa, synthetic construct
  REGION 1..599: note = Synthetic construct
EVQLVESGGGLVQPGGSLRLSCAASGFTFSSYAMSWVRQAPGKGLEWVSDITYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 336 - protein, 450 aa, synthetic construct; identical to SEQ ID NO: 308
  REGION 1..450: note = Synthetic construct

SEQ ID NO: 337 - protein, 451 aa, synthetic construct; identical to SEQ ID NO: 309
  REGION 1..451: note = Synthetic construct

SEQ ID NO: 338 - protein, 214 aa, synthetic construct; identical to SEQ ID NO: 310
  REGION 1..214: note = Synthetic construct

SEQ ID NO: 339 - protein, 597 aa, synthetic construct
  REGION 1..597: note = Synthetic construct
QVQLVQSGAEVKKPGSSVKVSCKASGGTFSSYAISWVRQAPGQGLEWMGGIIPGYATANYAQKFQGRVTITADESTSTAYMELSSLRSEDTAVYYCARDAAGIRLFADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 340 - protein, 448 aa, synthetic construct; identical to SEQ ID NO: 312
  REGION 1..448: note = Synthetic construct

SEQ ID NO: 341 - protein, 449 aa, synthetic construct; identical to SEQ ID NO: 313
  REGION 1..449: note = Synthetic construct

SEQ ID NO: 342 - protein, 214 aa, synthetic construct; identical to SEQ ID NO: 298
  REGION 1..214: note = Synthetic construct

SEQ ID NO: 343 - protein, 597 aa, synthetic construct; identical to SEQ ID NO: 339
  REGION 1..597: note = Synthetic construct

SEQ ID NO: 344 - protein, 448 aa, synthetic construct; identical to SEQ ID NO: 312
  REGION 1..448: note = Synthetic construct

SEQ ID NO: 345 - protein, 449 aa, synthetic construct; identical to SEQ ID NO: 313
  REGION 1..449: note = Synthetic construct

SEQ ID NO: 346 - protein, 215 aa, synthetic construct; identical to SEQ ID NO: 302
  REGION 1..215: note = Synthetic construct

SEQ ID NO: 347 - protein, 599 aa, synthetic construct
  REGION 1..599: note = Synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 348 - protein, 450 aa, synthetic construct; identical to SEQ ID NO: 320
  REGION 1..450: note = Synthetic construct

SEQ ID NO: 349 - protein, 451 aa, synthetic construct; identical to SEQ ID NO: 321
  REGION 1..451: note = Synthetic construct

SEQ ID NO: 350 - protein, 215 aa, synthetic construct; identical to SEQ ID NO: 302
  REGION 1..215: note = Synthetic construct

SEQ ID NO: 351 - protein, 599 aa, synthetic construct
  REGION 1..599: note = Synthetic construct
EVQLVESGGGLVQPGGSLRLSCAASGFTFSSYAMSWVRQAPGKGLEWVSDISYAGGSTAYADSVKGRFTISRDNAKNSLYLQMNSLRAEDTAVYYCARSNAYAWDDALDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGSAPTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 352 - protein, 450 aa, synthetic construct; identical to SEQ ID NO: 324
  REGION 1..450: note = Synthetic construct

SEQ ID NO: 353 - protein, 451 aa, synthetic construct; identical to SEQ ID NO: 325
  REGION 1..451: note = Synthetic construct

SEQ ID NO: 354 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 355 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISTINVIVLELKGSETTFMCEYADETATIVEFLNRWITFAQSIISTLT

SEQ ID NO: 356 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISRINVIVLELKGSETTFMCEYADETATIVEFLNRWITFAQSIISTLT

SEQ ID NO: 357 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 358 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISTINVIVLELKGSETTFMCEYADETATIVEFLNRWITFCQSIISTLT

SEQ ID NO: 359 - protein, 133 aa, synthetic construct
  REGION 1..133: note = Synthetic construct
APTSSSTKKTQLQLEHLLLDLQMILNGINNYKNPKLTEMLTAKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 360 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 361 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISTINVIVLELKGSETTFMCEYADETATIVEFLNRWITFCQSIISTLT

SEQ ID NO: 362 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 363 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 364 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISTINVIVLELKGSETTFMCEYADETATIVEFLNRWITFCQSIISTLT

SEQ ID NO: 365 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 366 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISRINVIVLELKGSETTFMCEYADETATIVEFLNRWITFCQSIISTLT

SEQ ID NO: 367 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISTINVIVLELKGSETTFMCEYADETATIVEFLNRWITFCQSIISTLT

SEQ ID NO: 368 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISDINVIVLELKGSETTFMCEYADETATIVEFLNRWITFCQSIISTLT

SEQ ID NO: 369 - protein, 133 aa, synthetic construct; identical to SEQ ID NO: 356
  REGION 1..133: note = Synthetic construct

SEQ ID NO: 370 - protein, 133 aa, synthetic construct; identical to SEQ ID NO: 355
  REGION 1..133: note = Synthetic construct

SEQ ID NO: 371 - protein, 133 aa, synthetic construct; identical to SEQ ID NO: 354
  REGION 1..133: note = Synthetic construct

SEQ ID NO: 372 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISRINVIVLELKGSETTFMCEYADETATIVEFLNRWITFAQSIISTLT

SEQ ID NO: 373 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISTINVIVLELKGSETTFMCEYADETATIVEFLNRWITFAQSIISTLT

SEQ ID NO: 374 - protein, 133 aa, synthetic construct
  REGION 1..133: note = Synthetic construct
APTSSSTKKTQLQLEHLLLDLQMILNGINNYKNPKLTDMLTAKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 375 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISRINVIVLELKGSETTFMCEYADETATIVEFLNRWITFAQSIISTLT

SEQ ID NO: 376 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISTINVIVLELKGSETTFMCEYADETATIVEFLNRWITFAQSIISTLT

SEQ ID NO: 377 - protein, 133 aa, synthetic construct
  REGION 1..133: note = Synthetic construct
APTSSSTKKTQLQLEHLLLDLQMILNGINNYKNPKLTAMLTKKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 378 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISRINVIVLELKGSETTFMCEYADETATIVEFLNRWITFAQSIISTLT

SEQ ID NO: 379 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISTINVIVLELKGSETTFMCEYADETATIVEFLNRWITFAQSIISTLT

SEQ ID NO: 380 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEQLKPLEEVLNLAQSKNFHLRPRDLISDINVIVLELKGSETTFMCEYADETATIVEFLNRWITFAQSIISTLT

SEQ ID NO: 381 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEELKPLEEVLNLAQSKNFHLRPRDLISRINVIVLELKGSETTFMCEYADETATIVEFLNRWITFAQSIISTLT

SEQ ID NO: 382 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEELKPLEEVLNLAQSKNFHLRPRDLISTINVIVLELKGSETTFMCEYADETATIVEFLNRWITFAQSIISTLT

SEQ ID NO: 383 - protein, 133 aa, synthetic construct
  REGION 1..133: note = Synthetic construct
APTSSSTKKTQLQLEHLLLDLQMILNGINNYKNPKLTRMLTAKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 384 - protein, 149 aa, synthetic construct
  REGION 1..149: note = Synthetic construct
APTSSSTSSSTAEAQQQQQQQQQQQQHLEQLLMDLQELLSRMENYRNLKLPEMLTAKFALPKQATELKDLQCLEDELGPLRHVLDLTQSKSFQLEDAENFISSIRVTVVKLKGSDNTFECQFDDESATVVDFLRRWIAFAQSIISTSPQ

SEQ ID NO: 385 - protein, 149 aa, Mus musculus
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 386 - protein, 6 aa, synthetic construct
  REGION 1..6: note = Synthetic construct
  VARIANT 2..5: note = can be present in repeats of any integer from 1 to 12
  VARIANT 6: note = can be absent or present in repeats of any integer from 1 to 3
SGGGSG

SEQ ID NO: 387 - protein, 7 aa, synthetic construct
  REGION 1..7: note = Synthetic construct
  VARIANT 2..6: note = can be present in repeats of any integer from 1 to 12
  VARIANT 7: note = can be absent or present in repeats of any integer from 1 to 3
SGGGGSG

SEQ ID NO: 388 - protein, 8 aa, synthetic construct
  REGION 1..8: note = Synthetic construct
  VARIANT 2..7: note = can be present in repeats of any integer from 1 to 12
  VARIANT 8: note = can be absent or present in repeats of any integer from 1 to 3
SGGGGGSG

SEQ ID NO: 389 - protein, 16 aa, synthetic construct
  REGION 1..16: note = Synthetic construct
SGGGGSGGGGSGGGGS

SEQ ID NO: 390 - protein, 149 aa, Mus musculus
APTSSSTSSSTAEAQQQQQQQQQQQQHLEQLLMDLQELLSRMENYRNLKLPEMLTAKFALPKQATELKDLQCLEDELGPLRHVLDLTQSKSFQLEDAENFISNIRVTVVKLKGSDNTFECQFDDESATVVDFLRRWIAFAQSIISTSPQ

SEQ ID NO: 391 - protein, 8 aa, synthetic construct
  REGION 1..8: note = Synthetic construct
MGLKFRQL

(19) United States
(12) Patent Application Publication
(10) Pub. No.: US 2026/0098090 A1
(43) Pub. Date: Apr. 9, 2026

(54) FUSIONS WITH CD8 ANTIGEN BINDING MOLECULES FOR TREATING CHRONIC VIRAL INFECTION

(71) Applicant: Asher Biotherapeutics, Inc., South San Francisco, CA (US)

(72) Inventors: Kelly Dare MOYNIHAN, San Francisco, CA (US); Yik Andy YEUNG, South San Francisco, CA (US); Ivana DJURETIC, Pacifica, CA (US)

(21) Appl. No.: 19/114,281

(22) PCT Filed: Sep. 28, 2023
(86) PCT No.: PCT/US2023/075376

§ 371 Date: Mar. 21, 2025

Related U.S. Application Data
(60) Provisional application No. 63/411,321, filed on Sep. 29, 2022.
(60) Provisional application No. 63/421,879, filed on Nov. 2, 2022.

(51) Int. Cl. C07K 16/28; A61K 45/06; A61P 31/20; C07K 14/55

(52) U.S. Cl. CPC C07K 16/2815; A61K 45/06; A61P 31/20; C07K 14/55; C07K 2317/24; C07K 2317/31; C07K 2317/522; C07K 2317/524; C07K 2317/526; C07K 2317/53; C07K 2317/565; C07K 2317/569; C07K 2317/622; C07K 2317/92; C07K 2319/30

(57) ABSTRACT

Provided herein are methods of treating chronic viral infection (e.g., hepatitis B virus (HBV) and/or human immunodeficiency virus (HIV) infection) comprising administering to an individual in need thereof an effective amount of a fusion protein that comprises: (a) a first moiety comprising an antibody or antigen-binding fragment thereof that specifically binds human CD8b and/or human CD8ab with at least 10-fold higher affinity than its binding to human CD8a and/or human CD8aa; and (b) a second moiety comprising a cytokine, chemokine, or growth factor.

### CROSS-REFERENCE TO RELATED APPLICATIONS

This application claims the priority benefit of U.S. Provisional Application Nos. 63/411,321, filed Sep. 29, 2022; and 63/421,879, filed Nov. 2, 2022; each of which is hereby incorporated by reference in its entirety.

### REFERENCE TO AN ELECTRONIC SEQUENCE LISTING

The content of the electronic sequence listing (182842000740seqlist.xml; Size: 560, 146 bytes; and Date of Creation: Sep. 27, 2023) is herein incorporated by reference in its entirety.

### FIELD

The present disclosure provides methods of treating chronic viral infection (e.g., hepatitis B virus infection and/or human immunodeficiency virus infection) comprising administering to an individual in need thereof an effective amount of a fusion protein that comprises: (a) a first moiety comprising an antibody or antigen-binding fragment thereof that specifically binds human CD8b and/or human CD8ab with at least 10-fold higher affinity than its binding to human CD8a and/or human CD8aa; and (b) a second moiety comprising a cytokine, chemokine, or growth factor.

### BACKGROUND

HBV is a DNA virus that causes acute and chronic liver disease. In HBV infection, T cell and antibody-mediated responses are responsible for resolution of acute infection. CD4+ T cells promote induction and maintenance of both CD8+ T cells and neutralizing antibodies via interactions with B cells, and effector CD8+ T cells induce viral clearance by secreting antiviral cytokines and killing infected hepatocytes. Neutralizing antibodies may contain viral spread from residual cells that are not eliminated by CD8+ T cells.

Although CD8+ T cells are critical for anti-viral activity against HBV, their responses against HBV antigens may be deficient in chronically infected patients (Iannacone, M. and Guidotti, L. G. (2022) Nat Rev Imm 22 (1): 19-32; Thimme, R. et al. (2003) J. Virol. 77 (1): 68-76; Rehermann, B. et al. (1995) J. Exp. Med. 181 (3): 1047-1058). CD8+ T cells recognize HBV antigens on major histocompatibility complex (MHC) on infected hepatocytes; however activation/priming of CD8+ T cells in the liver is thought to result in T cell unresponsiveness or dysfunction and inability of CD8+ T cells to clear infected hepatocytes.

In preclinical HBV models that recapitulate HBV-induced CD8+ T cell dysfunction, IL-2-based treatment, but not checkpoint blockade, reversed this CD8+ T cell defect (Bénéchet, A. P. et al. (2019) Nature 574 (7777): 200-205), suggesting that IL-2 therapy may be a promising approach to reinvigorate immunity against HBV. However, IL-2-based therapeutics are limited by pleiotropy due to the expression of IL-2 receptors on many immune cell types, including immunosuppressive regulatory T cells (Tregs) and toxicity-promoting natural killer (NK) cells. Dosing of untargeted IL-2-based therapeutics may be limited in patients due to toxicity, and therefore, the ability of these therapeutics to deliver sufficient activation of CD8+ T cells may be compromised in patients.

Human immunodeficiency viruses HIV-1 and HIV-2 are enveloped retroviruses that cause acquired immunodeficiency syndrome (AIDS). In 2021, more than 38 million people were thought to be living with HIV infection. It is also thought that 5-20% of all people infected with HIV globally are also co-infected with HBV (see, e.g., Singh, K. P. et al. (2017) AIDS 31 (15): 2035-2052). Liver-related mortality in this co-infected population can be 19-fold higher than that in populations with HBV infection alone, and 8-fold higher than that in populations with HIV alone, and overall mortality and risk of hepatocellular carcinoma (HCC) are also higher. While CD8+ T cells are thought to be able to recognize and kill HIV-infected T cells, HIV can escape the immune system with its high mutation rate, ability to downregulate surface MHC-I expression, and disruption of CD8+ T cell signaling (Gulzar, N. and Copeland, K. F. T. (2004) Curr. HIV Res. 2 (1): 23-37).

Therefore, a need remains to provide cytokine/chemokine/growth factor-based therapeutics for treatment (e.g., for HBV and/or HIV infection) that activate a CD8+ T cell response without activation of other immune subtypes such as Tregs and/or NK cells. For example, such therapeutics may comprise fusion proteins that activate CD8+ T cells in a targeted manner, such as by delivering cytokines including without limitation IL-2 and IL-21.

All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF SUMMARY

The present disclosure describes, inter alia, methods of treating chronic viral infection. As noted above, there is a need for better treatments for HBV (e.g., chronic HBV) and HIV infections, including HBV/HIV co-infection. While IL-2-based therapies are promising, IL-2 receptors are expressed on many immune cell types, and therefore broad activation of IL-2 could have pleiotropic effects. The present disclosure demonstrates that a cis-targeted IL-2 fusion protein which selectively acts on CD8+ T cells and is sufficient for anti-viral activity in a mouse model of viral infection (HBV).

In some aspects, the present disclosure provides methods of treating hepatitis B virus (HBV) and/or human immunodeficiency virus (HIV) infection, comprising administering to an individual in need thereof an effective amount of a fusion protein that comprises: (a) a first moiety comprising an antibody or antigen-binding fragment thereof that specifically binds human CD8b and/or human CD8ab with at least 10-fold higher affinity than its binding to human CD8a and/or human CD8aa; and (b) a second moiety comprising a cytokine, chemokine, or growth factor. In some embodiments, the first moiety is fused to the second moiety directly or via a linker. In some aspects, the present disclosure provides a fusion protein that comprises: (a) a first moiety comprising an antibody or antigen-binding fragment thereof that specifically binds human CD8b and/or human CD8ab with at least 10-fold higher affinity than its binding to human CD8a and/or human CD8aa; and (b) a second moiety comprising a cytokine, chemokine, or growth factor for use in a method of treating HBV and/or HIV infection in an individual in need thereof, said method comprising administering to the individual an effective amount of the fusion protein. In some aspects, the present disclosure provides a fusion protein that comprises: (a) a first moiety comprising an antibody or antigen-binding fragment thereof that specifically binds human CD8b and/or human CD8ab with at least 10-fold higher affinity than its binding to human CD8a and/or human CD8aa; and (b) a second moiety comprising a cytokine, chemokine, or growth factor for use in the manufacture of a medicament for treating HBV and/or HIV infection in an individual in need thereof.

In some aspects, the present disclosure provides methods of treating HBV infection, comprising administering to an individual in need thereof an effective amount of a fusion protein that comprises: (a) a first moiety comprising an antibody or antigen-binding fragment thereof that specifically binds human CD8b and/or human CD8ab with at least 10-fold higher affinity than its binding to human CD8a and/or human CD8aa; and (b) a second moiety comprising a cytokine, chemokine, or growth factor. In some embodiments, the first moiety is fused to the second moiety directly or via a linker. In some aspects, the present disclosure provides a fusion protein that comprises: (a) a first moiety comprising an antibody or antigen-binding fragment thereof that specifically binds human CD8b and/or human CD8ab with at least 10-fold higher affinity than its binding to human CD8a and/or human CD8aa; and (b) a second moiety comprising a cytokine, chemokine, or growth factor for use in a method of treating HBV infection in an individual in need thereof, said method comprising administering to the individual an effective amount of the fusion protein. In some aspects, the present disclosure provides a fusion protein that comprises: (a) a first moiety comprising an antibody or antigen-binding fragment thereof that specifically binds human CD8b and/or human CD8ab with at least 10-fold higher affinity than its binding to human CD8a and/or human CD8aa; and (b) a second moiety comprising a cytokine, chemokine, or growth factor for use in the manufacture of a medicament for treating HBV infection in an individual in need thereof.

In some embodiments according to any of the embodiments described herein, the HBV infection is chronic HBV infection. In some embodiments, the administration results in an increase in HBV-reactive CD8+ T cells in the liver of the individual. In some embodiments, the administration results in reduced levels of serum HBV DNA of the individual.

In some embodiments according to any of the embodiments described herein, the HIV infection is HIV-1 or HIV-2 infection. In some embodiments, the administration results in an increase in HIV-reactive CD8+ T cells in the individual. In some embodiments, the administration results in reduced levels of serum HIV RNA and/or serum HIV antigens of the individual. In some embodiments, the infection is HBV/HIV co-infection.

In some embodiments according to any of the embodiments described herein, the individual is a human. In some embodiments, the second moiety induces activation of CD8+ T cells. In some embodiments, the fusion protein induces activation of cells expressing a human CD8ab heterodimer with at least 10-fold higher potency than activation of cells expressing a human CD8aa homodimer. In some embodiments, the fusion protein induces activation of CD8+ T cells with at least 10-fold higher potency than activation of NK cells. In some embodiments, potency of activation is measured by EC50, as assessed by cell proliferation, STAT5 phosphorylation, and/or cellular cytotoxic function. In some embodiments, cellular cytotoxic function comprises expression of IFNγ and/or granzyme B. In some embodiments, e.g., prior to administration of the fusion protein, the individual has or has been diagnosed with HBV infection.

In some embodiments according to any of the embodiments described herein, the first moiety specifically binds human CD8b and/or human CD8ab with at least 10-fold higher affinity than its binding to human CD8a and/or human CD8aa. In some embodiments, the first moiety specifically binds the extracellular domain(s) of human CD8b and/or human CD8ab with at least 10-fold higher affinity than its binding to the extracellular domain(s) of human CD8a and/or human CD8aa. In some embodiments, the first moiety binds to a cell expressing a human CD8ab heterodimer on its surface with an EC50 that is less than 1000 nM. In some embodiments, the first moiety binds human CD8+ T cells. In some embodiments, the first moiety specifically binds human CD8b and/or human CD8ab with at least 10-fold higher affinity than its binding to human CD8a and/or human CD8aa expressed on the surface of an natural killer (NK) cell. In some embodiments, the first moiety specifically binds a cell expressing human CD8b and/or human CD8ab on its surface (e.g., a T cell) with at least 10-fold higher affinity than its binding to a cell expressing human CD8a and/or human CD8aa on its surface (e.g., an NK cell).

In some embodiments according to any of the embodiments described herein, the first moiety comprises an antibody or antigen-binding fragment (e.g., a humanized or human antibody or antigen-binding fragment). In some embodiments, the antibody or fragment comprises a heavy chain variable (VH) domain and a light chain variable (VL) domain; wherein the VH domain comprises a CDR-H1 comprising the amino acid sequence of SEQ ID NO: 13, a CDR-H2 comprising the amino acid sequence of SEQ ID NO:14, and a CDR-H3 comprising the amino acid sequence of SEQ ID NO: 15; and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:18. In some embodiments, the antibody or fragment comprises a heavy chain variable (VH) domain and a light chain variable (VL) domain; wherein the VH domain comprises a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO: 15; and wherein the VL domain comprises a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO: 18. In some embodiments, the VH domain comprises an amino acid sequence that is at least 90%, at least 95%, at least 99%, or 100% identical to the sequence of SEQ ID NO:62, and wherein the VL domain comprises an amino acid sequence that is at least 90%, at least 95%, at least 99%, or 100% identical to the sequence of SEQ ID NO:63. In some embodiments, the antibody or fragment comprises a heavy chain variable (VH) domain and a light chain variable (VL) domain; wherein the VH domain comprises a CDR-H1 comprising the amino acid sequence of SEQ ID NO: 19, a CDR-H2 comprising the amino acid sequence of SEQ ID NO:20, and a CDR-H3 comprising the amino acid sequence of SEQ ID NO:21; and wherein the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24. In some embodiments, the antibody or fragment comprises a heavy chain variable (VH) domain and a light chain variable (VL) domain; wherein the VH domain comprises a CDR-H1 comprising the amino acid sequence of SEQ ID NO:53, a CDR-H2 comprising the amino acid sequence of SEQ ID NO: 52, and a CDR-H3 comprising the amino acid sequence of SEQ ID NO:21; and wherein the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24. In some embodiments, the VH domain comprises an amino acid sequence that is at least 90%, at least 95%, at least 99%, or 100% identical to the sequence of SEQ ID NO:64, and wherein the VL domain comprises an amino acid sequence that is at least 90%, at least 95%, at least 99%, or 100% identical to the sequence of SEQ ID NO:65. In some embodiments, the antibody or fragment comprises a heavy chain variable (VH) domain and a light chain variable (VL) domain; wherein the VH domain comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; and wherein the VL domain comprises a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO: 30. In some embodiments, the antibody or fragment comprises a heavy chain variable (VH) domain and a light chain variable (VL) domain; wherein the VH domain comprises a CDR-H1 comprising the amino acid sequence of SEQ ID NO:49, a CDR-H2 comprising the amino acid sequence of SEQ ID NO:52, and a CDR-H3 comprising the amino acid sequence of SEQ ID NO:27; and wherein the VL domain comprises a CDR-L1 comprising the amino acid sequence of SEQ ID NO:28, a CDR-L2 comprising the amino acid sequence of SEQ ID NO: 29, and a CDR-L3 comprising the amino acid sequence of SEQ ID NO:30. In some embodiments, the VH domain comprises an amino acid sequence that is at least 90%, at least 95%, at least 99%, or 100% identical to the sequence of SEQ ID NO:66, and wherein the VL domain comprises an amino acid sequence that is at least 90%, at least 95%, at least 99%, or 100% identical to the sequence of SEQ ID NO:67. In some embodiments, the antibody or fragment comprises a heavy chain variable (VH) domain and a light chain variable (VL) domain; wherein the VH domain comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; and wherein the VL domain comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36. In some embodiments, the antibody or fragment comprises a heavy chain variable (VH) domain and a light chain variable (VL) domain; wherein the VH domain comprises a CDR-H1 comprising the amino acid sequence of SEQ ID NO: 54, a CDR-H2 comprising the amino acid sequence of SEQ ID NO:52, and a CDR-H3 comprising the amino acid sequence of SEQ ID NO:33; and wherein the VL domain comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36. In some embodiments, the VH domain comprises an amino acid sequence that is at least 90%, at least 95%, at least 99%, or 100% identical to the sequence of SEQ ID NO:68, and wherein the VL domain comprises an amino acid sequence that is at least 90%, at least 95%, at least 99%, or 100% identical to the sequence of SEQ ID NO: 69. In some embodiments, the antibody or fragment comprises a heavy chain variable (VH) domain and a light chain variable (VL) domain; wherein the VH domain comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39; and wherein the VL domain comprises a CDR-L1 comprising the amino acid sequence of SEQ ID NO:40, a CDR-L2 comprising the amino acid sequence of SEQ ID NO: 41, and a CDR-L3 comprising the amino acid sequence of SEQ ID NO:42. In some embodiments, the antibody or fragment comprises a heavy chain variable (VH) domain and a light chain variable (VL) domain; wherein the VH domain comprises a CDR-H1 comprising the amino acid sequence of SEQ ID NO:55, a CDR-H2 comprising the amino acid sequence of SEQ ID NO:56, and a CDR-H3 comprising the amino acid sequence of SEQ ID NO:39; and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:70, and wherein the VL domain comprises an amino acid sequence that is at least 90%, at least 95%, at least 99%, or 100% identical to the sequence of SEQ ID NO:71. In some embodiments, the antibody or fragment comprises a heavy chain variable (VH) domain and a light chain variable (VL) domain; wherein the VH domain comprises a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45; and wherein the VL domain comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO: 48. In some embodiments, the antibody or fragment comprises a heavy chain variable (VH) domain and a light chain variable (VL) domain; wherein the VH domain comprises a CDR-H1 comprising the amino acid sequence of SEQ ID NO:55, a CDR-H2 comprising the amino acid sequence of SEQ ID NO:57, and a CDR-H3 comprising the amino acid sequence of SEQ ID NO:45; and wherein the VL domain comprises a CDR-L1 comprising the amino acid sequence of SEQ ID NO:46, a CDR-L2 comprising the amino acid sequence of SEQ ID NO: 47, and a CDR-L3 comprising the amino acid sequence of SEQ ID NO:48. In some embodiments, the VH domain comprises an amino acid sequence that is at least 90%, at least 95%, at least 99%, or 100% identical to the sequence of SEQ ID NO:72, and wherein the VL domain comprises an amino acid sequence that is at least 90%, at least 95%, at least 99%, or 100% identical to the sequence of SEQ ID NO:73. In some embodiments, the antibody or fragment comprises a heavy chain variable (VH) domain and a light chain variable (VL) domain; wherein the VH domain comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3; and wherein the VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6. In some embodiments, the antibody or fragment comprises a heavy chain variable (VH) domain and a light chain variable (VL) domain; wherein the VH domain comprises a CDR-H1 comprising the amino acid sequence of SEQ ID NO:49, a CDR-H2 comprising the amino acid sequence of SEQ ID NO:50, and a CDR-H3 comprising the amino acid sequence of SEQ ID NO:3; and wherein the VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO: 6. In some embodiments, the antibody or fragment comprises a heavy chain variable (VH) domain and a light chain variable (VL) domain; wherein the VH domain comprises a CDR-H1 comprising the amino acid sequence of SEQ ID NO:177, a CDR-H2 comprising the amino acid sequence of SEQ ID NO:178, and a CDR-H3 comprising the amino acid sequence of SEQ ID NO: 179; and wherein the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182. In some embodiments, the antibody or fragment comprises a heavy chain variable (VH) domain and a light chain variable (VL) domain; wherein the VH domain comprises a CDR-H1 comprising the amino acid sequence of SEQ ID NO: 183, a CDR-H2 comprising the amino acid sequence of SEQ ID NO:184, and a CDR-H3 comprising the amino acid sequence of SEQ ID NO: 179; and wherein the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO:182. In some embodiments, the VH domain comprises an amino acid sequence that is at least 90%, at least 95%, at least 99%, or 100% identical to the sequence of SEQ ID NO:185, and wherein the VL domain comprises an amino acid sequence that is at least 90%, at least 95%, at least 99%, or 100% identical to the sequence of SEQ ID NO: 186.

In some embodiments, the antibody or fragment comprises a heavy chain variable (VH) domain and a light chain variable (VL) domain; wherein the VH domain comprises a CDR-H1 comprising the amino acid sequence of X₁X₂AIS, wherein X₁ is S, K, G, N, R, D, T, or G, and wherein X₂ is Y, L, H, or F (SEQ ID NO:259), a CDR-H2 comprising the amino acid sequence of X₁X₂X₃PX₄X₅X₆X₇X₈X₉YX₁₀QKFX₁₁G, wherein X₁ is G or H, X₂ is I or F, X₃ is I, N, or M, X₄ is G, N, H, S, R, I, or A, X₅ is A, N, H, S, T, F, or Y, X₆ is A, D, or G, X₇ is T, E, K, V, Q, or A, X₈ is A or T, X₉ is N or K, X₁₀ is A or N, and X₁₁ is Q or T (SEQ ID NO: 260), and a CDR-H3 comprising the amino acid sequence of X₁X₂X₃GX₄X₅LFX₆X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO:261); and wherein the VL domain comprises a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO:262), a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO:263), and a CDR-L3 comprising the amino acid sequence of QX₁X₂X₃X₄X₅PWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO:264). In some embodiments, the antibody or fragment comprises a heavy chain variable (VH) domain and a light chain variable (VL) domain; wherein the VH domain comprises a CDR-H1 comprising the amino acid sequence of SEQ ID NO:225, a CDR-H2 comprising the amino acid sequence of SEQ ID NO:226, and a CDR-H3 comprising the amino acid sequence of SEQ ID NO:227; and wherein the VL domain comprises a CDR-L1 comprising the amino acid sequence of SEQ ID NO: 16, a CDR-L2 comprising the amino acid sequence of SEQ ID NO:17, and a CDR-L3 comprising the amino acid sequence of SEQ ID NO: 228. In some embodiments, the VH domain comprises an amino acid sequence that is at least 90%, at least 95%, at least 99%, or 100% identical to the sequence of SEQ ID NO: 245, and wherein the VL domain comprises an amino acid sequence that is at least 90%, at least 95%, at least 99%, or 100% identical to the sequence of SEQ ID NO:246. In some embodiments, the antibody or fragment comprises a heavy chain variable (VH) domain and a light chain variable (VL) domain; wherein the VH domain comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233; and wherein the VL domain comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236. In some embodiments, the VH domain comprises an amino acid sequence that is at least 90%, at least 95%, at least 99%, or 100% identical to the sequence of SEQ ID NO:251, and wherein the VL domain comprises an amino acid sequence that is at least 90%, at least 95%, at least 99%, or 100% identical to the sequence of SEQ ID NO:252. In some embodiments, the VH domain comprises the amino acid sequence of SEQ ID NO:251; and the VL domain comprises the amino acid sequence of SEQ ID NO:252. In some embodiments, the antibody or fragment comprises a heavy chain variable (VH) domain and a light chain variable (VL) domain; wherein the VH domain comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233; and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:228. In some embodiments, the VH domain comprises an amino acid sequence that is at least 90%, at least 95%, at least 99%, or 100% identical to the sequence of SEQ ID NO:253, and wherein the VL domain comprises an amino acid sequence that is at least 90%, at least 95%, at least 99%, or 100% identical to the sequence of SEQ ID NO: 254. In some embodiments, the VH domain further comprises a FW-1 comprising the sequence QVQLVQSGAEVKKPGSSVKVSCKASGGTFS (SEQ ID NO:274), a FW-2 comprising the sequence WVRQAPGQGLEWMG (SEQ ID NO:275), a FW-3 comprising the sequence RVTITADESTSTAYMELSSLRSEDTAVYYCAR (SEQ ID NO:276), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, the antibody or fragment comprises a heavy chain variable (VH) domain and a light chain variable (VL) domain; wherein the VH domain comprises a CDR-H1 comprising the amino acid sequence of GX₁X₂FX₃X₄X₅, wherein X₁ is G, Y, S, or A, X₂ is T, S, G, R, N, or H, X₃ is S, T, R, H, Y, G, or P, X₄ is S, K, G, N, R, D, T, or G, and X₅ is Y, L, H, or F (SEQ ID NO:265), a CDR-H2 comprising the amino acid sequence of X₁PX₂X₃X₄X₅, wherein X₁ is I, N, or M, X₂ is G, N, H, S, R, I, or A, X₃ is A, N, H, S, T, F, or Y, X₄ is A, D, or G, and X₅ is T, E, K, V, Q, or A (SEQ ID NO:266), and a CDR-H3 comprising the amino acid sequence of X₁X₂X₃GX₄X₅LFX₆X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO:267); and wherein the VL domain comprises a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO:262), a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO:263), and a CDR-L3 comprising the amino acid sequence of QX₁X₂X₃X₄X₅PWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO:264). In some embodiments, the VH domain comprises a CDR-H1 comprising the amino acid sequence of SEQ ID NO:238, a CDR-H2 comprising the amino acid sequence of SEQ ID NO:239, and a CDR-H3 comprising the amino acid sequence of SEQ ID NO:233; and the VL domain comprises a CDR-L1 comprising the amino acid sequence of SEQ ID NO: 16, a CDR-L2 comprising the amino acid sequence of SEQ ID NO:17, and a CDR-L3 comprising the amino acid sequence of SEQ ID NO:228. In some embodiments, the VH domain comprises an amino acid sequence that is at least 90%, at least 95%, at least 99%, or 100% identical to the sequence of SEQ ID NO:245; and wherein the VL domain comprises an amino acid sequence that is at least 90%, at least 95%, at least 99%, or 100% identical to the sequence of SEQ ID NO: 246. In some embodiments, the VH domain comprises a CDR-H1 comprising the amino acid sequence of SEQ ID NO:238, a CDR-H2 comprising the amino acid sequence of SEQ ID NO: 243, and a CDR-H3 comprising the amino acid sequence of SEQ ID NO:233; and the VL domain comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236. In some embodiments, the VH domain comprises an amino acid sequence that is at least 90%, at least 95%, at least 99%, or 100% identical to the sequence of SEQ ID NO:251; and wherein the VL domain comprises an amino acid sequence that is at least 90%, at least 95%, at least 99%, or 100% identical to the sequence of SEQ ID NO:252. In some embodiments, the VH domain comprises the amino acid sequence of SEQ ID NO:251; and the VL domain comprises the amino acid sequence of SEQ ID NO:252. In some embodiments, the VH domain comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233; and the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:228. In some embodiments, the VH domain comprises an amino acid sequence that is at least 90%, at least 95%, at least 99%, or 100% identical to the sequence of SEQ ID NO:253; and wherein the VL domain comprises an amino acid sequence that is at least 90%, at least 95%, at least 99%, or 100% identical to the sequence of SEQ ID NO:254. In some embodiments, the VH domain further comprises a FW-1 comprising the sequence QVQLVQSGAEVKKPGSSVKVSCKAS (SEQ ID NO:278), a FW-2 comprising the sequence AISWVRQAPGQGLEWMGGI (SEQ ID NO:279), a FW-3 comprising the sequence ANYAQKFQGRVTITADESTSTAYMELSSLRSEDTAVYYCAR (SEQ ID NO:280), and/or a FW-4 comprising the sequence WGQGTLVTVSS (SEQ ID NO: 277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, the antibody or fragment comprises a heavy chain variable (VH) domain and a light chain variable (VL) domain; wherein the VH domain comprises a CDR-H1 comprising the amino acid sequence of X₁YX₂MS, wherein X₁ is S, D, E, A, or Q and X₂ is A, G, or T (SEQ ID NO:268), a CDR-H2 comprising the amino acid sequence of DIX₁X₂X₃GX₄X₅TX₆YADSVKG, wherein X₁ is T, N, S, Q, E, H, R, or A, X₂ is Y, W, F, or H, X₃ is A, S, Q, E, or T, X₄ is G or E, X₅ is S or I, and X₆ is A or G (SEQ ID NO:269), and a CDR-H3 comprising the amino acid sequence of X₁X₂X₃YX₄WX₅X₆AX₇DX₈, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO:270) and a VL domain comprising a CDR-L1 comprising the amino acid sequence of RASQSVSSNLA (SEQ ID NO:40), a CDR-L2 comprising the amino acid sequence of GASSRAT (SEQ ID NO: 41), and a CDR-L3 comprising the amino acid sequence of QQYGSSPPVT (SEQ ID NO: 42). In some embodiments, the antibody or fragment comprises a heavy chain variable (VH) domain and a light chain variable (VL) domain; wherein the VH domain comprises a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231; and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:247, and wherein the VL domain comprises an amino acid sequence that is at least 90%, at least 95%, at least 99%, or 100% identical to the sequence of SEQ ID NO:248. In some embodiments, the VH domain comprises an amino acid sequence that is at least 90%, at least 95%, at least 99%, or 100% identical to the sequence of SEQ ID NO:249, and wherein the VL domain comprises an amino acid sequence that is at least 90%, at least 95%, at least 99%, or 100% identical to the sequence of SEQ ID NO:250. In some embodiments, the VH domain comprises the amino acid sequence of SEQ ID NO:249, and wherein the VL domain comprises the amino acid sequence of SEQ ID NO:250. In some embodiments, the antibody or fragment comprises a heavy chain variable (VH) domain and a light chain variable (VL) domain; wherein the VH domain comprises a CDR-H1 comprising the amino acid sequence of SEQ ID NO: 229, a CDR-H2 comprising the amino acid sequence of SEQ ID NO:237, and a CDR-H3 comprising the amino acid sequence of SEQ ID NO:231; and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:255, and wherein the VL domain comprises an amino acid sequence that is at least 90%, at least 95%, at least 99%, or 100% identical to the sequence of SEQ ID NO: 256. In some embodiments, the VH domain comprises an amino acid sequence that is at least 90%, at least 95%, at least 99%, or 100% identical to the sequence of SEQ ID NO:257, and wherein the VL domain comprises an amino acid sequence that is at least 90%, at least 95%, at least 99%, or 100% identical to the sequence of SEQ ID NO:258. In some embodiments, the VH domain further comprises a FW-1 comprising the sequence EVOLVESGGGLVQPGGSLRLSCAASGFTFS (SEQ ID NO:281), a FW-2 comprising the sequence WVRQAPGKGLEWVS (SEQ ID NO:282), a FW-3 comprising the sequence RFTISRDNAKNSLYLQMNSLRAEDTAVYYCAR (SEQ ID NO:283), and/or a FW-4 comprising the sequence WGQGTMVTVSS (SEQ ID NO:284) or WGQGTLVTVSS (SEQ ID NO: 285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, the antibody or fragment comprises a heavy chain variable (VH) domain and a light chain variable (VL) domain; wherein the VH domain comprises a CDR-H1 comprising the amino acid sequence of GFTFX₁X₂Y, wherein X₁ is S, D, E, Q, S, or A and X₂ is S, D, E, A, or Q (SEQ ID NO:271), a CDR-H2 comprising the amino acid sequence of X₁X₂X₃GX₄X₅, wherein X₁ is T, N, S, Q, E, H, R or A, X₂ is Y, W, F, or H, X₃ is A, S, Q, E, or T, X₄ is G or E, and X₅ is S or I (SEQ ID NO:272), and a CDR-H3 comprising the amino acid sequence of X₁X₂X₃YX₄WX₅X₆AX-DX₈, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO:273); and wherein the VL domain comprises a CDR-L1 comprising the amino acid sequence of RASQSVSSNLA (SEQ ID NO: 40), a CDR-L2 comprising the amino acid sequence of GASSRAT (SEQ ID NO:41), and a CDR-L3 comprising the amino acid sequence of QQYGSSPPVT (SEQ ID NO:42). In some embodiments, the VH domain comprises a CDR-H1 comprising the amino acid sequence of SEQ ID NO:240, a CDR-H2 comprising the amino acid sequence of SEQ ID NO: 241, and a CDR-H3 comprising the amino acid sequence of SEQ ID NO:242; and wherein the VL domain comprises a CDR-L1 comprising the amino acid sequence of SEQ ID NO: 40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:247; and wherein the VL domain comprises an amino acid sequence that is at least 90%, at least 95%, at least 99%, or 100% identical to the sequence of SEQ ID NO:248. In some embodiments, the VH domain comprises an amino acid sequence that is at least 90%, at least 95%, at least 99%, or 100% identical to the sequence of SEQ ID NO:249; and wherein the VL domain comprises an amino acid sequence that is at least 90%, at least 95%, at least 99%, or 100% identical to the sequence of SEQ ID NO: 250. In some embodiments, the VH domain comprises the amino acid sequence of SEQ ID NO: 249, and wherein the VL domain comprises the amino acid sequence of SEQ ID NO: 250. In some embodiments, the VH domain comprises a CDR-H1 comprising the amino acid sequence of SEQ ID NO:240, a CDR-H2 comprising the amino acid sequence of SEQ ID NO: 244, and a CDR-H3 comprising the amino acid sequence of SEQ ID NO:242; and wherein the VL domain comprises a CDR-L1 comprising the amino acid sequence of SEQ ID NO: 40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO:255; and wherein the VL domain comprises an amino acid sequence that is at least 90%, at least 95%, at least 99%, or 100% identical to the sequence of SEQ ID NO:256. In some embodiments, the VH domain comprises an amino acid sequence that is at least 90%, at least 95%, at least 99%, or 100% identical to the sequence of SEQ ID NO:257; and wherein the VL domain comprises an amino acid sequence that is at least 90%, at least 95%, at least 99%, or 100% identical to the sequence of SEQ ID NO: 258. In some embodiments, the VH domain further comprises a FW-1 comprising the sequence EVOLVESGGGLVQPGGSLRLSCAAS (SEQ ID NO:286), a FW-2 comprising the sequence AMSWVRQAPGKGLEWVSDI (SEQ ID NO:287), a FW-3 comprising the sequence TAYADSVKGRFTISRDNAKNSLYLQMNSLRAEDTAVYYCAR (SEQ ID NO: 288), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO: 293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO: 295), and a FW-4 comprising the sequence FGQGTKVEIK (SEQ ID NO:296).

In some embodiments according to any of the embodiments described herein, the antibody or fragment is a multispecific antibody or fragment (e.g., a bispecific antibody or fragment).

In some embodiments according to any of the embodiments described herein, the fusion protein comprises a first moiety according to any one of the above embodiments and a second moiety comprising a cytokine, chemokine, or growth factor. In some embodiments, the first moiety is fused to the second moiety directly or via a linker. In some embodiments, the first moiety comprises a human or humanized antibody or antigen-binding fragment thereof that specifically binds CD8b and/or CD8ab, wherein the antibody or fragment comprises a heavy chain variable (VH) domain and a light chain variable (VL) domain, wherein: the VH domain comprises a CDR-H1 comprising the amino acid sequence of SEQ ID NO: 13, a CDR-H2 comprising the amino acid sequence of SEQ ID NO: 14, and a CDR-H3 comprising the amino acid sequence of SEQ ID NO: 15; and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:18; the VH domain comprises a CDR-H1 comprising the amino acid sequence of SEQ ID NO:19, a CDR-H2 comprising the amino acid sequence of SEQ ID NO: 20, and a CDR-H3 comprising the amino acid sequence of SEQ ID NO:21; and wherein the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24; the VH domain comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO: 27; and wherein the VL domain comprises a CDR-L1 comprising the amino acid sequence of SEQ ID NO:28, a CDR-L2 comprising the amino acid sequence of SEQ ID NO: 29, and a CDR-L3 comprising the amino acid sequence of SEQ ID NO:30; the VH domain comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; and wherein the VL domain comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO: 36; the VH domain comprises a CDR-H1 comprising the amino acid sequence of SEQ ID NO: 37, a CDR-H2 comprising the amino acid sequence of SEQ ID NO:38, and a CDR-H3 comprising the amino acid sequence of SEQ ID NO:39; and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain comprises a CDR-H1 comprising the amino acid sequence of SEQ ID NO:43, a CDR-H2 comprising the amino acid sequence of SEQ ID NO: 44, and a CDR-H3 comprising the amino acid sequence of SEQ ID NO:45; and wherein the VL domain comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; the VH domain comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO: 3; and wherein the VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6; the VH domain comprises a CDR-H1 comprising the amino acid sequence of SEQ ID NO:177, a CDR-H2 comprising the amino acid sequence of SEQ ID NO:178, and a CDR-H3 comprising the amino acid sequence of SEQ ID NO: 179; and wherein the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO:182; the VH domain comprises a CDR-H1 comprising the amino acid sequence of SEQ ID NO: 225, a CDR-H2 comprising the amino acid sequence of SEQ ID NO:226, and a CDR-H3 comprising the amino acid sequence of SEQ ID NO:227; and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:228; the VH domain comprises a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231; and wherein the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42); the VH domain comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233; and wherein the VL domain comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236; the VH domain comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233; and wherein the VL domain comprises a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO:228; or the VH domain comprises a CDR-H1 comprising the amino acid sequence of SEQ ID NO: 229, a CDR-H2 comprising the amino acid sequence of SEQ ID NO:237, and a CDR-H3 comprising the amino acid sequence of SEQ ID NO:231, and wherein the VL domain comprises a CDR-L1 comprising the amino acid sequence of RASQSVSSNLA (SEQ ID NO: 40), a CDR-L2 comprising the amino acid sequence of GASSRAT (SEQ ID NO:41), and a CDR-L3 comprising the amino acid sequence of QQYGSSPPVT (SEQ ID NO:42). In some embodiments, the first moiety comprises a human or humanized antibody or antigen-binding fragment thereof that specifically binds CD8b and/or CD8ab, wherein the antibody or fragment comprises a heavy chain variable (VH) domain and a light chain variable (VL) domain, wherein: the VH domain comprises a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO: 15, and wherein the VL domain comprises a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO: 18; the VH domain comprises a CDR-H1 comprising the amino acid sequence of SEQ ID NO:53, a CDR-H2 comprising the amino acid sequence of SEQ ID NO:52, and a CDR-H3 comprising the amino acid sequence of SEQ ID NO:21, and wherein the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24; the VH domain comprises a CDR-H1 comprising the amino acid sequence of SEQ ID NO:49, a CDR-H2 comprising the amino acid sequence of SEQ ID NO:52, and a CDR-H3 comprising the amino acid sequence of SEQ ID NO:27, and wherein the VL domain comprises a CDR-L1 comprising the amino acid sequence of SEQ ID NO:28, a CDR-L2 comprising the amino acid sequence of SEQ ID NO: 29, and a CDR-L3 comprising the amino acid sequence of SEQ ID NO:30; the VH domain comprises a CDR-H1 comprising the amino acid sequence of SEQ ID NO:54, a CDR-H2 comprising the amino acid sequence of SEQ ID NO:52, and a CDR-H3 comprising the amino acid sequence of SEQ ID NO:33, and wherein the VL domain comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO: 36; the VH domain comprises a CDR-H1 comprising the amino acid sequence of SEQ ID NO: 55, a CDR-H2 comprising the amino acid sequence of SEQ ID NO:56, and a CDR-H3 comprising the amino acid sequence of SEQ ID NO:39, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain comprises a CDR-H1 comprising the amino acid sequence of SEQ ID NO:55, a CDR-H2 comprising the amino acid sequence of SEQ ID NO: 57, and a CDR-H3 comprising the amino acid sequence of SEQ ID NO:45, and wherein the VL domain comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; the VH domain comprises a CDR-H1 comprising the amino acid sequence of SEQ ID NO:49, a CDR-H2 comprising the amino acid sequence of SEQ ID NO:50, and a CDR-H3 comprising the amino acid sequence of SEQ ID NO: 3, and wherein the VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6; the VH domain comprises a CDR-H1 comprising the amino acid sequence of SEQ ID NO:183, a CDR-H2 comprising the amino acid sequence of SEQ ID NO: 184, and a CDR-H3 comprising the amino acid sequence of SEQ ID NO: 179, and wherein the VL domain comprises a CDR-L1 comprising the amino acid sequence of SEQ ID NO:180, a CDR-L2 comprising the amino acid sequence of SEQ ID NO:181, and a CDR-L3 comprising the amino acid sequence of SEQ ID NO:182; the VH domain comprises a CDR-H1 comprising the amino acid sequence of SEQ ID NO:238, a CDR-H2 comprising the amino acid sequence of SEQ ID NO:239, and a CDR-H3 comprising the amino acid sequence of SEQ ID NO:233, and wherein the VL domain comprises a CDR-L1 comprising the amino acid sequence of SEQ ID NO: 16, a CDR-L2 comprising the amino acid sequence of SEQ ID NO:17, and a CDR-L3 comprising the amino acid sequence of SEQ ID NO:228; the VH domain comprises a CDR-H1 comprising the amino acid sequence of SEQ ID NO:240, a CDR-H2 comprising the amino acid sequence of SEQ ID NO:241, and a CDR-H3 comprising the amino acid sequence of SEQ ID NO:242, and wherein the VL domain comprises a CDR-L1 comprising the amino acid sequence of SEQ ID NO:40, a CDR-L2 comprising the amino acid sequence of SEQ ID NO: 41, and a CDR-L3 comprising the amino acid sequence of SEQ ID NO:42; the VH domain comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233, and wherein the VL domain comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236; the VH domain comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233, and wherein the VL domain comprises a CDR-L1 comprising the amino acid sequence of SEQ ID NO: 16, a CDR-L2 comprising the amino acid sequence of SEQ ID NO:17, and a CDR-L3 comprising the amino acid sequence of SEQ ID NO:228; or the VH domain comprises a CDR-H1 comprising the amino acid sequence of SEQ ID NO:240, a CDR-H2 comprising the amino acid sequence of SEQ ID NO:244, and a CDR-H3 comprising the amino acid sequence of SEQ ID NO:242, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO: 42.

In some embodiments according to any of the embodiments described herein (e.g., a fusion protein of the present disclosure), the second moiety induces activation of CD8+ T cells. In some embodiments, the fusion protein induces activation of cells expressing a human CD8ab heterodimer with at least 10-fold higher potency than activation of cells expressing a human CD8aa homodimer. In some embodiments, the fusion protein induces activation of CD8+ T cells with at least 10-fold higher potency than activation of NK cells. In some embodiments, potency of activation is measured by EC50, as assessed by cell proliferation, STAT5 phosphorylation, and/or cellular cytotoxic function. In some embodiments, the first moiety comprises two antibody heavy chain polypeptides comprising a structure according to formula [I], from N-terminus to C-terminus:

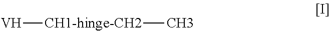

and two antibody light chain polypeptides comprising a structure according to formula [II], from N-terminus to C-terminus:

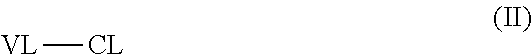

wherein VH is an antibody heavy chain variable (VH) domain, wherein CH1 is an antibody CH1 domain, wherein hinge is an antibody hinge domain, wherein CH2-CH3 is an antibody Fc domain, wherein VL is an antibody light chain variable (VL) domain, and wherein CL is an antibody constant light chain domain; and wherein the N-terminus of the second moiety is fused to the C-terminus of one of the two CH3 domains (e.g., via a linker of the present disclosure). In some embodiments, the first moiety comprises a first antibody heavy chain polypeptide comprising a structure according to formula [I], from N-terminus to C-terminus:

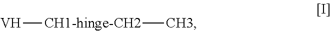

an antibody light chain polypeptide comprising a structure according to formula [II], from N-terminus to C-terminus:

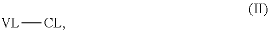

and a second antibody heavy chain polypeptide comprising a structure according to formula [III], from N-terminus to C-terminus:

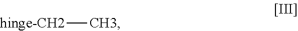

wherein VH is an antibody heavy chain variable (VH) domain, wherein CH1 is an antibody CH1 domain, wherein hinge is an antibody hinge domain, wherein CH2-CH3 is an antibody Fc domain, wherein VL is an antibody light chain variable (VL) domain, and wherein CL is an antibody constant light chain domain; and wherein the N-terminus of the second moiety is fused to the C-terminus of the CH3 domain of the second antibody heavy chain polypeptide (e.g., via a linker of the present disclosure). In some embodiments, the N-terminus of the second moiety is fused to the C-terminus of the CH3 domain of the first antibody heavy chain polypeptide (e.g., via a linker of the present disclosure). In some embodiments, the first moiety comprises a first antibody heavy chain polypeptide comprising a structure according to formula [I], from N-terminus to C-terminus:

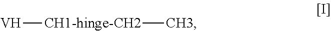

an antibody light chain polypeptide comprising a structure according to formula [II], from N-terminus to C-terminus:

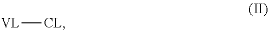

and a second antibody heavy chain polypeptide comprising a structure according to formula [III], from N-terminus to C-terminus:

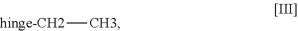

wherein VH is an antibody heavy chain variable (VH) domain, wherein CH1 is an antibody CH1 domain, wherein hinge is an antibody hinge domain, wherein CH2-CH3 is an antibody Fc domain, wherein VL is an antibody light chain variable (VL) domain, and wherein CL is an antibody constant light chain domain; and wherein the C-terminus of the second moiety is fused to the N-terminus of the hinge domain of the second antibody heavy chain polypeptide (e.g., via a linker of the present disclosure). In some embodiments, the first moiety comprises one or two antibody heavy chain polypeptides and one or two antibody light chain polypeptides. In some embodiments, the first moiety comprises a single chain antibody or single chain variable fragment (scFv). In some embodiments, the first moiety comprises a VHH antibody. In some embodiments according to any of the embodiments described herein (e.g., the fusion proteins described above), VH and VL form an antigen binding site (e.g., that specifically binds CD8b and/or CD8ab). In some embodiments, the first moiety comprises a first antibody heavy chain polypeptide comprising a structure according to formula [I], from N-terminus to C-terminus:

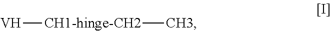

an antibody light chain polypeptide comprising a structure according to formula [II], from N-terminus to C-terminus:

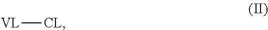

and a second antibody heavy chain polypeptide comprising a structure according to formula [III], from N-terminus to C-terminus:

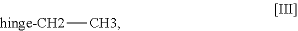

wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain; and wherein the N-terminus of the second moiety is fused to the C-terminus of the CH3 domain of the first antibody heavy chain polypeptide. In some embodiments, the VH domain of both antibody heavy chain polypeptides comprises a CDR-H1 comprising the amino acid sequence of SEQ ID NO: 13, a CDR-H2 comprising the amino acid sequence of SEQ ID NO: 14, and a CDR-H3 comprising the amino acid sequence of SEQ ID NO: 15, and wherein the VL domain of both antibody light chain polypeptides comprises a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO: 18; the VH domain of both antibody heavy chain polypeptides comprises a CDR-H1 comprising the amino acid sequence of SEQ ID NO: 19, a CDR-H2 comprising the amino acid sequence of SEQ ID NO:20, and a CDR-H3 comprising the amino acid sequence of SEQ ID NO:21, and wherein the VL domain of both antibody light chain polypeptides comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO: 24; the VH domain of both antibody heavy chain polypeptides comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO: 27, and wherein the VL domain of both antibody light chain polypeptides comprises a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO:30; the VH domain of both antibody heavy chain polypeptides comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33, and wherein the VL domain of both antibody light chain polypeptides comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36; the VH domain of both antibody heavy chain polypeptides comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39, and wherein the VL domain of both antibody light chain polypeptides comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain of both antibody heavy chain polypeptides comprises a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and wherein the VL domain of both antibody light chain polypeptides comprises a CDR-L1 comprising the amino acid sequence of SEQ ID NO: 46, a CDR-L2 comprising the amino acid sequence of SEQ ID NO:47, and a CDR-L3 comprising the amino acid sequence of SEQ ID NO:48; the VH domain of both antibody heavy chain polypeptides comprises a CDR-H1 comprising the amino acid sequence of SEQ ID NO: 1, a CDR-H2 comprising the amino acid sequence of SEQ ID NO:2, and a CDR-H3 comprising the amino acid sequence of SEQ ID NO:3, and wherein the VL domain of both antibody light chain polypeptides comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6; the VH domain of both antibody heavy chain polypeptides comprises a CDR-H1 comprising the amino acid sequence of SEQ ID NO: 177, a CDR-H2 comprising the amino acid sequence of SEQ ID NO:178, and a CDR-H3 comprising the amino acid sequence of SEQ ID NO: 179, and wherein the VL domain of both antibody light chain polypeptides comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182; the VH domain of both antibody heavy chain polypeptides comprises a CDR-H1 comprising the amino acid sequence of SEQ ID NO:225, a CDR-H2 comprising the amino acid sequence of SEQ ID NO:226, and a CDR-H3 comprising the amino acid sequence of SEQ ID NO:227, and wherein the VL domain of both antibody light chain polypeptides comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO: 228; the VH domain of both antibody heavy chain polypeptides comprises a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231, and wherein the VL domain of both antibody light chain polypeptides comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain of both antibody heavy chain polypeptides comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233, and wherein the VL domain of both antibody light chain polypeptides comprises a CDR-L1 comprising the amino acid sequence of SEQ ID NO: 234, a CDR-L2 comprising the amino acid sequence of SEQ ID NO:235, and a CDR-L3 comprising the amino acid sequence of SEQ ID NO:236; the VH domain of both antibody heavy chain polypeptides comprises a CDR-H1 comprising the amino acid sequence of SEQ ID NO: 225, a CDR-H2 comprising the amino acid sequence of SEQ ID NO:232, and a CDR-H3 comprising the amino acid sequence of SEQ ID NO:233, and wherein the VL domain of both antibody light chain polypeptides comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:228; or the VH domain of both antibody heavy chain polypeptides comprises a CDR-H1 comprising the amino acid sequence of SEQ ID NO:229, a CDR-H2 comprising the amino acid sequence of SEQ ID NO:237, and a CDR-H3 comprising the amino acid sequence of SEQ ID NO:231, and wherein the VL domain of both antibody light chain polypeptides comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO: 42. In some embodiments, the VH domain of both antibody heavy chain polypeptides comprises a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO: 15, and wherein the VL domain of both antibody light chain polypeptides comprises a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO: 18; the VH domain of both antibody heavy chain polypeptides comprises a CDR-H1 comprising the amino acid sequence of SEQ ID NO:53, a CDR-H2 comprising the amino acid sequence of SEQ ID NO:52, and a CDR-H3 comprising the amino acid sequence of SEQ ID NO:21, and wherein the VL domain of both antibody light chain polypeptides comprises a CDR-L1 comprising the amino acid sequence of SEQ ID NO: 22, a CDR-L2 comprising the amino acid sequence of SEQ ID NO:23, and a CDR-L3 comprising the amino acid sequence of SEQ ID NO:24; the VH domain of both antibody heavy chain polypeptides comprises a CDR-H1 comprising the amino acid sequence of SEQ ID NO: 49, a CDR-H2 comprising the amino acid sequence of SEQ ID NO:52, and a CDR-H3 comprising the amino acid sequence of SEQ ID NO:27, and wherein the VL domain of both antibody light chain polypeptides comprises a CDR-L1 comprising the amino acid sequence of SEQ ID NO:28, a CDR-L2 comprising the amino acid sequence of SEQ ID NO: 29, and a CDR-L3 comprising the amino acid sequence of SEQ ID NO:30; the VH domain of both antibody heavy chain polypeptides comprises a CDR-H1 comprising the amino acid sequence of SEQ ID NO:54, a CDR-H2 comprising the amino acid sequence of SEQ ID NO:52, and a CDR-H3 comprising the amino acid sequence of SEQ ID NO:33, and wherein the VL domain of both antibody light chain polypeptides comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO: 36; the VH domain of both antibody heavy chain polypeptides comprises a CDR-H1 comprising the amino acid sequence of SEQ ID NO:55, a CDR-H2 comprising the amino acid sequence of SEQ ID NO:56, and a CDR-H3 comprising the amino acid sequence of SEQ ID NO: 39, and wherein the VL domain of both antibody light chain polypeptides comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain of both antibody heavy chain polypeptides comprises a CDR-H1 comprising the amino acid sequence of SEQ ID NO:55, a CDR-H2 comprising the amino acid sequence of SEQ ID NO:57, and a CDR-H3 comprising the amino acid sequence of SEQ ID NO:45, and wherein the VL domain of both antibody light chain polypeptides comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; the VH domain of both antibody heavy chain polypeptides comprises a CDR-H1 comprising the amino acid sequence of SEQ ID NO:49, a CDR-H2 comprising the amino acid sequence of SEQ ID NO:50, and a CDR-H3 comprising the amino acid sequence of SEQ ID NO:3, and wherein the VL domain of both antibody light chain polypeptides comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6; the VH domain of both antibody heavy chain polypeptides comprises a CDR-H1 comprising the amino acid sequence of SEQ ID NO: 183, a CDR-H2 comprising the amino acid sequence of SEQ ID NO: 184, and a CDR-H3 comprising the amino acid sequence of SEQ ID NO: 179, and wherein the VL domain of both antibody light chain polypeptides comprises a CDR-L1 comprising the amino acid sequence of SEQ ID NO:180, a CDR-L2 comprising the amino acid sequence of SEQ ID NO: 181, and a CDR-L3 comprising the amino acid sequence of SEQ ID NO: 182; the VH domain of both antibody heavy chain polypeptides comprises a CDR-H1 comprising the amino acid sequence of SEQ ID NO:238, a CDR-H2 comprising the amino acid sequence of SEQ ID NO:239, and a CDR-H3 comprising the amino acid sequence of SEQ ID NO:233, and wherein the VL domain of both antibody light chain polypeptides comprises a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO:228; the VH domain of both antibody heavy chain polypeptides comprises a CDR-H1 comprising the amino acid sequence of SEQ ID NO:240, a CDR-H2 comprising the amino acid sequence of SEQ ID NO:241, and a CDR-H3 comprising the amino acid sequence of SEQ ID NO:242, and wherein the VL domain of both antibody light chain polypeptides comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO: 42; the VH domain of both antibody heavy chain polypeptides comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233, and wherein the VL domain of both antibody light chain polypeptides comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236; the VH domain of both antibody heavy chain polypeptides comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233, and wherein the VL domain of both antibody light chain polypeptides comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228; or the VH domain of both antibody heavy chain polypeptides comprises a CDR-H1 comprising the amino acid sequence of SEQ ID NO:240, a CDR-H2 comprising the amino acid sequence of SEQ ID NO:244, and a CDR-H3 comprising the amino acid sequence of SEQ ID NO:242, and wherein the VL domain of both antibody light chain polypeptides comprises a CDR-L1 comprising the amino acid sequence of SEQ ID NO:40, a CDR-L2 comprising the amino acid sequence of SEQ ID NO: 41, and a CDR-L3 comprising the amino acid sequence of SEQ ID NO:42. In some embodiments, the VH domain comprises a CDR-H1 comprising the amino acid sequence of SEQ ID NO:13, a CDR-H2 comprising the amino acid sequence of SEQ ID NO:14, and a CDR-H3 comprising the amino acid sequence of SEQ ID NO: 15, and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:18; the VH domain comprises a CDR-H1 comprising the amino acid sequence of SEQ ID NO: 19, a CDR-H2 comprising the amino acid sequence of SEQ ID NO:20, and a CDR-H3 comprising the amino acid sequence of SEQ ID NO:21, and wherein the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24; the VH domain comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27, and wherein the VL domain comprises a CDR-L1 comprising the amino acid sequence of SEQ ID NO:28, a CDR-L2 comprising the amino acid sequence of SEQ ID NO: 29, and a CDR-L3 comprising the amino acid sequence of SEQ ID NO:30; the VH domain comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33, and wherein the VL domain comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO: 36; the VH domain comprises a CDR-H1 comprising the amino acid sequence of SEQ ID NO: 37, a CDR-H2 comprising the amino acid sequence of SEQ ID NO:38, and a CDR-H3 comprising the amino acid sequence of SEQ ID NO:39, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain comprises a CDR-H1 comprising the amino acid sequence of SEQ ID NO:43, a CDR-H2 comprising the amino acid sequence of SEQ ID NO: 44, and a CDR-H3 comprising the amino acid sequence of SEQ ID NO:45, and wherein the VL domain comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; the VH domain comprises a CDR-H1 comprising the amino acid sequence of SEQ ID NO: 1, a CDR-H2 comprising the amino acid sequence of SEQ ID NO:2, and a CDR-H3 comprising the amino acid sequence of SEQ ID NO: 3, and wherein the VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6; the VH domain comprises a CDR-H1 comprising the amino acid sequence of SEQ ID NO:177, a CDR-H2 comprising the amino acid sequence of SEQ ID NO:178, and a CDR-H3 comprising the amino acid sequence of SEQ ID NO: 179, and wherein the VL domain comprises a CDR-L1 comprising the amino acid sequence of SEQ ID NO:180, a CDR-L2 comprising the amino acid sequence of SEQ ID NO:181, and a CDR-L3 comprising the amino acid sequence of SEQ ID NO: 182; the VH domain comprises a CDR-H1 comprising the amino acid sequence of SEQ ID NO: 225, a CDR-H2 comprising the amino acid sequence of SEQ ID NO:226, and a CDR-H3 comprising the amino acid sequence of SEQ ID NO:227, and wherein the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228; the VH domain comprises a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233, and wherein the VL domain comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236; the VH domain comprises a CDR-H1 comprising the amino acid sequence of SEQ ID NO: 225, a CDR-H2 comprising the amino acid sequence of SEQ ID NO:232, and a CDR-H3 comprising the amino acid sequence of SEQ ID NO:233, and wherein the VL domain comprises a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO:228; or the VH domain comprises a CDR-H1 comprising the amino acid sequence of SEQ ID NO:229, a CDR-H2 comprising the amino acid sequence of SEQ ID NO:237, and a CDR-H3 comprising the amino acid sequence of SEQ ID NO:231, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO: 15, and wherein the VL domain comprises a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO: 18; the VH domain comprises a CDR-H1 comprising the amino acid sequence of SEQ ID NO: 53, a CDR-H2 comprising the amino acid sequence of SEQ ID NO:52, and a CDR-H3 comprising the amino acid sequence of SEQ ID NO:21, and wherein the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24; the VH domain comprises a CDR-H1 comprising the amino acid sequence of SEQ ID NO:49, a CDR-H2 comprising the amino acid sequence of SEQ ID NO: 52, and a CDR-H3 comprising the amino acid sequence of SEQ ID NO:27, and wherein the VL domain comprises a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO:30; the VH domain comprises a CDR-H1 comprising the amino acid sequence of SEQ ID NO:54, a CDR-H2 comprising the amino acid sequence of SEQ ID NO:52, and a CDR-H3 comprising the amino acid sequence of SEQ ID NO: 33, and wherein the VL domain comprises a CDR-L1 comprising the amino acid sequence of SEQ ID NO:34, a CDR-L2 comprising the amino acid sequence of SEQ ID NO: 35, and a CDR-L3 comprising the amino acid sequence of SEQ ID NO:36; the VH domain comprises a CDR-H1 comprising the amino acid sequence of SEQ ID NO:55, a CDR-H2 comprising the amino acid sequence of SEQ ID NO:56, and a CDR-H3 comprising the amino acid sequence of SEQ ID NO:39, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO: 42; the VH domain comprises a CDR-H1 comprising the amino acid sequence of SEQ ID NO: 55, a CDR-H2 comprising the amino acid sequence of SEQ ID NO:57, and a CDR-H3 comprising the amino acid sequence of SEQ ID NO:45, and wherein the VL domain comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; the VH domain comprises a CDR-H1 comprising the amino acid sequence of SEQ ID NO:49, a CDR-H2 comprising the amino acid sequence of SEQ ID NO: 50, and a CDR-H3 comprising the amino acid sequence of SEQ ID NO:3, and wherein the VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6; the VH domain comprises a CDR-H1 comprising the amino acid sequence of SEQ ID NO:183, a CDR-H2 comprising the amino acid sequence of SEQ ID NO:184, and a CDR-H3 comprising the amino acid sequence of SEQ ID NO: 179, and wherein the VL domain comprises a CDR-L1 comprising the amino acid sequence of SEQ ID NO:180, a CDR-L2 comprising the amino acid sequence of SEQ ID NO:181, and a CDR-L3 comprising the amino acid sequence of SEQ ID NO:182; the VH domain comprises a CDR-H1 comprising the amino acid sequence of SEQ ID NO:238, a CDR-H2 comprising the amino acid sequence of SEQ ID NO:239, and a CDR-H3 comprising the amino acid sequence of SEQ ID NO:233, and wherein the VL domain comprises a CDR-L1 comprising the amino acid sequence of SEQ ID NO: 16, a CDR-L2 comprising the amino acid sequence of SEQ ID NO:17, and a CDR-L3 comprising the amino acid sequence of SEQ ID NO:228; the VH domain comprises a CDR-H1 comprising the amino acid sequence of SEQ ID NO: 240, a CDR-H2 comprising the amino acid sequence of SEQ ID NO:241, and a CDR-H3 comprising the amino acid sequence of SEQ ID NO:242, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42; the VH domain comprises a CDR-H1 comprising the amino acid sequence of SEQ ID NO:238, a CDR-H2 comprising the amino acid sequence of SEQ ID NO: 243, and a CDR-H3 comprising the amino acid sequence of SEQ ID NO:233, and wherein the VL domain comprises a CDR-L1 comprising the amino acid sequence of SEQ ID NO: 234, a CDR-L2 comprising the amino acid sequence of SEQ ID NO:235, and a CDR-L3 comprising the amino acid sequence of SEQ ID NO:236; the VH domain comprises a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233, and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:228; or the VH domain comprises a CDR-H1 comprising the amino acid sequence of SEQ ID NO: 240, a CDR-H2 comprising the amino acid sequence of SEQ ID NO:244, and a CDR-H3 comprising the amino acid sequence of SEQ ID NO:242, and wherein the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain of both antibody heavy chain polypeptides comprises an amino acid sequence that is at least 90%, at least 95%, at least 99%, or 100% identical to the sequence of SEQ ID NO:62, and wherein the VL domain of both antibody light chain polypeptides comprises an amino acid sequence that is at least 90%, at least 95%, at least 99%, or 100% identical to the sequence of SEQ ID NO:63; the VH domain of both antibody heavy chain polypeptides comprises an amino acid sequence that is at least 90%, at least 95%, at least 99%, or 100% identical to the sequence of SEQ ID NO: 64, and wherein the VL domain of both antibody light chain polypeptides comprises an amino acid sequence that is at least 90%, at least 95%, at least 99%, or 100% identical to the sequence of SEQ ID NO:65; the VH domain of both antibody heavy chain polypeptides comprises an amino acid sequence that is at least 90%, at least 95%, at least 99%, or 100% identical to the sequence of SEQ ID NO:66, and wherein the VL domain of both antibody light chain polypeptides comprises an amino acid sequence that is at least 90%, at least 95%, at least 99%, or 100% identical to the sequence of SEQ ID NO:67; the VH domain of both antibody heavy chain polypeptides comprises an amino acid sequence that is at least 90%, at least 95%, at least 99%, or 100% identical to the sequence of SEQ ID NO:68, and wherein the VL domain of both antibody light chain polypeptides comprises an amino acid sequence that is at least 90%, at least 95%, at least 99%, or 100% identical to the sequence of SEQ ID NO: 69; the VH domain of both antibody heavy chain polypeptides comprises an amino acid sequence that is at least 90%, at least 95%, at least 99%, or 100% identical to the sequence of SEQ ID NO:70, and wherein the VL domain of both antibody light chain polypeptides comprises an amino acid sequence that is at least 90%, at least 95%, at least 99%, or 100% identical to the sequence of SEQ ID NO:71; the VH domain of both antibody heavy chain polypeptides comprises an amino acid sequence that is at least 90%, at least 95%, at least 99%, or 100% identical to the sequence of SEQ ID NO:72, and wherein the VL domain of both antibody light chain polypeptides comprises an amino acid sequence that is at least 90%, at least 95%, at least 99%, or 100% identical to the sequence of SEQ ID NO:73; the VH domain of both antibody heavy chain polypeptides comprises an amino acid sequence that is at least 90%, at least 95%, at least 99%, or 100% identical to the sequence of SEQ ID NO: 185, and wherein the VL domain of both antibody light chain polypeptides comprises an amino acid sequence that is at least 90%, at least 95%, at least 99%, or 100% identical to the sequence of SEQ ID NO: 186; the VH domain of both antibody heavy chain polypeptides comprises an amino acid sequence that is at least 90%, at least 95%, at least 99%, or 100% identical to the sequence of SEQ ID NO:245, and wherein the VL domain of both antibody light chain polypeptides comprises an amino acid sequence that is at least 90%, at least 95%, at least 99%, or 100% identical to the sequence of SEQ ID NO:246; the VH domain of both antibody heavy chain polypeptides comprises an amino acid sequence that is at least 90%, at least 95%, at least 99%, or 100% identical to the sequence of SEQ ID NO:251, and wherein the VL domain of both antibody light chain polypeptides comprises an amino acid sequence that is at least 90%, at least 95%, at least 99%, or 100% identical to the sequence of SEQ ID NO: 252; the VH domain comprises the amino acid sequence of SEQ ID NO:251, and wherein the VL domain comprises the amino acid sequence of SEQ ID NO:252; the VH domain of both antibody heavy chain polypeptides comprises an amino acid sequence that is at least 90%, at least 95%, at least 99%, or 100% identical to the sequence of SEQ ID NO: 253, and wherein the VL domain of both antibody light chain polypeptides comprises an amino acid sequence that is at least 90%, at least 95%, at least 99%, or 100% identical to the sequence of SEQ ID NO:254; the VH domain of both antibody heavy chain polypeptides comprises an amino acid sequence that is at least 90%, at least 95%, at least 99%, or 100% identical to the sequence of SEQ ID NO:247, and wherein the VL domain of both antibody light chain polypeptides comprises an amino acid sequence that is at least 90%, at least 95%, at least 99%, or 100% identical to the sequence of SEQ ID NO:248; the VH domain of both antibody heavy chain polypeptides comprises an amino acid sequence that is at least 90%, at least 95%, at least 99%, or 100% identical to the sequence of SEQ ID NO:249, and wherein the VL domain of both antibody light chain polypeptides comprises an amino acid sequence that is at least 90%, at least 95%, at least 99%, or 100% identical to the sequence of SEQ ID NO: 250; the VH domain comprises the amino acid sequence of SEQ ID NO:249, and wherein the VL domain comprises the amino acid sequence of SEQ ID NO:250; the VH domain of both antibody heavy chain polypeptides comprises an amino acid sequence that is at least 90%, at least 95%, at least 99%, or 100% identical to the sequence of SEQ ID NO: 255, and wherein the VL domain of both antibody light chain polypeptides comprises an amino acid sequence that is at least 90%, at least 95%, at least 99%, or 100% identical to the sequence of SEQ ID NO:256; or the VH domain of both antibody heavy chain polypeptides comprises an amino acid sequence that is at least 90%, at least 95%, at least 99%, or 100% identical to the sequence of SEQ ID NO:257, and wherein the VL domain of both antibody light chain polypeptides comprises an amino acid sequence that is at least 90%, at least 95%, at least 99%, or 100% identical to the sequence of SEQ ID NO:258. In some embodiments, the VH domain comprises an amino acid sequence that is at least 90%, at least 95%, at least 99%, or 100% identical to the sequence of SEQ ID NO:62, and wherein the VL domain comprises an amino acid sequence that is at least 90%, at least 95%, at least 99%, or 100% identical to the sequence of SEQ ID NO:63; the VH domain comprises an amino acid sequence that is at least 90%, at least 95%, at least 99%, or 100% identical to the sequence of SEQ ID NO:64, and wherein the VL domain comprises an amino acid sequence that is at least 90%, at least 95%, at least 99%, or 100% identical to the sequence of SEQ ID NO:65; the VH domain comprises an amino acid sequence that is at least 90%, at least 95%, at least 99%, or 100% identical to the sequence of SEQ ID NO:66, and wherein the VL domain comprises an amino acid sequence that is at least 90%, at least 95%, at least 99%, or 100% identical to the sequence of SEQ ID NO:67; the VH domain comprises an amino acid sequence that is at least 90%, at least 95%, at least 99%, or 100% identical to the sequence of SEQ ID NO:68, and wherein the VL domain comprises an amino acid sequence that is at least 90%, at least 95%, at least 99%, or 100% identical to the sequence of SEQ ID NO:69; the VH domain comprises an amino acid sequence that is at least 90%, at least 95%, at least 99%, or 100% identical to the sequence of SEQ ID NO:70, and wherein the VL domain comprises an amino acid sequence that is at least 90%, at least 95%, at least 99%, or 100% identical to the sequence of SEQ ID NO:71; the VH domain comprises an amino acid sequence that is at least 90%, at least 95%, at least 99%, or 100% identical to the sequence of SEQ ID NO:72, and wherein the VL domain comprises an amino acid sequence that is at least 90%, at least 95%, at least 99%, or 100% identical to the sequence of SEQ ID NO: 73; the VH domain comprises an amino acid sequence that is at least 90%, at least 95%, at least 99%, or 100% identical to the sequence of SEQ ID NO:185, and wherein the VL domain comprises an amino acid sequence that is at least 90%, at least 95%, at least 99%, or 100% identical to the sequence of SEQ ID NO: 186; the VH domain comprises an amino acid sequence that is at least 90%, at least 95%, at least 99%, or 100% identical to the sequence of SEQ ID NO:245, and wherein the VL domain comprises an amino acid sequence that is at least 90%, at least 95%, at least 99%, or 100% identical to the sequence of SEQ ID NO:246; the VH domain comprises an amino acid sequence that is at least 90%, at least 95%, at least 99%, or 100% identical to the sequence of SEQ ID NO:251, and wherein the VL domain comprises an amino acid sequence that is at least 90%, at least 95%, at least 99%, or 100% identical to the sequence of SEQ ID NO:252; the VH domain comprises the amino acid sequence of SEQ ID NO:251, and wherein the VL domain comprises the amino acid sequence of SEQ ID NO:252; the VH domain comprises an amino acid sequence that is at least 90%, at least 95%, at least 99%, or 100% identical to the sequence of SEQ ID NO:253, and wherein the VL domain comprises an amino acid sequence that is at least 90%, at least 95%, at least 99%, or 100% identical to the sequence of SEQ ID NO:254; the VH domain comprises an amino acid sequence that is at least 90%, at least 95%, at least 99%, or 100% identical to the sequence of SEQ ID NO:247, and wherein the VL domain comprises an amino acid sequence that is at least 90%, at least 95%, at least 99%, or 100% identical to the sequence of SEQ ID NO:248; the VH domain comprises an amino acid sequence that is at least 90%, at least 95%, at least 99%, or 100% identical to the sequence of SEQ ID NO:249, and wherein the VL domain comprises an amino acid sequence that is at least 90%, at least 95%, at least 99%, or 100% identical to the sequence of SEQ ID NO:250; the VH domain comprises the amino acid sequence of SEQ ID NO:249, and wherein the VL domain comprises the amino acid sequence of SEQ ID NO:250; the VH domain comprises an amino acid sequence that is at least 90%, at least 95%, at least 99%, or 100% identical to the sequence of SEQ ID NO:255, and wherein the VL domain comprises an amino acid sequence that is at least 90%, at least 95%, at least 99%, or 100% identical to the sequence of SEQ ID NO: 256; or the VH domain comprises an amino acid sequence that is at least 90%, at least 95%, at least 99%, or 100% identical to the sequence of SEQ ID NO:257, and wherein the VL domain comprises an amino acid sequence that is at least 90%, at least 95%, at least 99%, or 100% identical to the sequence of SEQ ID NO:258. In some embodiments, one or both of the antibody heavy chain polypeptides comprise(s) the following amino acid substitutions: L234A, L235A, and G237A, numbering according to EU index. In some embodiments, a first of the antibody heavy chain polypeptides comprises amino acid substitutions Y349C and T366W, and a second of the antibody heavy chain polypeptides comprises amino acid substitutions S354C, T366S, L368A and Y407V, numbering according to EU index.

In some embodiments according to any of the embodiments described herein, the second moiety comprises an IL-2 polypeptide. In some embodiments, the IL-2 polypeptide is a mutant IL-2 polypeptide comprising one or more mutations relative to a human IL-2 polypeptide comprising the sequence of APTSSSTKKTQLQLEHLLLDLQMILNGINNYKNPKLTRMLTFKFYMPKKATELKHLQ CLEEELKPLEEVLNLAQSKNFHLRPRDLISNINVIVLELKGSETTFMCEYADETATIVE FLNRWITFCQSIISTLT (SEQ ID NO:81). In some embodiments, the mutant IL-2 polypeptide has a binding affinity to IL-2Ra that is reduced by 50% or more, compared to binding affinity of a wild-type IL-2 polypeptide comprising the sequence of SEQ ID: 81 for IL-2Ra. In some embodiments, the mutant IL-2 polypeptide has a binding affinity to IL-2RB that is reduced by 50% or more, compared to binding affinity of a wild-type IL-2 polypeptide comprising the sequence of SEQ ID: 81 for IL-2RB; and/or the mutant IL-2 polypeptide has a binding affinity to IL-2Ry that is reduced by 50% or more, compared to binding affinity of a wild-type IL-2 polypeptide comprising the sequence of SEQ ID: 81 for IL-2Ry. In some embodiments, the IL-2 polypeptide comprises the sequence of SEQ ID NO:81 with one, two, three, four, or five amino acid substitutions relative to SEQ ID NO:81, and wherein the one, two, three, four, or five substitution(s) comprise substitution(s) at positions of SEQ ID NO:81 selected from the group consisting of: Q11, H16, L18, L19, D20, Q22, R38, F42, K43, Y45, E62, P65, E68, V69, L72, D84, S87, N88, V91, 192, T123, Q126, S127, 1129, and S130. In some embodiments, the IL-2 polypeptide comprises the sequence of SEQ ID NO:81 with one of the following sets of amino acid substitutions (relative to the sequence of SEQ ID NO:81): R38E and F42A; R38D and F42A; F42A and E62Q; R38A and F42K; R38E, F42A, and N88S; R38E, F42A, and N88A; R38E, F42A, and N88G; R38E, F42A, and N88R; R38E, F42A, and N88T; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88R; R38D, F42A and N88T; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88R; R38A, F42K, and N88T; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88R; F42A, E62Q, and N88T; F42A, E62Q, and N88D; F42A, E62Q, and V91E; F42A, E62Q, and D84H; F42A, E62Q, and D84K; F42A, E62Q, and D84R; H16D, F42A, and E62Q; H16E, F42A, and E62Q; F42A, E62Q, and Q126S. In some embodiments, the IL-2 polypeptide comprises the sequence of SEQ ID NO:81 with a further amino acid substitution relative to SEQ ID NO:81 at position C125. In some embodiments, the IL-2 polypeptide comprises the sequence of SEQ ID NO:81 with one of the following sets of amino acid substitutions (relative to the sequence of SEQ ID NO:81): R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88R, and C125A; R38E, F42A, N88T, and C125A; R38E, F42A, N88D, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88R, and C125A; R38D, F42A, N88T, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88R, and C125A; R38A, F42K, N88T, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88R, and C125A; F42A, E62Q, N88T, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88R, and C125A; F42A, N88T, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S. In some embodiments, the IL-2 polypeptide comprises the sequence APTSSSTKKTQLQLEHLLLDLQMILNGINNYKNPKLTEMLTAKFYMPKKATELKHLQ CLEEELKPLEEVLNLAQSKNFHLRPRDLISAINVIVLELKGSETTFMCEYADETATIVE FLNRWITFAQSIISTLT (SEQ ID NO:80). In some embodiments, the IL-2 polypeptide comprises the sequence APTSSSTKKTQLQLEELLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFAQSIISTLT (SEQ ID NO:297). In some embodiments, the IL-2 polypeptide comprises a sequence selected from the group consisting of SEQ ID Nos: 85-155 and 190-216. In some embodiments, the IL-2 polypeptide comprises a sequence selected from the group consisting of SEQ ID Nos: 80, 85-155, 190-216, 297, and 354-383. In some embodiments, the second moiety comprises a polypeptide that induces signaling via IL2Rβγ. In some embodiments, the second moiety comprises an IL-21 polypeptide.

In some embodiments according to any of the embodiments described herein (e.g., a fusion protein of the present disclosure), one or both of the antibody Fc domains comprise(s) human IgG1 Fc domains with the following amino acid substitutions: L234A, L235A, G237A, and K322A, numbering according to EU index. In some embodiments, one or both of the antibody Fc domains do not have a C-terminal lysine. In some embodiments, one or both of the antibody Fc domains comprise(s) human IgG1 Fc domains with the following amino acid substitutions: L234A, L235A, and G237A, numbering according to EU index. In some embodiments, one or both of the antibody Fc domains do not have a C-terminal lysine. In some embodiments, a first of the two Fc domains comprises a human IgG1 Fc domain with amino acid substitutions Y349C and T366W, and a second of the two Fc domain comprises a human IgG1 Fc domain with amino acid substitutions S354C, T366S, L368A and Y407V, numbering according to EU index. In some embodiments, one or both of the antibody Fc domains do not have a C-terminal lysine. In some embodiments (e.g., a fusion protein of the present disclosure), the linker comprises the sequence (GGGS)xGn (SEQ ID NO:74), (GGGGS)xGn (SEQ ID NO:75), or (GGGGGS)xGn (SEQ ID NO:76), S(GGGS)xGn (SEQ ID NO:386), S(GGGGS)xGn (SEQ ID NO:387), or S(GGGGGS)xGn (SEQ ID NO:388), wherein x=1, 2, 3, 4, 5, 6, 7, 8, 9, 10, 11, or 12, and wherein n=0, 1, 2 or 3. In some embodiments, the linker comprises the sequence GGGGGGGGSGGGGS (SEQ ID NO: 79) or SGGGGSGGGGSGGGGS (SEQ ID NO:389). In some embodiments (e.g., in a fusion protein of the present disclosure), the linker connects a first moiety of the present disclosure (e.g., a human or humanized antibody or antigen-binding fragment thereof that specifically binds CD8b and/or CD8ab) and a second moiety of the present disclosure (e.g., an IL-2 polypeptide of the present disclosure, IL-21 polypeptide of the present disclosure, or a polypeptide that induces signaling via IL2Rβγ of the present disclosure).

In some embodiments according to any of the embodiments described herein, a fusion protein of the present disclosure comprises one or two light chains comprising the amino acid sequence of SEQ ID NO: 156, a heavy chain comprising the amino acid sequence of SEQ ID NO:157, and a heavy chain comprising the amino acid sequence of SEQ ID NO: 158. In some embodiments, a fusion protein of the present disclosure comprises one or two light chains comprising the amino acid sequence of SEQ ID NO:156, a heavy chain comprising the amino acid sequence of SEQ ID NO: 157, and a heavy chain comprising the amino acid sequence of SEQ ID NO:217. In some embodiments, a fusion protein of the present disclosure comprises one or two light chains comprising the amino acid sequence of SEQ ID NO:159, a heavy chain comprising the amino acid sequence of SEQ ID NO:160, and a heavy chain comprising the amino acid sequence of SEQ ID NO:161. In some embodiments, a fusion protein of the present disclosure comprises one or two light chains comprising the amino acid sequence of SEQ ID NO:159, a heavy chain comprising the amino acid sequence of SEQ ID NO: 160, and a heavy chain comprising the amino acid sequence of SEQ ID NO:218. In some embodiments, a fusion protein of the present disclosure comprises one or two light chains comprising the amino acid sequence of SEQ ID NO: 162, a heavy chain comprising the amino acid sequence of SEQ ID NO: 163, and a heavy chain comprising the amino acid sequence of SEQ ID NO: 164. In some embodiments, a fusion protein of the present disclosure comprises one or two light chains comprising the amino acid sequence of SEQ ID NO: 162, a heavy chain comprising the amino acid sequence of SEQ ID NO: 163, and a heavy chain comprising the amino acid sequence of SEQ ID NO:219. In some embodiments, a fusion protein of the present disclosure comprises one or two light chains comprising the amino acid sequence of SEQ ID NO: 165, a heavy chain comprising the amino acid sequence of SEQ ID NO:166, and a heavy chain comprising the amino acid sequence of SEQ ID NO:167. In some embodiments, a fusion protein of the present disclosure comprises one or two light chains comprising the amino acid sequence of SEQ ID NO: 165, a heavy chain comprising the amino acid sequence of SEQ ID NO:166, and a heavy chain comprising the amino acid sequence of SEQ ID NO:220. In some embodiments, a fusion protein of the present disclosure comprises one or two light chains comprising the amino acid sequence of SEQ ID NO: 168, a heavy chain comprising the amino acid sequence of SEQ ID NO: 169, and a heavy chain comprising the amino acid sequence of SEQ ID NO:170. In some embodiments, a fusion protein of the present disclosure comprises one or two light chains comprising the amino acid sequence of SEQ ID NO: 168, a heavy chain comprising the amino acid sequence of SEQ ID NO: 169, and a heavy chain comprising the amino acid sequence of SEQ ID NO:221. In some embodiments, a fusion protein of the present disclosure comprises one or two light chains comprising the amino acid sequence of SEQ ID NO:171, a heavy chain comprising the amino acid sequence of SEQ ID NO:172, and a heavy chain comprising the amino acid sequence of SEQ ID NO:173. In some embodiments, a fusion protein of the present disclosure comprises one or two light chains comprising the amino acid sequence of SEQ ID NO: 171, a heavy chain comprising the amino acid sequence of SEQ ID NO: 172, and a heavy chain comprising the amino acid sequence of SEQ ID NO:222. In some embodiments, a fusion protein of the present disclosure comprises one or two light chains comprising the amino acid sequence of SEQ ID NO:174, a heavy chain comprising the amino acid sequence of SEQ ID NO:175, and a heavy chain comprising the amino acid sequence of SEQ ID NO:176. In some embodiments, a fusion protein of the present disclosure comprises one or two light chains comprising the amino acid sequence of SEQ ID NO: 174, a heavy chain comprising the amino acid sequence of SEQ ID NO: 175, and a heavy chain comprising the amino acid sequence of SEQ ID NO:223. In some embodiments, a fusion protein of the present disclosure comprises one or two light chains comprising the amino acid sequence of SEQ ID NO:187, a heavy chain comprising the amino acid sequence of SEQ ID NO: 188, and a heavy chain comprising the amino acid sequence of SEQ ID NO:189. In some embodiments, a fusion protein of the present disclosure comprises one or two light chains comprising the amino acid sequence of SEQ ID NO:187, a heavy chain comprising the amino acid sequence of SEQ ID NO: 188, and a heavy chain comprising the amino acid sequence of SEQ ID NO:224. In some embodiments, a fusion protein of the present disclosure comprises one or two light chains comprising the amino acid sequence of SEQ ID NO:298, a heavy chain comprising the amino acid sequence of SEQ ID NO:299, and a heavy chain comprising the amino acid sequence of SEQ ID NO:300. In some embodiments, a fusion protein of the present disclosure comprises one or two light chains comprising the amino acid sequence of SEQ ID NO:298, a heavy chain comprising the amino acid sequence of SEQ ID NO:299, and a heavy chain comprising the amino acid sequence of SEQ ID NO:301. In some embodiments, a fusion protein of the present disclosure comprises one or two light chains comprising the amino acid sequence of SEQ ID NO:302, a heavy chain comprising the amino acid sequence of SEQ ID NO:303, and a heavy chain comprising the amino acid sequence of SEQ ID NO:304. In some embodiments, a fusion protein of the present disclosure comprises one or two light chains comprising the amino acid sequence of SEQ ID NO:302, a heavy chain comprising the amino acid sequence of SEQ ID NO:303, and a heavy chain comprising the amino acid sequence of SEQ ID NO:305. In some embodiments, a fusion protein of the present disclosure comprises one or two light chains comprising the amino acid sequence of SEQ ID NO:306, a heavy chain comprising the amino acid sequence of SEQ ID NO:307, and a heavy chain comprising the amino acid sequence of SEQ ID NO:308. In some embodiments, a fusion protein of the present disclosure comprises one or two light chains comprising the amino acid sequence of SEQ ID NO:306, a heavy chain comprising the amino acid sequence of SEQ ID NO:307, and a heavy chain comprising the amino acid sequence of SEQ ID NO:309. In some embodiments, a fusion protein of the present disclosure comprises one or two light chains comprising the amino acid sequence of SEQ ID NO:310, a heavy chain comprising the amino acid sequence of SEQ ID NO:311, and a heavy chain comprising the amino acid sequence of SEQ ID NO:312. In some embodiments, a fusion protein of the present disclosure comprises one or two light chains comprising the amino acid sequence of SEQ ID NO:310, a heavy chain comprising the amino acid sequence of SEQ ID NO:311, and a heavy chain comprising the amino acid sequence of SEQ ID NO:313. In some embodiments, a fusion protein of the present disclosure comprises one or two light chains comprising the amino acid sequence of SEQ ID NO:314, a heavy chain comprising the amino acid sequence of SEQ ID NO:315, and a heavy chain comprising the amino acid sequence of SEQ ID NO:316. In some embodiments, a fusion protein of the present disclosure comprises one or two light chains comprising the amino acid sequence of SEQ ID NO:314, a heavy chain comprising the amino acid sequence of SEQ ID NO:315, and a heavy chain comprising the amino acid sequence of SEQ ID NO:317. In some embodiments, a fusion protein of the present disclosure comprises one or two light chains comprising the amino acid sequence of SEQ ID NO:318, a heavy chain comprising the amino acid sequence of SEQ ID NO:319, and a heavy chain comprising the amino acid sequence of SEQ ID NO:320. In some embodiments, a fusion protein of the present disclosure comprises one or two light chains comprising the amino acid sequence of SEQ ID NO:318, a heavy chain comprising the amino acid sequence of SEQ ID NO:319, and a heavy chain comprising the amino acid sequence of SEQ ID NO:321. In some embodiments, a fusion protein of the present disclosure comprises one or two light chains comprising the amino acid sequence of SEQ ID NO:322, a heavy chain comprising the amino acid sequence of SEQ ID NO:323, and a heavy chain comprising the amino acid sequence of SEQ ID NO:324. In some embodiments, a fusion protein of the present disclosure comprises one or two light chains comprising the amino acid sequence of SEQ ID NO:322, a heavy chain comprising the amino acid sequence of SEQ ID NO:323, and a heavy chain comprising the amino acid sequence of SEQ ID NO:325. In some embodiments, a fusion protein of the present disclosure comprises one or two light chains comprising the amino acid sequence of SEQ ID NO:326, a heavy chain comprising the amino acid sequence of SEQ ID NO:327, and a heavy chain comprising the amino acid sequence of SEQ ID NO:328. In some embodiments, a fusion protein of the present disclosure comprises one or two light chains comprising the amino acid sequence of SEQ ID NO:326, a heavy chain comprising the amino acid sequence of SEQ ID NO:327, and a heavy chain comprising the amino acid sequence of SEQ ID NO:329. In some embodiments, a fusion protein of the present disclosure comprises one or two light chains comprising the amino acid sequence of SEQ ID NO:330, a heavy chain comprising the amino acid sequence of SEQ ID NO:331, and a heavy chain comprising the amino acid sequence of SEQ ID NO:332. In some embodiments, a fusion protein of the present disclosure comprises one or two light chains comprising the amino acid sequence of SEQ ID NO:330, a heavy chain comprising the amino acid sequence of SEQ ID NO:331, and a heavy chain comprising the amino acid sequence of SEQ ID NO:333. In some embodiments, a fusion protein of the present disclosure comprises one or two light chains comprising the amino acid sequence of SEQ ID NO:334, a heavy chain comprising the amino acid sequence of SEQ ID NO:335, and a heavy chain comprising the amino acid sequence of SEQ ID NO:336. In some embodiments, a fusion protein of the present disclosure comprises one or two light chains comprising the amino acid sequence of SEQ ID NO:334, a heavy chain comprising the amino acid sequence of SEQ ID NO:335, and a heavy chain comprising the amino acid sequence of SEQ ID NO:337. In some embodiments, a fusion protein of the present disclosure comprises one or two light chains comprising the amino acid sequence of SEQ ID NO:338, a heavy chain comprising the amino acid sequence of SEQ ID NO:339, and a heavy chain comprising the amino acid sequence of SEQ ID NO:340. In some embodiments, a fusion protein of the present disclosure comprises one or two light chains comprising the amino acid sequence of SEQ ID NO:338, a heavy chain comprising the amino acid sequence of SEQ ID NO:339, and a heavy chain comprising the amino acid sequence of SEQ ID NO:341. In some embodiments, a fusion protein of the present disclosure comprises one or two light chains comprising the amino acid sequence of SEQ ID NO:342, a heavy chain comprising the amino acid sequence of SEQ ID NO:343, and a heavy chain comprising the amino acid sequence of SEQ ID NO:344. In some embodiments, a fusion protein of the present disclosure comprises one or two light chains comprising the amino acid sequence of SEQ ID NO:342, a heavy chain comprising the amino acid sequence of SEQ ID NO:343, and a heavy chain comprising the amino acid sequence of SEQ ID NO:345. In some embodiments, a fusion protein of the present disclosure comprises one or two light chains comprising the amino acid sequence of SEQ ID NO:346, a heavy chain comprising the amino acid sequence of SEQ ID NO:347, and a heavy chain comprising the amino acid sequence of SEQ ID NO:348. In some embodiments, a fusion protein of the present disclosure comprises one or two light chains comprising the amino acid sequence of SEQ ID NO:346, a heavy chain comprising the amino acid sequence of SEQ ID NO:347, and a heavy chain comprising the amino acid sequence of SEQ ID NO:349. In some embodiments, a fusion protein of the present disclosure comprises one or two light chains comprising the amino acid sequence of SEQ ID NO:350, a heavy chain comprising the amino acid sequence of SEQ ID NO:351, and a heavy chain comprising the amino acid sequence of SEQ ID NO:352. In some embodiments, a fusion protein of the present disclosure comprises one or two light chains one or two light chains comprising the amino acid sequence of SEQ ID NO: 156, a heavy chain comprising the amino acid sequence of SEQ ID NO:157, and a heavy chain comprising the amino acid sequence of SEQ ID NO:217. In some embodiments, a fusion protein of the present disclosure comprises one or two light chains one or two light chains comprising the amino acid sequence of SEQ ID NO: 159, a heavy chain comprising the amino acid sequence of SEQ ID NO:160, and a heavy chain comprising the amino acid sequence of SEQ ID NO:218. In some embodiments, a fusion protein of the present disclosure comprises one or two light chains one or two light chains comprising the amino acid sequence of SEQ ID NO:162, a heavy chain comprising the amino acid sequence of SEQ ID NO: 163, and a heavy chain comprising the amino acid sequence of SEQ ID NO:219. In some embodiments, a fusion protein of the present disclosure comprises one or two light chains one or two light chains comprising the amino acid sequence of SEQ ID NO: 165, a heavy chain comprising the amino acid sequence of SEQ ID NO: 166, and a heavy chain comprising the amino acid sequence of SEQ ID NO:220. In some embodiments, a fusion protein of the present disclosure comprises one or two light chains one or two light chains comprising the amino acid sequence of SEQ ID NO: 168, a heavy chain comprising the amino acid sequence of SEQ ID NO:169, and a heavy chain comprising the amino acid sequence of SEQ ID NO:221. In some embodiments, a fusion protein of the present disclosure comprises one or two light chains one or two light chains comprising the amino acid sequence of SEQ ID NO: 171, a heavy chain comprising the amino acid sequence of SEQ ID NO:172, and a heavy chain comprising the amino acid sequence of SEQ ID NO:222. In some embodiments, a fusion protein of the present disclosure comprises one or two light chains one or two light chains comprising the amino acid sequence of SEQ ID NO: 174, a heavy chain comprising the amino acid sequence of SEQ ID NO:175, and a heavy chain comprising the amino acid sequence of SEQ ID NO:223. In some embodiments, a fusion protein of the present disclosure comprises one or two light chains one or two light chains comprising the amino acid sequence of SEQ ID NO: 187, a heavy chain comprising the amino acid sequence of SEQ ID NO: 188, and a heavy chain comprising the amino acid sequence of SEQ ID NO:224. In some embodiments, a fusion protein of the present disclosure comprises one or two light chains one or two light chains comprising the amino acid sequence of SEQ ID NO:298, a heavy chain comprising the amino acid sequence of SEQ ID NO:299, and a heavy chain comprising the amino acid sequence of SEQ ID NO:301. In some embodiments, a fusion protein of the present disclosure comprises one or two light chains one or two light chains comprising the amino acid sequence of SEQ ID NO:302, a heavy chain comprising the amino acid sequence of SEQ ID NO:303, and a heavy chain comprising the amino acid sequence of SEQ ID NO:305. In some embodiments, a fusion protein of the present disclosure comprises one or two light chains one or two light chains comprising the amino acid sequence of SEQ ID NO: 306, a heavy chain comprising the amino acid sequence of SEQ ID NO:307, and a heavy chain comprising the amino acid sequence of SEQ ID NO:309. In some embodiments, a fusion protein of the present disclosure comprises one or two light chains one or two light chains comprising the amino acid sequence of SEQ ID NO:310, a heavy chain comprising the amino acid sequence of SEQ ID NO:311, and a heavy chain comprising the amino acid sequence of SEQ ID NO:313. In some embodiments, a fusion protein of the present disclosure comprises one or two light chains one or two light chains comprising the amino acid sequence of SEQ ID NO:314, a heavy chain comprising the amino acid sequence of SEQ ID NO: 315, and a heavy chain comprising the amino acid sequence of SEQ ID NO:317. In some embodiments, a fusion protein of the present disclosure comprises one or two light chains one or two light chains comprising the amino acid sequence of SEQ ID NO:318, a heavy chain comprising the amino acid sequence of SEQ ID NO:319, and a heavy chain comprising the amino acid sequence of SEQ ID NO:321. In some embodiments, a fusion protein of the present disclosure comprises one or two light chains one or two light chains comprising the amino acid sequence of SEQ ID NO:322, a heavy chain comprising the amino acid sequence of SEQ ID NO:323, and a heavy chain comprising the amino acid sequence of SEQ ID NO:325. In some embodiments, a fusion protein of the present disclosure comprises one or two light chains one or two light chains comprising the amino acid sequence of SEQ ID NO: 326, a heavy chain comprising the amino acid sequence of SEQ ID NO:327, and a heavy chain comprising the amino acid sequence of SEQ ID NO:329. In some embodiments, a fusion protein of the present disclosure comprises one or two light chains one or two light chains comprising the amino acid sequence of SEQ ID NO:330, a heavy chain comprising the amino acid sequence of SEQ ID NO:331, and a heavy chain comprising the amino acid sequence of SEQ ID NO:333. In some embodiments, a fusion protein of the present disclosure comprises one or two light chains one or two light chains comprising the amino acid sequence of SEQ ID NO:334, a heavy chain comprising the amino acid sequence of SEQ ID NO: 335, and a heavy chain comprising the amino acid sequence of SEQ ID NO:337. In some embodiments, a fusion protein of the present disclosure comprises one or two light chains one or two light chains comprising the amino acid sequence of SEQ ID NO:338, a heavy chain comprising the amino acid sequence of SEQ ID NO:339, and a heavy chain comprising the amino acid sequence of SEQ ID NO:341. In some embodiments, a fusion protein of the present disclosure comprises one or two light chains one or two light chains comprising the amino acid sequence of SEQ ID NO:342, a heavy chain comprising the amino acid sequence of SEQ ID NO:343, and a heavy chain comprising the amino acid sequence of SEQ ID NO:345. In some embodiments, a fusion protein of the present disclosure comprises one or two light chains one or two light chains comprising the amino acid sequence of SEQ ID NO: 346, a heavy chain comprising the amino acid sequence of SEQ ID NO:347, and a heavy chain comprising the amino acid sequence of SEQ ID NO:349. In some embodiments, a fusion protein of the present disclosure comprises one or two light chains one or two light chains comprising the amino acid sequence of SEQ ID NO:350, a heavy chain comprising the amino acid sequence of SEQ ID NO:351, and a heavy chain comprising the amino acid sequence of SEQ ID NO:353. In some embodiments, the fusion protein comprises one or two antigen binding sites, each antigen binding site comprising a VL domain from one of the light chain(s) and a VH domain from one of the heavy chains (e.g., the fusion protein comprises two antigen binding sites: one comprising a VL domain from one of the two light chains and a VH domain from one of the heavy chains, and another comprising a VL domain from the other light chain and a VH domain from the other heavy chain).

In some embodiments according to any of the embodiments described herein, a fusion protein of the present disclosure comprises one or two polypeptides comprising the amino acid sequence of SEQ ID NO:156, a polypeptide comprising the amino acid sequence of SEQ ID NO: 157, and a polypeptide comprising the amino acid sequence of SEQ ID NO: 158. In some embodiments, a fusion protein of the present disclosure comprises one or two polypeptides comprising the amino acid sequence of SEQ ID NO: 156, a polypeptide comprising the amino acid sequence of SEQ ID NO:157, and a polypeptide comprising the amino acid sequence of SEQ ID NO:217. In some embodiments, a fusion protein of the present disclosure comprises one or two polypeptides comprising the amino acid sequence of SEQ ID NO: 159, a polypeptide comprising the amino acid sequence of SEQ ID NO:160, and a polypeptide comprising the amino acid sequence of SEQ ID NO:161. In some embodiments, a fusion protein of the present disclosure comprises one or two polypeptides comprising the amino acid sequence of SEQ ID NO: 159, a polypeptide comprising the amino acid sequence of SEQ ID NO: 160, and a polypeptide comprising the amino acid sequence of SEQ ID NO:218. In some embodiments, a fusion protein of the present disclosure comprises one or two polypeptides comprising the amino acid sequence of SEQ ID NO:162, a polypeptide comprising the amino acid sequence of SEQ ID NO: 163, and a polypeptide comprising the amino acid sequence of SEQ ID NO:164. In some embodiments, a fusion protein of the present disclosure comprises one or two polypeptides comprising the amino acid sequence of SEQ ID NO: 162, a polypeptide comprising the amino acid sequence of SEQ ID NO: 163, and a polypeptide comprising the amino acid sequence of SEQ ID NO:219. In some embodiments, a fusion protein of the present disclosure comprises one or two polypeptides comprising the amino acid sequence of SEQ ID NO: 165, a polypeptide comprising the amino acid sequence of SEQ ID NO:166, and a polypeptide comprising the amino acid sequence of SEQ ID NO: 167. In some embodiments, a fusion protein of the present disclosure comprises one or two polypeptides comprising the amino acid sequence of SEQ ID NO:165, a polypeptide comprising the amino acid sequence of SEQ ID NO:166, and a polypeptide comprising the amino acid sequence of SEQ ID NO:220. In some embodiments, a fusion protein of the present disclosure comprises one or two polypeptides comprising the amino acid sequence of SEQ ID NO:168, a polypeptide comprising the amino acid sequence of SEQ ID NO: 169, and a polypeptide comprising the amino acid sequence of SEQ ID NO:170. In some embodiments, a fusion protein of the present disclosure comprises one or two polypeptides comprising the amino acid sequence of SEQ ID NO:168, a polypeptide comprising the amino acid sequence of SEQ ID NO:169, and a polypeptide comprising the amino acid sequence of SEQ ID NO:221. In some embodiments, a fusion protein of the present disclosure comprises one or two polypeptides comprising the amino acid sequence of SEQ ID NO: 171, a polypeptide comprising the amino acid sequence of SEQ ID NO: 172, and a polypeptide comprising the amino acid sequence of SEQ ID NO:173. In some embodiments, a fusion protein of the present disclosure comprises one or two polypeptides comprising the amino acid sequence of SEQ ID NO: 171, a polypeptide comprising the amino acid sequence of SEQ ID NO: 172, and a polypeptide comprising the amino acid sequence of SEQ ID NO:222. In some embodiments, a fusion protein of the present disclosure comprises one or two polypeptides comprising the amino acid sequence of SEQ ID NO:174, a polypeptide comprising the amino acid sequence of SEQ ID NO: 175, and a polypeptide comprising the amino acid sequence of SEQ ID NO: 176. In some embodiments, a fusion protein of the present disclosure comprises one or two polypeptides comprising the amino acid sequence of SEQ ID NO:174, a polypeptide comprising the amino acid sequence of SEQ ID NO: 175, and a polypeptide comprising the amino acid sequence of SEQ ID NO:223. In some embodiments, a fusion protein of the present disclosure comprises one or two polypeptides comprising the amino acid sequence of SEQ ID NO:187, a polypeptide comprising the amino acid sequence of SEQ ID NO: 188, and a polypeptide comprising the amino acid sequence of SEQ ID NO: 189. In some embodiments, a fusion protein of the present disclosure comprises one or two polypeptides comprising the amino acid sequence of SEQ ID NO: 187, a polypeptide comprising the amino acid sequence of SEQ ID NO: 188, and a polypeptide comprising the amino acid sequence of SEQ ID NO:224. In some embodiments, a fusion protein of the present disclosure comprises one or two polypeptides comprising the amino acid sequence of SEQ ID NO:298, a polypeptide comprising the amino acid sequence of SEQ ID NO:299, and a polypeptide comprising the amino acid sequence of SEQ ID NO:300. In some embodiments, a fusion protein of the present disclosure comprises one or two polypeptides comprising the amino acid sequence of SEQ ID NO:298, a polypeptide comprising the amino acid sequence of SEQ ID NO:299, and a polypeptide comprising the amino acid sequence of SEQ ID NO:301. In some embodiments, a fusion protein of the present disclosure comprises one or two polypeptides comprising the amino acid sequence of SEQ ID NO:302, a polypeptide comprising the amino acid sequence of SEQ ID NO:303, and a polypeptide comprising the amino acid sequence of SEQ ID NO:304. In some embodiments, a fusion protein of the present disclosure comprises one or two polypeptides comprising the amino acid sequence of SEQ ID NO:302, a polypeptide comprising the amino acid sequence of SEQ ID NO:303, and a polypeptide comprising the amino acid sequence of SEQ ID NO:305. In some embodiments, a fusion protein of the present disclosure comprises one or two polypeptides comprising the amino acid sequence of SEQ ID NO:306, a polypeptide comprising the amino acid sequence of SEQ ID NO: 307, and a polypeptide comprising the amino acid sequence of SEQ ID NO:308. In some embodiments, a fusion protein of the present disclosure comprises one or two polypeptides comprising the amino acid sequence of SEQ ID NO:306, a polypeptide comprising the amino acid sequence of SEQ ID NO:307, and a polypeptide comprising the amino acid sequence of SEQ ID NO:309. In some embodiments, a fusion protein of the present disclosure comprises one or two polypeptides comprising the amino acid sequence of SEQ ID NO:310, a polypeptide comprising the amino acid sequence of SEQ ID NO:311, and a polypeptide comprising the amino acid sequence of SEQ ID NO:312. In some embodiments, a fusion protein of the present disclosure comprises one or two polypeptides comprising the amino acid sequence of SEQ ID NO:310, a polypeptide comprising the amino acid sequence of SEQ ID NO:311, and a polypeptide comprising the amino acid sequence of SEQ ID NO:313. In some embodiments, a fusion protein of the present disclosure comprises one or two polypeptides comprising the amino acid sequence of SEQ ID NO:314, a polypeptide comprising the amino acid sequence of SEQ ID NO:315, and a polypeptide comprising the amino acid sequence of SEQ ID NO:316. In some embodiments, a fusion protein of the present disclosure comprises one or two polypeptides comprising the amino acid sequence of SEQ ID NO:314, a polypeptide comprising the amino acid sequence of SEQ ID NO: 315, and a polypeptide comprising the amino acid sequence of SEQ ID NO:317. In some embodiments, a fusion protein of the present disclosure comprises one or two polypeptides comprising the amino acid sequence of SEQ ID NO:318, a polypeptide comprising the amino acid sequence of SEQ ID NO:319, and a polypeptide comprising the amino acid sequence of SEQ ID NO:320. In some embodiments, a fusion protein of the present disclosure comprises one or two polypeptides comprising the amino acid sequence of SEQ ID NO:318, a polypeptide comprising the amino acid sequence of SEQ ID NO:319, and a polypeptide comprising the amino acid sequence of SEQ ID NO:321. In some embodiments, a fusion protein of the present disclosure comprises one or two polypeptides comprising the amino acid sequence of SEQ ID NO:322, a polypeptide comprising the amino acid sequence of SEQ ID NO:323, and a polypeptide comprising the amino acid sequence of SEQ ID NO:324. In some embodiments, a fusion protein of the present disclosure comprises one or two polypeptides comprising the amino acid sequence of SEQ ID NO:322, a polypeptide comprising the amino acid sequence of SEQ ID NO:323, and a polypeptide comprising the amino acid sequence of SEQ ID NO:325. In some embodiments, a fusion protein of the present disclosure comprises one or two polypeptides comprising the amino acid sequence of SEQ ID NO:326, a polypeptide comprising the amino acid sequence of SEQ ID NO: 327, and a polypeptide comprising the amino acid sequence of SEQ ID NO:328. In some embodiments, a fusion protein of the present disclosure comprises one or two polypeptides comprising the amino acid sequence of SEQ ID NO:326, a polypeptide comprising the amino acid sequence of SEQ ID NO:327, and a polypeptide comprising the amino acid sequence of SEQ ID NO:329. In some embodiments, a fusion protein of the present disclosure comprises one or two polypeptides comprising the amino acid sequence of SEQ ID NO:330, a polypeptide comprising the amino acid sequence of SEQ ID NO:331, and a polypeptide comprising the amino acid sequence of SEQ ID NO:332. In some embodiments, a fusion protein of the present disclosure comprises one or two polypeptides comprising the amino acid sequence of SEQ ID NO:330, a polypeptide comprising the amino acid sequence of SEQ ID NO:331, and a polypeptide comprising the amino acid sequence of SEQ ID NO:333. In some embodiments, a fusion protein of the present disclosure comprises one or two polypeptides comprising the amino acid sequence of SEQ ID NO:334, a polypeptide comprising the amino acid sequence of SEQ ID NO:335, and a polypeptide comprising the amino acid sequence of SEQ ID NO:336. In some embodiments, a fusion protein of the present disclosure comprises one or two polypeptides comprising the amino acid sequence of SEQ ID NO:334, a polypeptide comprising the amino acid sequence of SEQ ID NO: 335, and a polypeptide comprising the amino acid sequence of SEQ ID NO:337. In some embodiments, a fusion protein of the present disclosure comprises one or two polypeptides comprising the amino acid sequence of SEQ ID NO:338, a polypeptide comprising the amino acid sequence of SEQ ID NO:339, and a polypeptide comprising the amino acid sequence of SEQ ID NO:340. In some embodiments, a fusion protein of the present disclosure comprises one or two polypeptides comprising the amino acid sequence of SEQ ID NO:338, a polypeptide comprising the amino acid sequence of SEQ ID NO:339, and a polypeptide comprising the amino acid sequence of SEQ ID NO:341. In some embodiments, a fusion protein of the present disclosure comprises one or two polypeptides comprising the amino acid sequence of SEQ ID NO:342, a polypeptide comprising the amino acid sequence of SEQ ID NO:343, and a polypeptide comprising the amino acid sequence of SEQ ID NO:344. In some embodiments, a fusion protein of the present disclosure comprises one or two polypeptides comprising the amino acid sequence of SEQ ID NO:342, a polypeptide comprising the amino acid sequence of SEQ ID NO:343, and a polypeptide comprising the amino acid sequence of SEQ ID NO:345. In some embodiments, a fusion protein of the present disclosure comprises one or two polypeptides comprising the amino acid sequence of SEQ ID NO:346, a polypeptide comprising the amino acid sequence of SEQ ID NO: 347, and a polypeptide comprising the amino acid sequence of SEQ ID NO:348. In some embodiments, a fusion protein of the present disclosure comprises one or two polypeptides comprising the amino acid sequence of SEQ ID NO:346, a polypeptide comprising the amino acid sequence of SEQ ID NO:347, and a polypeptide comprising the amino acid sequence of SEQ ID NO:349. In some embodiments, a fusion protein of the present disclosure comprises one or two polypeptides comprising the amino acid sequence of SEQ ID NO:350, a polypeptide comprising the amino acid sequence of SEQ ID NO:351, and a polypeptide comprising the amino acid sequence of SEQ ID NO:352. In some embodiments, a fusion protein of the present disclosure comprises one or two polypeptides comprising the amino acid sequence of SEQ ID NO:350, a polypeptide comprising the amino acid sequence of SEQ ID NO:351, and a polypeptide comprising the amino acid sequence of SEQ ID NO:353.

In some embodiments according to any of the embodiments described herein, the fusion protein comprises a first moiety that binds to a human CD8b and a second moiety comprising an IL2 polypeptide, wherein the fusion protein comprises four polypeptide chains, wherein: (1) the first polypeptide chain comprises the amino acid sequence of SEQ ID NO: 334, the second polypeptide chain comprises the amino acid sequence of SEQ ID NO: 335, the third polypeptide chain comprises the amino acid sequence of SEQ ID NO:336, and the fourth polypeptide chain comprises the amino acid sequence of SEQ ID NO:334; (2) the first polypeptide chain comprises the amino acid sequence of SEQ ID NO:334, the second polypeptide chain comprises the amino acid sequence of SEQ ID NO:335, the third polypeptide chain comprises the amino acid sequence of SEQ ID NO:337, and the fourth polypeptide chain comprises the amino acid sequence of SEQ ID NO:334; (3) the first polypeptide chain comprises the amino acid sequence of SEQ ID NO:338, the second polypeptide chain comprises the amino acid sequence of SEQ ID NO:339, the third polypeptide chain comprises the amino acid sequence of SEQ ID NO:340, and the fourth polypeptide chain comprises the amino acid sequence of SEQ ID NO:338; or (4) the first polypeptide chain comprises the amino acid sequence of SEQ ID NO:338, the second polypeptide chain comprises the amino acid sequence of SEQ ID NO:339, the third polypeptide chain comprises the amino acid sequence of SEQ ID NO:341, and the fourth polypeptide chain comprises the amino acid sequence of SEQ ID NO:338.

In some embodiments according to any of the embodiments described herein, the fusion protein is administered in a pharmaceutical composition comprising the fusion protein and a pharmaceutically acceptable carrier.

In some embodiments according to any of the embodiments described herein, the methods further comprise administering to the individual a second antiviral or immunomodulatory agent. In some embodiments, the second antiviral or immunomodulatory agent is a nucleoside or nucleotide analog, capsid assembly modulator or inhibitor, TLR agonist, vaccine, RNAi-based agent, interferon-α, HBV entry inhibitor, covalently closed circular DNA (cccDNA) disruptor, HBV transcription inhibitor, CD3 bispecific T-cell redirection agent, HBV antigen targeting agent, PD1 and/or PDL1 blocking agent, agent that reduces PDL1 expression in hepatocytes, RNA destabilizer, or Hepatitis B surface antigen (HBsAg) release inhibitor. In some embodiments, the second antiviral or immunomodulatory agent is an antiretroviral therapy (ART), PD1 and/or PDL1 blocking agent, or agent that reduces PDL1 expression. In some embodiments, the ART comprises a nucleoside reverse transcriptase inhibitor (NRTI), non-nucleoside reverse transcriptase inhibitor (NNRTI), protease inhibitor (PI), fusion inhibitor, CCR5 antagonist, integrase strand transfer inhibitor (INSTI), attachment inhibitor, post-attachment inhibitor, pharmacokinetic enhancer, or combination thereof.

It is to be understood that one, some, or all of the properties of the various embodiments described herein may be combined to form other embodiments of the present disclosure. These and other aspects of the disclosure will become apparent to one of skill in the art. These and other embodiments of the disclosure are further described by the detailed description that follows.

### BRIEF DESCRIPTION OF THE DRAWINGS

FIG. 1 shows a general mechanism for how targeted fusions of mutant IL-2 polypeptides with CD8 antigen binding molecules (CD8-targeted IL-2 fusions), and untargeted IL-2 fusions work to stimulate cells expressing or not expressing CD8 antigens.

FIG. 2A depicts three different fusion protein formats (formats A, B, and C), in accordance with some embodiments.

FIG. 2B depicts the in vitro activity profile of a murine CD8+ T cell targeted IL-2 fusion protein (mCD8-mIL2v1) and a control untargeted IL-2 fusion protein representative of the “not alpha” IL-2 class (CTRL-mIL2v2) in format A. Signaling as assessed by pSTAT5 staining of murine splenocytes following a 25 minute stimulation is shown.

FIG. 3 shows the study design in which CD8-targeted IL-2 fusion protein was tested in a mouse model of hepatitis B virus (HBV), as described in Example 3. Naïve T cell receptor (TCR) transgenic HBV core antigen-reactive CD8+ T cells (Cor93 T cells) were adoptively transferred into HBV replication-competent transgenic mice, which constitutively express all HBV antigens in the liver. Mice were treated with CD8-IL2 fusion protein or control intervention, and viral levels and CD8+ T cell responses were assessed.

FIGS. 4A-4D show the results from an experiment with HBV transgenic mice as depicted in FIG. 3. Mice received 105 Cor93 T cells and were treated with CD8-targeted IL-2 fusion protein, mCD8-mIL2v1, at 0.3 mg/kg intravenously on day 1. Shown are serum HBV Core DNA (FIG. 4A), serum ALT activity (FIG. 4B), body weight (FIG. 4C), and liver staining for HBV core antigen (HBcAg) by immunohistochemistry (IHC) (FIG. 4D) analyzed on day 5 following T cell transfer, comparing mice treated with CD8-IL2 fusion protein or control intervention.

FIGS. 5A-5D show additional results from the experiment in FIGS. 4A-4D describing HBV antigen-reactive CD8+ T cells in the liver. Shown are the number of HBV antigen-reactive CD8+ T cells (FIG. 5A), their PD-1 expression (FIG. 5B), percent of cells expressing IFNγ in response to ex vivo stimulation with HBV peptide (FIG. 5C), and percent of cells expressing GzmB (FIG. 5D) when analyzed on day 5 following T cell transfer.

FIGS. 6A-6C show the results from Example 4 using HBV transgenic mice as depicted in FIG. 3. Mice received 105 Cor93 T cells and were treated with mCD8-mIL2v1 fusion protein or a control untargeted “not α” IL-2 fusion protein, CTRL-mIL2v2, intravenously at 0.1 or 0.3 mg/kg on day 1. Shown are serum HBV Core DNA (FIG. 6A), serum ALT activity (FIG. 6B), and liver staining for HBV core antigen (HBcAg) by immunohistochemistry (IHC) in all the indicated treatment groups (FIG. 6C) analyzed on day 5 following T cell transfer.

FIGS. 7A-7C show additional results from Example 4 describing HBV antigen-reactive CD8+ T cells in the liver. Shown are the number of HBV antigen-reactive CD8+ T cells (FIG. 7A), percent of cells expressing IFNγ in response to ex vivo stimulation with HBV peptide (FIG. 7B), and percent of cells expressing GzmB (FIG. 7C) when analyzed on day 5 following T cell transfer.

FIGS. 8A & 8B show additional results from Example 4 describing NK cells in the liver and T regulatory (Treg) cells in the spleen. Shown are the frequency of NK cells as a fraction of intrahepatic leukocytes in the liver (FIG. 8A) and absolute Treg cell counts in the spleen (FIG. 8B).

### DETAILED DESCRIPTION

### Definitions

As used in this specification and the appended claims, the singular forms “a”, “an” and “the” include plural referents unless the content clearly dictates otherwise. Thus, for example, reference to “a molecule” optionally includes a combination of two or more such molecules, and the like.

It is understood that aspects and embodiments of the present disclosure include “comprising,” “consisting,” and “consisting essentially of” aspects and embodiments.

The term “about” as used herein refers to the usual error range for the respective value readily known to the skilled person in this technical field. Reference to “about” a value or parameter herein includes (and describes) embodiments that are directed to that value or parameter per se.

“Immune cells” as used here are cells of the immune system that react to organisms or other entities that are deemed foreign to the immune system of the host. They protect the host against foreign pathogens, organisms and diseases. Immune cells, also called leukocytes, are involved in both innate and adaptive and immune responses to fight pathogens. Innate immune responses occur immediately upon exposure to pathogens without additional priming or learning processes. Adaptive immune processes require initial priming, and subsequently create memory, which in turn leads to enhanced responsiveness during subsequent encounters with the same pathogen. Innate immune cells include, but are not limited to monocytes, macrophages, dendritic cells, innate lymphoid cells (ILCs) including natural killer (NK) cells, neutrophils, megakaryocytes, eosinophils and basophils. Adaptive immune cells include B and T lymphocytes/cells. T cells subsets include, but are not limited to, alpha beta CD4+ T (naïve CD4+, memory CD4+, effector memory CD4+, effector CD4+, regulatory CD4+), and alpha beta CD8+ T (naïve CD8+, memory CD8+, effector memory CD8+, effector CD8+). B cell subsets include, but is not limited to, naïve B, memory B, and plasma cells. NK T cells and T gamma delta (Tγδ) cells exhibit properties of both innate and adaptive lymphocytes.

“T cells” or “T lymphocytes” are immune cells that play a key role in the orchestration of immune responses in health and disease. Two major T cell subsets exist that have unique functions and properties: T cells that express the CD8 antigen (CD8⁺ T cells) are cytotoxic or killer T cells that can lyse target cells using the cytotoxic proteins such as granzymes and perforin; and T cells that express the CD4 antigen (CD4⁺ T cells) are helper T cells that are capable of regulating the function of many other immune cell types including that of CD8⁺ T cells, B cells, macrophages etc. Furthermore, CD4⁺ T cells are further subdivided into several subsets such as: T regulatory (Treg) cells that are capable of suppressing the immune response, and T helper 1 (Th1), T helper 2 (Th2), and T helper 17 (Th17) cells that regulate different types of immune responses by secreting immunomodulatory proteins such as cytokines. T cells recognize their targets via alpha beta T cell receptors that bind to unique antigen-specific motifs and this recognition mechanism is generally required in order to trigger their cytotoxic and cytokine-secreting functions. “Innate lymphocytes” can also exhibit properties of CD8⁺ and CD4⁺ T cells, such as the cytotoxic activity or the secretion of Th1, Th2, and Th17 cytokines. Some of these innate lymphocyte subsets include NK cells and ILC1, ILC2, and ILC3 cells; and innate-like T cells such as Tγδ cells; and NK T cells. Typically, these cells can rapidly respond to inflammatory stimuli from infected or injured tissues, such as immunomodulatory cytokines, but unlike alpha beta T cells, they can respond without the need to recognize antigen-specific patterns.

“Cytokine” is a form of immunomodulatory polypeptide that mediates cross-talk between initiating/primary cells and target/effector cells. It can function as a soluble form or cell-surface associated to bind the “cytokine receptor” on target immune cells to activate signaling. “Cytokine receptor” as used here is the polypeptide on the cell surface that activates intracellular signaling upon binding the cytokine on the extracellular cell surface. Cytokines includes, but are not limited to, chemokines, interferons, interleukins, lymphokines, and tumor necrosis factors. Cytokines are produced by a wide range of cells, including immune cells, endothelial cells, fibroblasts, and stromal cells. A given cytokine may be produced by more than one cell type. Cytokine are pleiotropic; since the receptors are expressed on multiple immune cell subsets, one cytokine can activate the signaling pathway in multiple cells. However, depending on the cell type, the signaling events for a cytokine can result in different downstream cellular events such as activation, proliferation, survival, apoptosis, effector function and secretion of other immunomodulatory proteins.

“Amino acid” as used here refers to naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

“Polypeptide” or “protein” as used here refers to a molecule where monomers (amino acids) are linearly linked to one another by peptide bonds (also known as amide bonds). The term “polypeptide” refers to any chain of two or more amino acids and does not refer to a specific length of the product. Thus, peptides, dipeptides, tripeptides, oligopeptides, “protein”, “amino acid chain”, or any other term used to refer to a chain of two or more amino acids, are included within the definition of “polypeptide”, and the term “polypeptide” may be used instead of, or interchangeably with any of these terms. The term “polypeptide” is also intended to refer to the products of A polypeptide may be derived from a natural biological source or produced by recombinant technology but is not necessarily translated from a designated nucleic acid sequence. It may be generated in any manner, including by chemical synthesis. Polypeptides normally have a defined three-dimensional structure, but they do not necessarily have such structure. A polypeptide of the present disclosure may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides with a defined three-dimensional structure are referred to as folded, and polypeptides which do not possess a defined three-dimensional structure, but rather can adopt many different conformations and are referred to as unfolded. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. The terms “polypeptide” and “protein” also refer to modified polypeptides/proteins wherein the post-expression modification is affected including without limitation glycosylation, acetylation, phosphorylation, amidation, derivatization by known protecting/blocking groups, proteolytic cleavage, or modification by non-naturally occurring amino acids.

“Residue” as used herein is meant a position in a protein and its associated amino acid identity. For example, Leu 234 (also referred to as Leu234 or L234) is a residue at position 234 in the human antibody IgG1.

“Wild-type” herein means an amino acid sequence or a nucleotide sequence that is found in nature, including allelic variations. A wild-type protein has an amino acid sequence or a nucleotide sequence that has not been intentionally modified.

“Substitution” or “mutation” refers to a change to the polypeptide backbone wherein an amino acid occurring naturally in the wild-type sequence of a polypeptide is substituted to another amino acid not naturally occurring at the same position in the said polypeptide. Preferably, a mutation or mutations are introduced to modify polypeptide's affinity to its receptor thereby altering its activity such that it becomes different from the affinity and activity of the wild-type cognate polypeptide. Mutations can also improve polypeptide's biophysical properties. Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

“CD8” as used here refers to any native human CD8. Unless otherwise indicated expressly or by context, references to “CD8” refer to CD8aa and/or CD8ab. The amino acid sequence of an exemplary human CD8b, the beta chain of human CD8, is described under UniProt P10966 (CD8B_HUMAN). “CD8a” refers to the alpha chain of human CD8 (e.g., as is described under UniProt P01732 (CD8A_HUMAN)). “CD8aa” refers to a homodimer of CD8a. “CD8ab” refers to a heterodimer of CD8a and CD8b. “CD8,” “CD8a,” “CD8b,” “CD8aa,” and “CD8ab” encompass unprocessed forms as well as mature forms that result from processing in the cell. “CD8,” “CD8a,” “CD8b,” “CD8aa,” and “CD8ab” also include but are not limited to naturally occurring variants, e.g. allelic or splice variants or variants.

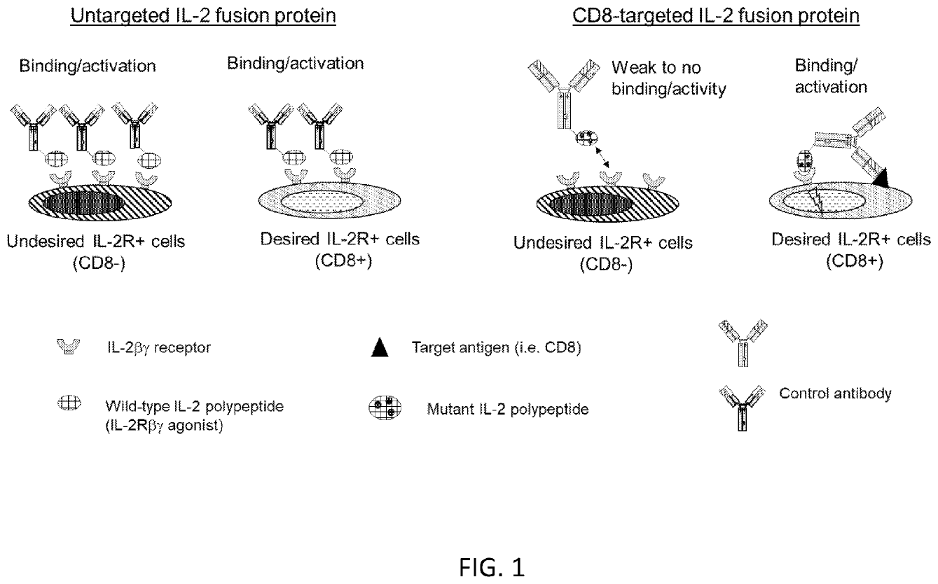

“Interleukin-2” or “IL-2” as used here refers to any native human IL-2, unless otherwise indicated. “IL-2” encompasses unprocessed IL-2 as well as “mature IL-2” which is a form of IL-2 that results from processing in the cell. The sequence of “mature IL-2” is depicted in FIG. 1A. One exemplary form of unprocessed human IL-2 comprises of an additional N-terminal amino acid signal peptide attached to mature IL-2. “IL-2” also includes but is not limited to naturally occurring variants of IL-2, e.g. allelic or splice variants or variants. The amino acid sequence of an exemplary human IL-2 is described under UniProt P60568 (IL2_HUMAN).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity can generally be represented by the dissociation constant (K_{D}), which is the ratio of dissociation and association rate constants (koff and kon, respectively). Thus, equivalent affinities may comprise different rate constants, as long as the ratio of the rate constants remains the same. Affinity can be measured by common methods known in the art, such as enzyme-linked immunosorbent assay (ELISA), surface plasmon resonance (SPR) technologies (e.g. BIAcore), BioLayer Interferometry (BLI) technologies (e.g. Octet) and other traditional binding assays (Heeley, Endocr Res 28, 217-229 (2002).

“Binding” or “specific binding” as used here, refers the ability of a polypeptide or an antigen binding molecule to selectively interact with the receptor for the polypeptide or target antigen, respectively, and this specific interaction can be distinguished from non-targeted or undesired or non-specific interactions. Examples of specific binding include but are not limited to IL-2 cytokine binding to its specific receptors (e.g. IL-2Rα, IL-2Rβ and IL-2Rγ) and an antigen binding molecule binding to a specific antigen (e.g. CD8 or PD-1).

“Mutant IL-2 polypeptide” refers to IL-2 polypeptide that has reduced affinity to its receptor wherein such decreased affinity will result in reduced biological activity of the mutant. Reduction in affinity and thereby activity can be obtained by introducing a small number of amino acid mutations or substitutions. The mutant IL-2 polypeptides can also have other modifications to the peptide backbone, including but not limited to amino acid deletion, permutation, cyclization, disulfide bonds, or the post-translational modifications (e.g. glycosylation or altered carbohydrate) of a polypeptide, chemical or enzymatic modifications to the polypeptide (e.g. attaching PEG to the polypeptide backbone), addition of peptide tags or labels, or fusion to proteins or protein domains to generate a final construct with desired characteristics, such as reduced affinity to IL-2Rβγ. Desired activity may also include improved biophysical properties compared to the wild-type IL-2 polypeptide. Multiple modifications may be combined to achieve desired activity modification, such as reduction in affinity or improved biophysical properties. As a non-limiting example, amino acid sequences for consensus N-link glycosylation may be incorporated into the polypeptide to allow for glycosylation. Another non-limiting example is that a lysine may be incorporated onto the polypeptide to enable pegylation. Preferably, a mutation or mutations are introduced to the polypeptide to modify its activity.

The terms “antibody” and “immunoglobulin” are used interchangeably and herein are used in the broadest sense and encompasses various antibody structures, including but not limited to monoclonal antibodies (e.g., full length or intact monoclonal antibodies), polyclonal antibodies, multispecific antibodies (e.g. bispecific antibodies), antibody fragments and single domain antibody (as described in greater detail herein), so long as they exhibit the desired antigen binding activity.

Antibodies (immunoglobulins) refer to a protein having a structure substantially similar to a native antibody structure. “Native antibodies” refer to naturally occurring immunoglobulin molecules with varying structures. For example, native immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The subunit structures and three-dimensional configurations of the different classes of immunoglobulins are well known and described generally, for example, in Abbas et al., 2000, Cellular and Mol, and Kindt et al., Kuby Immunology, 6th ed., W. H. Freeman and Co., page 91 (2007). Antibodies (immunoglobulins) are assigned to different classes, depending on the amino acid sequences of the heavy chain constant domains. There are five major classes of antibodies: α (IgA), δ (IgD), ϵ (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

“Fc” or “Fc region” or “Fc domain” as used herein refers to the C-terminal region of an antibody heavy chain that contains at least a portion of the constant region. The term includes native sequence Fc regions and variant Fc regions. An Fc can refer to the last two constant region immunoglobulin domains (e.g., CH2 and CH3) of IgA, IgD, and IgG, the last three constant region immunoglobulin domains of IgE and IgM, and optionally, all or a portion of the flexible hinge N-terminal to these domains. For IgA and IgM, Fc may include the J chain. An IgG Fc region comprises an IgG CH2 and an IgG CH3 domain and in some cases, inclusive of the hinge. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991. The “hinge” region usually extends from amino acid residue at about position 216 to amino acid residue at about position 230. The hinge region herein may be a native hinge domain or variant hinge domain. The “CH2 domain” of a human IgG Fc region usually extends from an amino acid residue at about position 231 to an amino acid residue at about position 340. The CH2 domain herein may be a native sequence CH2 domain or variant CH2 domain. The “CH3 domain” comprises the stretch of residues C-terminal to a CH2 domain in an Fc region, from an amino acid residue at about position 341 to an amino acid residue at about position 447 of an IgG. The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Thus, the definition of “Fc domain” includes both amino acids 231-447 (CH2-CH3) or 216-447 (hinge-CH2-CH3), or fragments thereof. An “Fc fragment” in this context may contain fewer amino acids from either or both of the N- and C-termini but still retains the ability to form a dimer with another Fc domain or Fc fragment as can be detected using standard methods, generally based on size (e.g. non-denaturing chromatography, size exclusion chromatography, etc.). Human IgG Fc domains are of particular use in the present disclosure, and can be the Fc domain from human IgG1, IgG2 or IgG4.

A “variant Fc domain” or “Fc variant” or “variant Fc” contains amino acid modifications (e.g. substitution, addition, and deletion) as compared to a parental Fc domain. The term also includes naturally occurring allelic variants of the Fc region of an immunoglobulin. In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. For example, one or more amino acids can be deleted from the N-terminus or C-terminus of the Fc region of an immunoglobulin without substantial loss of biological function. Additionally, as discussed herein, the variant Fc domains herein still retain the ability to form a dimer with another Fc domain as measured using known techniques as described herein, such as non-denaturing gel electrophoresis.

“Fc gamma receptor”, “FcγR” or “Fc gamma R” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “effector function” as used herein is meant a biochemical event that results from the interaction of an antibody Fc region with an Fc receptor or ligand, which vary with the antibody isotype. Effector functions include but are not limited to antibody-dependent cell-mediated cytotoxicity (ADCC), antibody-dependent cell-mediated phagocytosis (ADCP), complement-dependent cytotoxicity (CDC), cytokine secretion, immune complex-mediated antigen uptake by antigen presenting cells, down regulation of cell surface receptors (e.g. B cell receptor), and B cell activation. “Antibody-dependent cell-mediated cytotoxicity” or “ADCC” refer to a cell-mediated reaction in which nonspecific cytotoxic cells that express FcRs (such as Natural Killer (NK) cells, neutrophils, and macrophages) recognize bound antibody on a target cell and subsequently cause lysis of the target cell. ADCC is correlated with binding to FcγRIIIa; increased binding to FcγRIIIa leads to an increase in ADCC activity. To assess ADCC activity of a molecule of interest, an in vitro ADCC assay such as that described in U.S. Pat. No. 5,500,362 or 5,821,337 may be performed. “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

“Fc null” and “Fc null variant” are used interchangeably and used herein to describe a modified Fc which have reduced or abolished effector functions. Such Fc null or Fc null variant have reduced or abolished to FcγRs and/or complement receptors. Preferably, such Fc null or Fc null variant has abolished effector functions. Exemplary methods for the modification include but not limited to chemical alteration, amino acid residue substitution, insertion and deletions. Exemplary amino acid positions on Fc molecules where one or more modifications were introduced to decrease effector function of the resulting variant (numbering based on the EU numbering scheme) at position i) IgG1: C220, C226, C229, E233, L234, L235, G237, P238, S239 D265, S267, N297, L328, P331, K322, A327 and P329, ii) IgG2: V234, G237, D265, H268, N297, V309, A330, A331, K322 and iii) IgG4: L235, G237, D265 and E318. Exemplary Fc molecules having decreased effector function include those having one or more of the following substitutions: i) IgG1: N297A, N297Q, N297G, D265A/N297A, D265A/N297Q, C220S/C226S/C229S/P238S, S267E/L328F, C226S/C229S/E233P/L234V/L235A, L234F/L235E/P331S, L234A/L235A, L234A/L235A/G237A, L234A/L235A/G237A/K322A, L234A/L235A/G237A/A330S/A331S, L234A/L235A/P329G,E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, L234A/L235A/G237deleted; ii) IgG2: A330S/A331S, V234A/G237A, V234A/G237A/D265A, D265A/A330S/A331S, V234A/G237A/D265A/A330S/A331S, and H268Q/V309L/A330S/A331S; iii) IgG4: L235A/G237A/E318A, D265A, L235A/G237A/D265A and L235A/G237A/D265A/E318A.

“Epitope” as used herein refers to a determinant capable of specific binding to the variable region of an antibody molecule known as a paratope. Epitopes are groupings of molecules such as amino acids or sugar side chains and usually have specific structural characteristics, as well as specific charge characteristics. A single antigen may have more than one epitope. The epitope may comprise amino acid residues directly involved in the binding and other amino acid residues, which are not directly involved in the binding, such as amino acid residues which are effectively blocked by the antigen binding peptide (in other words, the amino acid residue is within the footprint of the antigen binding peptide). Epitopes may be either conformational or linear. An epitope typically includes at least 3, and more usually, at least 5 or 8-10 amino acids. Antibodies that recognize the same epitope can be verified in a simple immunoassay showing the ability of one antibody to block the binding of another antibody to a target antigen, for example “binning”.

“Linker” as used herein refers to a molecule that connect two polypeptide chains. Linker can be a polypeptide linker or a synthetic chemical linker (for example, see disclosed in Protein Engineering, 9 (3), 299-305, 1996). The length and sequence of the polypeptide linkers is not particularly limited and can be selected according to the purpose by those skilled in the art. Polypeptide linker comprises one or more amino acids. In some embodiments, the polypeptide linker is a peptide with a length of at least 5 amino acids, preferably with a length of 5 to 100, more preferably of 10 to 50 amino acids. In one embodiment, said peptide linker is G, S, GS, SG, SGG, GGS, and GSG (with G=glycine and S=serine). In another embodiment, said peptide linker is (GGGS)xGn (SEQ ID NO:74) or (GGGGS)xGn (SEQ ID NO:75) or (GGGGGS)xGn (SEQ ID NO:76) or S(GGGS)xGn (SEQ ID NO: 386) or S(GGGGS)xGn (SEQ ID NO:387) or S(GGGGGS)xGn (SEQ ID NO:388), with x=1, 2, 3, 4, 5, 6, 7, 8, 9, 10, 11, or 12 and n=0, 1, 2 or 3. Preferably, the said linker is (GGGGS)xGn with x=2,3, or 4 and n=0 (SEQ ID NO:77); more preferably the said linker is (GGGGS)xGn with x=3 and n=0 (SEQ ID NO:78). In some embodiments, the linker comprises the sequence GGGGSGGGGSGGGGS (SEQ ID NO:79) or SGGGGSGGGGSGGGGS (SEQ ID NO:389). Synthetic chemical linkers include crosslinking agents that are routinely used to crosslink peptides, for example, N-hydroxy succinimide (NHS), disuccinimidyl suberate (DSS), bis(succinimidyl) suberate (BS3), dithiobis(succinimidyl propionate) (DSP), dithiobis(succinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis[2-(succinimidoxycarbonyloxy)ethyl]sulfone (BSOCOES), and bis[2-(succinimidoxycarbonyloxy)ethyl]sulfone (sulfo-BSOCOES).

The term “polynucleotide” refers to an isolated nucleic acid molecule or construct, e.g. messenger RNA (mRNA), virally-derived RNA, or plasmid DNA (pDNA) encoding the polypeptides of the present disclosure. A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide. In some aspects, one or more vectors (particularly expression vectors) comprising such nucleic acids are provided. In one aspect, a method for making a polypeptide of the present disclosure is provided, wherein the methods comprises culturing a host cell comprising a nucleic acid encoding the polypeptide under conditions suitable for expression of the polypeptide and recovering the polypeptide from the host cell. “Recombinant” means the proteins are generated using recombinant nucleic acid techniques in exogeneous host cells. Recombinantly produced proteins expressed in host cells are considered isolated for the purpose of the present disclosure, as are native or recombinant proteins which have been separated, fractionated, or partially or substantially purified by any suitable technique.

“Isolated,” when used to describe the various polypeptides disclosed herein, means a polypeptide that has been identified and separated and/or recovered from a cell or cell culture from which it was expressed. Typically, an isolated polypeptide will be purified by at least one purification step. There is no required level of purity; “purification” or “purified” refers to increase of the target protein concentration relative to the concentration of contaminants in a composition as compared to the starting material. An “isolated protein,” as used herein refers to a target protein which is substantially free of other proteins having different binding specificities.

### Methods

Certain aspects of the present disclosure relate to methods of treating chronic viral infection. In some embodiments, the methods comprise administering to an individual in need thereof an effective amount of a fusion protein of the present disclosure. In some embodiments, the fusion protein comprises a first moiety comprising an antibody or antigen-binding fragment thereof that specifically binds human CD8b and/or human CD8ab with at least 10-fold higher affinity than its binding to human CD8a and/or human CD8aa; and a second moiety comprising a cytokine, chemokine, or growth factor, wherein the first moiety is fused to the second moiety directly or via a linker. In some embodiments, the individual is a human.

In some embodiments, the chronic viral infection is a chronic HBV infection. In some embodiments, a chronic HBV infection of the present disclosure refers to an HBV infection that is present in an individual for 6 months or longer.

In some embodiments, e.g., prior to administration of the fusion protein, the individual has or has been diagnosed with HBV infection, e.g., chronic HBV infection. Assays for detection of HBV and diagnosis of HBV infection are known in the art and include, without limitation, detection of HBV antigen(s) in a blood or plasma sample, such as detection of HBV nucleic acids (e.g., HBV DNA) and/or proteins, or detection of anti-HBV antigen antibodies. Exemplary HBV serological and virological markers include, without limitation, HBsAg, HBeAg, HBV DNA, anti-HBc IgM or IgG antibodies, anti-HBs, and anti-HBe.

In some embodiments, the methods result in an increase in HBV-reactive CD8+ T cells in the liver of the individual. In some embodiments, the methods result in reduced levels of serum HBV DNA in the individual, e.g., reduced levels of HBV DNA in a serum sample obtained from the individual (for example, as compared to levels of HBV DNA in a serum sample obtained from the individual prior to the administration, or as compared to a reference or reference value).

In some embodiments, the chronic viral infection is a human immunodeficiency virus (HIV) infection. In some embodiments, the HIV is HIV-1 or HIV-2.

In some embodiments, e.g., prior to administration of the fusion protein, the individual has or has been diagnosed with HIV infection. Assays for detection of HIV and diagnosis of HIV infection are known in the art and include, without limitation, detection of HIV antigen(s) in a blood or plasma sample (e.g., p24), such as detection of HIV nucleic acids (e.g., HIV RNA) and/or proteins, or detection of anti-HIV antigen antibodies.

In some embodiments, the methods result in an increase in HIV-reactive CD8+ T cells in the individual. In some embodiments, the methods result in reduced levels of serum HIV RNA in the individual and/or reduced levels of serum HIV antigen in the individual, e.g., as compared to levels of HIV RNA or HIV antigen in a serum sample obtained from the individual prior to the administration, or as compared to a reference or reference value).

In some embodiments, the chronic viral infection is an HBV/HIV co-infection. It is thought that 5-20% of all people infected with HIV globally are also co-infected with HBV (see, e.g., Singh, K. P. et al. (2017) AIDS 31 (15): 2035-2052). Liver-related mortality in this co-infected population can be 19-fold higher than that in populations with HBV infection alone, and 8-fold higher than that in populations with HIV alone, and overall mortality and risk of hepatocellular carcinoma (HCC) are also higher. In some embodiments, e.g., prior to administration of the fusion protein, the individual has or has been diagnosed with HBV/HIV co-infection.

In some embodiments, the methods of the present disclosure comprise administering to an individual in need thereof an effective amount of a fusion protein described herein. In some embodiments, the fusion protein comprises any one of the anti-CD8 antibodies, antigen binding domains, or antibody fragments disclosed herein. Exemplary fusion proteins and properties thereof are provided infra.

In some embodiments, the fusion protein induces activation of cells expressing a human CD8ab heterodimer with at least 2-fold, at least 5-fold, or at least 10-fold higher potency than activation of cells expressing a human CD8aa homodimer. In some embodiments, the fusion protein induces activation of CD8+ T cells with at least 2-fold, at least 5-fold, or at least 10-fold higher potency than activation of NK cells. In some embodiments, potency of activation is measured by EC50, as assessed by cell proliferation, STAT5 phosphorylation, and/or cellular cytotoxic function (e.g., expression of IFNγ and/or granzyme B). Exemplary assays are further described herein.

Preferential activity of the targeted IL-2 fusion proteins comprising the mutant IL-2 polypeptides on antigen-expressing cells is demonstrated in assays that contain antigen-expressing and antigen-non expressing cells that also express IL-2Rβγ or IL-2Rαβγ. One such assay is an in vitro assay that measures STAT5 (pSTAT5)phosphorylation and/or expression of the proliferation marker Ki-67 in human immune cells, such as human peripheral blood and/or tumor-infiltrating immune cells upon exposure to IL-2 polypeptides. In one format of the assay, the activity of the targeted IL-2 fusion protein is measured on antigen-expressing and non-expressing cells to demonstrate the selectivity on antigen-expressing cells. In another format of the assay, the activity of the targeted IL-2 fusion protein comprising the mutant IL-2 polypeptide on antigen-expressing cells is compared to that of the untargeted IL-2 fusion protein comprising the same mutant IL-2 polypeptide and a control antibody not recognizing any antigens on antigen-expressing cells to demonstrate the magnitude of rescue in signaling of the mutant IL-2 polypeptide when fused to an antigen binding molecule.

In some embodiments, the fusion protein of the present disclosure containing CD8b antigen binding molecules activates CD8b+IL-2Rβ+ cells over CD8b-IL-2Rβ+ cells by at least 10-fold, at least 50-fold, or at least 100-fold. In some embodiments, said fusion protein activates CD8b+IL-2Rβ+ cells more than 50-fold, 100 fold, or 200 fold compared to a fusion molecule comprising the said IL-2 mutant polypeptide and a control antibody not binding to any antigens expressed on said cells. Said cell activation by the IL-2 fusion protein is determined by measuring the expression of pSTAT5 or the cell proliferation marker Ki67 in said cells following the treatment with said IL-2 fusion protein.

In some embodiments, a fusion protein of the present disclosure displays one or more of the following: binds human CD8 and does not block an interaction of CD8 with MHC class I; and activates CD8+ T cells with at least 10-fold, 25-fold, 50-fold, 100-fold, 250-fold, 500-fold, or 1000-fold greater potency, e.g., as compared to activation of NK cells. In some embodiments, whether an anti-CD8 antibody or fusion protein of the present disclosure blocks the interaction of CD8 with MHC class I can be assayed, e.g., by assaying activation status of CD8+ T cells (e.g., upon antigen stimulation) in the presence or absence of the anti-CD8 antibody or fusion protein. In some embodiments, activation of CD8+ T cells and/or NK cells can be measured, e.g., by assaying one or more markers (e.g., proportion of treated cells expressing one or more markers) of proliferation (e.g., Ki67), IL-2Rβ/γ downstream signaling, and/or STAT5 downstream signaling.

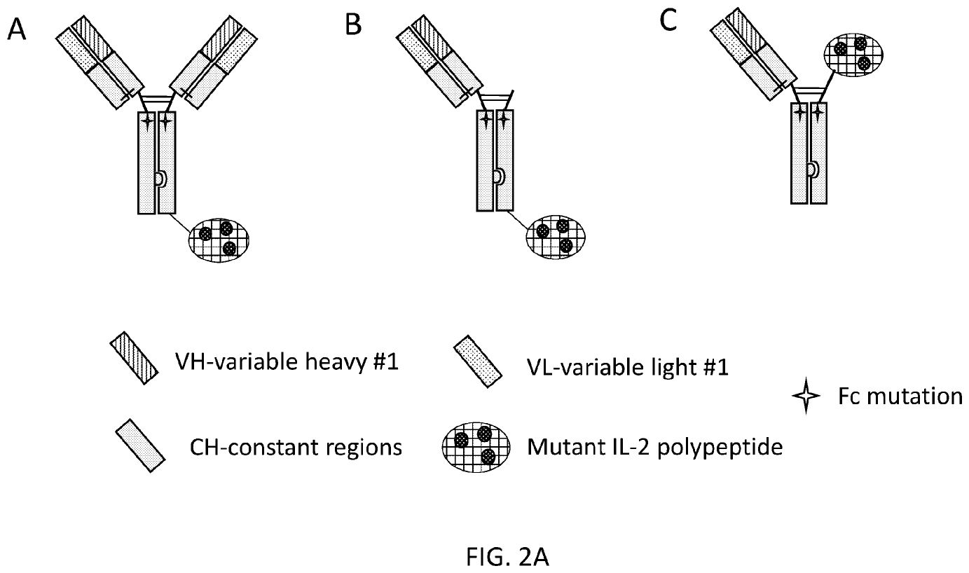

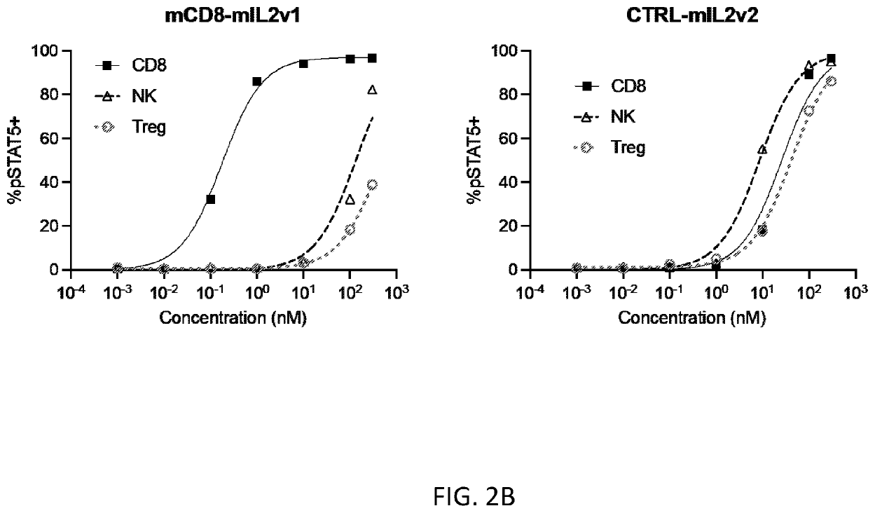

In some embodiments, a fusion protein of the present disclosure comprises a first moiety comprising a human or humanized antibody or antigen-binding fragment thereof that specifically binds CD8b and/or CD8ab (e.g., any one of the anti-CD8 antibodies described supra) and a second moiety comprising a cytokine, chemokine, or growth factor. In some embodiments, the first moiety is fused to the second moiety directly. In some embodiments, the first moiety is fused to the second moiety via a linker. Exemplary and non-limiting illustrations of fusion proteins of the present disclosure are depicted in FIG. 2A. Any of the anti-CD8 antibodies of the present disclosure (e.g., that specifically bind human CD8b and/or human CD8ab) may find use in the methods and fusion proteins disclosed herein.

In some embodiments, the anti-CD8 antibody of the present disclosure specifically binds human CD8b and/or human CD8ab with at least 10-fold, at least 20-fold, at least 30-fold, at least 40-fold, at least 50-fold, at least 60-fold, at least 70-fold, at least 80-fold, at least 90-fold, at least 100-fold, or at least 200-fold higher affinity than its binding to human CD8a and/or human CD8aa, e.g., as expressed on natural killer (NK) cells (e.g., human NK cells). In some embodiments, the anti-CD8 antibody of the present disclosure specifically binds human CD8b and/or human CD8ab with at least 10-fold higher affinity than its binding to human CD8a and/or human CD8aa, e.g., as expressed on natural killer (NK) cells. In some embodiments, the human CD8b and/or human CD8ab are expressed on the surface of a human cell, e.g., a human T cell.

In some embodiments, the anti-CD8 antibody of the present disclosure specifically binds to a cell expressing a human CD8ab heterodimer on its surface (e.g., a human T cell) with an EC50 that is less than 1000 nM. In some embodiments, the anti-CD8 antibody of the present disclosure specifically binds to human CD8+ T cells.

In some embodiments, the anti-CD8 antibody of the present disclosure is a human antibody or antibody fragment. In some embodiments, a human antibody or antibody fragment comprises human-derived CDRs and framework sequences in the variable domain, e.g., as isolated from a human or generated using a library with human antibody sequences (e.g., CDR sequences). In some embodiments, the anti-CD8 antibody of the present disclosure is a humanized antibody or antibody fragment. In some embodiments, a humanized antibody or antibody fragment comprises non-human-derived CDRs (e.g., from a mouse, rabbit, goat, etc.) and human-derived framework sequences in the variable domain. In some embodiments, a human or humanized antibody further comprises a human Fc region. In some embodiments, the human Fc region further comprises one or more Fc mutations, e.g., as disclosed herein. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called α, δ, ε, γ, and μ, respectively. The subunit structures and three-dimensional configurations of different classes of immunoglobulins are well known.

Multiple definitions for the CDR sequences of antibody variable domains are known in the art. Unless otherwise specified, CDR sequences are described herein according to the definition of Kabat (see, e.g., Kabat et al., Sequences of Proteins of Immunological Interest, Fifth Edition, NIH Publication 91-3242, Bethesda MD (1991), vols. 1-3). However, other definitions are known and contemplated for use. For example, in some embodiments, CDR sequences can be described by the definition of Chothia (see, e.g., Chothia and Lesk, J. Mol. Biol. 196:901-917 (1987). Depending upon the specific CDR definition used, the precise framework sequences can also vary, but, as is known in the art, the first framework sequence (FW-1) refers to the sequence from the N-terminus of the VH or VL domain to the beginning of CDR-H1/-L1, the second framework sequence (FW-2) refers to the sequence from end of CDR-H1/-L1 to the beginning of CDR-H2/-L2, the third framework sequence (FW-3) refers to the sequence from end of CDR-H2/-L2 to the beginning of CDR-H3/-L3, and the fourth framework sequence (FW-4) refers to the sequence from end of CDR-H3/-L3 to the C-terminal boundary of the VH or VL domain.

In some embodiments, an anti-CD8 antibody of the present disclosure comprises a VH domain comprising a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3 and a VL domain comprising a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO: 6. In some embodiments, an anti-CD8 antibody of the present disclosure comprises 1, 2, or 3 heavy chain CDRs of antibody xhCD8v1 (e.g., as shown in Tables 1-3) and/or 1, 2, or 3 light chain CDRs of antibody xhCD8v1 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:58 and a VL domain comprising a CDR-L1, CDR-L2, and CDR-L3 from the sequence of SEQ ID NO:59.

In some embodiments, an anti-CD8 antibody of the present disclosure comprises a VH domain comprising a CDR-H1 comprising the amino acid sequence of SEQ ID NO:177, a CDR-H2 comprising the amino acid sequence of SEQ ID NO:178, and a CDR-H3 comprising the amino acid sequence of SEQ ID NO: 179 and a VL domain comprising a CDR-L1 comprising the amino acid sequence of SEQ ID NO: 180, a CDR-L2 comprising the amino acid sequence of SEQ ID NO:181, and a CDR-L3 comprising the amino acid sequence of SEQ ID NO: 182. In some embodiments, an anti-CD8 antibody of the present disclosure comprises 1, 2, or 3 heavy chain CDRs of antibody xhCD8v8 (e.g., as shown in Tables 1-3) and/or 1, 2, or 3 light chain CDRs of antibody xhCD8v8 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 185 and a VL domain comprising a CDR-L1, CDR-L2, and CDR-L3 from the sequence of SEQ ID NO:186.

In some embodiments, an anti-CD8 antibody of the present disclosure comprises a VH domain comprising a CDR-H1 comprising the amino acid sequence of SEQ ID NO:13, a CDR-H2 comprising the amino acid sequence of SEQ ID NO: 14, and a CDR-H3 comprising the amino acid sequence of SEQ ID NO: 15 and a VL domain comprising a CDR-L1 comprising the amino acid sequence of SEQ ID NO: 16, a CDR-L2 comprising the amino acid sequence of SEQ ID NO:17, and a CDR-L3 comprising the amino acid sequence of SEQ ID NO: 18. In some embodiments, the VH domain comprises an amino acid sequence that is at least 90%, at least 95%, at least 99%, or 100% identical to the sequence of SEQ ID NO:62 and/or the VL domain comprises an amino acid sequence that is at least 90%, at least 95%, at least 99%, or 100% identical to the sequence of SEQ ID NO:63. In some embodiments, an anti-CD8 antibody of the present disclosure comprises 1, 2, or 3 heavy chain CDRs of antibody xhCD8v2 (e.g., as shown in Tables 1-3) and/or 1, 2, or 3 light chain CDRs of antibody xhCD8v2 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 62 and a VL domain comprising a CDR-L1, CDR-L2, and CDR-L3 from the sequence of SEQ ID NO:63.

In some embodiments, an anti-CD8 antibody of the present disclosure comprises a VH domain comprising a CDR-H1 comprising the amino acid sequence of SEQ ID NO:19, a CDR-H2 comprising the amino acid sequence of SEQ ID NO:20, and a CDR-H3 comprising the amino acid sequence of SEQ ID NO:21 and a VL domain comprising a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO: 24. In some embodiments, the VH domain comprises an amino acid sequence that is at least 90%, at least 95%, at least 99%, or 100% identical to the sequence of SEQ ID NO:64 and/or the VL domain comprises an amino acid sequence that is at least 90%, at least 95%, at least 99%, or 100% identical to the sequence of SEQ ID NO:65. In some embodiments, an anti-CD8 antibody of the present disclosure comprises 1, 2, or 3 heavy chain CDRs of antibody xhCD8v3 (e.g., as shown in Tables 1-3) and/or 1, 2, or 3 light chain CDRs of antibody xhCD8v3 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 64 and a VL domain comprising a CDR-L1, CDR-L2, and CDR-L3 from the sequence of SEQ ID NO:65.

In some embodiments, an anti-CD8 antibody of the present disclosure comprises a VH domain comprising a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27 and a VL domain comprising a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO: 30. In some embodiments, the VH domain comprises an amino acid sequence that is at least 90%, at least 95%, at least 99%, or 100% identical to the sequence of SEQ ID NO:66 and/or the VL domain comprises an amino acid sequence that is at least 90%, at least 95%, at least 99%, or 100% identical to the sequence of SEQ ID NO:67. In some embodiments, an anti-CD8 antibody of the present disclosure comprises 1, 2, or 3 heavy chain CDRs of antibody xhCD8v4 (e.g., as shown in Tables 1-3) and/or 1, 2, or 3 light chain CDRs of antibody xhCD8v4 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 66 and a VL domain comprising a CDR-L1, CDR-L2, and CDR-L3 from the sequence of SEQ ID NO:67.

In some embodiments, an anti-CD8 antibody of the present disclosure comprises a VH domain comprising a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33 and a VL domain comprising a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO: 36. In some embodiments, the VH domain comprises an amino acid sequence that is at least 90%, at least 95%, at least 99%, or 100% identical to the sequence of SEQ ID NO:68 and/or the VL domain comprises an amino acid sequence that is at least 90%, at least 95%, at least 99%, or 100% identical to the sequence of SEQ ID NO:69. In some embodiments, an anti-CD8 antibody of the present disclosure comprises 1, 2, or 3 heavy chain CDRs of antibody xhCD8v5 (e.g., as shown in Tables 1-3) and/or 1, 2, or 3 light chain CDRs of antibody xhCD8v5 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO: 68 and a VL domain comprising a CDR-L1, CDR-L2, and CDR-L3 from the sequence of SEQ ID NO:69.

In some embodiments, an anti-CD8 antibody of the present disclosure comprises a VH domain comprising a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO: 42. In some embodiments, the VH domain comprises an amino acid sequence that is at least 90%, at least 95%, at least 99%, or 100% identical to the sequence of SEQ ID NO:70 and/or the VL domain comprises an amino acid sequence that is at least 90%, at least 95%, at least 99%, or 100% identical to the sequence of SEQ ID NO:71. In some embodiments, an anti-CD8 antibody of the present disclosure comprises 1, 2, or 3 heavy chain CDRs of antibody xhCD8v6 (e.g., as shown in Tables 1-3) and/or 1, 2, or 3 light chain CDRs of antibody xhCD8v6 (e.g., as shown in Tables 1-3). In some embodiments, the antibody is human. In some embodiments, an anti-CD8 antibody of the present disclosure comprises a VH domain comprising a CDR-H1, CDR-H2, and CDR-H3 from the sequence of SEQ ID NO: 70 and a VL domain comprising a CDR-L1, CDR-L2, and CDR-L3 from the sequence of SEQ ID NO:71.

In some embodiments, an anti-CD8 antibody of the present disclosure comprises a VH domain comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45 and a VL domain comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO: 48. In some embodiments, the VH domain comprises an amino acid sequence that is at least 90%, at least 95%, at least 99%, or 100% identical to the sequence of SEQ ID NO: 72 and/or the VL domain comprises an amino acid sequence that is at least 90%, at least 95%, at least 99%, or 100% identical to the sequence of SEQ ID NO:73. In some embodiments, an anti-CD8 antibody of the present disclosure comprises 1, 2, or 3 heavy chain CDRs of antibody xhCD8v7 (e.g., as shown in Tables 1-3) and/or 1, 2, or 3 light chain CDRs of antibody xhCD8v7 (e.g., as shown in Tables 1-3). In some embodiments, the antibody is human. In some embodiments, an anti-CD8 antibody of the present disclosure comprises a VH domain comprising a CDR-H1, CDR-H2, and CDR-H3 from the sequence of SEQ ID NO: 72 and a VL domain comprising a CDR-L1, CDR-L2, and CDR-L3 from the sequence of SEQ ID NO:73.

In some embodiments, an anti-CD8 antibody of the present disclosure comprises a VH domain comprising a CDR-H1 comprising the amino acid sequence of X₁X₂AIS, wherein X₁ is S, K, G, N, R, D, T, or G, and wherein X₂ is Y, L, H, or F (SEQ ID NO:259), a CDR-H2 comprising the amino acid sequence of X₁X₂X₃PX₄X₅X₆X₇X₈X₉YX₁₀QKFX₁₁G, wherein X₁ is G or H, X₂ is I or F, X₃ is I, N, or M, X₄ is G, N, H, S, R, I, or A, X₅ is A, N, H, S, T, F, or Y, X₆ is A, D, or G, X₇ is T, E, K, V, Q, or A, X₈ is A or T, X₉ is N or K, X₁₀ is A or N, and X₁₁ is Q or T (SEQ ID NO:260), and a CDR-H3 comprising the amino acid sequence of X₁X₂X₃GX₄X₅LFX₆X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO: 261) and a VL domain comprising a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO:262), a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO:263), and a CDR-L3 comprising the amino acid sequence of QX₁X₂X₃X₄X₅PWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO:264). In some embodiments, the VH domain further comprises a FW-1 comprising the sequence QVQLVQSGAEVKKPGSSVKVSCKASGGTFS (SEQ ID NO: 274), a FW-2 comprising the sequence WVRQAPGQGLEWMG (SEQ ID NO:275), a FW-3 comprising the sequence RVTITADESTSTAYMELSSLRSEDTAVYYCAR (SEQ ID NO: 276), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:225, a CDR-H2 comprising the amino acid sequence of SEQ ID NO:226, and a CDR-H3 comprising the amino acid sequence of SEQ ID NO:227 and a VL domain comprising a CDR-L1 comprising the amino acid sequence of SEQ ID NO: 16, a CDR-L2 comprising the amino acid sequence of SEQ ID NO:17, and a CDR-L3 comprising the amino acid sequence of SEQ ID NO:228. In some embodiments, the VH domain comprises an amino acid sequence that is at least 90%, at least 95%, at least 99%, or 100% identical to the sequence of SEQ ID NO:245 and/or the VL domain comprises an amino acid sequence that is at least 90%, at least 95%, at least 99%, or 100% identical to the sequence of SEQ ID NO:246. In some embodiments, an anti-CD8 antibody of the present disclosure comprises 1, 2, or 3 heavy chain CDRs of antibody xhCD8v9 (e.g., as shown in Tables 1-3) and/or 1, 2, or 3 light chain CDRs of antibody xhCD8v9 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:245 and a VL domain comprising a CDR-L1, CDR-L2, and CDR-L3 from the sequence of SEQ ID NO:246. In some embodiments, the VH domain further comprises a FW-1 comprising the sequence QVQLVQSGAEVKKPGSSVKVSCKASGGTFS (SEQ ID NO:274), a FW-2 comprising the sequence WVRQAPGQGLEWMG (SEQ ID NO:275), a FW-3 comprising the sequence RVTITADESTSTAYMELSSLRSEDTAVYYCAR (SEQ ID NO:276), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233 and a VL domain comprising a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236. In some embodiments, the VH domain comprises an amino acid sequence that is at least 90%, at least 95%, at least 99%, or 100% identical to the sequence of SEQ ID NO:251 and/or the VL domain comprises an amino acid sequence that is at least 90%, at least 95%, at least 99%, or 100% identical to the sequence of SEQ ID NO:252. In some embodiments, the VH domain comprises the amino acid sequence of SEQ ID NO:251; and the VL domain comprises the amino acid sequence of SEQ ID NO:252. In some embodiments, an anti-CD8 antibody of the present disclosure comprises 1, 2, or 3 heavy chain CDRs of antibody xhCD8v12 (e.g., as shown in Tables 1-3) and/or 1, 2, or 3 light chain CDRs of antibody xhCD8v12 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:251 and a VL domain comprising a CDR-L1, CDR-L2, and CDR-L3 from the sequence of SEQ ID NO:252. In some embodiments, the VH domain further comprises a FW-1 comprising the sequence QVQLVQSGAEVKKPGSSVKVSCKASGGTFS (SEQ ID NO:274), a FW-2 comprising the sequence WVRQAPGQGLEWMG (SEQ ID NO:275), a FW-3 comprising the sequence RVTITADESTSTAYMELSSLRSEDTAVYYCAR (SEQ ID NO:276), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233 and a VL domain comprising a CDR-L1 comprising the amino acid sequence of SEQ ID NO: 16, a CDR-L2 comprising the amino acid sequence of SEQ ID NO:17, and a CDR-L3 comprising the amino acid sequence of SEQ ID NO:228. In some embodiments, the VH domain comprises an amino acid sequence that is at least 90%, at least 95%, at least 99%, or 100% identical to the sequence of SEQ ID NO:253 and/or the VL domain comprises an amino acid sequence that is at least 90%, at least 95%, at least 99%, or 100% identical to the sequence of SEQ ID NO:254. In some embodiments, an anti-CD8 antibody of the present disclosure comprises 1, 2, or 3 heavy chain CDRs of antibody xhCD8v13 (e.g., as shown in Tables 1-3) and/or 1, 2, or 3 light chain CDRs of antibody xhCD8v13 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:253 and a VL domain comprising a CDR-L1, CDR-L2, and CDR-L3 from the sequence of SEQ ID NO:254. In some embodiments, the VH domain further comprises a FW-1 comprising the sequence QVQLVQSGAEVKKPGSSVKVSCKASGGTFS (SEQ ID NO:274), a FW-2 comprising the sequence WVRQAPGQGLEWMG (SEQ ID NO:275), a FW-3 comprising the sequence RVTITADESTSTAYMELSSLRSEDTAVYYCAR (SEQ ID NO:276), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of X₁YX₂MS, wherein X₁ is S, D, E, A, or Q and X₂ is A, G, or T (SEQ ID NO:268), a CDR-H2 comprising the amino acid sequence of DIX₁X₂X₃GX₄X₅TX₆YADSVKG, wherein X₁ is T, N, S, Q, E, H, R, or A, X₂ is Y, W, F, or H, X₃ is A, S, Q, E, or T, X₄ is G or E, X₅ is S or I, and X₆ is A or G (SEQ ID NO:269), and a CDR-H3 comprising the amino acid sequence of X₁X₂X₃YX₄WX₅X₆AX-DX₈, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO:270) and a VL domain comprising a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42). In some embodiments, the VH domain further comprises a FW-1 comprising the sequence EVOLVESGGGLVQPGGSLRLSCAASGFTFS (SEQ ID NO:281), a FW-2 comprising the sequence WVRQAPGKGLEWVS (SEQ ID NO:282), a FW-3 comprising the sequence RFTISRDNAKNSLYLQMNSLRAEDTAVYYCAR (SEQ ID NO:283), and/or a FW-4 comprising the sequence WGQGTMVTVSS (SEQ ID NO:284) or WGQGTLVTVSS (SEQ ID NO: 285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO: 247 and/or the VL domain comprises an amino acid sequence that is at least 90%, at least 95%, at least 99%, or 100% identical to the sequence of SEQ ID NO:248. In some embodiments, an anti-CD8 antibody of the present disclosure comprises 1, 2, or 3 heavy chain CDRs of antibody xhCD8v10 (e.g., as shown in Tables 1-3) and/or 1, 2, or 3 light chain CDRs of antibody xhCD8v10 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:247 and a VL domain comprising a CDR-L1, CDR-L2, and CDR-L3 from the sequence of SEQ ID NO:248. In some embodiments, the VH domain further comprises a FW-1 comprising the sequence EVOLVESGGGLVQPGGSLRLSCAASGFTFS (SEQ ID NO:281), a FW-2 comprising the sequence WVRQAPGKGLEWVS (SEQ ID NO: 282), a FW-3 comprising the sequence RFTISRDNAKNSLYLQMNSLRAEDTAVYYCAR (SEQ ID NO:283), and/or a FW-4 comprising the sequence WGQGTMVTVSS (SEQ ID NO:284) or WGQGTLVTVSS (SEQ ID NO: 285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO: 249 and/or the VL domain comprises an amino acid sequence that is at least 90%, at least 95%, at least 99%, or 100% identical to the sequence of SEQ ID NO:250. In some embodiments, the VH domain comprises the amino acid sequence of SEQ ID NO:249; and the VL domain comprises the amino acid sequence of SEQ ID NO:250. In some embodiments, an anti-CD8 antibody of the present disclosure comprises 1, 2, or 3 heavy chain CDRs of antibody xhCD8v11 (e.g., as shown in Tables 1-3) and/or 1, 2, or 3 light chain CDRs of antibody xhCD8v11 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:249 and a VL domain comprising a CDR-L1, CDR-L2, and CDR-L3 from the sequence of SEQ ID NO:250. In some embodiments, the VH domain further comprises a FW-1 comprising the sequence EVOLVESGGGLVQPGGSLRLSCAASGFTFS (SEQ ID NO:281), a FW-2 comprising the sequence WVRQAPGKGLEWVS (SEQ ID NO: 282), a FW-3 comprising the sequence RFTISRDNAKNSLYLQMNSLRAEDTAVYYCAR (SEQ ID NO:283), and/or a FW-4 comprising the sequence WGQGTMVTVSS (SEQ ID NO:284) or WGQGTLVTVSS (SEQ ID NO: 285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:229, a CDR-H2 comprising the amino acid sequence of SEQ ID NO:237, and a CDR-H3 comprising the amino acid sequence of SEQ ID NO:231 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO: 255 and/or the VL domain comprises an amino acid sequence that is at least 90%, at least 95%, at least 99%, or 100% identical to the sequence of SEQ ID NO:256. In some embodiments, an anti-CD8 antibody of the present disclosure comprises 1, 2, or 3 heavy chain CDRs of antibody xhCD8v14 (e.g., as shown in Tables 1-3) and/or 1, 2, or 3 light chain CDRs of antibody xhCD8v14 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:255 and a VL domain comprising a CDR-L1, CDR-L2, and CDR-L3 from the sequence of SEQ ID NO:256. In some embodiments, the VH domain further comprises a FW-1 comprising the sequence EVOLVESGGGLVQPGGSLRLSCAASGFTFS (SEQ ID NO:281), a FW-2 comprising the sequence WVRQAPGKGLEWVS (SEQ ID NO: 282), a FW-3 comprising the sequence RFTISRDNAKNSLYLQMNSLRAEDTAVYYCAR (SEQ ID NO:283), and/or a FW-4 comprising the sequence WGQGTMVTVSS (SEQ ID NO:284) or WGQGTLVTVSS (SEQ ID NO: 285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:229, a CDR-H2 comprising the amino acid sequence of SEQ ID NO:237, and a CDR-H3 comprising the amino acid sequence of SEQ ID NO:231 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain comprises an amino acid sequence that is at least 90%, at least 95%, at least 99%, or 100% identical to the sequence of SEQ ID NO: 257 and/or the VL domain comprises an amino acid sequence that is at least 90%, at least 95%, at least 99%, or 100% identical to the sequence of SEQ ID NO:258. In some embodiments, an anti-CD8 antibody of the present disclosure comprises 1, 2, or 3 heavy chain CDRs of antibody xhCD8v15 (e.g., as shown in Tables 1-3) and/or 1, 2, or 3 light chain CDRs of antibody xhCD8v15 (e.g., as shown in Tables 1-3). In some embodiments, the antibody is humanized. In some embodiments, an anti-CD8 antibody of the present disclosure comprises a VH domain comprising a CDR-H1, CDR-H2, and CDR-H3 from the sequence of SEQ ID NO:257 and a VL domain comprising a CDR-L1, CDR-L2, and CDR-L3 from the sequence of SEQ ID NO:258. In some embodiments, the VH domain further comprises a FW-1 comprising the sequence EVOLVESGGGLVQPGGSLRLSCAASGFTFS (SEQ ID NO:281), a FW-2 comprising the sequence WVRQAPGKGLEWVS (SEQ ID NO: 282), a FW-3 comprising the sequence RFTISRDNAKNSLYLQMNSLRAEDTAVYYCAR (SEQ ID NO:283), and/or a FW-4 comprising the sequence WGQGTMVTVSS (SEQ ID NO:284) or WGQGTLVTVSS (SEQ ID NO: 285). In some embodiments, the VL domain further comprises a FW-1 comprising the sequence EIVLTQSPGTLSLSPGERATLSC (SEQ ID NO:293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO:295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:49, a CDR-H2 comprising the amino acid sequence of SEQ ID NO:50, and a CDR-H3 comprising the amino acid sequence of SEQ ID NO:3 and a VL domain comprising a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO: 6. In some embodiments, an anti-CD8 antibody of the present disclosure comprises a VH domain comprising a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO: 15 and a VL domain comprising a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO: 18. In some embodiments, an anti-CD8 antibody of the present disclosure comprises a VH domain comprising a CDR-H1 comprising the amino acid sequence of SEQ ID NO:53, a CDR-H2 comprising the amino acid sequence of SEQ ID NO:52, and a CDR-H3 comprising the amino acid sequence of SEQ ID NO:21 and a VL domain comprising a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO: 24. In some embodiments, an anti-CD8 antibody of the present disclosure comprises a VH domain comprising a CDR-H1 comprising the amino acid sequence of SEQ ID NO:49, a CDR-H2 comprising the amino acid sequence of SEQ ID NO:52, and a CDR-H3 comprising the amino acid sequence of SEQ ID NO:27 and a VL domain comprising a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO: 30. In some embodiments, an anti-CD8 antibody of the present disclosure comprises a VH domain comprising a CDR-H1 comprising the amino acid sequence of SEQ ID NO:54, a CDR-H2 comprising the amino acid sequence of SEQ ID NO:52, and a CDR-H3 comprising the amino acid sequence of SEQ ID NO:33 and a VL domain comprising a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO: 36. In some embodiments, an anti-CD8 antibody of the present disclosure comprises a VH domain comprising a CDR-H1 comprising the amino acid sequence of SEQ ID NO:55, a CDR-H2 comprising the amino acid sequence of SEQ ID NO:56, and a CDR-H3 comprising the amino acid sequence of SEQ ID NO:39 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO: 42. In some embodiments, an anti-CD8 antibody of the present disclosure comprises a VH domain comprising a CDR-H1 comprising the amino acid sequence of SEQ ID NO:55, a CDR-H2 comprising the amino acid sequence of SEQ ID NO:57, and a CDR-H3 comprising the amino acid sequence of SEQ ID NO:45 and a VL domain comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO: 48. In some embodiments, an anti-CD8 antibody of the present disclosure comprises a VH domain comprising a CDR-H1 comprising the amino acid sequence of SEQ ID NO: 183, a CDR-H2 comprising the amino acid sequence of SEQ ID NO: 184, and a CDR-H3 comprising the amino acid sequence of SEQ ID NO:179 and a VL domain comprising a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO: 182.

In some embodiments, an anti-CD8 antibody of the present disclosure comprises a VH domain comprising a CDR-H1 comprising the amino acid sequence of GX₁X₂FX₃X₄X₅, wherein X₁ is G, Y, S, or A, X₂ is T, S, G, R, N, or H, X₃ is S, T, R, H, Y, G, or P, X₄ is S, K, G, N, R, D, T, or G, and X₅ is Y, L, H, or F (SEQ ID NO:265), a CDR-H2 comprising the amino acid sequence of X₁PX₂X₃X₄X₅, wherein X₁ is I, N, or M, X₂ is G, N, H, S, R, I, or A, X₃ is A, N, H, S, T, F, or Y, X₄ is A, D, or G, and X₅ is T, E, K, V, Q, or A (SEQ ID NO: 266), and a CDR-H3 comprising the amino acid sequence of X₁X₂X₃GX₄X₅LFX₆X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO:267) and a VL domain comprising a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO:262), a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO:263), and a CDR-L3 comprising the amino acid sequence of QX₁X₂X₃X₄X₅PWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO:264). In some embodiments, the VH domain further comprises a FW-1 comprising the sequence QVQLVQSGAEVKKPGSSVKVSCKAS (SEQ ID NO:278), a FW-2 comprising the sequence AISWVRQAPGQGLEWMGGI (SEQ ID NO:279), a FW-3 comprising the sequence ANYAQKFQGRVTITADESTSTAYMELSSLRSEDTAVYYCAR (SEQ ID NO: 280), and/or a FW-4 comprising the sequence WGQGTLVTVSS (SEQ ID NO:277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:238, a CDR-H2 comprising the amino acid sequence of SEQ ID NO:239, and a CDR-H3 comprising the amino acid sequence of SEQ ID NO:233 and a VL domain comprising a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO:228. In some embodiments, the VH domain further comprises a FW-1 comprising the sequence QVQLVQSGAEVKKPGSSVKVSCKAS (SEQ ID NO:278), a FW-2 comprising the sequence AISWVRQAPGQGLEWMGGI (SEQ ID NO:279), a FW-3 comprising the sequence ANYAQKFQGRVTITADESTSTAYMELSSLRSEDTAVYYCAR (SEQ ID NO:280), and/or a FW-4 comprising the sequence WGQGTLVTVSS (SEQ ID NO: 277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233 and a VL domain comprising a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236. In some embodiments, the VH domain further comprises a FW-1 comprising the sequence QVQLVQSGAEVKKPGSSVKVSCKAS (SEQ ID NO:278), a FW-2 comprising the sequence AISWVRQAPGQGLEWMGGI (SEQ ID NO:279), a FW-3 comprising the sequence ANYAQKFQGRVTITADESTSTAYMELSSLRSEDTAVYYCAR (SEQ ID NO:280), and/or a FW-4 comprising the sequence WGQGTLVTVSS (SEQ ID NO: 277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of SEQ ID NO:238, a CDR-H2 comprising the amino acid sequence of SEQ ID NO:243, and a CDR-H3 comprising the amino acid sequence of SEQ ID NO:233 and a VL domain comprising a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228. In some embodiments, the VH domain further comprises a FW-1 comprising the sequence QVQLVQSGAEVKKPGSSVKVSCKAS (SEQ ID NO:278), a FW-2 comprising the sequence AISWVRQAPGQGLEWMGGI (SEQ ID NO:279), a FW-3 comprising the sequence ANYAQKFQGRVTITADESTSTAYMELSSLRSEDTAVYYCAR (SEQ ID NO:280), and/or a FW-4 comprising the sequence WGQGTLVTVSS (SEQ ID NO: 277). In some embodiments, the VL domain further comprises a FW-1 comprising the sequence DIQMTQSPSSLSASVGDRVTITC (SEQ ID NO:289), a FW-2 comprising the sequence WYQQKPGKAPKLLIY (SEQ ID NO:290), a FW-3 comprising the sequence GVPSRFSGSGSGTDFTLTISSLQPEDFATYYC (SEQ ID NO:291), and/or a FW-4 comprising the sequence FGGGTKVEIK (SEQ ID NO:292).

In some embodiments, an anti-CD8 antibody of the present disclosure comprises a VH domain comprising a CDR-H1 comprising the amino acid sequence of GFTFX₁X₂Y, wherein X₁ is S, D, E, Q, S, or A and X₂ is S, D, E, A, or Q (SEQ ID NO:271), a CDR-H2 comprising the amino acid sequence of X₁X₂X₃GX₄X₅, wherein X₁ is T, N, S, Q, E, H, R or A, X₂ is Y, W, F, or H, X₃ is A, S, Q, E, or T, X₄ is G or E, and X₅ is S or I (SEQ ID NO: 272), and a CDR-H3 comprising the amino acid sequence of X₁X₂X₃YXAWX₅X₆AX₇DX₈, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO:273) and a VL domain comprising a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42). In some embodiments, the VH domain further comprises a FW-1 comprising the sequence EVOLVESGGGLVQPGGSLRLSCAAS (SEQ ID NO:286), a FW-2 comprising the sequence AMSWVRQAPGKGLEWVSDI (SEQ ID NO: 287), a FW-3 comprising the sequence TAYADSVKGRFTISRDNAKNSLYLQMNSLRAEDTAVYYCAR (SEQ ID NO:288), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO: 293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO: 295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:240, a CDR-H2 comprising the amino acid sequence of SEQ ID NO:241, and a CDR-H3 comprising the amino acid sequence of SEQ ID NO:242 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain further comprises a FW-1 comprising the sequence EVOLVESGGGLVQPGGSLRLSCAAS (SEQ ID NO:286), a FW-2 comprising the sequence AMSWVRQAPGKGLEWVSDI (SEQ ID NO:287), a FW-3 comprising the sequence TAYADSVKGRFTISRDNAKNSLYLQMNSLRAEDTAVYYCAR (SEQ ID NO:288), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO: 293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO: 295), and/or a FW-4 comprising the sequence FGQGTKVEIK (SEQ ID NO:296).

In some embodiments, an anti-CD8 antibody of the present disclosure comprises a VH domain comprising a CDR-H1 comprising the amino acid sequence of SEQ ID NO:240, a CDR-H2 comprising the amino acid sequence of SEQ ID NO:244, and a CDR-H3 comprising the amino acid sequence of SEQ ID NO:242 and a VL domain comprising a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42. In some embodiments, the VH domain further comprises a FW-1 comprising the sequence EVQLVESGGGLVQPGGSLRLSCAAS (SEQ ID NO:286), a FW-2 comprising the sequence AMSWVRQAPGKGLEWVSDI (SEQ ID NO:287), a FW-3 comprising the sequence TAYADSVKGRFTISRDNAKNSLYLQMNSLRAEDTAVYYCAR (SEQ ID NO:288), and/or a FW-4 comprising the sequence WGQGTMVTVSS (SEQ ID NO:284) or WGQGTLVTVSS (SEQ ID NO:285). In some embodiments, the VL domain further comprises a FW-1 comprising the sequence EIVLTQSPGTLSLSPGERATLSC (SEQ ID NO: 293), a FW-2 comprising the sequence WYQQKPGQAPRLLIY (SEQ ID NO:294), a FW-3 comprising the sequence GIPDRFSGSGSGTDFTLTISRLEPEDFAVYYC (SEQ ID NO: 295), and/or a FW-4 comprising the sequence FGQGTKVEIK (SEQ ID NO:296). In some embodiments, the present disclosure provides an anti-CD8 antibody comprising a VH domain comprising CDR-H1, CDR-H2, and CDR-H3 sequences of a single antibody listed in Table 1 and a VL domain comprising CDR-L1, CDR-L2, and CDR-L3 sequences of the single antibody listed in Table 1. For example, the anti-CD8 antibody comprises the six CDRs of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, xhCD8v15, V9 family, or V11 family shown in Table 1. In some embodiments, the present disclosure provides an anti-CD8 antibody comprising a VH domain comprising CDR-H1, CDR-H2, and CDR-H3 sequences of a single antibody listed in Table 2 and a VL domain comprising CDR-L1, CDR-L2, and CDR-L3 sequences of the single antibody listed in Table 2. For example, the anti-CD8 antibody comprises the six CDRs of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, xhCD8v15, V9 family, or V11 family shown in Table 2. In some embodiments, the present disclosure provides a fusion protein comprising an anti-CD8 antibody comprising a VH domain comprising CDR-H1, CDR-H2, and CDR-H3 sequences of the single antibody listed in Table 1 and a VL domain comprising CDR-L1, CDR-L2, and CDR-L3 sequences of a single antibody listed in Table 1. For example, the anti-CD8 antibody of the fusion protein comprises the six CDRs of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, xhCD8v15, V9 family, or V11 family shown in Table 1. In some embodiments, the present disclosure provides a fusion protein comprising an anti-CD8 antibody comprising a VH domain comprising CDR-H1, CDR-H2, and CDR-H3 sequences of a single antibody listed in Table 2 and a VL domain comprising CDR-L1, CDR-L2, and CDR-L3 sequences of the single antibody listed in Table 2. For example, the anti-CD8 antibody of the fusion protein comprises the six CDRs of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, xhCD8v15, V9 family, or V11 family shown in Table 2. In some embodiments, the present disclosure provides an anti-CD8 antibody comprising a VH domain comprising CDR-H1, CDR-H2, and CDR-H3 sequences of a VH domain listed in Table 3 and a VL domain comprising CDR-L1, CDR-L2, and CDR-L3 sequences of a VL domain listed in Table 3 (in some embodiments, the VH and VL domains are from the same single antibody listed in Table 3). For example, the anti-CD8 antibody comprises the VH and VL of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, or xhCD8v15 shown in Table 3. In some embodiments, the present disclosure provides a fusion protein comprising an anti-CD8 antibody comprising a VH domain comprising CDR-H1, CDR-H2, and CDR-H3 sequences of a VH domain listed in Table 3 and a VL domain comprising CDR-L1, CDR-L2, and CDR-L3 sequences of a VL domain listed in Table 3 (in some embodiments, the VH and VL domains are from the same single antibody listed in Table 3). In some embodiments, the present disclosure provides an anti-CD8 antibody comprising a VH domain sequence and a VL domain sequence for a single antibody as listed in Table 3. In some embodiments, the present disclosure provides a fusion protein comprising an anti-CD8 antibody comprising a VH domain sequence and a VL domain sequence for a single antibody as listed in Table 3. For example, the anti-CD8 antibody of the fusion protein comprises the VH and VL of antibody xhCD8v1, xhCD8v1.1, xhCD8v2, xhCD8v3, xhCD8v4, xhCD8v5, xhCD8v6, xhCD8v7, xhCD8v8, xhCD8v9, xhCD8v10, xhCD8v11, xhCD8v12, xhCD8v13, xhCD8v14, or xhCD8v15 shown in Table 3.

TABLE 1
Anti-CD8 antibody CDRs (Kabat)

| Name | CDR-H1 | CDR-H2 | CDR-H3 | CDR-L1 | CDR-L2 | CDR-L3 |
|---|---|---|---|---|---|---|
| xhCD8v1 | KYTMH | HFNPNND | DGLGLRL | GASENIY | GATNLAD | QNILDTP |
| | (SEQ ID | ETKYNQK | FAD | GALN | (SEQ ID | WT |
| | NO: 1) | FTG | (SEQ ID | (SEQ ID | NO: 5) | (SEQ ID |
| | | (SEQ ID | NO: 3) | NO: 4) | | NO: 6) |
| | | NO: 2) | | | | |
| xhCD8v1.1 | KYAIS | HFNPNND | DGLGLRL | RASENIY | GATNLAD | QNILDTP |
| | (SEQ ID | ETKYNQK | FAD | GALN | (SEQ ID | WT |
| | NO: 7) | FQG | (SEQ ID | (SEQ ID | NO: 11) | (SEQ ID |
| | | (SEQ ID | NO: 9) | NO: 10) | | NO: 12) |
| | | NO: 8) | | | | |
| xhCD8v2 | NFAIS | GIIPGHAK | DGLGIRL | RASQEIY | GATNLQS | QDIYDAP |
| | (SEQ ID | ANYAQK | FAD | GALN | (SEQ ID | WT |
| | NO: 13) | FQG | (SEQ ID | (SEQ ID | NO: 17) | (SEQ ID |
| | | (SEQ ID | NO: 15) | NO: 16) | | NO: 18) |
| | | NO: 14) | | | | |
| xhCD8v3 | KFAIS | GIIPGHAK | DGLGIRL | RASQEIY | GATNLQS | QDIYDAP |
| | (SEQ ID | ANYAQK | FAD | GALN | (SEQ ID | WT |
| | NO: 19) | FQG | (SEQ ID | (SEQ ID | NO: 23) | (SEQ ID |
| | | (SEQ ID | NO: 21) | NO: 22) | | NO: 24) |
| | | NO: 20) | | | | |
| xhCD8v4 | KYAIS | GIIPGHAK | DGLGIRL | RASQKIY | GATNLQS | QNTYDTP |
| | (SEQ ID | ANYAQK | FAD | GALN | (SEQ ID | WT |
| | NO: 25) | FQG | (SEQ ID | (SEQ ID | NO: 29) | (SEQ ID |
| | | (SEQ ID | NO: 27) | NO: 28) | | NO: 30) |
| | | NO: 26) | | | | |
| xhCD8v5 | GHAIS | GIIPGHAK | DGLGIRL | RASQKIY | GATNLQS | QNTYDTP |
| | (SEQ ID | ANYAQK | FAD | GALN | (SEQ ID | WT |
| | NO: 31) | FQG | (SEQ ID | (SEQ ID | NO: 35) | (SEQ ID |
| | | (SEQ ID | NO: 33) | NO: 34) | | NO: 36) |
| | | NO: 32) | | | | |
| xhCD8v6 | DYGMS | DINWSGE | SNSYRW | RASQSVS | GASSRAT | QQYGSSP |
| | (SEQ ID | ITAYADS | DDALDI | SNLA | (SEQ ID | PVT |
| | NO: 37) | VKG | (SEQ ID | (SEQ ID | NO: 41) | (SEQ ID |
| | | (SEQ ID | NO: 39) | NO: 40) | | NO: 42) |
| | | NO: 38) | | | | |
| xhCD8v7 | DYAMH | VISYDGS | DRIGWYD | RASHSVG | DASNRAT | QQRSNW |
| | (SEQ ID | NKYYAD | YDAFDI | SNLA | (SEQ ID | PPT |
| | NO: 43) | SVKG | (SEQ ID | (SEQ ID | NO: 47) | (SEQ ID |
| | | (SEQ ID | NO: 45) | NO: 46) | | NO: 48) |
| | | NO: 44) | | | | |
| xhCD8v8 | SYWMN | QIYPGDG | SGAAFSS | RASENIY | AATNLAD | QHFWGTP |
| | (SEQ ID | DTNYNG | YYAMDY | SNLA | (SEQ ID | WT (SEQ |
| | NO: 177) | KFKG | (SEQ ID | (SEQ ID | NO: 181) | ID NO: 182) |
| | | (SEQ ID | NO: 179) | NO: 180) | | |
| | | NO: 178) | | | | |
| xhCD8v9 | SYAIS | GIIPGAAT | DAAGIRL | RASQEIY | GATNLQS | QSTYDAP |
| | (SEQ ID | ANYAQK | FAD | GALN | (SEQ ID | WT (SEQ |
| | NO: 225) | FQG | (SEQ ID | (SEQ ID | NO: 17) | ID NO: 228) |
| | | (SEQ ID | NO: 227) | NO: 16) | | |
| | | NO: 226) | | | | |
| xhCD8v10 | SYAMS | DITYAGG | SNAYAW | RASQSVS | GASSRAT | QQYGSSP |
| | (SEQ ID | STAYADS | DDALDI | SNLA | (SEQ ID | PVT |
| | NO: 229) | VKG (SEQ | (SEQ ID | (SEQ ID | NO: 41) | (SEQ ID |
| | | ID NO: 230) | NO: 231) | NO: 40) | | NO: 42) |
| xhCD8v11 | SYAMS | DITYAGG | SNAYAW | RASQSVS | GASSRAT | QQYGSSP |
| | (SEQ ID | STAYADS | DDALDI | SNLA | (SEQ ID | PVT |
| | NO: 229) | VKG (SEQ | (SEQ ID | (SEQ ID | NO: 41) | (SEQ ID |
| | | ID NO: 230) | NO: 231) | NO: 40) | | NO: 42) |
| xhCD8v12 | SYAIS | GIIPGYAT | DAAGIRL | RASQSIY | GASNLQS | QSTYTAP |
| | (SEQ ID | ANYAQK | FAD (SEQ | GALN | (SEQ ID | |
| | NO: 225) | FQG (SEQ | ID NO: 233) | (SEQ ID | NO: 235) | WT (SEQ |
| | | ID NO: 232) | | NO: 234) | | ID NO: 236) |
| xhCD8v13 | SYAIS | GIIPGYAT | DAAGIRL | RASQEIY | GATNLQS | QSTYDAP |
| | (SEQ ID | ANYAQK | FAD (SEQ | GALN | (SEQ ID | WT (SEQ |
| | NO: 225) | FQG (SEQ | ID NO: 233) | (SEQ ID | NO: 17) | ID NO: 228) |
| | | ID NO: 232) | | NO: 16) | | |
| xhCD8v14 | SYAMS | DISYAGG | SNAYAW | RASQSVS | GASSRAT | QQYGSSP |
| | (SEQ ID | STAYADS | DDALDI | SNLA | (SEQ ID | PVT |
| | NO: 229) | VKG (SEQ | (SEQ ID | (SEQ ID | NO: 41) | (SEQ ID |
| | | ID NO: 237) | NO: 231) | NO: 40) | | NO: 42) |
| xhCD8v15 | SYAMS | DISYAGG | SNAYAW | RASQSVS | GASSRAT | QQYGSSP |
| | (SEQ ID | STAYADS | DDALDI | SNLA | (SEQ ID | PVT |
| | NO: 229) | VKG (SEQ | (SEQ ID | (SEQ ID | NO: 41) | (SEQ ID |
| | | ID NO: 237) | NO: 231) | NO: 40) | | NO: 42) |
| V9 family | X₁X₂AIS | X₁X₂X₃PX₄ | X₁X₂X₃GX₄ | X₁X₂SX₃X₄ | GX₁X₂X₃L | QX₁X₂X₃X₄ |
| | X₁ is S, K, | X₅X₆X₇X₈ | X₅LFX₆X₇ | IX₅GX₆LN | X₄X₅ | X₅PWT |
| | G, N, R, D, | X₉YX₁₀QK | X₁ is D or | X₁ is R or | X₁ is A or | X₁ is S, N, |
| | T, or G | FX₁₁G | A, | G, | S, X₂ is T, | D, Q, A, or |
| | X₂ is Y, L, | X₁ is Gor | X₂ is A, G, | X₂ is A or | S, E, Q, or | E, |
| | H, or F | H, X₂ is I or | E, R, Y, K, | T, | D, X₃ is N, | X₂ is T, I, |
| | (SEQ ID | F, | N, Q, L, or | X₃ is Q or | R, A, E, or | or S, |
| | NO: 259) | X₃ is I, N, | F, | E, | H, X₄ is Q | X₃ is Y, L, |
| | | or M, | X₃ is A, L, | X₄ is E, N, | or A, | or F, |
| | | X₄ is G, N, | P, or Y, | T, S, A, K, | X₅ is S or D | X₄ is D, G, |
| | | H, S, R, I, | X₄ is I or L, | D, G, R, or | (SEQ ID | T, E, Q, A, |
| | | or A, X₅ is | X₅ is R, A, | Q, | NO: 263) | or Y, |
| | | A, N, H, S, | Q, or S, | X₅ is Y or | | X₅ is A, T, |
| | | T, F, or Y, | X₆ is A or | S, X₆ is A | | R, S, K, or |
| | | X₆ is A, D, | D, | or V (SEQ | | Y |
| | | or G, | X₇ is D, E, | ID NO: 262) | | (SEQ ID |
| | | X₇ is T, E, | A, or S | | | NO: 264) |
| | | K, V, Q, or | (SEQ ID | | | |
| | | A, | NO: 261) | | | |
| | | X₈ is A or | | | | |
| | | T, X₉ is N | | | | |
| | | or K, X₁₀ is | | | | |
| | | A or N, X₁₁ | | | | |
| | | is Q or T | | | | |
| | | (SEQ ID | | | | |
| | | NO: 260) | | | | |
| V11 family | X₁YX₂MS | DIX₁X₂X₃ | X₁X₂X₃YX₄ | RASQSVS | GASSRAT | QQYGSSP |
| | X₁ is S, D, | GX₄X₅TX₆ | WX₅X₆AX₇ | SNLA | (SEQ ID | PVT |
| | E, A, or Q | YADSVK | DX₈ | (SEQ ID | NO: 41) | (SEQ ID |
| | X₂ is A, G, | G | X₁ is S or | NO: 40) | | NO: 42) |
| | or T (SEQ | X1 is T, N, | A, X₂ is N, | | | |
| | ID NO: 268) | S, Q, E, H, | H, A, D, L, | | | |
| | | R, or A, | Q, Y, or R, | | | |
| | | X₂ is Y, W, | X₃ is A, N, | | | |
| | | F, or H, | S, or G, | | | |
| | | X₃ is A, S, | X₄ is A, V, | | | |
| | | Q, E, or T, | R, E, or S, | | | |
| | | X₄ is G or | X₅ is D or | | | |
| | | E, X₅ is S | S, X₆ is D, | | | |
| | | or I, | N, Q, E, S, | | | |
| | | X₆ is A or | T, or L, | | | |
| | | G (SEQ ID | X₇ is L, F, | | | |
| | | NO: 269) | or M, | | | |
| | | | X₈ is I, Y, | | | |
| | | | or V | | | |
| | | | (SEQ ID | | | |
| | | | NO: 270) | | | |

TABLE 2
Anti-CD8 antibody CDRs (Chothia)

| Name | CDR-H1 | CDR-H2 | CDR-H3 | CDR-L1 | CDR-L2 | CDR-L3 |
|---|---|---|---|---|---|---|
| xhCD8v1 | GYTFTKY | NPNNDE | DGLGLRL | GASENIY | GATNLAD | QNILDTP |
| | (SEQ ID | (SEQ ID | FAD | GALN | (SEQ ID | WT |
| | NO: 49) | NO: 50) | (SEQ ID | (SEQ ID | NO: 5) | (SEQ ID |
| | | | NO: 3) | NO: 4) | | NO: 6) |
| xhCD8v1.1 | GYTFTKY | NPNNDE | DGLGLRL | RASENIY | GATNLAD | QNILDTP |
| | (SEQ ID | (SEQ ID | FAD | GALN | (SEQ ID | WT |
| | NO: 49) | NO: 50) | (SEQ ID | (SEQ ID | NO: 11) | (SEQ ID |
| | | | NO: 9) | NO: 10) | | NO: 12) |
| xhCD8v2 | GYRFHNF | IPGHAK | DGLGIRL | RASQEIY | GATNLQS | QDIYDAP |
| | (SEQ ID | (SEQ ID | FAD | GALN | (SEQ ID | WT |
| | NO: 51) | NO: 52) | (SEQ ID | (SEQ ID | NO: 17) | (SEQ ID |
| | | | NO: 15) | NO: 16) | | NO: 18) |
| xhCD8v3 | GSRFYKF | IPGHAK | DGLGIRL | RASQEIY | GATNLQS | QDIYDAP |
| | (SEQ ID | (SEQ ID | FAD | GALN | (SEQ ID | WT |
| | NO: 53) | NO: 52) | (SEQ ID | (SEQ ID | NO: 23) | (SEQ ID |
| | | | NO: 21) | NO: 22) | | NO: 24) |
| xhCD8v4 | GYTFTKY | IPGHAK | DGLGIRL | RASQKIY | GATNLQS | QNTYDTP |
| | (SEQ ID | (SEQ ID | FAD | GALN | (SEQ ID | WT |
| | NO: 49) | NO: 52) | (SEQ ID | (SEQ ID | NO: 29) | (SEQ ID |
| | | | NO: 27) | NO: 28) | | NO: 30) |
| xhCD8v5 | GSGFRGH | IPGHAK | DGLGIRL | RASQKIY | GATNLQS | QNTYDTP |
| | (SEQ ID | (SEQ ID | FAD | GALN | (SEQ ID | WT |
| | NO: 54) | NO: 52) | (SEQ ID | (SEQ ID | NO: 35) | (SEQ ID |
| | | | NO: 33) | NO: 34) | | NO: 36) |
| xhCD8v6 | GFTFDDY | NWSGEI | SNSYRW | RASQSVS | GASSRAT | QQYGSSP |
| | (SEQ ID | (SEQ ID | DDALDI | SNLA | (SEQ ID | PVT |
| | NO: 55) | NO: 56) | (SEQ ID | (SEQ ID | NO: 41) | (SEQ ID |
| | | | NO: 39) | NO: 40) | | NO: 42) |
| xhCD8v7 | GFTFDDY | SYDGSN | DRIGWYD | RASHSVG | DASNRAT | QQRSNW |
| | (SEQ ID | (SEQ ID | YDAFDI | SNLA | (SEQ ID | PPT |
| | NO: 55) | NO: 57) | (SEQ ID | (SEQ ID | NO: 47) | (SEQ ID |
| | | | NO: 45) | NO: 46) | | NO: 48) |
| xhCD8v8 | GYAFSSY | YPGDGD | SGAAFSS | RASENIY | AATNLAD | QHFWGTP |
| | (SEQ ID | (SEQ ID | YYAMDY | SNLA | (SEQ ID | WT (SEQ |
| | NO: 183) | NO: 184) | (SEQ ID | (SEQ ID | NO: 181) | ID NO: 182) |
| | | | NO: 179) | NO: 180) | | |
| xhCD8v9 | GGTFSSY | IPGAAT | DAAGIRL | RASQEIY | GATNLQS | QSTYDAP |
| | (SEQ ID | (SEQ ID | FAD (SEQ | GALN | (SEQ ID | WT (SEQ |
| | NO: 238) | NO: 239) | ID NO: 233) | (SEQ ID | NO: 17) | ID NO: 228) |
| | | | | NO: 16) | | |
| xhCD8v10 | GFTFSSY | TYAGGS | SNAYAW | RASQSVS | GASSRAT | QQYGSSP |
| | (SEQ ID | (SEQ ID | DDALDI | SNLA | (SEQ ID | PVT |
| | NO: 240) | NO: 241) | (SEQ ID | (SEQ ID | NO: 41) | (SEQ ID |
| | | | NO: 242) | NO: 40) | | NO: 42) |
| xhCD8v11 | GFTFSSY | TYAGGS | SNAYAW | RASQSVS | GASSRAT | QQYGSSP |
| | (SEQ ID | (SEQ ID | DDALDI | SNLA | (SEQ ID | PVT |
| | NO: 240) | NO: 241) | (SEQ ID | (SEQ ID | NO: 41) | (SEQ ID |
| | | | NO: 242) | NO: 40) | | NO: 42) |
| xhCD8v12 | GGTFSSY | IPGYAT | DAAGIRL | RASQSIY | GASNLQS | QSTYTAP |
| | (SEQ ID | (SEQ ID | FAD (SEQ | GALN | (SEQ ID | WT (SEQ |
| | NO: 238) | NO: 243) | ID NO: 233) | (SEQ ID | NO: 235) | ID NO: 236) |
| | | | | NO: 234) | | |
| xhCD8v13 | GGTFSSY | IPGYAT | DAAGIRL | RASQEIY | GATNLQS | QSTYDAP |
| | (SEQ ID | (SEQ ID | FAD (SEQ | GALN | (SEQ ID | WT (SEQ |
| | NO: 238) | NO: 243) | ID NO: 233) | (SEQ ID | NO: 17) | ID NO: 228) |
| | | | | NO: 16) | | |
| xhCD8v14 | GFTFSSY | SYAGGS | SNAYAW | RASQSVS | GASSRAT | QQYGSSP |
| | (SEQ ID | (SEQ ID | DDALDI | SNLA | (SEQ ID | PVT |
| | NO: 240) | NO: 244) | (SEQ ID | (SEQ ID | NO: 41) | (SEQ ID |
| | | | NO: 242) | NO: 40) | | NO: 42) |
| xhCD8v15 | GFTFSSY | SYAGGS | SNAYAW | RASQSVS | GASSRAT | QQYGSSP |
| | (SEQ ID | (SEQ ID | DDALDI | SNLA | (SEQ ID | PVT |
| | NO: 240) | NO: 244) | (SEQ ID | (SEQ ID | NO: 41) | (SEQ ID |
| | | | NO: 242) | NO: 40) | | NO: 42) |
| V9 family | GX₁X₂FX₃ | X₁PX₂X₃X₄ | X₁X₂X₃GX₄ | X₁X₂SX₃X₄ | GX₁X₂X₃L | QX₁X₂X₃X₄ |
| | X₄X₅ | X₅ | X₅LFX₆X₇ | IX₅GX₆LN | X₄X₅ | X₅PWT |
| | X₁ is G, Y, | X₁ is I, N, | X₁ is D or | X₁ is R or | X₁ is A or | X₁ is S, N, |
| | S, or A, | or M, | A, | G, | S, X₂ is T, | D, Q, A, or |
| | X₂ is T, S, | X₂ is G, N, | X₂ is A, G, | X₂ is A or | S, E, Q, or | E, |
| | G, R, N, or | H, S, R, I, | E, R, Y, K, | T, | D, X₃ is N, | X₂ is T, I, |
| | H, | or A, | N, Q, L, or | X₃ is Q or | R, A, E, or | or S, |
| | X₃ is S, T, | X₃ is A, N, | F, | E, | H, X₄ is Q | X₃ is Y, L, |
| | R, H, Y, G, | H, S, T, F, | X₃ is A, L, | X₄ is E, N, | or A, | or F, |
| | or P, | or Y, | P, or Y, | T, S, A, K, | X₅ is S or D | X₄ is D, G, |
| | X₄ is S, K, | X₄ is A, D, | X₄ is I or L, | D, G, R, or | (SEQ ID | T, E, Q, A, |
| | G, N, R, D, | or G, | X₅ is R, A, | Q, | NO: 263) | or Y, |
| | T, or G, | X₅ is T, E, | Q, or S, | X₅ is Y or | | X₅ is A, T, |
| | X₅ is Y, L, | K, V, Q, or | X₆ is A or | S, X₆ is A | | R, S, K, or |
| | H, or F | A | D, | or V (SEQ | | Y |
| | (SEQ ID | (SEQ ID | X₇ is D, E, | ID NO: 262) | | (SEQ ID |
| | NO: 265) | NO: 266) | A, or S | | | NO: 264) |
| | | | (SEQ ID | | | |
| | | | NO: 267) | | | |
| V11 family | GFTFX₁X₂ | X₁X₂X₃GX₄ | X₁X2X₃YX₄ | RASQSVS | GASSRAT | QQYGSSP |
| | Y | X₅ | WX₅X₆AX₇ | SNAL | (SEQ ID | PV7 |
| | X₁ is S, D, | X₁ is T, N, | DX₈ | (SEQ ID | NO: 41) | (SEQ ID |
| | E, Q, S, or | S, Q, E, H, | X₁ is S or | NO: 40) | | NO: 42) |
| | A | R or A, | A, X₂ is N, | | | |
| | X₂ is S, D, | X₂ is Y, W, | H, A, D, L, | | | |
| | E, A, or Q | F, or H, | Q, Y, or R, | | | |
| | (SEQ ID | X₃ is A, S, | X₃ is A, N, | | | |
| | NO: 271) | Q, E, or T, | S, or G, | | | |
| | | X₄ is G or | X₄ is A, V, | | | |
| | | E, | R, E, or S, | | | |
| | | X₅ is S or I | X₅ is D or | | | |
| | | (SEQ ID | S, X₆ is D, | | | |
| | | NO: 272) | N, Q, E, S, | | | |
| | | | T, or L, | | | |
| | | | X₇ is L, F, | | | |
| | | | or M, | | | |
| | | | X₈ is I, Y, | | | |
| | | | or V | | | |
| | | | (SEQ ID | | | |
| | | | NO: 273) | | | |

TABLE 3
Anti-CD8 antibody variable domain sequences

| Name | VH | VL |
|---|---|---|
| xhCD8v1 | QVHLQQSGPELVKPGASVKMSCKT | DIQMTQSPASLSASVGETVTITCGA |
| | SGYTFTKYTMHWVKQGHEESLEWI | SENIYGALNWYQRKQGKSPQLLIFG |
| | GHFNPNNDETKYNQKFTGKATLTV | ATNLADGVSSRFSGSGSDRQYSLKI |
| | DKSSTTAYMELRSLTSDDSALYYC | SSLHPDDVATYYCONILDTPWTFG |
| | ARDGLGLRLFADWGQGTLITVSA | GGTKLEIK |
| | (SEQ ID NO: 58) | (SEQ ID NO: 59) |
| xhCD8v1.1 | QVQLVQSGAEVKKPGSSVKVSCKA | DIQMTQSPSSLSASVGDRVTITCRA |
| | SGYTFTKYAISWVRQAPGQGLEW | SENIYGALNWYQQKPGKAPKLLIY |
| | MGHFNPNNDETKYNQKFQGRVTIT | GATNLADGVPSRFSGSGSGTDFTLT |
| | ADESTSTAYMELSSLRSEDTAVYY | |
| | CARDGLGLRLFADWGQGT | ISSLQPEDFATYYCQNILDTPWTFG |
| | LVTVSS | GGTKLEIK |
| | (SEQ ID NO: 60) | (SEQ ID NO: 61) |
| xhCD8v2 | QVQLVQSGAEVKKPGSSVKVSCKA | DIQMTQSPSSLSASVGDRVTITCRA |
| | SGYRFHNFAISWVRQAPGQGLEW | SQEIYGALNWYQQKPGKAPKLLIY |
| | MGGIIPGHAKANYAQKFQGRVTIT | GATNLQSGVPSRFSGSGSGTDFTLTI |
| | ADESTSTAYMELSSLRSEDTAVYY | SSLQPEDFATYYCQDIYDAPWTFG |
| | CARDGLGIRLFADWGQGTLVTVSS | GGTKVEIK |
| | (SEQ ID NO: 62) | (SEQ ID NO: 63) |
| xhCD8v3 | QVQLVQSGAEVKKPGSSVKVSCKA | DIQMTQSPSSLSASVGDRVTITCRA |
| | SGSRFYKFAISWVRQAPGQGLEWM | SQEIYGALNWYQQKPGKAPKLLIY |
| | GGIIPGHAKANYAQKFQGRVTITAD | GATNLQSGVPSRFSGSGSGTDFTLTI |
| | ESTSTAYMELSSLRSEDTA VYYCAR | SSLQPEDFATYYCQDIYDAPWTFG |
| | DGLGIRLFADWGQGTLVTVSS | GGTKVEIK |
| | (SEQ ID NO: 64) | (SEQ ID NO: 65) |
| xhCD8v4 | QVQLVQSGAEVKKPGSSVKVSCKA | DIQMTQSPSSLSASVGDRVTITCRA |
| | SGYTFTKYAISWVRQAPGQGLEW | SQKIYGALNWYQQKPGKAPKLLIY |
| | MGGIIPGHAKANYAQKFQGRVTIT | GATNLQSGVPSRFSGSGSGTDFTLTI |
| | ADESTSTAYMELSSLRSEDTAVYY | SSLQPEDFATYYCQNTYDTPWTFG |
| | CARDGLGIRLFADWGQGTLVTVSS | GGTKVEIK |
| | (SEQ ID NO: 66) | (SEQ ID NO: 67) |
| xhCD8v5 | QVQLVQSGAEVKKPGSSVKVSCKA | DIQMTQSPSSLSASVGDRVTITCRA |
| | SGSGFRGHAISWVRQAPGQGLEW | SQKIYGALNWYQQKPGKAPKLLIY |
| | MGGIIPGHAKANYAQKFQGRVTIT | GATNLQSGVPSRFSGSGSGTDFTLTI |
| | ADESTSTAYMELSSLRSEDTAVYY | SSLQPEDFATYYCQNTYDTPWTFG |
| | CARDGLGIRLFADWGQGTLVTVSS | GGTKVEIK |
| | (SEQ ID NO: 68) | (SEQ ID NO: 69) |
| xhCD8v6 | EVOLVESGGGAVRPGGSLRLSCAA | EIVLTQSPATLSVSPGERATLSCRAS |
| | SGFTFDDYGMSWVRQAPGKGLEW | QSVSSNLAWYQQKPGQAPRLLIYG |
| | VSDINWSGEITAYADSVKGRFTISR | ASSRATGIPDRFSGSGSGTDFTLTIS |
| | DNAKNSLYLQMNSLRAEDTAVYY | RLEPEDFAVYYCQQYGSSPPVTFGQ |
| | CARSNSYRWDDALDIWGQGTMVT | GTKVEIK |
| | VSS | (SEQ ID NO: 71) |
| | (SEQ ID NO: 70) | |
| xhCD8v7 | EVOLVESGGGLVQPGRSLRLSCAA | EIVLTQSPATLSVTPGEGATLSCRAS |
| | SGFTFDDYAMHWVRQAPGKGLEW | HSVGSNLAWYQQKPGQAPRLLIYD |
| | VAVISYDGSNKYYADSVKGRFTISR | ASNRATGIPARFSGSGSGTDFTLTIS |
| | DNSKNTLYLQMNSLRAEDTAVYY | SLEPEDLAVYYCQQRSNWPPTFGQ |
| | CAKDRIGWYDYDAFDIWGQGTMV | GTRLEIK |
| | TVSS | (SEQ ID NO: 73) |
| | (SEQ ID NO: 72) | |
| xhCD8v8 | QVQLQQSGAELVRPGSSVKISCKAS | DIQMTQSPASLSVSVGETVTITCRA |
| | GYAFSSYWMNWVKQRPGQGLEWI | SENIYSNLAWYQQKQGKSPQLLVY |
| | GQIYPGDGDTNYNGKFKGKATLTA | AATNLADGVPSRFSGSGSGTQYSL |
| | DKSSSTAYMQLSSLTSEDSAVYFCA | KINSLQSEDFGSYYCQHFWGTPWT |
| | RSGAAFSSYYAMDYWGQGTSVTV | FGGGTKLEIK (SEQ ID NO: 186) |
| | SS (SEQ ID NO: 185) | |
| xhCD8v9 | QVQLVQSGAEVKKPGSSVKVSCKA | DIQMTQSPSSLSASVGDRVTITCRA |
| | SGGTFSSYAISWVRQAPGQGLEWM | SQEIYGALNWYQQKPGKAPKLLIY |
| | GGIIPGAATANYAQKFQGRVTITAD | GATNLQSGVPSRFSGSGSGTDFTLTI |
| | ESTSTAYMELSSLRSEDTA VYYCAR | SSLQPEDFATYYCQSTYDAPWTFG |
| | DAAGIRLFADWGQGTLVTVSS | GGTKVEIK (SEQ ID NO: 246) |
| | (SEQ ID NO: 245) | |
| xhCD8v10 | EVOLVESGGGLVQPGGSLRLSCAA | EIVLTQSPGTLSLSPGERATLSCRAS |
| | SGFTFSSYAMSWVRQAPGKGLEW | QSVSSNLAWYQQKPGQAPRLLIYG |
| | VSDITY AGGSTAYADSVKGRFTISR | ASSRATGIPDRFSGSGSGTDFTLTIS |
| | DNAKNSLYLQMNSLRAEDTAVYY | RLEPEDFAVYYCQQYGSSPPVTFGQ |
| | CARSNAYAWDDALDIWGQGTMVT | GTKVEIK (SEQ ID NO: 248) |
| | VSS (SEQ ID NO: 247) | |
| xhCD8v11 | EVOLVESGGGLVQPGGSLRLSCAA | EIVLTQSPGTLSLSPGERATLSCRAS |
| | SGFTFSSYAMSWVRQAPGKGLEW | QSVSSNLAWYQQKPGQAPRLLIYG |
| | VSDITY AGGSTAYADSVKGRFTISR | ASSRATGIPDRFSGSGSGTDFTLTIS |
| | DNAKNSLYLQMNSLRAEDTAVYY | RLEPEDFAVYYCQQYGSSPPVTFGQ |
| | CARSNAYAWDDALDIWGQGTLVT | GTKVEIK (SEQ ID NO: 250) |
| | VSS (SEQ ID NO: 249) | |
| xhCD8v12 | QVQLVQSGAEVKKPGSSVKVSCKA | DIQMTQSPSSLSASVGDRVTITCRA |
| | SGGTFSSYAISWVRQAPGQGLEWM | SQSIYGALNWYQQKPGKAPKLLIY |
| | GGIIPGYATANYAQKFQGRVTITAD | GASNLQSGVPSRFSGSGSGTDFTLTI |
| | ESTSTAYMELSSLRSEDTA VYYCAR | SSLQPEDFATYYCQSTYTAPWTFG |
| | DAAGIRLFADWGQGTLVTVSS | GGTKVEIK (SEQ ID NO: 252) |
| | (SEQ ID NO: 251) | |
| xhCD8v13 | QVQLVQSGAEVKKPGSSVKVSCKA | DIQMTQSPSSLSASVGDRVTITCRA |
| | SGGTFSSYAISWVRQAPGQGLEWM | SQEIYGALNWYQQKPGKAPKLLIY |
| | GGIIPGYATANYAQKFQGRVTITAD | GATNLQSGVPSRFSGSGSGTDFTLTI |
| | ESTSTAYMELSSLRSEDTA VYYCAR | SSLQPEDFATYYCQSTYDAPWTFG |
| | DAAGIRLFADWGQGTLVTVSS | GGTKVEIK (SEQ ID NO: 254) |
| | (SEQ ID NO: 253) | |
| xhCD8v14 | EVOLVESGGGLVQPGGSLRLSCAA | EIVLTQSPGTLSLSPGERATLSCRAS |
| | SGFTFSSYAMSWVRQAPGKGLEW | QSVSSNLAWYQQKPGQAPRLLIYG |
| | VSDISYAGGSTAYADSVKGRFTISR | ASSRATGIPDRFSGSGSGTDFTLTIS |
| | DNAKNSLYLQMNSLRAEDTAVYY | RLEPEDFAVYYCQQYGSSPPVTFGQ |
| | CARSNAYAWDDALDIWGQGTMVT | GTKVEIK (SEQ ID NO: 256) |
| | VSS (SEQ ID NO: 255) | |
| xhCD8v15 | EVOLVESGGGLVQPGGSLRLSCAA | EIVLTQSPGTLSLSPGERATLSCRAS |
| | SGFTFSSYAMSWVRQAPGKGLEW | QSVSSNLAWYQQKPGQAPRLLIYG |
| | VSDISYAGGSTAYADSVKGRFTISR | ASSRATGIPDRFSGSGSGTDFTLTIS |
| | DNAKNSLYLQMNSLRAEDTAVYY | RLEPEDFAVYYCQQYGSSPPVTFGQ |
| | CARSNAYAWDDALDIWGQGTLVT | GTKVEIK (SEQ ID NO: 258) |
| | VSS (SEQ ID NO: 257) | |

In some embodiments, the first moiety comprises an antibody (e.g., an anti-CD8 antibody of the present disclosure). In some embodiments, the first moiety comprises an antibody fragment (e.g., an anti-CD8 antibody fragment of the present disclosure). In some embodiments, the first moiety comprises a single chain antibody or single chain variable fragment (scFv). In some embodiments, the first moiety comprises a VHH antibody. In some embodiments, the first moiety comprises one or two antibody heavy chain polypeptides and one or two antibody light chain polypeptides (e.g., of an anti-CD8 antibody of the present disclosure). In some embodiments, the first moiety comprises the 3 heavy chain CDRs and/or 3 light chain CDRs of a single anti-CD8 antibody of the present disclosure, e.g., as shown in Tables 1-3. In some embodiments, the first moiety comprises the VH and/or VL domain(s) of a single anti-CD8 antibody of the present disclosure, e.g., as shown in Table 3. In some embodiments, the first moiety further comprises one or two human IgG Fc domains. In some embodiments, the one or two human IgG Fc domains are IgG1, IgG2, IgG3 or IgG4 Fc domains. In some embodiments, the one or two human IgG Fc domains do not have the C-terminus lysine residue. In some embodiments, the one or two human IgG Fc domains comprise amino acid modifications (such as substitutions, deletions, additions, etc.). In some embodiments, the Fc domain modifications promote heterodimeric formation (e.g., as shown in Table 4). In some embodiments, the one or two Fc domains comprise Fc gamma-null mutations.

In some embodiments, the first moiety comprises two antibody heavy chain polypeptides comprising a structure according to formula [I], from N-terminus to C-terminus:

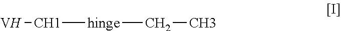

and two antibody light chain polypeptides comprising a structure according to formula [II], from N-terminus to C-terminus:

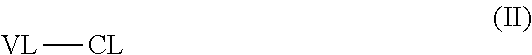

wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain. In some embodiments, the N-terminus of the second moiety is fused to the C-terminus of one of the two CH3 domains (see, e.g., format A in FIG. 2A).

In some embodiments, the first moiety comprises a first antibody heavy chain polypeptide comprising a structure according to formula [I], from N-terminus to C-terminus:

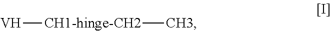

an antibody light chain polypeptide comprising a structure according to formula [II], from N-terminus to C-terminus:

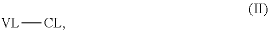

and a second antibody heavy chain polypeptide comprising a structure according to formula [III], from N-terminus to C-terminus:

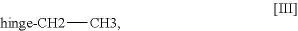

wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain. In some embodiments, the N-terminus of the second moiety is fused to the C-terminus of the CH3 domain of the second antibody heavy chain polypeptide (see, e.g., format B in FIG. 2A). In some embodiments, the N-terminus of the second moiety is fused to the C-terminus of the CH3 domain of the first antibody heavy chain polypeptide.

In some embodiments, the first moiety comprises a first antibody heavy chain polypeptide comprising a structure according to formula [I], from N-terminus to C-terminus:

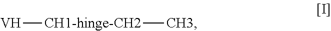

an antibody light chain polypeptide comprising a structure according to formula [II], from N-terminus to C-terminus:

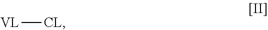

and a second antibody heavy chain polypeptide comprising a structure according to formula [III], from N-terminus to C-terminus:

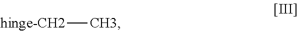

wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain. In some embodiments, the C-terminus of the second moiety is fused to the N-terminus of the hinge domain of the second antibody heavy chain polypeptide (see, e.g., format C in FIG. 2A).

In some embodiments, an anti-CD8 antibody of the present disclosure is a multispecific (e.g., bispecific) antibody or antibody fragment. For example, in some embodiments, the multispecific antibody (e.g., bispecific antibody) comprises a first antigen binding domain that binds to CD8 (e.g., as described supra) and a second antigen binding domain that binds a target of interest. In some embodiments, a bispecific antibody can be generated via fusion of an additional binding site to either the heavy or light chain of an immunoglobulin. Examples of the additional binding site include but not limited to variable regions, scFv, Fab, VHH, and peptide.

In some embodiments, the recombinant bispecific antibodies disclosed herein can be very roughly classified in two categories, namely i) formats resulting from the combination of variable regions only and ii) formats combining variable regions with Fc domains. Representatives of the first category are tandem scFv (taFv), diabodies (Db), DART, single-chain diabodies (scDbs), Fab-Fc, tandem Fab, Dual variable region Fab and tandem dAb/VHH. The two variable regions can be linked together via covalent bonds or non-covalent interaction.

Noncovalent interaction may involve the use of heterodimerization modules such as leucine zipper, dock-and-lock methods of using regulatory subunit of cAMP-dependent protein kinase (PKA) and the anchoring domains of A kinase anchor proteins (AKAPs) or knob-into-holes CH3 domain (U.S. Pat. Nos. 5,731,168; 7,695,936; Ridgway et al., Prot Eng 9, 617-621 (1996) and Carter, J Immunol Meth 248, 7-15 (2001)) to pair up the variable regions.

In some embodiments, bispecific antibodies are generated on the natural immunoglobulin architecture containing two pairs of heavy chain and light chain combination with each pair having distinct binding specificity. Homodimerization of the two heavy chains in an IgG is mediated by the CH3 interaction. To promote heterodimeric formation, genetic modifications are introduced to the two respective CH3 regions. There heterodimerization mutations often involve steric repulsion, charge steering interaction, or interchain disulfide bond formation. Exemplary and non-limiting Fc modifications to promote heterodimerization include the following:

TABLE 4
Exemplary Fc modifications to promote heterodimerization.

| | CH3 | CH3 | |
|---|---|---|---|
| Strategy | domain 1 | domain 2 | References |
| knobs-into-holes 1 | T366Y | Y407T | Brinkmann & |
| knobs-into-holes 2 | T366W | T366S-L368A- | Kontermann, MAbs. |
| | | Y407V | 2017 February-March; |
| | | | 9(2): 182-212; |
| knobs-into-holes 3 | S354C, T366W | Y349C, T366S, | Atwell et al, J Mol |
| | | L368A, Y407V | Biol 1997; 270: 26-35; |
| | | | Merchant et al, |
| | | | Nat Biotechnol 1998; |
| knobs-into-holes 4 | Y349C, T366W | S354C, T366S, | 16: 677-681 |
| | | L368A, Y407V | |
| HA-TF | S364H, F405A | Y349T, T394F | Moore et al, MAbs 2011; |
| | | | 3: 546-557 |
| ZW1 | T350V, L351Y, | T350V, T366L, | Von Kreudenstein et al, |
| | F405A, Y407V | K392L, T394W | MAbs 2013; 5: 646-54 |
| CH3 charge pairs | K392D, K409D | E356K, D399K | Gunasekaran et al, J Biol |
| (DD-KK) | | | Chem 2010; 285: 19637-46 |
| IgG1 hinge/CH3 charge | IgG1: D221E, | IgG1: D221R, | Strop P et al, J Mol |
| pairs (EEE-RRR) | P228E, L368E | P228R, K409R | Biol 2012; 420: 204-19 |
| IgG2 hinge/CH3 charge | IgG2: C223E, | IgG2: C223R, | |
| pairs (EEE-RRRR) | P228E, L368E | E225R, P228R, | |
| | | K409R | |
| EW-RVT | K360E, K409W, | Q347R, D399V, | Choi et al, Mol Cancer |
| | | F405T | Ther 2013; 12: 2748-59 |
| EW-RVT_{S-S} | K360E, K409W, | Q347R, D399V, | Choi et al, Mol |
| | Y349C | F405T, S354C | Immunol 2015; 65: 377-83 |
| Biclonic | 366K (+351K) | 351D or E or D at | Geuijen et al, ournal of |
| | | 349, 368, 349, | Clinical Oncology 2014; |
| | | or 349 + 355 | 32: suppl: 560 |
| DuoBody (L-R) 1 | F405L | K409R | Labrijn et al, Nat; |
| DuoBody (L-R) 2 | F405L-R409K | WT (R409) | Protoc 2014; 9: 2450-63 |
| | | | Labrijn et al, PNAS |
| | | | 2013; 110(13): 5145-50 |
| SEEDbody | IgG/A | IgG/A | Davis et al, |
| | chimera | chimera | Protein Eng Des |
| | | | Sel 2010; 23:195-202 |
| BEAT | residues from | residues from | Moretti et al, BMC |
| | TCRα | TCRβ | Proceedings 2013; |
| | interface | interface | 7(Suppl 6): O9 |
| Mixed interface (MI) | IgG-CH3 | igA/D/M | Skegro et al, J Biol |
| heterodimers | variants | CH3 variants | Chem. 2017 292(23): |
| | | or IgM CH4 | 9745-9759. |
| | | variants | |
| XmAb | E357Q-S364K | L368D-K370S | Moore et al, Methods. |
| | | | 2019 154: 38-50 |
| DEKK Fc | L351D-L368E | L351K-T366K | De Nardis et al., J Biol |
| | | | Chem. 2017; 292(35): |
| | | | 14706-14717. |
| Charge pair | E356K or E357K | K370E, K409D, | Igawa T, Tsunoda H. |
| | or D399K | K439E | WO2006106905. 2006. |
| KKA-DDW | D356K-D399K- | K392D-K409D- | Zhou et al, |
| | Y407A | T366W | WO2014079000A1 |
| Charge pair | L368E-Y407E | E357K-D399K | Labrijn et al, Nat |
| | | | Review Drug Discovery |
| | | | 2019; 18, 585-608 |
| Knob-hole-electrostatic | S354C-T366W- | Y349C-T366S- | Wei et al., Oncotarget. |
| | K409A | L368A-Y407-F405K | 2017; 8(31): 51037-51049 |
| KA | F405K | K409A | Wei et al., Oncotarget. |
| | | | 2017; 8(31): 51037-51049 |
| PPV-TPP | P395K-P396K- | T394D-P395D- | Wenjun Zhang |
| | V397K, P395K- | P396D, T394C- | U.S. Pat. No. 10,538,595B2 |
| | P396K-V397C or | P395D-P396D | |
| | P395R-P396R- | or T394E- | |
| | V397R | P395E-P396E | |

In some embodiments, bispecific antibody can be generated by post-production assembly from half-antibodies, thereby solving the issues of heavy and light chain mispairing. These antibodies often contain modification to favor heterodimerization of half-antibodies. Exemplary systems include but not limited to the knob-into-hole, IgG1 (EEE-RRR), IgG2 (EEE-RRRR) (Strop et al. J Mol Biol (2012)) and DuoBody (F405L-K409R), listed in Table 5. In such case, half-antibody is individually produced in separate cell line and purified. The purified antibodies were then subjected to mild reduction to obtain half-antibodies, which were then assembled into bispecific antibodies. Heterodimeric bispecific antibody was then purified from the mixture using conventional purifications methods.

In some embodiments, strategies on bispecific antibody generation that do not rely on the preferential chain pairing can also be employed. These strategies typically involve introducing genetic modification on the antibody in such a manner that the heterodimer will have distinct biochemical or biophysical properties from the homodimers; thus the post-assembled or expressed heterodimer can be selectively purified from the homodimers. One example was to introduce H435R/Y436F in IgG1 CH3 domain to abolish the Fc binding to protein A resin and then co-express the H435R/Y436F variant with a wildtype Fc. The resulting homodimeric antibodies containing two copies of H435R/Y436F cannot bind to the Protein A column, while heterodimeric antibody comprising one copy of H435R/Y436F mutation will have a decreased affinity for protein A as compared to the strong interaction from homodimeric wildtype antibody (Tustian et al Mabs 2016). Other examples include kappa/lambda antibody (Fischer et al., Nature Communication 2015) and introduction of differential charges (E357Q, S267K or N208D/Q295E/N384D/Q418E/N421D) on the respective chains (US 2018/0142040 A1; (Strop et al. J Mol Biol (2012)).

In some embodiments, bispecific antibody can be generated via fusion of an additional binding site to either the heavy or light chain of an immunoglobulin. Examples of the additional binding site include but not limited to variable regions, scFv, Fab, VHH, and peptide.

In some embodiments, an antibody or fusion protein of the present disclosure comprises an Fc region. In some embodiments, the Fc region comprises one or more mutations that reduce or eliminate FcγR binding and/or effector function. In some embodiments, the Fc region (e.g., an IgG1 Fc region) comprises a substitution at one or more of the following positions: C220, C226, C229, E233, L234, L235, G237, P238, S239 D265, S267, N297, L328, P331, K322, A327 and P329. In some embodiments, the Fc region (e.g., an IgG2 Fc region) comprises a substitution at one or more of the following positions: V234, G237, D265, H268, N297, V309, A330, A331, K322. In some embodiments, the Fc region (e.g., an IgG4 Fc region) comprises a substitution at one or more of the following positions: L235, G237, D265 and E318. In some embodiments, the Fc region (e.g., an IgG1 Fc region) comprises one or more of the following mutations or groups of mutations: N297A, N297Q, N297G, D265A/N297A, D265A/N297Q, C220S/C226S/C229S/P238S, S267E/L328F, C226S/C229S/E233P/L234V/L235A, L234F/L235E/P331S, L234A/L235A, L234A/L235A/G237A, L234A/L235A/G237A/K322A, L234A/L235A/G237A/A330S/A331S, L234A/L235A/P329G,E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, L234A/L235A/G237deleted. In some embodiments, the Fc region (e.g., an IgG2 Fc region) comprises one or more of the following mutations or groups of mutations: A330S/A331S, V234A/G237A, V234A/G237A/D265A, D265A/A330S/A331S, V234A/G237A/D265A/A330S/A331S, and H268Q/V309L/A330S/A331S. In some embodiments, the Fc region (e.g., an IgG4 Fc region) comprises one or more of the following mutations or groups of mutations: L235A/G237A/E318A, D265A, L235A/G237A/D265A and L235A/G237A/D265A/E318A. In some embodiments, the Fc region comprises one, two, three, or all of the following mutations: L234A, L235A, G237A, and K322A, numbering according to EU index. In some embodiments, the Fc region comprises one, two, or all of the following mutations: L234A, L235A, and G237A, numbering according to EU index.

In some embodiments, said first and second Fc domains of the fusion protein contain one or more of the following Fc mutations to decrease effector function according to EU numbering: L234A, L235A, G237A, and K322A. In some embodiments, said first and second Fc domains of the fusion protein contain the following Fc mutations to decrease effector function according to EU numbering: L234A, L235A, and G237A. In some embodiments, said first and second Fc domains of the fusion protein contain the following Fc mutations to decrease effector function according to EU numbering: L234A, L235A, G237A, and K322A. In some embodiments, said first and second Fc domains of the fusion protein contain the following amino acid substitutions to facilitate heterodimeric formation: Y349C/T366W (knob) and S354C, T366S, L368A and Y407V (hole).

In some embodiments, the heterodimeric mutations and/or mutations to modify Fc gamma receptor binding resulted in reduction of Fc stability. Therefore, additional mutation(s) was added to the Fc region to increase its stability. For example, one or more pairs of disulfide bonds such as A287C and L306C, V259C and L306C, R292C and V302C, and V323C and 1332C are introduced into the Fc region. Another example is to introduce S228P to IgG4 based bispecific antibodies to stabilize the hinge disulfide. Additional example includes introducing K338I, A339K, and K340S mutations to enhance Fc stability and aggregation resistance (Gao et al, 2019 Mol Pharm. 2019; 16:3647).

In some embodiments, the second moiety induces activation of CD8+ T cells. In some embodiments, the second moiety comprises a polypeptide that induces signaling via IL2Rβγ. For example, in some embodiments, the second moiety comprises an IL-15 polypeptide (e.g., a human IL-15 polypeptide or derivative thereof) or a neoleukin. In some embodiments, the second moiety comprises an IL-21 polypeptide (e.g., a human IL-21 polypeptide or derivative thereof).

In some embodiments, the second moiety comprises an IL-2 polypeptide (e.g., a human IL-2 polypeptide or derivative thereof).

In some embodiments, the mutant IL-2 polypeptide has a binding affinity to IL-2Ra that is reduced by 50% or more, compared to binding affinity of a wild-type IL-2 polypeptide comprising the sequence of SEQ ID: 81 for IL-2Rα. In some embodiments, the mutant IL-2 polypeptide has a binding affinity to IL-2Rβ that is reduced by 50% or more, compared to binding affinity of a wild-type IL-2 polypeptide comprising the sequence of SEQ ID: 81 for IL-2Rβ. In some embodiments, the mutant IL-2 polypeptide has a binding affinity to IL-2Rγ that is reduced by 50% or more, compared to binding affinity of a wild-type IL-2 polypeptide comprising the sequence of SEQ ID: 81 for IL-2Rγ. Differences in binding affinity of wild-type and disclosed mutant polypeptide for IL-2Rα and IL-2Rβ can be measured, e.g., in standard surface plasmon resonance (SPR) assays that measure affinity of protein-protein interactions familiar to those skilled in the art. Differences in binding affinity of wild-type and disclosed mutant polypeptide for IL-2Rγ cannot reliably be measured by SPR assays as the affinity of wild-type IL-2 polypeptide for IL-2Rγ is very low. Instead, their reduced affinity to IL-2Rγ can be deduced by performing an in vitro assay that measures pSTAT5 and compares the activity of IL-2 polypeptides with and without the IL-2Rγ affinity-reducing substitution on IL-2R-expressing cells. Exemplary sequences for IL-2Rα, IL-2Rβ, and IL-2Rγ are provided below.

IL-2Rα:
(SEQ ID NO: 82)
MDSYLLMWGLLTFIMVPGCQAELCDDDPPEIPHATFKAMAYKEGTMLN
CECKRGFRRIKSGSLYMLCTGNSSHSSWDNQCQCTSSATRNTTKQVTP
QPEEQKERKTTEMQSPMQPVDQASLPGHCREPPPWENEATERIYHFVV
GQMVYYQCVQGYRALHRGPAESVCKMTHGKTRWTQPQLICTGEMETSQ
FPGEEKPQASPEGRPESETSCLVTTTDFQIQTEMAATMETSIFTTEYQ
VAVAGCVFLLISVLLLSGLTWQRRQRKSRRTI
IL-2β:
(SEQ ID NO: 83)
SCQVHAWPDRRRWNQTCELLPVSQASWACNLILGAPDSQKLTTVDIVT
LRVLCREGVRWRVMAIQDFKPFENLRLMAPISLQVVHVETHRCNISWE
ISQASHYFERHLEFEARTLSPGHTWEEAPLLTLKQKQEWICLETLTPD
TQYEFQVRVKPLQGEFTTWSPWSQPLAFRTKPAALGKDTIPWLGHLLV
GLSGAFGFIILVYLLINCRNTGPWLKKVLKCNTPDPSKFFSQLSSEHG
GDVQKWLSSPFPSSSFSPGGLAPEISPLEVLERDKVTQLLLQQDKVPE
PASLSSNHSLTSCFTNQGYFFFHLPDALEIEACQVYFTYDPYSEEDPD
EGVAGAPTGSSPQPLQPLSGEDDAYCTFPSRDDLLLFSPSLLGGPSPP
STAPGGSGAGEERMPPSLQERVPRDWDPQPLGPPTPGVPDLVDFQPPP
ELVLREAGEEVPDAGPREGVSFPWSRPPGQGEFRALNARLPLNTDAYL
SLQELQGQDPTHLV
IL-2Rγ:
(SEQ ID NO: 84)
MLKPSLPFTSLLFLQLPLLGVGLNTTILTPNGNEDTTADFFLTTMPTD
SLSVSTLPLPEVQCFVFNVEYMNCTWNSSSEPQPTNLTLHYWYKNSDN
DKVQKCSHYLFSEEITSGCQLQKKEIHLYQTFVVQLQDPREPRRQATQ
MLKLQNLVIPWAPENLTLHKLSESQLELNWNNRFLNHCLEHLVQYRTD
WDHSWTEQSVDYRHKFSLPSVDGQKRYTFRVRSRFNPLCGSAQHWSEW
SHPIHWGSNTSKENPFLFALEAVVISVGSMGLIISLLCVYFWLERTMP
RIPTLKNLEDLVTEYHGNFSAWSGVSKGLAESLQPDYSERLCLVSEIP
PKGGALGEGPGASPCNQHSPYWAPPCYTLKPET

In some embodiments, the IL-2 polypeptide is a mutant IL-2 polypeptide comprising one or more mutations relative to a human IL-2 polypeptide comprising the sequence of APTSSSTKKTQLQLEHLLLDLQMILNGINNYKNPKLTRMLTFKFYMPKKATELKHLQ CLEEELKPLEEVLNLAQSKNFHLRPRDLISNINVIVLELKGSETTFMCEYADETATIVE FLNRWITFCQSIISTLT (SEQ ID NO:81). In some embodiments, the mutant IL-2 polypeptides of the present disclosure have one or more, two or more, or three or more affinity-reducing amino acid substitutions relative to the wild-type mature IL-2 polypeptide, e.g., having the amino acid sequence of SEQ ID NO:81. In some embodiments, one or more, two or more, or three or more substituted residues, are selected from the following group: Q11, H16, L18, L19, D20, D84, S87, Q22, R38, F42, K43, Y45, E62, P65, E68, V69, L72, D84, S87, N88, V91, 192, T123, Q126, S127, 1129, and S130. Decreased affinity to IL-2Rα may be obtained by substituting one or more of the following residues in the sequence of the wild-type mature IL-2 polypeptide: R38, F42, K43, Y45, E62, P65, E68, V69, and L72. Decreased affinity to IL-2Rβ may be obtained by substituting one or more of the following residues: E15, H16, L19, D20, D84, S87, N88, V91, and 192. Decreased affinity to IL-2Rγ may be obtained by substituting one or more of the following residues in the sequence of the wild-type mature IL-2 polypeptide: Q11, L18, Q22, T123, Q126, S127, 1129, and S130.

In some embodiments, the mutant IL-2 polypeptide comprises an F42A or F42K amino acid substitution relative to the wild-type mature IL-2 amino acid sequence. In some embodiments, the mutant IL-2 polypeptide comprises an F42A or F42K amino acid substitution and an R38A, R38D, R38E, E62Q, E68A, E68Q, E68K, or E68R amino acid substitution relative to the wild-type mature IL-2 amino acid sequence. For example, in some embodiments, the mutant IL-2 polypeptide comprises F42A; R38A and F42A; R38D and F42A; R38E and F42A; F42A and E62Q; F42A and E68A; F42A and E68Q; F42A and E68K; F42A and E68R; or R38A and F42K amino acid substitution(s) relative to the wild-type mature IL-2 amino acid sequence, e.g., as shown in SEQ ID NO:81. In some embodiments, the mutant IL-2 polypeptide comprises R38E and F42A amino acid substitutions relative to the wild-type IL-2 amino acid sequence. In some embodiments, the mutant IL-2 polypeptide comprises R38D and F42A amino acid substitutions relative to the wild-type IL-2 amino acid sequence. In some embodiments, the mutant IL-2 polypeptide comprises F42A and E62Q amino acid substitutions relative to the wild-type IL-2 amino acid sequence. In some embodiments, the mutant IL-2 polypeptide comprises R38A and F42K amino acid substitutions relative to the wild-type IL-2 amino acid sequence. In some embodiments, the mutant IL-2 polypeptide comprises R38D and F42A amino acid substitutions relative to the wild-type IL-2 amino acid sequence. In some embodiments, the mutant IL-2 polypeptide comprises R38A and F42K amino acid substitutions relative to the wild-type IL-2 amino acid sequence. In some embodiments, the mutant IL-2 polypeptide comprises F42A and E62Q amino acid substitutions relative to the wild-type IL-2 amino acid sequence. In some embodiments, the mutant IL-2 polypeptide comprises an H16E, H16D, D20N, M23A, M23R, M23K, D84L, D84N, D84V, D84H, D84Y, D84R, D84K, S87K, S87A, N88A, N88D, N88G, N88S, N88T, N88R, N88I, V91A, V91T, V91E, 192A, E95S, E95A, E95R, T123A, T123E, T123K, T123Q, Q126A, Q126S, Q126T, Q126E, S127A, S127E, S127K, or S127Q amino acid substitution relative to the wild-type IL-2 amino acid sequence. In some embodiments, the mutant IL-2 polypeptide comprises F42A; R38A and F42A; R38D and F42A; R38E and F42A; F42A and E62Q; F42A and E68A; F42A and E68Q; F42A and E68K; F42A and E68R; or R38A and F42K amino acid substitution(s) relative to the wild-type mature IL-2 amino acid sequence and an H16E, H16D, D20N, M23A, M23R, M23K, D84L, D84N, D84V, D84H, D84Y, D84R, D84K, S87K, S87A, N88A, N88D, N88G, N88S, N88T, N88R, N88I, V91A, V91T, V91E, 192A, E95S, E95A, E95R, T123A, T123E, T123K, T123Q, Q126A, Q126S, Q126T, Q126E, S127A, S127E, S127K, or S127Q amino acid substitution relative to the wild-type mature IL-2 amino acid sequence, e.g., as shown in SEQ ID NO:81. For example, in some embodiments, the mutant IL-2 polypeptide comprises R38E, F42A, and H16E amino acid substitutions relative to the wild-type IL-2 amino acid sequence. In some embodiments, the mutant IL-2 polypeptide comprises R38E, F42A, and H16D amino acid substitutions relative to the wild-type IL-2 amino acid sequence. In some embodiments, the mutant IL-2 polypeptide comprises R38E, F42A, and D84K amino acid substitutions relative to the wild-type IL-2 amino acid sequence. In some embodiments, the mutant IL-2 polypeptide comprises R38E, F42A, and D84R amino acid substitutions relative to the wild-type IL-2 amino acid sequence. In some embodiments, the mutant IL-2 polypeptide comprises R38E, F42A, and N88S amino acid substitutions relative to the wild-type IL-2 amino acid sequence. In some embodiments, the mutant IL-2 polypeptide comprises R38E, F42A, and N88A amino acid substitutions relative to the wild-type IL-2 amino acid sequence. In some embodiments, the mutant IL-2 polypeptide comprises R38E, F42A, and N88G amino acid substitutions relative to the wild-type IL-2 amino acid sequence. In some embodiments, the mutant IL-2 polypeptide comprises R38E, F42A, and N88R amino acid substitutions relative to the wild-type IL-2 amino acid sequence. In some embodiments, the mutant IL-2 polypeptide comprises R38E, F42A, and N88T amino acid substitutions relative to the wild-type IL-2 amino acid sequence. In some embodiments, the mutant IL-2 polypeptide comprises R38E, F42A, and N88D amino acid substitutions relative to the wild-type IL-2 amino acid sequence. In some embodiments, the mutant IL-2 polypeptide comprises R38E, F42A, and V91E amino acid substitutions relative to the wild-type IL-2 amino acid sequence. In some embodiments, the mutant IL-2 polypeptide comprises R38E, F42A, and Q126S amino acid substitutions relative to the wild-type IL-2 amino acid sequence. In some embodiments, the mutant IL-2 polypeptide comprises the amino acid sequence of SEQ ID NO:81 with one of the following sets of amino acid substitutions (relative to the sequence of SEQ ID NO:81): R38E and F42A; R38D and F42A; F42A and E62Q; R38A and F42K; R38E, F42A, and N88S; R38E, F42A, and N88A; R38E, F42A, and N88G; R38E, F42A, and N88R; R38E, F42A, and N88T; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88R; R38D, F42A and N88T; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88R; R38A, F42K, and N88T; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88R; F42A, E62Q, and N88T; F42A, E62Q, and N88D; F42A, E62Q, and V91E; F42A, E62Q, and D84H; F42A, E62Q, and D84K; F42A, E62Q, and D84R; H16D, F42A, and E62Q; H16E, F42A, and E62Q; F42A, E62Q, and Q126S; R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88R, and C125A; R38E, F42A, N88T, and C125A; R38E, F42A, N88D, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88R, and C125A; R38D, F42A, N88T, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88A, and C125A; R38A, F42K, N88G, and C125A; R38A, F42K, N88R, and C125A; R38A, F42K, N88T, and C125A; R38A, F42K, N88D, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88R, and C125A; F42A, E62Q, N88T, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88R, and C125A; F42A, N88T, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S. In some embodiments, the IL-2 polypeptide comprises the sequence of SEQ ID NO:81 with one, two, three, four, or five amino acid substitutions relative to SEQ ID NO:81, and wherein the one, two, three, four, or five substitution(s) comprise substitution(s) at positions of SEQ ID NO:81 selected from the group consisting of: Q11, H16, L18, L19, D20, Q22, R38, F42, K43, Y45, E62, P65, E68, V69, L72, D84, S87, N88, V91, 192, T123, Q126, S127, 1129, and S130. In some embodiments, the IL-2 polypeptide comprises the sequence of SEQ ID NO:81 with one of the following sets of amino acid substitutions (relative to the sequence of SEQ ID NO:81): R38E and F42A; R38D and F42A; F42A and E62Q; R38A and F42K; R38E, F42A, and N88S; R38E, F42A, and N88A; R38E, F42A, and N88G; R38E, F42A, and N88R; R38E, F42A, and N88T; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88R; R38D, F42A and N88T; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88R; R38A, F42K, and N88T; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88R; F42A, E62Q, and N88T; F42A, E62Q, and N88D; F42A, E62Q, and V91E; F42A, E62Q, and D84H; F42A, E62Q, and D84K; and F42A, E62Q, and D84R.

In some embodiments, the IL-2 polypeptide comprises the sequence of SEQ ID NO: 81 with a further amino acid substitution relative to SEQ ID NO:81 at position C125. In some embodiments, the IL-2 polypeptide comprises the sequence of SEQ ID NO:81 with one of the following sets of amino acid substitutions (relative to the sequence of SEQ ID NO:81): R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88R, and C125A; R38E, F42A, N88D, and C125A; R38E, F42A, N88T, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88R, and C125A; R38D, F42A, N88T, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88R, and C125A; R38A, F42K, N88T, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88R, and C125A; F42A, E62Q, N88T, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88R, and C125A; F42A, N88T, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S.

In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISAINVIVLELKGSETTFMCEYADETATIVE FLNRWITFAQSIISTLT (SEQ ID NO:80). In some embodiments, the mutant IL-2 polypeptide comprises the amino acid sequence of

APTSSSTKKTQLQLEHLLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFCQSIISTLT (SEQ ID NO:85). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISNINVIVLELKGSETTFMCEYADETATIV EFLNRWITFCQSIISTLT (SEQ ID NO:86). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISNINVIVLELKGSETTFMCEYADETATIVE FLNRWITFCQSIISTLT (SEQ ID NO:87). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISNINVIVLELKGSETTFMCEYADETATIV EFLNRWITFCQSIISTLT (SEQ ID NO:88). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISSINVIVLELKGSETTFMCEYADETATIVE FLNRWITFCQSIISTLT (SEQ ID NO:89). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISAINVIVLELKGSETTFMCEYADETATIVE FLNRWITFCQSIISTLT (SEQ ID NO:90). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISNINEIVLELKGSETTFMCEYADETATIVE FLNRWITFCQSIISTLT (SEQ ID NO:91). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRHLISNINVIVLELKGSETTFMCEYADETATIVE FLNRWITFCQSIISTLT (SEQ ID NO:92). In some embodiments, the mutant IL-2 polypeptide comprises the amino acid sequence of APTSSSTKKTQLQLEDLLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFCQSIISTLT (SEQ ID NO:93). In some embodiments, the mutant IL-2 polypeptide comprises the amino acid sequence of APTSSSTKKTQLQLEELLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFCQSIISTLT (SEQ ID NO:94). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFCSSIISTLT (SEQ ID NO:95). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISSINVIVLELKGSETTFMCEYADETATIV EFLNRWITFCQSIISTLT (SEQ ID NO:96). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISAINVIVLELKGSETTFMCEYADETATIV EFLNRWITFCQSIISTLT (SEQ ID NO:97). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISNINEIVLELKGSETTFMCEYADETATIV EFLNRWITFCQSIISTLT (SEQ ID NO:98). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRHLISNINVIVLELKGSETTFMCEYADETATIV EFLNRWITFCQSIISTLT (SEQ ID NO:99). In some embodiments, the mutant IL-2 polypeptide comprises the amino acid sequence of APTSSSTKKTQLQLEDLLLDLQMILNGINNYKNPKLTDMLTAKFYMPKKATELKHL QCLEEELKPLEEVLNLAQSKNFHLRPRDLISNINVIVLELKGSETTFMCEYADETATIV EFLNRWITFCQSIISTLT (SEQ ID NO:100). In some embodiments, the mutant IL-2 polypeptide comprises the amino acid sequence of APTSSSTKKTQLQLEELLLDLQMILNGINNYKNPKLTDMLTAKFYMPKKATELKHLQ CLEEELKPLEEVLNLAQSKNFHLRPRDLISNINVIVLELKGSETTFMCEYADETATIVE FLNRWITFCQSIISTLT (SEQ ID NO:101). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISNINVIVLELKGSETTFMCEYADETATIV EFLNRWITFCSSIISTLT (SEQ ID NO:102). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISSINVIVLELKGSETTFMCEYADETATIV EFLNRWITFCQSIISTLT (SEQ ID NO:103). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISAINVIVLELKGSETTFMCEYADETATIV EFLNRWITFCQSIISTLT (SEQ ID NO:104). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISNINEIVLELKGSETTFMCEYADETATIV EFLNRWITFCQSIISTLT (SEQ ID NO:105). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRHLISNINVIVLELKGSETTFMCEYADETATIV EFLNRWITFCQSIISTLT (SEQ ID NO:106). In some embodiments, the mutant IL-2 polypeptide comprises the amino acid sequence of APTSSSTKKTQLQLEDLLLDLQMILNGINNYKNPKLTAMLTKKFYMPKKATELKHL QCLEEELKPLEEVLNLAQSKNFHLRPRDLISNINVIVLELKGSETTFMCEYADETATIV EFLNRWITFCQSIISTLT (SEQ ID NO:107). In some embodiments, the mutant IL-2 polypeptide comprises the amino acid sequence of APTSSSTKKTQLQLEELLLDLQMILNGINNYKNPKLTAMLTKKFYMPKKATELKHLQ CLEEELKPLEEVLNLAQSKNFHLRPRDLISNINVIVLELKGSETTFMCEYADETATIVE FLNRWITFCQSIISTLT (SEQ ID NO:108). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISNINVIVLELKGSETTFMCEYADETATIV EFLNRWITFCSSIISTLT (SEQ ID NO:109). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISSINVIVLELKGSETTFMCEYADETATIVE FLNRWITFCQSIISTLT (SEQ ID NO:110). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISAINVIVLELKGSETTFMCEYADETATIVE FLNRWITFCQSIISTLT (SEQ ID NO:111). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISNINEIVLELKGSETTFMCEYADETATIVE FLNRWITFCQSIISTLT (SEQ ID NO:112). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRHLISNINVIVLELKGSETTFMCEYADETATIVE FLNRWITFCQSIISTLT (SEQ ID NO:113). In some embodiments, the mutant IL-2 polypeptide comprises the amino acid sequence of APTSSSTKKTQLQLEDLLLDLQMILNGINNYKNPKLTRMLTAKFYMPKKATELKHLQ CLEEQLKPLEEVLNLAQSKNFHLRPRDLISNINVIVLELKGSETTFMCEYADETATIVE FLNRWITFCQSIISTLT (SEQ ID NO:114). In some embodiments, the mutant IL-2 polypeptide comprises the amino acid sequence of APTSSSTKKTQLQLEELLLDLQMILNGINNYKNPKLTRMLTAKFYMPKKATELKHLQ CLEEQLKPLEEVLNLAQSKNFHLRPRDLISNINVIVLELKGSETTFMCEYADETATIVE FLNRWITFCQSIISTLT (SEQ ID NO:115). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISNINVIVLELKGSETTFMCEYADETATIVE FLNRWITFCSSIISTLT (SEQ ID NO:116). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFAQSIISTLT (SEQ ID NO:117). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISNINVIVLELKGSETTFMCEYADETATIV EFLNRWITFAQSIISTLT (SEQ ID NO:118). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISNINVIVLELKGSETTFMCEYADETATIVE FLNRWITFAQSIISTLT (SEQ ID NO:119). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISNINVIVLELKGSETTFMCEYADETATIV EFLNRWITFAQSIISTLT (SEQ ID NO:120). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISSINVIVLELKGSETTFMCEYADETATIVE FLNRWITFAQSIISTLT (SEQ ID NO:121). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISAINVIVLELKGSETTFMCEYADETATIVE FLNRWITFAQSIISTLT (SEQ ID NO:122). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISNINEIVLELKGSETTFMCEYADETATIVE FLNRWITFAQSIISTLT (SEQ ID NO: 123). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRHLISNINVIVLELKGSETTFMCEYADETATIVE FLNRWITFAQSIISTLT (SEQ ID NO:124). In some embodiments, the mutant IL-2 polypeptide comprises the amino acid sequence of APTSSSTKKTQLQLEDLLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFAQSIISTLT (SEQ ID NO: 125). In some embodiments, the mutant IL-2 polypeptide comprises the amino acid sequence of APTSSSTKKTQLQLEELLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFAQSIISTLT (SEQ ID NO:126). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFASSIISTLT (SEQ ID NO:127). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISSINVIVLELKGSETTFMCEYADETATIV EFLNRWITFAQSIISTLT (SEQ ID NO:128). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISAINVIVLELKGSETTFMCEYADETATIV EFLNRWITFAQSIISTLT (SEQ ID NO:129). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISNINEIVLELKGSETTFMCEYADETATIV EFLNRWITFAQSIISTLT (SEQ ID NO:130). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRHLISNINVIVLELKGSETTFMCEYADETATIV EFLNRWITFAQSIISTLT (SEQ ID NO:131). In some embodiments, the mutant IL-2 polypeptide comprises the amino acid sequence of APTSSSTKKTQLQLEDLLLDLQMILNGINNYKNPKLTDMLTAKFYMPKKATELKHL QCLEEELKPLEEVLNLAQSKNFHLRPRDLISNINVIVLELKGSETTFMCEYADETATIV EFLNRWITFAQSIISTLT (SEQ ID NO: 132). In some embodiments, the mutant IL-2 polypeptide comprises the amino acid sequence of APTSSSTKKTQLQLEELLLDLQMILNGINNYKNPKLTDMLTAKFYMPKKATELKHLQ CLEEELKPLEEVLNLAQSKNFHLRPRDLISNINVIVLELKGSETTFMCEYADETATIVE FLNRWITFAQSIISTLT (SEQ ID NO: 133). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISNINVIVLELKGSETTFMCEYADETATIV EFLNRWITFASSIISTLT (SEQ ID NO:134). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISSINVIVLELKGSETTFMCEYADETATIV EFLNRWITFAQSIISTLT (SEQ ID NO:135). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISAINVIVLELKGSETTFMCEYADETATIV EFLNRWITFAQSIISTLT (SEQ ID NO:136). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISNINEIVLELKGSETTFMCEYADETATIV EFLNRWITFAQSIISTLT (SEQ ID NO:137). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRHLISNINVIVLELKGSETTFMCEYADETATIV EFLNRWITFAQSIISTLT (SEQ ID NO:138). In some embodiments, the mutant IL-2 polypeptide comprises the amino acid sequence of APTSSSTKKTQLQLEDLLLDLQMILNGINNYKNPKLTAMLTKKFYMPKKATELKHL QCLEEELKPLEEVLNLAQSKNFHLRPRDLISNINVIVLELKGSETTFMCEYADETATIV EFLNRWITFAQSIISTLT (SEQ ID NO:139). In some embodiments, the mutant IL-2 polypeptide comprises the amino acid sequence of APTSSSTKKTQLQLEELLLDLQMILNGINNYKNPKLTAMLTKKFYMPKKATELKHLQ CLEEELKPLEEVLNLAQSKNFHLRPRDLISNINVIVLELKGSETTFMCEYADETATIVE FLNRWITFAQSIISTLT (SEQ ID NO:140). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISNINVIVLELKGSETTFMCEYADETATIV EFLNRWITFASSIISTLT (SEQ ID NO:141). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISSINVIVLELKGSETTFMCEYADETATIVE FLNRWITFAQSIISTLT (SEQ ID NO:142). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISAINVIVLELKGSETTFMCEYADETATIVE FLNRWITFAQSIISTLT (SEQ ID NO:143). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISNINEIVLELKGSETTFMCEYADETATIVE FLNRWITFAQSIISTLT (SEQ ID NO:144). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRHLISNINVIVLELKGSETTFMCEYADETATIVE FLNRWITFAQSIISTLT (SEQ ID NO:145). In some embodiments, the mutant IL-2 polypeptide comprises the amino acid sequence of APTSSSTKKTQLQLEDLLLDLQMILNGINNYKNPKLTRMLTAKFYMPKKATELKHLQ CLEEQLKPLEEVLNLAQSKNFHLRPRDLISNINVIVLELKGSETTFMCEYADETATIVE FLNRWITFAQSIISTLT (SEQ ID NO:146). In some embodiments, the mutant IL-2 polypeptide comprises the amino acid sequence of APTSSSTKKTQLQLEELLLDLQMILNGINNYKNPKLTRMLTAKFYMPKKATELKHLQ CLEEQLKPLEEVLNLAQSKNFHLRPRDLISNINVIVLELKGSETTFMCEYADETATIVE FLNRWITFAQSIISTLT (SEQ ID NO:147). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISNINVIVLELKGSETTFMCEYADETATIVE FLNRWITFASSIISTLT (SEQ ID NO:148). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRDLISSINVIVLELKGSETTFMCEYADETATIVE FLNRWITFAQSIISTLT (SEQ ID NO:149). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRDLISAINVIVLELKGSETTFMCEYADETATIVE FLNRWITFAQSIISTLT (SEQ ID NO:150). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRDLISNINEIVLELKGSETTFMCEYADETATIVE FLNRWITFAQSIISTLT (SEQ ID NO:151). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRHLISNINVIVLELKGSETTFMCEYADETATIVE FLNRWITFAQSIISTLT (SEQ ID NO:152). In some embodiments, the mutant IL-2 polypeptide comprises the amino acid sequence of APTSSSTKKTQLQLEDLLLDLQMILNGINNYKNPKLTRMLTAKFYMPKKATELKHLQ CLEEELKPLEEVLNLAQSKNFHLRPRDLISNINVIVLELKGSETTFMCEYADETATIVE FLNRWITFAQSIISTLT (SEQ ID NO:153). In some embodiments, the mutant IL-2 polypeptide comprises the amino acid sequence of APTSSSTKKTQLQLEELLLDLQMILNGINNYKNPKLTRMLTAKFYMPKKATELKHLQ CLEEELKPLEEVLNLAQSKNFHLRPRDLISNINVIVLELKGSETTFMCEYADETATIVE FLNRWITFAQSIISTLT (SEQ ID NO:154). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRDLISNINVIVLELKGSETTFMCEYADETATIVE FLNRWITFASSIISTLT (SEQ ID NO:155). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISGINVIVLELKGSETTFMCEYADETATIVE FLNRWITFCQSIISTLT (SEQ ID NO:190). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRKLISNINVIVLELKGSETTFMCEYADETATIVE FLNRWITFCQSIISTLT (SEQ ID NO: 191). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRRLISNINVIVLELKGSETTFMCEYADETATIVE FLNRWITFCQSIISTLT (SEQ ID NO: 192). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISGINVIVLELKGSETTFMCEYADETATIV EFLNRWITFCQSIISTLT (SEQ ID NO:193). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRKLISNINVIVLELKGSETTFMCEYADETATIV EFLNRWITFCQSIISTLT (SEQ ID NO:194). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRRLISNINVIVLELKGSETTFMCEYADETATIV EFLNRWITFCQSIISTLT (SEQ ID NO:195). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISGINVIVLELKGSETTFMCEYADETATIV EFLNRWITFCQSIISTLT (SEQ ID NO:196). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRKLISNINVIVLELKGSETTFMCEYADETATIV EFLNRWITFCQSIISTLT (SEQ ID NO:197). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRRLISNINVIVLELKGSETTFMCEYADETATIV EFLNRWITFCQSIISTLT (SEQ ID NO:198). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISGINVIVLELKGSETTFMCEYADETATIVE FLNRWITFCQSIISTLT (SEQ ID NO:199). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRKLISNINVIVLELKGSETTFMCEYADETATIVE FLNRWITFCQSIISTLT (SEQ ID NO:200). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRRLISNINVIVLELKGSETTFMCEYADETATIVE FLNRWITFCQSIISTLT (SEQ ID NO:201). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISGINVIVLELKGSETTFMCEYADETATIVE FLNRWITFAQSIISTLT (SEQ ID NO:202). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRKLISNINVIVLELKGSETTFMCEYADETATIVE FLNRWITFAQSIISTLT (SEQ ID NO:203). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRRLISNINVIVLELKGSETTFMCEYADETATIVE FLNRWITFAQSIISTLT (SEQ ID NO:204). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISGINVIVLELKGSETTFMCEYADETATIV EFLNRWITFAQSIISTLT (SEQ ID NO:205). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRKLISNINVIVLELKGSETTFMCEYADETATIV EFLNRWITFAQSIISTLT (SEQ ID NO:206). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRRLISNINVIVLELKGSETTFMCEYADETATIV EFLNRWITFAQSIISTLT (SEQ ID NO:207). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISGINVIVLELKGSETTFMCEYADETATIV EFLNRWITFAQSIISTLT (SEQ ID NO:208). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRKLISNINVIVLELKGSETTFMCEYADETATIV EFLNRWITFAQSIISTLT (SEQ ID NO:209). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRRLISNINVIVLELKGSETTFMCEYADETATIV EFLNRWITFAQSIISTLT (SEQ ID NO:210). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISGINVIVLELKGSETTFMCEYADETATIVE FLNRWITFAQSIISTLT (SEQ ID NO:211). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRKLISNINVIVLELKGSETTFMCEYADETATIVE FLNRWITFAQSIISTLT (SEQ ID NO:212). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRRLISNINVIVLELKGSETTFMCEYADETATIVE FLNRWITFAQSIISTLT (SEQ ID NO:213). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRDLISGINVIVLELKGSETTFMCEYADETATIVE FLNRWITFAQSIISTLT (SEQ ID NO:214). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRKLISNINVIVLELKGSETTFMCEYADETATIVE FLNRWITFAQSIISTLT (SEQ ID NO:215). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRRLISNINVIVLELKGSETTFMCEYADETATIVE FLNRWITFAQSIISTLT (SEQ ID NO:216). In some embodiments, the mutant IL-2 polypeptide comprises the amino acid sequence of APTSSSTKKTQLQLEELLLDLQMILNGINNYKNPKLTEMLTAKFYMPKKATELKHLQ CLEEELKPLEEVLNLAQSKNFHLRPRDLISNINVIVLELKGSETTFMCEYADETATIVE FLNRWITFAQSIISTLT (SEQ ID NO:297). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISDINVIVLELKGSETTFMCEYADETATIVE FLNRWITFAQSIISTLT (SEQ ID NO:354). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISTINVIVLELKGSETTFMCEYADETATIVE FLNRWITFAQSIISTLT (SEQ ID NO:355). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISRINVIVLELKGSETTFMCEYADETATIVE FLNRWITFAQSIISTLT (SEQ ID NO:356). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISRINVIVLELKGSETTFMCEYADETATIVE FLNRWITFCQSIISTLT (SEQ ID NO:357). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISTINVIVLELKGSETTFMCEYADETATIVE FLNRWITFCQSIISTLT (SEQ ID NO:358). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISDINVIVLELKGSETTFMCEYADETATIVE FLNRWITFCQSIISTLT (SEQ ID NO:359). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISRINVIVLELKGSETTFMCEYADETATIV EFLNRWITFCQSIISTLT (SEQ ID NO:360). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISTINVIVLELKGSETTFMCEYADETATIV EFLNRWITFCQSIISTLT (SEQ ID NO:361). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISDINVIVLELKGSETTFMCEYADETATIV EFLNRWITFCQSIISTLT (SEQ ID NO:362). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISRINVIVLELKGSETTFMCEYADETATIV EFLNRWITFCQSIISTLT (SEQ ID NO:363). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISTINVIVLELKGSETTFMCEYADETATIV EFLNRWITFCQSIISTLT (SEQ ID NO:364). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISDINVIVLELKGSETTFMCEYADETATIV EFLNRWITFCQSIISTLT (SEQ ID NO:365). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISRINVIVLELKGSETTFMCEYADETATIVE FLNRWITFCQSIISTLT (SEQ ID NO:366). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISTINVIVLELKGSETTFMCEYADETATIVE FLNRWITFCQSIISTLT (SEQ ID NO:367). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISDINVIVLELKGSETTFMCEYADETATIVE FLNRWITFCQSIISTLT (SEQ ID NO:368). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISRINVIVLELKGSETTFMCEYADETATIVE FLNRWITFAQSIISTLT (SEQ ID NO:369). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISTINVIVLELKGSETTFMCEYADETATIVE FLNRWITFAQSIISTLT (SEQ ID NO:370). In some embodiments, the mutant IL-2 polypeptide comprises the amino acid sequence of APTSSSTKKTQLQLEHLLLDLQMILNGINNYKNPKLTEMLTAKFYMPKKATELKHLQ CLEEELKPLEEVLNLAQSKNFHLRPRDLISDINVIVLELKGSETTFMCEYADETATIVE FLNRWITFAQSIISTLT (SEQ ID NO:371). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISRINVIVLELKGSETTFMCEYADETATIV EFLNRWITFAQSIISTLT (SEQ ID NO:372). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISTINVIVLELKGSETTFMCEYADETATIV EFLNRWITFAQSIISTLT (SEQ ID NO:373). In some embodiments, the mutant IL-2 polypeptide comprises the amino acid sequence of APTSSSTKKTQLQLEHLLLDLQMILNGINNYKNPKLTDMLTAKFYMPKKATELKHL QCLEEELKPLEEVLNLAQSKNFHLRPRDLISDINVIVLELKGSETTFMCEYADETATIV EFLNRWITFAQSIISTLT (SEQ ID NO:374). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISRINVIVLELKGSETTFMCEYADETATIV EFLNRWITFAQSIISTLT (SEQ ID NO:375). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISTINVIVLELKGSETTFMCEYADETATIV EFLNRWITFAQSIISTLT (SEQ ID NO:376). In some embodiments, the mutant IL-2 polypeptide comprises the amino acid sequence of APTSSSTKKTQLQLEHLLLDLQMILNGINNYKNPKLTAMLTKKFYMPKKATELKHL QCLEEELKPLEEVLNLAQSKNFHLRPRDLISDINVIVLELKGSETTFMCEYADETATIV EFLNRWITFAQSIISTLT (SEQ ID NO:377). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISRINVIVLELKGSETTFMCEYADETATIVE FLNRWITFAQSIISTLT (SEQ ID NO:378). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISTINVIVLELKGSETTFMCEYADETATIVE FLNRWITFAQSIISTLT (SEQ ID NO:379). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEQLKPLEEVLNLAQSKNFHLRPRDLISDINVIVLELKGSETTFMCEYADETATIVE FLNRWITFAQSIISTLT (SEQ ID NO:380). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRDLISRINVIVLELKGSETTFMCEYADETATIVE FLNRWITFAQSIISTLT (SEQ ID NO:381). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRDLISTINVIVLELKGSETTFMCEYADETATIVE FLNRWITFAQSIISTLT (SEQ ID NO:382). In some embodiments, the mutant IL-2 polypeptide comprises the amino acid sequence of APTSSSTKKTQLQLEHLLLDLQMILNGINNYKNPKLTRMLTAKFYMPKKATELKHLQ CLEEELKPLEEVLNLAQSKNFHLRPRDLISDINVIVLELKGSETTFMCEYADETATIVE FLNRWITFAQSIISTLT (SEQ ID NO:383). In some embodiments, the mutant IL-2 polypeptide comprises the amino acid sequence of an IL-2 polypeptide listed in Table 7.

TABLE 7
Exemplary IL-2 polypeptide sequences

| | | SEQ |
|---|---|---|
| IL-2 | | ID |
| ID | Sequence | NO |
| m1 | APTSSSTKKTQLQLEHLLLDLQMILNGINNYKNPKLTEMLTAKFYMPKKATEL | 80 |
| | KHLQCLEEELKPLEEVLNLAQSKNFHLRPRDLISAINVIVLELKGSETTFMCE | |
| | YADETATIVEFLNRWITFAQSIISTLT | |
| m2 | APTSSSTKKTQLQLEHLLLDLQMILNGINNYKNPKLTEMLTAKFYMPKKATEL | 121 |
| | KHLQCLEEELKPLEEVLNLAQSKNFHLRPRDLISSINVIVLELKGSETTFMCE | |
| | YADETATIVEFLNRWITFAQSIISTLT | |
| m3 | APTSSSTKKTQLQLEDLLLDLQMILNGINNYKNPKLTEMLTAKFYMPKKATEL | 125 |
| | KHLQCLEEELKPLEEVLNLAQSKNFHLRPRDLISNINVIVLELKGSETTFMCE | |
| | YADETATIVEFLNRWITFAQSIISTLT | |
| m4 | APTSSSTKKTQLQLEELLLDLQMILNGINNYKNPKLTEMLTAKFYMPKKATEL | 297 |
| | KHLQCLEEELKPLEEVLNLAQSKNFHLRPRDLISNINVIVLELKGSETTFMCE | |
| | YADETATIVEFLNRWITFAQSIISTLT | |
| m5 | APTSSSTKKTQLQLEHLLLDLQMILNGINNYKNPKLTEMLTAKFYMPKKATEL | 202 |
| | KHLQCLEEELKPLEEVLNLAQSKNFHLRPRDLISGINVIVLELKGSETTFMCE | |
| | YADETATIVEFLNRWITFAQSIISTLT | |
| m6 | APTSSSTKKTQLQLEHLLLDLQMILNGINNYKNPKLTEMLTAKFYMPKKATEL | 117 |
| | KHLQCLEEELKPLEEVLNLAQSKNFHLRPRDLISNINVIVLELKGSETTFMCE | |
| | YADETATIVEFLNRWITFAQSIISTLT | |
| m7 | APTSSSTKKTQLQLEHLLLDLQMILNGINNYKNPKLTEMLTAKFYMPKKATEL | 123 |
| | KHLQCLEEELKPLEEVLNLAQSKNFHLRPRDLISNINEIVLELKGSETTFMCE | |
| | YADETATIVEFLNRWITFAQSIISTLT | |
| m8 | APTSSSTKKTQLQLEHLLLDLQMILNGINNYKNPKLTEMLTAKFYMPKKATEL | 124 |
| | KHLQCLEEELKPLEEVLNLAQSKNFHLRPRHLISNINVIVLELKGSETTFMCE | |
| | YADETATIVEFLNRWITFAQSIISTLT | |
| m9 | APTSSSTKKTQLQLEHLLLDLQMILNGINNYKNPKLTEMLTAKFYMPKKATEL | 203 |
| | KHLQCLEEELKPLEEVLNLAQSKNFHLRPRKLISNINVIVLELKGSETTFMCE | |
| | YADETATIVEFLNRWITFAQSIISTLT | |
| m10 | APTSSSTKKTQLQLEHLLLDLQMILNGINNYKNPKLTEMLTAKFYMPKKATEL | 204 |
| | KHLQCLEEELKPLEEVLNLAQSKNFHLRPRRLISNINVIVLELKGSETTFMCE | |
| | YADETATIVEFLNRWITFAQSIISTLT | |
| m11 | APTSSSTKKTQLQLEHLLLDLQMILNGINNYKNPKLTEMLTAKFYMPKKATEL | 354 |
| | KHLQCLEEELKPLEEVLNLAQSKNFHLRPRDLISDINVIVLELKGSETTFMCE | |
| | YADETATIVEFLNRWITFAQSIISTLT | |
| m12 | APTSSSTKKTQLQLEHLLLDLQMILNGINNYKNPKLTEMLTAKFYMPKKATEL | 355 |
| | KHLQCLEEELKPLEEVLNLAQSKNFHLRPRDLISTINVIVLELKGSETTFMCE | |
| | YADETATIVEFLNRWITFAQSIISTLT | |
| m13 | APTSSSTKKTQLQLEHLLLDLQMILNGINNYKNPKLTEMLTAKFYMPKKATEL | 356 |
| | KHLQCLEEELKPLEEVLNLAQSKNFHLRPRDLISRINVIVLELKGSETTFMCE | |
| | YADETATIVEFLNRWITFAQSIISTLT | |

In some embodiments, the mutant IL-2 polypeptides of the present disclosure also contain other modifications, including but not limited to mutations and deletions, that provide additional advantages such as improved biophysical properties. Improved biophysical properties include but are not limited to improved thermostability, aggregation propensity, acid reversibility, viscosity, and production in a mammalian or bacterial or yeast cell. For example, residue C125 may be replaced with a neutral amino acid such as serine, alanine, threonine or valine; and N terminal Al residue could be deleted, both of which were described in U.S. Pat. No. 4,518,584. Mutant IL-2 polypeptides may also include a mutation of the residue M104, such as M104A, as described in U.S. Pat. No. 5,206,344. Thus, in certain embodiments the mutant IL-2 polypeptide of the present disclosure comprises the amino acid substitution C125A. In other embodiments, one, two, or three N-terminal residues are deleted.

In some embodiments, a fusion protein of the present disclosure comprises a linker. In some embodiments, the linker is a chemical linker (for example, see disclosed in Protein Engineering, 9 (3), 299-305, 1996). Synthetic chemical linkers include crosslinking agents that are routinely used to crosslink peptides, for example, N-hydroxy succinimide (NHS), disuccinimidyl suberate (DSS), bis(succinimidyl) suberate (BS3), dithiobis(succinimidyl propionate) (DSP), dithiobis(succinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis[2-(succinimidoxycarbonyloxy)ethyl]sulfone (BSOCOES), and bis[2-(succinimidoxycarbonyloxy)ethyl]sulfone (sulfo-BSOCOES).

In some embodiments, the linker is an amino acid- or peptide-based linker. In some embodiments, the polypeptide linker is a peptide with a length of at least 5 amino acids, preferably with a length of 5 to 100, more preferably of 10 to 50 amino acids. In one embodiment, said peptide linker is G, S, GS, SG, SGG, GGS, and GSG (with G=glycine and S=serine). In some embodiments, the linker comprises the sequence (GGGS)xGn (SEQ ID NO: 74), (GGGGS)xGn (SEQ ID NO:75), (GGGGGS)xGn (SEQ ID NO:76), S(GGGS)xGn (SEQ ID NO:386), S(GGGGS)xGn (SEQ ID NO:387), or S(GGGGGS)xGn (SEQ ID NO: 388), wherein x=1, 2, 3, 4, 5, 6, 7, 8, 9, 10, 11, or 12, and wherein n=0, 1, 2 or 3. In some embodiments, the linker comprises the sequence GGGGSGGGGSGGGGS (SEQ ID NO: 79) or SGGGGSGGGGSGGGGS (SEQ ID NO:389).

In some embodiments, a fusion protein of the present disclosure comprises one, two, or all three polypeptides shown for a single fusion protein in Table 5. In some embodiments, a fusion protein of the present disclosure comprises two light chains, a heavy chain comprising an IL-2 fusion, and a heavy chain not comprising an IL-2 fusion for a single fusion protein in Table 5. As is known in the art, the C-terminal lysine of some antibody heavy chain species may be cleaved off in some fraction of molecules. Therefore, in some embodiments, a fusion protein of the present disclosure comprises two light chains, a heavy chain comprising an IL-2 fusion, and a heavy chain not comprising an IL-2 fusion for a single fusion protein in Table 5, wherein the heavy chain not comprising the IL-2 fusion comprises the sequence of SEQ ID Nos: 158, 161, 164, 167, 170, 173, 176, 189, 300, 304, 308, 312, 326, 320, 324, 328, 332, 336, 340, 344, 348, or 352 for the respective fusion protein. In some embodiments, a fusion protein of the present disclosure comprises two light chains, a heavy chain comprising an IL-2 fusion, and a heavy chain not comprising an IL-2 fusion for a single fusion protein in Table 5, wherein the heavy chain not comprising the IL-2 fusion comprises the sequence of SEQ ID Nos: 217-224, 301, 305, 309, 313, 317, 321, 325, 329, 333, 337, 341, 345, 349, or 353 for the respective fusion protein. In some embodiments, the present disclosure provides a plurality of fusion proteins of the present disclosure (e.g., in a mixture), wherein each fusion protein of the plurality comprises two light chains, a heavy chain comprising an IL-2 fusion, and a heavy chain not comprising an IL-2 fusion for a single fusion protein in Table 5, wherein the heavy chain not comprising the IL-2 fusion comprises the sequence of SEQ ID Nos: 158, 161, 164, 167, 170, 173, 176, 189, 300, 304, 308, 312, 326, 320, 324, 328, 332, 336, 340, 344, 348, or 352 for the respective fusion protein, or the sequence of SEQ ID Nos: 217-224, 301, 305, 309, 313, 317, 321, 325, 329, 333, 337, 341, 345, 349, or 353 for the respective fusion protein, or the plurality comprises a mixture of species representing cleaved (e.g., comprising the sequence of SEQ ID Nos: 158, 161, 164, 167, 170, 173, 176, 189, 300, 304, 308, 312, 326, 320, 324, 328, 332, 336, 340, 344, 348, or 352 for the respective fusion protein) and uncleaved (e.g., comprising the sequence of SEQ ID Nos: 217-224, 301, 305, 309, 313, 317, 321, 325, 329, 333, 337, 341, 345, 349, or 353 for the respective fusion protein) species.

For example, in some embodiments, a fusion protein of the present disclosure comprises one or two light chains comprising the amino acid sequence of SEQ ID NO:156, a heavy chain comprising the amino acid sequence of SEQ ID NO: 157, and a heavy chain comprising the amino acid sequence of SEQ ID NO:158. In some embodiments, a fusion protein of the present disclosure comprises one or two light chains comprising the amino acid sequence of SEQ ID NO:159, a heavy chain comprising the amino acid sequence of SEQ ID NO: 160, and a heavy chain comprising the amino acid sequence of SEQ ID NO: 161. In some embodiments, a fusion protein of the present disclosure comprises one or two light chains comprising the amino acid sequence of SEQ ID NO: 162, a heavy chain comprising the amino acid sequence of SEQ ID NO:163, and a heavy chain comprising the amino acid sequence of SEQ ID NO: 164. In some embodiments, a fusion protein of the present disclosure comprises one or two light chains comprising the amino acid sequence of SEQ ID NO: 165, a heavy chain comprising the amino acid sequence of SEQ ID NO: 166, and a heavy chain comprising the amino acid sequence of SEQ ID NO:167. In some embodiments, a fusion protein of the present disclosure comprises one or two light chains comprising the amino acid sequence of SEQ ID NO: 168, a heavy chain comprising the amino acid sequence of SEQ ID NO: 169, and a heavy chain comprising the amino acid sequence of SEQ ID NO: 170. In some embodiments, a fusion protein of the present disclosure comprises one or two light chains comprising the amino acid sequence of SEQ ID NO:171, a heavy chain comprising the amino acid sequence of SEQ ID NO: 172, and a heavy chain comprising the amino acid sequence of SEQ ID NO: 173. In some embodiments, a fusion protein of the present disclosure comprises one or two light chains comprising the amino acid sequence of SEQ ID NO:174, a heavy chain comprising the amino acid sequence of SEQ ID NO:175, and a heavy chain comprising the amino acid sequence of SEQ ID NO:176. In some embodiments, a fusion protein of the present disclosure comprises one or two light chains comprising the amino acid sequence of SEQ ID NO: 187, a heavy chain comprising the amino acid sequence of SEQ ID NO: 188, and a heavy chain comprising the amino acid sequence of SEQ ID NO:189. In some embodiments, a fusion protein of the present disclosure comprises one or two light chains comprising the amino acid sequence of SEQ ID NO:298, a heavy chain comprising the amino acid sequence of SEQ ID NO:299, and a heavy chain comprising the amino acid sequence of SEQ ID NO:300. In some embodiments, a fusion protein of the present disclosure comprises one or two light chains comprising the amino acid sequence of SEQ ID NO:302, a heavy chain comprising the amino acid sequence of SEQ ID NO:303, and a heavy chain comprising the amino acid sequence of SEQ ID NO:304. In some embodiments, a fusion protein of the present disclosure comprises one or two light chains comprising the amino acid sequence of SEQ ID NO:306, a heavy chain comprising the amino acid sequence of SEQ ID NO:307, and a heavy chain comprising the amino acid sequence of SEQ ID NO:308. In some embodiments, a fusion protein of the present disclosure comprises one or two light chains comprising the amino acid sequence of SEQ ID NO:310, a heavy chain comprising the amino acid sequence of SEQ ID NO:311, and a heavy chain comprising the amino acid sequence of SEQ ID NO:312. In some embodiments, a fusion protein of the present disclosure comprises one or two light chains comprising the amino acid sequence of SEQ ID NO:314, a heavy chain comprising the amino acid sequence of SEQ ID NO:315, and a heavy chain comprising the amino acid sequence of SEQ ID NO:316. In some embodiments, a fusion protein of the present disclosure comprises one or two light chains comprising the amino acid sequence of SEQ ID NO:318, a heavy chain comprising the amino acid sequence of SEQ ID NO:319, and a heavy chain comprising the amino acid sequence of SEQ ID NO:320. In some embodiments, a fusion protein of the present disclosure comprises one or two light chains comprising the amino acid sequence of SEQ ID NO:322, a heavy chain comprising the amino acid sequence of SEQ ID NO:323, and a heavy chain comprising the amino acid sequence of SEQ ID NO:324. In some embodiments, a fusion protein of the present disclosure comprises one or two light chains comprising the amino acid sequence of SEQ ID NO:326, a heavy chain comprising the amino acid sequence of SEQ ID NO:327, and a heavy chain comprising the amino acid sequence of SEQ ID NO:328. In some embodiments, a fusion protein of the present disclosure comprises one or two light chains comprising the amino acid sequence of SEQ ID NO:334, a heavy chain comprising the amino acid sequence of SEQ ID NO:335, and a heavy chain comprising the amino acid sequence of SEQ ID NO:336. In some embodiments, a fusion protein of the present disclosure comprises one or two light chains comprising the amino acid sequence of SEQ ID NO:338, a heavy chain comprising the amino acid sequence of SEQ ID NO:339, and a heavy chain comprising the amino acid sequence of SEQ ID NO:340. In some embodiments, a fusion protein of the present disclosure comprises one or two light chains comprising the amino acid sequence of SEQ ID NO:342, a heavy chain comprising the amino acid sequence of SEQ ID NO:343, and a heavy chain comprising the amino acid sequence of SEQ ID NO:344. In some embodiments, a fusion protein of the present disclosure comprises one or two light chains comprising the amino acid sequence of SEQ ID NO:346, a heavy chain comprising the amino acid sequence of SEQ ID NO:347, and a heavy chain comprising the amino acid sequence of SEQ ID NO:348. In some embodiments, a fusion protein of the present disclosure comprises one or two light chains comprising the amino acid sequence of SEQ ID NO:350, a heavy chain comprising the amino acid sequence of SEQ ID NO:351, and a heavy chain comprising the amino acid sequence of SEQ ID NO:352.

In some embodiments, a fusion protein of the present disclosure comprises one or two light chains comprising the amino acid sequence of SEQ ID NO:156, a heavy chain comprising the amino acid sequence of SEQ ID NO: 157, and a heavy chain comprising the amino acid sequence of SEQ ID NO:217. In some embodiments, a fusion protein of the present disclosure comprises one or two light chains comprising the amino acid sequence of SEQ ID NO:159, a heavy chain comprising the amino acid sequence of SEQ ID NO:160, and a heavy chain comprising the amino acid sequence of SEQ ID NO:218. In some embodiments, a fusion protein of the present disclosure comprises one or two light chains comprising the amino acid sequence of SEQ ID NO: 162, a heavy chain comprising the amino acid sequence of SEQ ID NO:163, and a heavy chain comprising the amino acid sequence of SEQ ID NO:219. In some embodiments, a fusion protein of the present disclosure comprises one or two light chains comprising the amino acid sequence of SEQ ID NO: 165, a heavy chain comprising the amino acid sequence of SEQ ID NO:166, and a heavy chain comprising the amino acid sequence of SEQ ID NO:220. In some embodiments, a fusion protein of the present disclosure comprises one or two light chains comprising the amino acid sequence of SEQ ID NO:168, a heavy chain comprising the amino acid sequence of SEQ ID NO: 169, and a heavy chain comprising the amino acid sequence of SEQ ID NO:221. In some embodiments, a fusion protein of the present disclosure comprises one or two light chains comprising the amino acid sequence of SEQ ID NO: 171, a heavy chain comprising the amino acid sequence of SEQ ID NO: 172, and a heavy chain comprising the amino acid sequence of SEQ ID NO:222. In some embodiments, a fusion protein of the present disclosure comprises one or two light chains comprising the amino acid sequence of SEQ ID NO: 174, a heavy chain comprising the amino acid sequence of SEQ ID NO:175, and a heavy chain comprising the amino acid sequence of SEQ ID NO:223. In some embodiments, a fusion protein of the present disclosure comprises one or two light chains comprising the amino acid sequence of SEQ ID NO: 187, a heavy chain comprising the amino acid sequence of SEQ ID NO:188, and a heavy chain comprising the amino acid sequence of SEQ ID NO:224. In some embodiments, a fusion protein of the present disclosure comprises one or two light chains comprising the amino acid sequence of SEQ ID NO:298, a heavy chain comprising the amino acid sequence of SEQ ID NO:299, and a heavy chain comprising the amino acid sequence of SEQ ID NO:301. In some embodiments, a fusion protein of the present disclosure comprises one or two light chains comprising the amino acid sequence of SEQ ID NO:302, a heavy chain comprising the amino acid sequence of SEQ ID NO:303, and a heavy chain comprising the amino acid sequence of SEQ ID NO:305. In some embodiments, a fusion protein of the present disclosure comprises one or two light chains comprising the amino acid sequence of SEQ ID NO:306, a heavy chain comprising the amino acid sequence of SEQ ID NO:307, and a heavy chain comprising the amino acid sequence of SEQ ID NO:309. In some embodiments, a fusion protein of the present disclosure comprises one or two light chains comprising the amino acid sequence of SEQ ID NO:310, a heavy chain comprising the amino acid sequence of SEQ ID NO:311, and a heavy chain comprising the amino acid sequence of SEQ ID NO:313. In some embodiments, a fusion protein of the present disclosure comprises one or two light chains comprising the amino acid sequence of SEQ ID NO:314, a heavy chain comprising the amino acid sequence of SEQ ID NO:315, and a heavy chain comprising the amino acid sequence of SEQ ID NO:317. In some embodiments, a fusion protein of the present disclosure comprises one or two light chains comprising the amino acid sequence of SEQ ID NO:318, a heavy chain comprising the amino acid sequence of SEQ ID NO:319, and a heavy chain comprising the amino acid sequence of SEQ ID NO:321. In some embodiments, a fusion protein of the present disclosure comprises one or two light chains comprising the amino acid sequence of SEQ ID NO:322, a heavy chain comprising the amino acid sequence of SEQ ID NO:323, and a heavy chain comprising the amino acid sequence of SEQ ID NO:325. In some embodiments, a fusion protein of the present disclosure comprises one or two light chains comprising the amino acid sequence of SEQ ID NO:326, a heavy chain comprising the amino acid sequence of SEQ ID NO:327, and a heavy chain comprising the amino acid sequence of SEQ ID NO:329. In some embodiments, a fusion protein of the present disclosure comprises one or two light chains comprising the amino acid sequence of SEQ ID NO:330, a heavy chain comprising the amino acid sequence of SEQ ID NO:331, and a heavy chain comprising the amino acid sequence of SEQ ID NO:333. In some embodiments, a fusion protein of the present disclosure comprises one or two light chains comprising the amino acid sequence of SEQ ID NO:334, a heavy chain comprising the amino acid sequence of SEQ ID NO:335, and a heavy chain comprising the amino acid sequence of SEQ ID NO:337. In some embodiments, a fusion protein of the present disclosure comprises one or two light chains comprising the amino acid sequence of SEQ ID NO:338, a heavy chain comprising the amino acid sequence of SEQ ID NO:339, and a heavy chain comprising the amino acid sequence of SEQ ID NO:341. In some embodiments, a fusion protein of the present disclosure comprises one or two light chains comprising the amino acid sequence of SEQ ID NO:342, a heavy chain comprising the amino acid sequence of SEQ ID NO:343, and a heavy chain comprising the amino acid sequence of SEQ ID NO:345. In some embodiments, a fusion protein of the present disclosure comprises one or two light chains comprising the amino acid sequence of SEQ ID NO:346, a heavy chain comprising the amino acid sequence of SEQ ID NO:347, and a heavy chain comprising the amino acid sequence of SEQ ID NO:349. In some embodiments, a fusion protein of the present disclosure comprises one or two light chains comprising the amino acid sequence of SEQ ID NO:350, a heavy chain comprising the amino acid sequence of SEQ ID NO:351, and a heavy chain comprising the amino acid sequence of SEQ ID NO:353.

In some embodiments, the present disclosure provides a mixture of fusion protein species, wherein each species comprises one or two light chains comprising the amino acid sequence of SEQ ID NO: 156, a heavy chain comprising the amino acid sequence of SEQ ID NO: 157, and a heavy chain comprising the amino acid sequence of SEQ ID NO: 158 or 217. In some embodiments, the present disclosure provides a mixture of fusion protein species, wherein each species comprises one or two light chains comprising the amino acid sequence of SEQ ID NO: 159, a heavy chain comprising the amino acid sequence of SEQ ID NO:160, and a heavy chain comprising the amino acid sequence of SEQ ID NO:161 or 218. In some embodiments, the present disclosure provides a mixture of fusion protein species, wherein each species comprises one or two light chains comprising the amino acid sequence of SEQ ID NO: 162, a heavy chain comprising the amino acid sequence of SEQ ID NO:163, and a heavy chain comprising the amino acid sequence of SEQ ID NO: 164 or 219. In some embodiments, the present disclosure provides a mixture of fusion protein species, wherein each species comprises one or two light chains comprising the amino acid sequence of SEQ ID NO: 165, a heavy chain comprising the amino acid sequence of SEQ ID NO:166, and a heavy chain comprising the amino acid sequence of SEQ ID NO: 167 or 220. In some embodiments, the present disclosure provides a mixture of fusion protein species, wherein each species comprises one or two light chains comprising the amino acid sequence of SEQ ID NO: 168, a heavy chain comprising the amino acid sequence of SEQ ID NO: 169, and a heavy chain comprising the amino acid sequence of SEQ ID NO: 170 or 221. In some embodiments, the present disclosure provides a mixture of fusion protein species, wherein each species comprises one or two light chains comprising the amino acid sequence of SEQ ID NO: 171, a heavy chain comprising the amino acid sequence of SEQ ID NO:172, and a heavy chain comprising the amino acid sequence of SEQ ID NO:173 or 222. In some embodiments, the present disclosure provides a mixture of fusion protein species, wherein each species comprises one or two light chains comprising the amino acid sequence of SEQ ID NO: 174, a heavy chain comprising the amino acid sequence of SEQ ID NO: 175, and a heavy chain comprising the amino acid sequence of SEQ ID NO: 176 or 223. In some embodiments, the present disclosure provides a mixture of fusion protein species, wherein each species comprises one or two light chains comprising the amino acid sequence of SEQ ID NO: 187, a heavy chain comprising the amino acid sequence of SEQ ID NO: 188, and a heavy chain comprising the amino acid sequence of SEQ ID NO: 189 or 224. In some embodiments, the present disclosure provides a mixture of fusion protein species, wherein each species comprises one or two light chains comprising the amino acid sequence of SEQ ID NO: 298, a heavy chain comprising the amino acid sequence of SEQ ID NO:299, and a heavy chain comprising the amino acid sequence of SEQ ID NO:300 or 301. In some embodiments, the present disclosure provides a mixture of fusion protein species, wherein each species comprises one or two light chains comprising the amino acid sequence of SEQ ID NO: 302, a heavy chain comprising the amino acid sequence of SEQ ID NO:303, and a heavy chain comprising the amino acid sequence of SEQ ID NO:304 or 305. In some embodiments, the present disclosure provides a mixture of fusion protein species, wherein each species comprises one or two light chains comprising the amino acid sequence of SEQ ID NO: 306, a heavy chain comprising the amino acid sequence of SEQ ID NO:307, and a heavy chain comprising the amino acid sequence of SEQ ID NO:308 or 309. In some embodiments, the present disclosure provides a mixture of fusion protein species, wherein each species comprises one or two light chains comprising the amino acid sequence of SEQ ID NO: 310, a heavy chain comprising the amino acid sequence of SEQ ID NO:311, and a heavy chain comprising the amino acid sequence of SEQ ID NO:312 or 313. In some embodiments, the present disclosure provides a mixture of fusion protein species, wherein each species comprises one or two light chains comprising the amino acid sequence of SEQ ID NO: 314, a heavy chain comprising the amino acid sequence of SEQ ID NO:315, and a heavy chain comprising the amino acid sequence of SEQ ID NO:316 or 317. In some embodiments, the present disclosure provides a mixture of fusion protein species, wherein each species comprises one or two light chains comprising the amino acid sequence of SEQ ID NO: 318, a heavy chain comprising the amino acid sequence of SEQ ID NO:319, and a heavy chain comprising the amino acid sequence of SEQ ID NO:320 or 321. In some embodiments, the present disclosure provides a mixture of fusion protein species, wherein each species comprises one or two light chains comprising the amino acid sequence of SEQ ID NO: 322, a heavy chain comprising the amino acid sequence of SEQ ID NO:323, and a heavy chain comprising the amino acid sequence of SEQ ID NO:324 or 325. In some embodiments, the present disclosure provides a mixture of fusion protein species, wherein each species comprises one or two light chains comprising the amino acid sequence of SEQ ID NO: 326, a heavy chain comprising the amino acid sequence of SEQ ID NO:327, and a heavy chain comprising the amino acid sequence of SEQ ID NO:328 or 329. In some embodiments, the present disclosure provides a mixture of fusion protein species, wherein each species comprises one or two light chains comprising the amino acid sequence of SEQ ID NO: 330, a heavy chain comprising the amino acid sequence of SEQ ID NO:331, and a heavy chain comprising the amino acid sequence of SEQ ID NO:332 or 333. In some embodiments, the present disclosure provides a mixture of fusion protein species, wherein each species comprises one or two light chains comprising the amino acid sequence of SEQ ID NO: 334, a heavy chain comprising the amino acid sequence of SEQ ID NO:335, and a heavy chain comprising the amino acid sequence of SEQ ID NO:336 or 337. In some embodiments, the present disclosure provides a mixture of fusion protein species, wherein each species comprises one or two light chains comprising the amino acid sequence of SEQ ID NO: 338, a heavy chain comprising the amino acid sequence of SEQ ID NO:339, and a heavy chain comprising the amino acid sequence of SEQ ID NO:340 or 341. In some embodiments, the present disclosure provides a mixture of fusion protein species, wherein each species comprises one or two light chains comprising the amino acid sequence of SEQ ID NO: 342, a heavy chain comprising the amino acid sequence of SEQ ID NO:343, and a heavy chain comprising the amino acid sequence of SEQ ID NO:344 or 345. In some embodiments, the present disclosure provides a mixture of fusion protein species, wherein each species comprises one or two light chains comprising the amino acid sequence of SEQ ID NO: 346, a heavy chain comprising the amino acid sequence of SEQ ID NO:347, and a heavy chain comprising the amino acid sequence of SEQ ID NO:348 or 349. In some embodiments, the present disclosure provides a mixture of fusion protein species, wherein each species comprises one or two light chains comprising the amino acid sequence of SEQ ID NO: 350, a heavy chain comprising the amino acid sequence of SEQ ID NO:351, and a heavy chain comprising the amino acid sequence of SEQ ID NO:352 or 353.

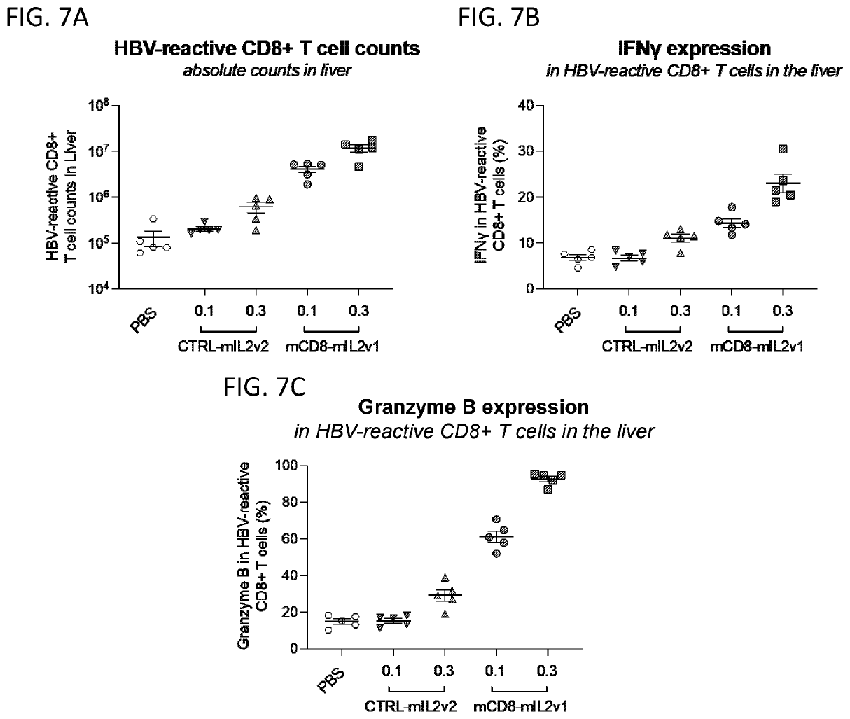

In some embodiments, the present disclosure provides a fusion protein comprising two heavy chain sequences and two light chain sequences of a single fusion protein listed in Table 5, wherein one of the heavy chain sequences has an IL2 fusion and the other heavy chain sequence is without an IL2 fusion, and wherein the two light chain sequences are identical. In some embodiments, the heavy chain sequence without an IL2 fusion comprises a lysine at the C terminus. In some embodiments, the fusion protein is of format A shown in FIG. 7. For example, in some embodiments, the fusion protein comprises four polypeptide chains, wherein (1) the first polypeptide chain comprises the amino acid sequence of SEQ ID NO: 334, the second polypeptide chain comprises the amino acid sequence of SEQ ID NO: 335, the third polypeptide chain comprises the amino acid sequence of SEQ ID NO:336, and the fourth polypeptide chain comprises the amino acid sequence of SEQ ID NO:334; (2) the first polypeptide chain comprises the amino acid sequence of SEQ ID NO:334, the second polypeptide chain comprises the amino acid sequence of SEQ ID NO:335, the third polypeptide chain comprises the amino acid sequence of SEQ ID NO:337, and the fourth polypeptide chain comprises the amino acid sequence of SEQ ID NO:334; (3) the first polypeptide chain comprises the amino acid sequence of SEQ ID NO:338, the second polypeptide chain comprises the amino acid sequence of SEQ ID NO:339, the third polypeptide chain comprises the amino acid sequence of SEQ ID NO:340, and the fourth polypeptide chain comprises the amino acid sequence of SEQ ID NO:338; or (4) the first polypeptide chain comprises the amino acid sequence of SEQ ID NO:338, the second polypeptide chain comprises the amino acid sequence of SEQ ID NO:339, the third polypeptide chain comprises the amino acid sequence of SEQ ID NO:341, and the fourth polypeptide chain comprises the amino acid sequence of SEQ ID NO:338.

TABLE 5
Anti-CD8: IL2 fusion protein sequences

| | | | | Heavy chain |
|---|---|---|---|---|
| | | | | sequence |
| | | Heavy chain | Heavy chain | (without |
| | | sequence | sequence | IL2 fusion) |
| | Light chain | (with IL2 | (without | plus C- |
| Name | sequence | fusion) | IL2 fusion) | term lysine |
| xhCD8v1- | DIQMTQSPASLS | QVHLQQSGPELVKPG | QVHLQQSGPELV | QVHLQQSGPE |
| IL-2ml | ASVGETVTITC | ASVKMSCKTSGYTFT | KPGASVKMSCK | LVKPGASVKM |
| | GASENIYGALN | KYTMHWVKQGHEES | TSGYTFTKYTM | SCKTSGYTFTK |
| | WYQRKQGKSP | LEWIGHFNPNNDETK | HWVKQGHEESL | YTMHWVKQG |
| | QLLIFGATNLA | YNQKFTGKATLTVD | EWIGHFNPNNDE | HEESLEWIGHF |
| | DGVSSRFSGSG | KSSTTAYMELRSLTS | TKYNQKFTGKA | NPNNDETKYN |
| | SDRQYSLKISSL | DDSALYYCARDGLG | TLTVDKSSTTAY | QKFTGKATLT |
| | HPDDVATYYC | LRLFADWGQGTLVT | MELRSLTSDDSA | VDKSSTTAYM |
| | QNILDTPWTFG | VSSASTKGPSVFPLAP | LYYCARDGLGL | ELRSLTSDDSA |
| | GGTKLEIKRTV | SSKSTSGGTAALGCL | RLFADWGQGTL | LYYCARDGLG |
| | AAPSVFIFPPSD | VKDYFPEPVTVSWNS | VTVSSASTKGPS | LRLFADWGQG |
| | EQLKSGTASVV | GALTSGVHTFPAVLQ | VFPLAPSSKSTSG | TLVTVSSASTK |
| | CLLNNFYPREA | SSGLYSLSSVVTVPSS | GTAALGCLVKD | GPSVFPLAPSS |
| | KVQWKVDNAL | SLGTQTYICNVNHKP | YFPEPVTVSWNS | KSTSGGTAAL |
| | QSGNSQESVTE | SNTKVDKKVEPKSCD | GALTSGVHTFPA | GCLVKDYFPEP |
| | QDSKDSTYSLS | KTHTCPPCPAPEAAG | VLQSSGLYSLSS | VTVSWNSGAL |
| | STLTLSKADYE | APSVFLFPPKPKDTL | VVTVPSSSLGTQ | TSGVHTFPAVL |
| | KHKVYACEVT | MISRTPEVTCVVVDV | TYICNVNHKPSN | QSSGLYSLSSV |
| | HQGLSSPVTKS | SHEDPEVKFNWYVD | TKVDKKVEPKS | VTVPSSSLGTQ |
| | FNRGEC | GVEVHNAKTKPREE | CDKTHTCPPCPA | TYICNVNHKPS |
| | (SEQ ID | QYNSTYRVVSVLTVL | PEAAGAPSVFLF | NTKVDKKVEP |
| | NO: 156) | HQDWLNGKEYKCKV | PPKPKDTLMISR | KSCDKTHTCPP |
| | | SNKALPAPIEKTISKA | TPEVTCVVVDVS | CPAPEAAGAPS |
| | | KGQPREPQVYTLPPC | HEDPEVKFNWY | VFLFPPKPKDT |
| | | REEMTKNQVSLSCA | VDGVEVHNAKT | LMISRTPEVTC |
| | | VKGFYPSDIAVEWES | KPREEQYNSTYR | VVVDVSHEDP |
| | | NGQPENNYKTTPPVL | VVSVLTVLHQD | EVKFNWYVDG |
| | | DSDGSFFLVSKLTVD | WLNGKEYKCKV | VEVHNAKTKP |
| | | KSRWQQGNVFSCSV | SNKALPAPIEKTI | REEQYNSTYR |
| | | MHEALHNHYTQKSL | SKAKGQPREPQV | VVSVLTVLHQ |
| | | SLSPGSGGGGSGGGG | CTLPPSREEMTK | DWLNGKEYKC |
| | | SGGGGSAPTSSSTKK | NQVSLWCLVKG | KVSNKALPAPI |
| | | TQLQLEHLLLDLQMI | FYPSDIAVEWES | EKTISKAKGQP |
| | | LNGINNYKNPKLTEM | NGQPENNYKTTP | REPQVCTLPPS |
| | | LTAKFYMPKKATEL | PVLDSDGSFFLY | REEMTKNQVS |
| | | KHLQCLEEELKPLEE | SKLTVDKSRWQ | LWCLVKGFYP |
| | | VLNLAQSKNFHLRPR | QGNVFSCSVMH | SDIAVEWESNG |
| | | DLISAINVIVLELKGS | EALHNHYTQKS | QPENNYKTTPP |
| | | ETTFMCEYADETATI | LSLSPG | VLDSDGSFFLY |
| | | VEFLNRWITFAQSIIS | (SEQ ID | SKLTVDKSRW |
| | | TLT | NO: 158) | QQGNVFSCSV |
| | | (SEQ ID NO: 157) | | MHEALHNHYT |
| | | | | QKSLSLSPGK |
| | | | | (SEQ ID |
| | | | | NO: 217) |
| xhCD8v2- | DIQMTQSPSSLS | QVQLVQSGAEVKKP | QVQLVQSGAEV | QVQLVQSGAE |
| IL-2m1 | ASVGDRVTITC | GSSVKVSCKASGYRF | KKPGSSVKVSCK | VKKPGSSVKV |
| | RASQEIYGALN | HNFAISWVRQAPGQ | ASGYRFHNFAIS | SCKASGYRFH |
| | WYQQKPGKAP | GLEWMGGIIPGHAKA | WVRQAPGQGLE | NFAISWVRQAP |
| | KLLIYGATNLQ | NYAQKFQGRVTITAD | WMGGIIPGHAK | GQGLEWMGGI |
| | SGVPSRFSGSGS | ESTSTAYMELSSLRSE | ANYAQKFQGRV | IPGHAKANYA |
| | GTDFTLTISSLQ | DTAVYYCARDGLGIR | TITADESTSTAY | QKFQGRVTITA |
| | PEDFATYYCQD | LFADWGQGTLVTVS | MELSSLRSEDTA | DESTSTAYMEL |
| | IYDAPWTFGGG | SASTKGPSVFPLAPSS | VYYCARDGLGIR | SSLRSEDTAVY |
| | TKVEIKRTVAA | KSTSGGTAALGCLVK | LFADWGQGTLV | YCARDGLGIRL |
| | PSVFIFPPSDEQ | DYFPEPVTVSWNSGA | TVSSASTKGPSV | FADWGQGTLV |
| | LKSGTASVVCL | LTSGVHTFPAVLQSS | FPLAPSSKSTSGG | TVSSASTKGPS |
| | LNNFYPREAKV | GLYSLSSVVTVPSSSL | TAALGCLVKDY | VFPLAPSSKST |
| | QWKVDNALQS | GTQTYICNVNHKPSN | FPEPVTVSWNSG | SGGTAALGCL |
| | GNSQESVTEQD | TKVDKKVEPKSCDK | ALTSGVHTFPAV | VKDYFPEPVTV |
| | SKDSTYSLSSTL | THTCPPCPAPEAAGA | LQSSGLYSLSSV | SWNSGALTSG |
| | TLSKADYEKHK | PSVFLFPPKPKDTLMI | VTVPSSSLGTQT | VHTFPAVLQSS |
| | VYACEVTHQGL | SRTPEVTCVVVDVSH | YICNVNHKPSNT | GLYSLSSVVTV |
| | SSPVTKSFNRGE | EDPEVKFNWYVDGV | KVDKKVEPKSC | PSSSLGTQTYIC |
| | C | EVHNAKTKPREEQY | DKTHTCPPCPAP | NVNHKPSNTK |
| | (SEQ ID | NSTYRVVSVLTVLHQ | EAAGAPSVFLFP | VDKKVEPKSC |
| | NO: 159) | DWLNGKEYKCKVSN | PKPKDTLMISRT | DKTHTCPPCPA |
| | | KALPAPIEKTISKAKG | PEVTCVVVDVS | PEAAGAPSVFL |
| | | QPREPQVYTLPPCRE | HEDPEVKFNWY | FPPKPKDTLMI |
| | | EMTKNQVSLSCAVK | VDGVEVHNAKT | SRTPEVTCVVV |
| | | GFYPSDIAVEWESNG | KPREEQYNSTYR | DVSHEDPEVKF |
| | | QPENNYKTTPPVLDS | VVSVLTVLHQD | NWYVDGVEV |
| | | DGSFFLVSKLTVDKS | WLNGKEYKCKV | HNAKTKPREE |
| | | RWQQGNVFSCSVMH | SNKALPAPIEKTI | QYNSTYRVVS |
| | | EALHNHYTQKSLSLS | SKAKGQPREPQV | VLTVLHQDWL |
| | | PGSGGGGSGGGGSG | CTLPPSREEMTK | NGKEYKCKVS |
| | | GGGSAPTSSSTKKTQ | NQVSLWCLVKG | NKALPAPIEKTI |
| | | LQLEHLLLDLQMILN | FYPSDIAVEWES | SKAKGQPREP |
| | | GINNYKNPKLTEMLT | NGQPENNYKTTP | QVCTLPPSREE |
| | | AKFYMPKKATELKH | PVLDSDGSFFLY | MTKNQVSLWC |
| | | LQCLEEELKPLEEVL | SKLTVDKSRWQ | LVKGFYPSDIA |
| | | NLAQSKNFHLRPRDL | QGNVFSCSVMH | VEWESNGQPE |
| | | ISAINVIVLELKGSET | EALHNHYTQKS | NNYKTTPPVL |
| | | TFMCEYADETATIVE | LSLSPG | DSDGSFFLYSK |
| | | FLNRWITFAQSIISTLT | (SEQ ID | LTVDKSRWQQ |
| | | (SEQ ID NO: 160) | NO: 161) | GNVFSCSVMH |
| | | | | EALHNHYTQK |
| | | | | SLSLSPGK |
| | | | | (SEQ ID |
| | | | | NO: 218) |
| xhCD8v3- | DIQMTQSPSSLS | QVQLVQSGAEVKKP | QVQLVQSGAEV | QVQLVQSGAE |
| IL-2m1 | ASVGDRVTITC | GSSVKVSCKASGSRF | KKPGSSVKVSCK | VKKPGSSVKV |
| | RASQEIYGALN | YKFAISWVRQAPGQ | ASGSRFYKFAIS | SCKASGSRFYK |
| | WYQQKPGKAP | GLEWMGGIIPGHAKA | WVRQAPGQGLE | FAISWVRQAPG |
| | KLLIYGATNLQ | NYAQKFQGRVTITAD | WMGGIIPGHAK | QGLEWMGGIIP |
| | SGVPSRFSGSGS | ESTSTAYMELSSLRSE | ANYAQKFQGRV | GHAKANYAQK |
| | GTDFTLTISSLQ | DTAVYYCARDGLGIR | TITADESTSTAY | FQGRVTITADE |
| | PEDFATYYCQD | LFADWGQGTLVTVS | MELSSLRSEDTA | STSTAYMELSS |
| | IYDAPWTFGGG | SASTKGPSVFPLAPSS | VYYCARDGLGIR | LRSEDTAVYY |
| | TKVEIKRTVAA | KSTSGGTAALGCLVK | LFADWGQGTLV | CARDGLGIRLF |
| | PSVFIFPPSDEQ | DYFPEPVTVSWNSGA | TVSSASTKGPSV | ADWGQGTLVT |
| | LKSGTASVVCL | LTSGVHTFPAVLQSS | FPLAPSSKSTSGG | VSSASTKGPSV |
| | LNNFYPREAKV | GLYSLSSVVTVPSSSL | TAALGCLVKDY | FPLAPSSKSTS |
| | QWKVDNALQS | GTQTYICNVNHKPSN | FPEPVTVSWNSG | GGTAALGCLV |
| | GNSQESVTEQD | TKVDKKVEPKSCDK | ALTSGVHTFPAV | KDYFPEPVTVS |
| | SKDSTYSLSSTL | THTCPPCPAPEAAGA | LQSSGLYSLSSV | WNSGALTSGV |
| | TLSKADYEKHK | PSVFLFPPKPKDTLMI | VTVPSSSLGTQT | HTFPAVLQSSG |
| | VYACEVTHQGL | SRTPEVTCVVVDVSH | YICNVNHKPSNT | LYSLSSVVTVP |
| | SSPVTKSFNRGE | EDPEVKFNWYVDGV | KVDKKVEPKSC | SSSLGTQTYIC |
| | C | EVHNAKTKPREEQY | DKTHTCPPCPAP | NVNHKPSNTK |
| | (SEQ ID | NSTYRVVSVLTVLHQ | EAAGAPSVFLFP | VDKKVEPKSC |
| | NO: 162) | DWLNGKEYKCKVSN | PKPKDTLMISRT | DKTHTCPPCPA |
| | | KALPAPIEKTISKAKG | PEVTCVVVDVS | PEAAGAPSVFL |
| | | QPREPQVYTLPPCRE | HEDPEVKFNWY | FPPKPKDTLMI |
| | | EMTKNQVSLSCAVK | VDGVEVHNAKT | SRTPEVTCVVV |
| | | GFYPSDIAVEWESNG | KPREEQYNSTYR | DVSHEDPEVKF |
| | | QPENNYKTTPPVLDS | VVSVLTVLHQD | NWYVDGVEV |
| | | DGSFFLVSKLTVDKS | WLNGKEYKCKV | HNAKTKPREE |
| | | RWQQGNVFSCSVMH | SNKALPAPIEKTI | QYNSTYRVVS |
| | | EALHNHYTQKSLSLS | SKAKGQPREPQV | VLTVLHQDWL |
| | | PGSGGGGSGGGGSG | CTLPPSREEMTK | NGKEYKCKVS |
| | | GGGSAPTSSSTKKTQ | NQVSLWCLVKG | NKALPAPIEKTI |
| | | LQLEHLLLDLQMILN | FYPSDIAVEWES | SKAKGQPREP |
| | | GINNYKNPKLTEMLT | NGQPENNYKTTP | QVCTLPPSREE |
| | | AKFYMPKKATELKH | PVLDSDGSFFLY | MTKNQVSLWC |
| | | LQCLEEELKPLEEVL | SKLTVDKSRWQ | LVKGFYPSDIA |
| | | NLAQSKNFHLRPRDL | QGNVFSCSVMH | VEWESNGQPE |
| | | ISAINVIVLELKGSET | EALHNHYTQKS | NNYKTTPPVL |
| | | TFMCEYADETATIVE | LSLSPG | DSDGSFFLYSK |
| | | FLNRWITFAQSIISTLT | (SEQ ID | LTVDKSRWQQ |
| | | (SEQ ID NO: 163) | NO: 164) | GNVFSCSVMH |
| | | | | EALHNHYTQK |
| | | | | SLSLSPGK |
| | | | | (SEQ ID |
| | | | | NO: 219) |
| xhCD8v4- | DIQMTQSPSSLS | QVQLVQSGAEVKKP | QVQLVQSGAEV | QVQLVQSGAE |
| IL-2m1 | ASVGDRVTITC | GSSVKVSCKASGYTF | KKPGSSVKVSCK | VKKPGSSVKV |
| | RASQKIYGALN | TKYAISWVRQAPGQ | ASGYTFTKYAIS | SCKASGYTFTK |
| | WYQQKPGKAP | GLEWMGGIIPGHAKA | WVRQAPGQGLE | YAISWVRQAP |
| | KLLIYGATNLQ | NYAQKFQGRVTITAD | WMGGIIPGHAK | GQGLEWMGGI |
| | SGVPSRFSGSGS | ESTSTAYMELSSLRSE | ANYAQKFQGRV | IPGHAKANYA |
| | GTDFTLTISSLQ | DTAVYYCARDGLGIR | TITADESTSTAY | QKFQGRVTITA |
| | PEDFATYYCON | LFADWGQGTLVTVS | MELSSLRSEDTA | DESTSTAYMEL |
| | TYDTPWTFGGG | SASTKGPSVFPLAPSS | VYYCARDGLGIR | SSLRSEDTAVY |
| | TKVEIKRTVAA | KSTSGGTAALGCLVK | LFADWGQGTLV | YCARDGLGIRL |
| | PSVFIFPPSDEQ | DYFPEPVTVSWNSGA | TVSSASTKGPSV | FADWGQGTLV |
| | LKSGTASVVCL | LTSGVHTFPAVLQSS | FPLAPSSKSTSGG | TVSSASTKGPS |
| | LNNFYPREAKV | GLYSLSSVVTVPSSSL | TAALGCLVKDY | VFPLAPSSKST |
| | QWKVDNALQS | GTQTYICNVNHKPSN | FPEPVTVSWNSG | SGGTAALGCL |
| | GNSQESVTEQD | TKVDKKVEPKSCDK | ALTSGVHTFPAV | VKDYFPEPVTV |
| | SKDSTYSLSSTL | THTCPPCPAPEAAGA | LQSSGLYSLSSV | SWNSGALTSG |
| | TLSKADYEKHK | PSVFLFPPKPKDTLMI | VTVPSSSLGTQT | VHTFPAVLQSS |
| | VYACEVTHQGL | SRTPEVTCVVVDVSH | YICNVNHKPSNT | GLYSLSSVVTV |
| | SSPVTKSFNRGE | EDPEVKFNWYVDGV | KVDKKVEPKSC | PSSSLGTQTYIC |
| | C | EVHNAKTKPREEQY | DKTHTCPPCPAP | NVNHKPSNTK |
| | (SEQ ID | NSTYRVVSVLTVLHQ | EAAGAPSVFLFP | VDKKVEPKSC |
| | NO: 165) | DWLNGKEYKCKVSN | PKPKDTLMISRT | DKTHTCPPCPA |
| | | KALPAPIEKTISKAKG | PEVTCVVVDVS | PEAAGAPSVFL |
| | | QPREPQVYTLPPCRE | HEDPEVKFNWY | FPPKPKDTLMI |
| | | EMTKNQVSLSCAVK | VDGVEVHNAKT | SRTPEVTCVVV |
| | | GFYPSDIAVEWESNG | KPREEQYNSTYR | DVSHEDPEVKF |
| | | QPENNYKTTPPVLDS | VVSVLTVLHQD | NWYVDGVEV |
| | | DGSFFLVSKLTVDKS | WLNGKEYKCKV | HNAKTKPREE |
| | | RWQQGNVFSCSVMH | SNKALPAPIEKTI | QYNSTYRVVS |
| | | EALHNHYTQKSLSLS | SKAKGQPREPQV | VLTVLHQDWL |
| | | PGSGGGGSGGGGSG | CTLPPSREEMTK | NGKEYKCKVS |
| | | GGGSAPTSSSTKKTQ | NQVSLWCLVKG | NKALPAPIEKTI |
| | | LQLEHLLLDLQMILN | FYPSDIAVEWES | SKAKGQPREP |
| | | GINNYKNPKLTEMLT | NGQPENNYKTTP | QVCTLPPSREE |
| | | AKFYMPKKATELKH | PVLDSDGSFFLY | MTKNQVSLWC |
| | | LQCLEEELKPLEEVL | SKLTVDKSRWQ | LVKGFYPSDIA |
| | | NLAQSKNFHLRPRDL | QGNVFSCSVMH | VEWESNGQPE |
| | | ISAINVIVLELKGSET | EALHNHYTQKS | NNYKTTPPVL |
| | | TFMCEYADETATIVE | LSLSPG | DSDGSFFLYSK |
| | | FLNRWITFAQSIISTLT | (SEQ ID | LTVDKSRWQQ |
| | | (SEQ ID NO: 166) | NO: 167) | GNVFSCSVMH |
| | | | | EALHNHYTQK |
| | | | | SLSLSPGK |
| | | | | (SEQ ID |
| | | | | NO: 220) |
| xhCD8v5- | DIQMTQSPSSLS | QVQLVQSGAEVKKP | QVQLVQSGAEV | QVQLVQSGAE |
| IL-2m1 | ASVGDRVTITC | GSSVKVSCKASGSGF | KKPGSSVKVSCK | VKKPGSSVKV |
| | RASQKIYGALN | RGHAISWVRQAPGQ | ASGSGFRGHAIS | SCKASGSGFRG |
| | WYQQKPGKAP | GLEWMGGIIPGHAKA | WVRQAPGQGLE | HAISWVRQAP |
| | KLLIYGATNLQ | NYAQKFQGRVTITAD | WMGGIIPGHAK | GQGLEWMGGI |
| | SGVPSRFSGSGS | ESTSTAYMELSSLRSE | ANYAQKFQGRV | IPGHAKANYA |
| | GTDFTLTISSLQ | DTAVYYCARDGLGIR | TITADESTSTAY | QKFQGRVTITA |
| | PEDFATYYCQN | LFADWGQGTLVTVS | MELSSLRSEDTA | DESTSTAYMEL |
| | TYDTPWTFGGG | SASTKGPSVFPLAPSS | VYYCARDGLGIR | SSLRSEDTAVY |
| | TKVEIKRTVAA | KSTSGGTAALGCLVK | LFADWGQGTLV | YCARDGLGIRL |
| | PSVFIFPPSDEQ | DYFPEPVTVSWNSGA | TVSSASTKGPSV | FADWGQGTLV |
| | LKSGTASVVCL | LTSGVHTFPAVLQSS | FPLAPSSKSTSGG | TVSSASTKGPS |
| | LNNFYPREAKV | GLYSLSSVVTVPSSSL | TAALGCLVKDY | VFPLAPSSKST |
| | QWKVDNALQS | GTQTYICNVNHKPSN | FPEPVTVSWNSG | SGGTAALGCL |
| | GNSQESVTEQD | TKVDKKVEPKSCDK | ALTSGVHTFPAV | VKDYFPEPVTV |
| | SKDSTYSLSSTL | THTCPPCPAPEAAGA | LQSSGLYSLSSV | SWNSGALTSG |
| | TLSKADYEKHK | PSVFLFPPKPKDTLMI | VTVPSSSLGTQT | VHTFPAVLQSS |
| | VYACEVTHQGL | SRTPEVTCVVVDVSH | YICNVNHKPSNT | GLYSLSSVVTV |
| | SSPVTKSFNRGE | EDPEVKFNWYVDGV | KVDKKVEPKSC | PSSSLGTQTYIC |
| | C | EVHNAKTKPREEQY | DKTHTCPPCPAP | NVNHKPSNTK |
| | (SEQ ID NO:168) | NSTYRVVSVLTVLHQ | EAAGAPSVFLFP | VDKKVEPKSC |
| | | DWLNGKEYKCKVSN | PKPKDTLMISRT | DKTHTCPPCPA |
| | | KALPAPIEKTISKAKG | PEVTCVVVDVS | PEAAGAPSVFL |
| | | QPREPQVYTLPPCRE | HEDPEVKFNWY | FPPKPKDTLMI |
| | | EMTKNQVSLSCAVK | VDGVEVHNAKT | SRTPEVTCVVV |
| | | GFYPSDIAVEWESNG | KPREEQYNSTYR | DVSHEDPEVKF |
| | | QPENNYKTTPPVLDS | VVSVLTVLHQD | NWYVDGVEV |
| | | DGSFFLVSKLTVDKS | WLNGKEYKCKV | HNAKTKPREE |
| | | RWQQGNVFSCSVMH | SNKALPAPIEKTI | QYNSTYRVVS |
| | | EALHNHYTQKSLSLS | SKAKGQPREPQV | VLTVLHQDWL |
| | | PGSGGGGSGGGGSG | CTLPPSREEMTK | NGKEYKCKVS |
| | | GGGSAPTSSSTKKTQ | NQVSLWCLVKG | NKALPAPIEKTI |
| | | LQLEHLLLDLQMILN | FYPSDIAVEWES | SKAKGQPREP |
| | | GINNYKNPKLTEMLT | NGQPENNYKTTP | QVCTLPPSREE |
| | | AKFYMPKKATELKH | PVLDSDGSFFLY | MTKNQVSLWC |
| | | LQCLEEELKPLEEVL | SKLTVDKSRWQ | LVKGFYPSDIA |
| | | NLAQSKNFHLRPRDL | QGNVFSCSVMH | VEWESNGQPE |
| | | ISAINVIVLELKGSET | EALHNHYTQKS | NNYKTTPPVL |
| | | TFMCEYADETATIVE | LSLSPG | DSDGSFFLYSK |
| | | FLNRWITFAQSIISTLT | (SEQ ID | LTVDKSRWQQ |
| | | (SEQ ID NO: 169) | NO: 170) | GNVFSCSVMH |
| | | | | EALHNHYTQK |
| | | | | SLSLSPGK |
| | | | | (SEQ ID |
| | | | | NO: 221) |
| xhCD8v6- | EIVLTQSPATLS | EVQLVESGGGAVRP | EVQLVESGGGA | EVQLVESGGG |
| IL-2m1 | VSPGERATLSC | GGSLRLSCAASGFTF | VRPGGSLRLSCA | AVRPGGSLRLS |
| | RASQSVSSNLA | DDYGMSWVRQAPG | ASGFTFDDYGM | CAASGFTFDD |
| | WYQQKPGQAP | KGLEWVSDINWSGEI | SWVRQAPGKGL | YGMSWVRQAP |
| | RLLIYGASSRAT | TAYADSVKGRFTISR | EWVSDINWSGEI | GKGLEWVSDI |
| | GIPDRFSGSGSG | DNAKNSLYLQMNSL | TAYADSVKGRF | NWSGEITAYA |
| | TDFTLTISRLEP | RAEDTAVYYCARSN | TISRDNAKNSLY | DSVKGRFTISR |
| | EDFAVYYCQQ | SYRWDDALDIWGQG | LQMNSLRAEDT | DNAKNSLYLQ |
| | YGSSPPVTFGQ | TMVTVSSASTKGPSV | AVYYCARSNSY | MNSLRAEDTA |
| | GTKVEIKRTVA | FPLAPSSKSTSGGTAA | RWDDALDIWGQ | VYYCARSNSY |
| | APSVFIFPPSDE | LGCLVKDYFPEPVTV | GTMVTVSSASTK | RWDDALDIWG |
| | QLKSGTASVVC | SWNSGALTSGVHTFP | GPSVFPLAPSSKS | QGTMVTVSSA |
| | LLNNFYPREAK | AVLQSSGLYSLSSVV | TSGGTAALGCLV | STKGPSVFPLA |
| | VQWKVDNALQ | TVPSSSLGTQTYICNV | KDYFPEPVTVSW | PSSKSTSGGTA |
| | SGNSQESVTEQ | NHKPSNTKVDKKVE | NSGALTSGVHTF | ALGCLVKDYF |
| | DSKDSTYSLSST | PKSCDKTHTCPPCPA | PAVLQSSGLYSL | PEPVTVSWNS |
| | LTLSKADYEKH | PEAAGAPSVFLFPPKP | SSVVTVPSSSLG | GALTSGVHTFP |
| | KVYACEVTHQ | KDTLMISRTPEVTCV | TQTYICNVNHKP | AVLQSSGLYSL |
| | GLSSPVTKSFNR | VVDVSHEDPEVKFN | SNTKVDKKVEP | SSVVTVPSSSL |
| | GEC | WYVDGVEVHNAKT | KSCDKTHTCPPC | GTQTYICNVN |
| | (SEQ ID NO: 171) | KPREEQYNSTYRVVS | PAPEAAGAPSVF | HKPSNTKVDK |
| | | VLTVLHQDWLNGKE | LFPPKPKDTLMIS | KVEPKSCDKT |
| | | YKCKVSNKALPAPIE | RTPEVTCVVVD | HTCPPCPAPEA |
| | | KTISKAKGQPREPQV | VSHEDPEVKFN | AGAPSVFLFPP |
| | | YTLPPCREEMTKNQV | WYVDGVEVHN | KPKDTLMISRT |
| | | SLSCAVKGFYPSDIA | AKTKPREEQYNS | PEVTCVVVDV |
| | | VEWESNGQPENNYK | TYRVVSVLTVLH | SHEDPEVKFN |
| | | TTPPVLDSDGSFFLVS | QDWLNGKEYKC | WYVDGVEVH |
| | | KLTVDKSRWQQGNV | KVSNKALPAPIE | NAKTKPREEQ |
| | | FSCSVMHEALHNHY | KTISKAKGQPRE | YNSTYRVVSV |
| | | TQKSLSLSPGSGGGG | PQVCTLPPSREE | LTVLHQDWLN |
| | | SGGGGSGGGGSAPTS | MTKNQVSLWCL | GKEYKCKVSN |
| | | SSTKKTQLQLEHLLL | VKGFYPSDIAVE | KALPAPIEKTIS |
| | | DLQMILNGINNYKNP | WESNGQPENNY | KAKGQPREPQ |
| | | KLTEMLTAKFYMPK | KTTPPVLDSDGS | VCTLPPSREEM |
| | | KATELKHLQCLEEEL | FFLYSKLTVDKS | TKNQVSLWCL |
| | | KPLEEVLNLAQSKNF | RWQQGNVFSCS | VKGFYPSDIAV |
| | | HLRPRDLISAINVIVL | VMHEALHNHYT | EWESNGQPEN |
| | | ELKGSETTFMCEYAD | QKSLSLSPG | NYKTTPPVLDS |
| | | ETATIVEFLNRWITFA | (SEQ ID | DGSFFLYSKLT |
| | | QSIISTLT | NO: 173) | VDKSRWQQGN |
| | | (SEQ ID NO: 172) | | VFSCSVMHEA |
| | | | | LHNHYTQKSL |
| | | | | SLSPGK |
| | | | | (SEQ ID |
| | | | | NO: 222) |
| xhCD8v7- | EIVLTQSPATLS | EVQLVESGGGLVQPG | EVQLVESGGGL | EVQLVESGGG |
| IL-2m1 | VTPGEGATLSC | RSLRLSCAASGFTFD | VQPGRSLRLSCA | LVQPGRSLRLS |
| | RASHSVGSNLA | DYAMHWVRQAPGK | ASGFTFDDYAM | CAASGFTFDD |
| | WYQQKPGQAP | GLEWVAVISYDGSN | HWVRQAPGKGL | YAMHWVRQA |
| | RLLIYDASNRA | KYYADSVKGRFTISR | EWVAVISYDGS | PGKGLEWVAV |
| | TGIPARFSGSGS | DNSKNTLYLQMNSL | NKYYADSVKGR | ISYDGSNKYYA |
| | GTDFTLTISSLE | RAEDTAVYYCAKDRI | FTISRDNSKNTL | DSVKGRFTISR |
| | PEDLAVYYCQQ | GWYDYDAFDIWGQG | YLQMNSLRAED | DNSKNTLYLQ |
| | RSNWPPTFGQG | TMVTVSSASTKGPSV | TAVYYCAKDRI | MNSLRAEDTA |
| | TRLEIKRTVAAP | FPLAPSSKSTSGGTAA | GWYDYDAFDIW | VYYCAKDRIG |
| | SVFIFPPSDEQL | LGCLVKDYFPEPVTV | GQGTMVTVSSA | WYDYDAFDIW |
| | KSGTASVVCLL | SWNSGALTSGVHTFP | STKGPSVFPLAPS | GQGTMVTVSS |
| | NNFYPREAKVQ | AVLQSSGLYSLSSVV | SKSTSGGTAALG | ASTKGPSVFPL |
| | WKVDNALQSG | TVPSSSLGTQTYICNV | CLVKDYFPEPVT | APSSKSTSGGT |
| | NSQESVTEQDS | NHKPSNTKVDKKVE | VSWNSGALTSG | AALGCLVKDY |
| | KDSTYSLSSTLT | PKSCDKTHTCPPCPA | VHTFPAVLQSSG | FPEPVTVSWNS |
| | LSKADYEKHKV | PEAAGAPSVFLFPPKP | LYSLSSVVTVPS | GALTSGVHTFP |
| | YACEVTHQGLS | KDTLMISRTPEVTCV | SSLGTQTYICNV | AVLQSSGLYSL |
| | SPVTKSFNRGE | VVDVSHEDPEVKFN | NHKPSNTKVDK | SSVVTVPSSSL |
| | C | WYVDGVEVHNAKT | KVEPKSCDKTHT | GTQTYICNVN |
| | (SEQ ID NO: 174) | KPREEQYNSTYRVVS | CPPCPAPEAAGA | HKPSNTKVDK |
| | | VLTVLHQDWLNGKE | PSVFLFPPKPKDT | KVEPKSCDKT |
| | | YKCKVSNKALPAPIE | LMISRTPEVTCV | HTCPPCPAPEA |
| | | KTISKAKGQPREPQV | VVDVSHEDPEV | AGAPSVFLFPP |
| | | YTLPPCREEMTKNQV | KFNWYVDGVEV | KPKDTLMISRT |
| | | SLSCAVKGFYPSDIA | HNAKTKPREEQ | PEVTCVVVDV |
| | | VEWESNGQPENNYK | YNSTYRVVSVLT | SHEDPEVKFN |
| | | TTPPVLDSDGSFFLVS | VLHQDWLNGKE | WYVDGVEVH |
| | | KLTVDKSRWQQGNV | YKCKVSNKALP | NAKTKPREEQ |
| | | FSCSVMHEALHNHY | APIEKTISKAKG | YNSTYRVVSV |
| | | TQKSLSLSPGSGGGG | QPREPQVCTLPP | LTVLHQDWLN |
| | | SGGGGSGGGGSAPTS | SREEMTKNQVSL | GKEYKCKVSN |
| | | SSTKKTQLQLEHLLL | WCLVKGFYPSDI | KALPAPIEKTIS |
| | | DLQMILNGINNYKNP | AVEWESNGQPE | KAKGQPREPQ |
| | | KLTEMLTAKFYMPK | NNYKTTPPVLDS | VCTLPPSREEM |
| | | KATELKHLQCLEEEL | DGSFFLYSKLTV | TKNQVSLWCL |
| | | KPLEEVLNLAQSKNF | DKSRWQQGNVF | VKGFYPSDIAV |
| | | HLRPRDLISAINVIVL | SCSVMHEALHN | EWESNGQPEN |
| | | ELKGSETTFMCEYAD | HYTQKSLSLSPG | NYKTTPPVLDS |
| | | ETATIVEFLNRWITFA | (SEQ ID | DGSFFLYSKLT |
| | | QSIISTLT | NO: 176) | VDKSRWQQGN |
| | | (SEQ ID NO: 175) | | VFSCSVMHEA |
| | | | | LHNHYTQKSL |
| | | | | SLSPGK |
| | | | | (SEQ ID |
| | | | | NO: 223) |
| xhCD8v8- | DIQMTQSPASLS | QVQLQQSGAELVRP | QVQLQQSGAEL | QVQLQQSGAE |
| IL-2m1 | VSVGETVTITCR | GSSVKISCKASGYAF | VRPGSSVKISCK | LVRPGSSVKIS |
| | ASENIYSNLAW | SSYWMNWVKQRPG | ASGYAFSSYWM | CKASGYAFSSY |
| | YQQKQGKSPQL | QGLEWIGQIYPGDGD | NWVKQRPGQGL | WMNWVKQRP |
| | LVYAATNLAD | TNYNGKFKGKATLT | EWIGQIYPGDGD | GQGLEWIGQIY |
| | GVPSRFSGSGS | ADKSSSTAYMQLSSL | TNYNGKFKGKA | PGDGDTNYNG |
| | GTQYSLKINSL | TSEDSAVYFCARSGA | TLTADKSSSTAY | KFKGKATLTA |
| | QSEDFGSYYCQ | AFSSYYAMDYWGQG | MQLSSLTSEDSA | DKSSSTAYMQ |
| | HFWGTPWTFG | TSVTVSSASTKGPSVF | VYFCARSGAAFS | LSSLTSEDSAV |
| | GGTKLEIKRTV | PLAPSSKSTSGGTAA | SYYAMDYWGQ | YFCARSGAAFS |
| | AAPSVFIFPPSD | LGCLVKDYFPEPVTV | GTSVTVSSASTK | SYYAMDYWG |
| | EQLKSGTASVV | SWNSGALTSGVHTFP | GPSVFPLAPSSKS | QGTSVTVSSAS |
| | CLLNNFYPREA | AVLQSSGLYSLSSVV | TSGGTAALGCLV | TKGPSVFPLAP |
| | KVQWKVDNAL | TVPSSSLGTQTYICNV | KDYFPEPVTVSW | SSKSTSGGTAA |
| | QSGNSQESVTE | NHKPSNTKVDKKVE | NSGALTSGVHTF | LGCLVKDYFPE |
| | QDSKDSTYSLS | PKSCDKTHTCPPCPA | PAVLQSSGLYSL | PVTVSWNSGA |
| | STLTLSKADYE | PEAAGAPSVFLFPPKP | SSVVTVPSSSLG | LTSGVHTFPAV |
| | KHKVYACEVT | KDTLMISRTPEVTCV | TQTYICNVNHKP | LQSSGLYSLSS |
| | HQGLSSPVTKS | VVDVSHEDPEVKFN | SNTKVDKKVEP | VVTVPSSSLGT |
| | FNRGEC | WYVDGVEVHNAKT | KSCDKTHTCPPC | QTYICNVNHKP |
| | (SEQ ID NO: 187) | KPREEQYNSTYRVVS | PAPEAAGAPSVF | SNTKVDKKVE |
| | | VLTVLHQDWLNGKE | LFPPKPKDTLMIS | PKSCDKTHTCP |
| | | YKCKVSNKALPAPIE | RTPEVTCVVVD | PCPAPEAAGAP |
| | | KTISKAKGQPREPQV | VSHEDPEVKFN | SVFLFPPKPKD |
| | | YTLPPCREEMTKNQV | WYVDGVEVHN | TLMISRTPEVT |
| | | SLSCAVKGFYPSDIA | AKTKPREEQYNS | CVVVDVSHED |
| | | VEWESNGQPENNYK | TYRVVSVLTVLH | PEVKFNWYVD |
| | | TTPPVLDSDGSFFLVS | QDWLNGKEYKC | GVEVHNAKTK |
| | | KLTVDKSRWQQGNV | KVSNKALPAPIE | PREEQYNSTYR |
| | | FSCSVMHEALHNHY | KTISKAKGQPRE | VVSVLTVLHQ |
| | | TQKSLSLSPGSGGGG | PQVCTLPPSREE | DWLNGKEYKC |
| | | SGGGGSGGGGSAPTS | MTKNQVSLWCL | KVSNKALPAPI |
| | | SSTKKTQLQLEHLLL | VKGFYPSDIAVE | EKTISKAKGQP |
| | | DLQMILNGINNYKNP | WESNGQPENNY | REPQVCTLPPS |
| | | KLTEMLTAKFYMPK | KTTPPVLDSDGS | REEMTKNQVS |
| | | KATELKHLQCLEEEL | FFLYSKLTVDKS | LWCLVKGFYP |
| | | KPLEEVLNLAQSKNF | RWQQGNVFSCS | SDIAVEWESNG |
| | | HLRPRDLISAINVIVL | VMHEALHNHYT | QPENNYKTTPP |
| | | ELKGSETTFMCEYAD | QKSLSLSPG | VLDSDGSFFLY |
| | | ETATIVEFLNRWITFA | (SEQ ID | SKLTVDKSRW |
| | | QSIISTLT | NO: 189) | QQGNVFSCSV |
| | | (SEQ ID NO: 188) | | MHEALHNHYT |
| | | | | QKSLSLSPGK |
| | | | | (SEQ ID |
| | | | | NO: 224) |
| xhCD8v9- | DIQMTQSPSSLS | QVQLVQSGAEVKKP | QVQLVQSGAEV | QVQLVQSGAE |
| IL-2m1 | ASVGDRVTITC | GSSVKVSCKASGGTF | KKPGSSVKVSCK | VKKPGSSVKV |
| | RASQEIYGALN | SSYAISWVRQAPGQG | ASGGTFSSYAIS | SCKASGGTFSS |
| | WYQQKPGKAP | LEWMGGIIPGAATAN | WVRQAPGQGLE | YAISWVRQAP |
| | KLLIYGATNLQ | YAQKFQGRVTITADE | WMGGIIPGAATA | GQGLEWMGGI |
| | SGVPSRFSGSGS | STSTAYMELSSLRSE | NYAQKFQGRVTI | IPGAATANYA |
| | GTDFTLTISSLQ | DTAVYYCARDAAGI | TADESTSTAYME | QKFQGRVTITA |
| | PEDFATYYCQS | RLFADWGQGTLVTV | LSSLRSEDTAVY | DESTSTAYMEL |
| | TYDAPWTFGG | SSASTKGPSVFPLAPS | YCARDAAGIRLF | SSLRSEDTAVY |
| | GTKVEIKRTVA | SKSTSGGTAALGCLV | ADWGQGTLVTV | YCARDAAGIR |
| | APSVFIFPPSDE | KDYFPEPVTVSWNSG | SSASTKGPSVFPL | LFADWGQGTL |
| | QLKSGTASVVC | ALTSGVHTFPAVLQS | APSSKSTSGGTA | VTVSSASTKGP |
| | LLNNFYPREAK | SGLYSLSSVVTVPSSS | ALGCLVKDYFPE | SVFPLAPSSKS |
| | VQWKVDNALQ | LGTQTYICNVNHKPS | PVTVSWNSGAL | TSGGTAALGC |
| | SGNSQESVTEQ | NTKVDKKVEPKSCD | TSGVHTFPAVLQ | LVKDYFPEPVT |
| | DSKDSTYSLSST | KTHTCPPCPAPEAAG | SSGLYSLSSVVT | VSWNSGALTS |
| | LTLSKADYEKH | APSVFLFPPKPKDTL | VPSSSLGTQTYIC | GVHTFPAVLQS |
| | KVYACEVTHQ | MISRTPEVTCVVVDV | NVNHKPSNTKV | SGLYSLSSVVT |
| | GLSSPVTKSFNR | SHEDPEVKFNWYVD | DKKVEPKSCDK | VPSSSLGTQTY |
| | GEC | GVEVHNAKTKPREE | THTCPPCPAPEA | ICNVNHKPSNT |
| | (SEQ ID NO: 298) | QYNSTYRVVSVLTVL | AGAPSVFLFPPK | KVDKKVEPKS |
| | | HQDWLNGKEYKCKV | PKDTLMISRTPE | CDKTHTCPPCP |
| | | SNKALPAPIEKTISKA | VTCVVVDVSHE | APEAAGAPSVF |
| | | KGQPREPQVYTLPPC | DPEVKFNWYVD | LFPPKPKDTLM |
| | | REEMTKNQVSLSCA | GVEVHNAKTKP | ISRTPEVTCVV |
| | | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSAPTSSSTKK | NQVSLWCLVKG | SNKALPAPIEK |
| | | TQLQLEHLLLDLQMI | FYPSDIAVEWES | TISKAKGQPRE |
| | | LNGINNYKNPKLTEM | NGQPENNYKTTP | PQVCTLPPSRE |
| | | LTAKFYMPKKATEL | PVLDSDGSFFLY | EMTKNQVSLW |
| | | KHLQCLEEELKPLEE | SKLTVDKSRWQ | CLVKGFYPSDI |
| | | VLNLAQSKNFHLRPR | QGNVFSCSVMH | AVEWESNGQP |
| | | DLISAINVIVLELKGS | EALHNHYTQKS | ENNYKTTPPVL |
| | | ETTFMCEYADETATI | LSLSPG | DSDGSFFLYSK |
| | | VEFLNRWITFAQSIIS | (SEQ ID | LTVDKSRWQQ |
| | | TLT | NO: 300) | GNVFSCSVMH |
| | | (SEQ ID NO: 299) | | EALHNHYTQK |
| | | | | SLSLSPGK |
| | | | | (SEQ ID |
| | | | | NO: 301) |
| xhCD8v10- | EIVLTQSPGTLS | EVQLVESGGGLVQPG | EVQLVESGGGL | EVQLVESGGG |
| IL-2m1 | LSPGERATLSCR | GSLRLSCAASGFTFSS | VQPGGSLRLSCA | LVQPGGSLRLS |
| | ASQSVSSNLAW | YAMSWVRQAPGKGL | ASGFTFSSYAMS | CAASGFTFSSY |
| | YQQKPGQAPRL | EWVSDITYAGGSTAY | WVRQAPGKGLE | AMSWVRQAPG |
| | LIYGASSRATGI | ADSVKGRFTISRDNA | WVSDITYAGGST | KGLEWVSDIT |
| | PDRFSGSGSGT | KNSLYLQMNSLRAE | AYADSVKGRFTI | YAGGSTAYAD |
| | DFTLTISRLEPE | DTAVYYCARSNAYA | SRDNAKNSLYL | SVKGRFTISRD |
| | DFAVYYCQQY | WDDALDIWGQGTM | QMNSLRAEDTA | NAKNSLYLQM |
| | GSSPPVTFGQG | VTVSSASTKGPSVFPL | VYYCARSNAYA | NSLRAEDTAV |
| | TKVEIKRTVAA | APSSKSTSGGTAALG | WDDALDIWGQG | YYCARSNAYA |
| | PSVFIFPPSDEQ | CLVKDYFPEPVTVSW | TMVTVSSASTKG | WDDALDIWGQ |
| | LKSGTASVVCL | NSGALTSGVHTFPAV | PSVFPLAPSSKST | GTMVTVSSAS |
| | LNNFYPREAKV | LQSSGLYSLSSVVTV | SGGTAALGCLV | TKGPSVFPLAP |
| | QWKVDNALQS | PSSSLGTQTYICNVN | KDYFPEPVTVSW | SSKSTSGGTAA |
| | GNSQESVTEQD | HKPSNTKVDKKVEP | NSGALTSGVHTF | LGCLVKDYFPE |
| | SKDSTYSLSSTL | KSCDKTHTCPPCPAP | PAVLQSSGLYSL | PVTVSWNSGA |
| | TLSKADYEKHK | EAAGAPSVFLFPPKP | SSVVTVPSSSLG | LTSGVHTFPAV |
| | VYACEVTHQGL | KDTLMISRTPEVTCV | TQTYICNVNHKP | LOSSGLYSLSS |
| | SSPVTKSFNRGE | VVDVSHEDPEVKFN | SNTKVDKKVEP | VVTVPSSSLGT |
| | C | WYVDGVEVHNAKT | KSCDKTHTCPPC | QTYICNVNHKP |
| | (SEQ ID | KPREEQYNSTYRVVS | PAPEAAGAPSVF | SNTKVDKKVE |
| | NO: 302) | VLTVLHQDWLNGKE | LFPPKPKDTLMIS | PKSCDKTHTCP |
| | | YKCKVSNKALPAPIE | RTPEVTCVVVD | PCPAPEAAGAP |
| | | KTISKAKGQPREPQV | VSHEDPEVKFN | SVFLFPPKPKD |
| | | YTLPPCREEMTKNQV | WYVDGVEVHN | TLMISRTPEVT |
| | | SLSCAVKGFYPSDIA | AKTKPREEQYNS | CVVVDVSHED |
| | | VEWESNGQPENNYK | TYRVVSVLTVLH | PEVKFNWYVD |
| | | TTPPVLDSDGSFFLVS | QDWLNGKEYKC | GVEVHNAKTK |
| | | KLTVDKSRWQQGNV | KVSNKALPAPIE | PREEQYNSTYR |
| | | FSCSVMHEALHNHY | KTISKAKGQPRE | VVSVLTVLHQ |
| | | TQKSLSLSPGSGGGG | PQVCTLPPSREE | DWLNGKEYKC |
| | | SGGGGSGGGGSAPTS | MTKNQVSLWCL | KVSNKALPAPI |
| | | SSTKKTQLQLEHLLL | VKGFYPSDIAVE | EKTISKAKGQP |
| | | DLQMILNGINNYKNP | WESNGQPENNY | REPQVCTLPPS |
| | | KLTEMLTAKFYMPK | KTTPPVLDSDGS | REEMTKNQVS |
| | | KATELKHLQCLEEEL | FFLYSKLTVDKS | LWCLVKGFYP |
| | | KPLEEVLNLAQSKNF | RWQQGNVFSCS | SDIAVEWESNG |
| | | HLRPRDLISAINVIVL | VMHEALHNHYT | QPENNYKTTPP |
| | | ELKGSETTFMCEYAD | QKSLSLSPG | VLDSDGSFFLY |
| | | ETATIVEFLNRWITFA | (SEQ ID | SKLTVDKSRW |
| | | QSIISTLT | NO: 304) | QQGNVFSCSV |
| | | (SEQ ID NO: 303) | | MHEALHNHYT |
| | | | | QKSLSLSPGK |
| | | | | (SEQ ID |
| | | | | NO: 305) |
| xhCD8v11- | EIVLTQSPGTLS | EVQLVESGGGLVQPG | EVQLVESGGGL | EVQLVESGGG |
| IL-2m1 | LSPGERATLSCR | GSLRLSCAASGFTFSS | VQPGGSLRLSCA | LVQPGGSLRLS |
| | ASQSVSSNLAW | YAMSWVRQAPGKGL | ASGFTFSSYAMS | CAASGFTFSSY |
| | YQQKPGQAPRL | EWVSDITYAGGSTAY | WVRQAPGKGLE | AMSWVRQAPG |
| | LIYGASSRATGI | ADSVKGRFTISRDNA | WVSDITYAGGST | KGLEWVSDIT |
| | PDRFSGSGSGT | KNSLYLQMNSLRAE | AYADSVKGRFTI | YAGGSTAYAD |
| | DFTLTISRLEPE | DTAVYYCARSNAYA | SRDNAKNSLYL | SVKGRFTISRD |
| | DFAVYYCQQY | WDDALDIWGQGTLV | QMNSLRAEDTA | NAKNSLYLQM |
| | GSSPPVTFGQG | TVSSASTKGPSVFPLA | VYYCARSNAYA | NSLRAEDTAV |
| | TKVEIKRTVAA | PSSKSTSGGTAALGC | WDDALDIWGQG | YYCARSNAYA |
| | PSVFIFPPSDEQ | LVKDYFPEPVTVSWN | TLVTVSSASTKG | WDDALDIWGQ |
| | LKSGTASVVCL | SGALTSGVHTFPAVL | PSVFPLAPSSKST | GTLVTVSSAST |
| | LNNFYPREAKV | QSSGLYSLSSVVTVPS | SGGTAALGCLV | KGPSVFPLAPS |
| | QWKVDNALQS | SSLGTQTYICNVNHK | KDYFPEPVTVSW | SKSTSGGTAAL |
| | GNSQESVTEQD | PSNTKVDKKVEPKSC | NSGALTSGVHTF | GCLVKDYFPEP |
| | SKDSTYSLSSTL | DKTHTCPPCPAPEAA | PAVLQSSGLYSL | VTVSWNSGAL |
| | TLSKADYEKHK | GAPSVFLFPPKPKDTL | SSVVTVPSSSLG | TSGVHTFPAVL |
| | VYACEVTHQGL | MISRTPEVTCVVVDV | TQTYICNVNHKP | QSSGLYSLSSV |
| | SSPVTKSFNRGE | SHEDPEVKFNWYVD | SNTKVDKKVEP | VTVPSSSLGTQ |
| | C | GVEVHNAKTKPREE | KSCDKTHTCPPC | TYICNVNHKPS |
| | (SEQ ID | QYNSTYRVVSVLTVL | PAPEAAGAPSVF | NTKVDKKVEP |
| | NO: 306) | HQDWLNGKEYKCKV | LFPPKPKDTLMIS | KSCDKTHTCPP |
| | | SNKALPAPIEKTISKA | RTPEVTCVVVD | CPAPEAAGAPS |
| | | KGQPREPQVYTLPPC | VSHEDPEVKFN | VFLFPPKPKDT |
| | | REEMTKNQVSLSCA | WYVDGVEVHN | LMISRTPEVTC |
| | | VKGFYPSDIAVEWES | AKTKPREEQYNS | VVVDVSHEDP |
| | | NGQPENNYKTTPPVL | TYRVVSVLTVLH | EVKFNWYVDG |
| | | DSDGSFFLVSKLTVD | QDWLNGKEYKC | VEVHNAKTKP |
| | | KSRWQQGNVFSCSV | KVSNKALPAPIE | REEQYNSTYR |
| | | MHEALHNHYTQKSL | KTISKAKGQPRE | VVSVLTVLHQ |
| | | SLSPGSGGGGSGGGG | PQVCTLPPSREE | DWLNGKEYKC |
| | | SGGGGSAPTSSSTKK | MTKNQVSLWCL | KVSNKALPAPI |
| | | TQLQLEHLLLDLQMI | VKGFYPSDIAVE | EKTISKAKGQP |
| | | LNGINNYKNPKLTEM | WESNGQPENNY | REPQVCTLPPS |
| | | LTAKFYMPKKATEL | KTTPPVLDSDGS | REEMTKNQVS |
| | | KHLQCLEEELKPLEE | FFLYSKLTVDKS | LWCLVKGFYP |
| | | VLNLAQSKNFHLRPR | RWQQGNVFSCS | SDIAVEWESNG |
| | | DLISAINVIVLELKGS | VMHEALHNHYT | QPENNYKTTPP |
| | | ETTFMCEYADETATI | QKSLSLSPG | VLDSDGSFFLY |
| | | VEFLNRWITFAQSIIS | (SEQ ID | SKLTVDKSRW |
| | | TLT | NO: 308) | QQGNVFSCSV |
| | | (SEQ ID NO: 307) | | MHEALHNHYT |
| | | | | QKSLSLSPGK |
| | | | | (SEQ ID |
| | | | | NO: 309) |
| xhCD8v12- | DIQMTQSPSSLS | QVQLVQSGAEVKKP | QVQLVQSGAEV | QVQLVQSGAE |
| IL-2m1 | ASVGDRVTITC | GSSVKVSCKASGGTF | KKPGSSVKVSCK | VKKPGSSVKV |
| | RASQSIYGALN | SSYAISWVRQAPGQG | ASGGTFSSYAIS | SCKASGGTFSS |
| | WYQQKPGKAP | LEWMGGIIPGYATAN | WVRQAPGQGLE | YAISWVRQAP |
| | KLLIYGASNLQ | YAQKFQGRVTITADE | WMGGIIPGYATA | GQGLEWMGGI |
| | SGVPSRFSGSGS | STSTAYMELSSLRSE | NYAQKFQGRVTI | IPGYATANYA |
| | GTDFTLTISSLQ | DTAVYYCARDAAGI | TADESTSTAYME | QKFQGRVTITA |
| | PEDFATYYCQS | RLFADWGQGTLVTV | LSSLRSEDTAVY | DESTSTAYMEL |
| | TYTAPWTFGGG | SSASTKGPSVFPLAPS | YCARDAAGIRLF | SSLRSEDTAVY |
| | TKVEIKRTVAA | SKSTSGGTAALGCLV | ADWGQGTLVTV | YCARDAAGIR |
| | PSVFIFPPSDEQ | KDYFPEPVTVSWNSG | SSASTKGPSVFPL | LFADWGQGTL |
| | LKSGTASVVCL | ALTSGVHTFPAVLQS | APSSKSTSGGTA | VTVSSASTKGP |
| | LNNFYPREAKV | SGLYSLSSVVTVPSSS | ALGCLVKDYFPE | SVFPLAPSSKS |
| | QWKVDNALQS | LGTQTYICNVNHKPS | PVTVSWNSGAL | TSGGTAALGC |
| | GNSQESVTEQD | NTKVDKKVEPKSCD | TSGVHTFPAVLQ | LVKDYFPEPVT |
| | SKDSTYSLSSTL | KTHTCPPCPAPEAAG | SSGLYSLSSVVT | VSWNSGALTS |
| | TLSKADYEKHK | APSVFLFPPKPKDTL | VPSSSLGTQTYIC | GVHTFPAVLQS |
| | VYACEVTHQGL | MISRTPEVTCVVVDV | NVNHKPSNTKV | SGLYSLSSVVT |
| | SSPVTKSFNRGE | SHEDPEVKFNWYVD | DKKVEPKSCDK | VPSSSLGTQTY |
| | C | GVEVHNAKTKPREE | THTCPPCPAPEA | ICNVNHKPSNT |
| | (SEQ ID | QYNSTYRVVSVLTVL | AGAPSVFLFPPK | KVDKKVEPKS |
| | NO: 310) | HQDWLNGKEYKCKV | PKDTLMISRTPE | CDKTHTCPPCP |
| | | SNKALPAPIEKTISKA | VTCVVVDVSHE | APEAAGAPSVF |
| | | KGQPREPQVYTLPPC | DPEVKFNWYVD | LFPPKPKDTLM |
| | | REEMTKNQVSLSCA | GVEVHNAKTKP | ISRTPEVTCVV |
| | | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSAPTSSSTKK | NQVSLWCLVKG | SNKALPAPIEK |
| | | TQLQLEHLLLDLQMI | FYPSDIAVEWES | TISKAKGQPRE |
| | | LNGINNYKNPKLTEM | NGQPENNYKTTP | PQVCTLPPSRE |
| | | LTAKFYMPKKATEL | PVLDSDGSFFLY | EMTKNQVSLW |
| | | KHLQCLEEELKPLEE | SKLTVDKSRWQ | CLVKGFYPSDI |
| | | VLNLAQSKNFHLRPR | QGNVFSCSVMH | AVEWESNGQP |
| | | DLISAINVIVLELKGS | EALHNHYTQKS | ENNYKTTPPVL |
| | | ETTFMCEYADETATI | LSLSPG | DSDGSFFLYSK |
| | | VEFLNRWITFAQSIIS | (SEQ ID | LTVDKSRWQQ |
| | | TLT | NO: 312) | GNVFSCSVMH |
| | | (SEQ ID NO: 311) | | EALHNHYTQK |
| | | | | SLSLSPGK |
| | | | | (SEQ ID |
| | | | | NO: 313) |
| xhCD8v13- | DIQMTQSPSSLS | QVQLVQSGAEVKKP | QVQLVQSGAEV | QVQLVQSGAE |
| IL-2m1 | ASVGDRVTITC | GSSVKVSCKASGGTF | KKPGSSVKVSCK | VKKPGSSVKV |
| | RASQEIYGALN | SSYAISWVRQAPGQG | ASGGTFSSYAIS | SCKASGGTFSS |
| | WYQQKPGKAP | LEWMGGIIPGYATAN | WVRQAPGQGLE | YAISWVRQAP |
| | KLLIYGATNLQ | YAQKFQGRVTITADE | WMGGIIPGYATA | GQGLEWMGGI |
| | SGVPSRFSGSGS | STSTAYMELSSLRSE | NYAQKFQGRVTI | IPGYATANYA |
| | GTDFTLTISSLQ | DTAVYYCARDAAGI | TADESTSTAYME | QKFQGRVTITA |
| | PEDFATYYCQS | RLFADWGQGTLVTV | LSSLRSEDTAVY | DESTSTAYMEL |
| | TYDAPWTFGG | SSASTKGPSVFPLAPS | YCARDAAGIRLF | SSLRSEDTAVY |
| | GTKVEIKRTVA | SKSTSGGTAALGCLV | ADWGQGTLVTV | YCARDAAGIR |
| | APSVFIFPPSDE | KDYFPEPVTVSWNSG | SSASTKGPSVFPL | LFADWGQGTL |
| | QLKSGTASVVC | ALTSGVHTFPAVLQS | APSSKSTSGGTA | VTVSSASTKGP |
| | LLNNFYPREAK | SGLYSLSSVVTVPSSS | ALGCLVKDYFPE | SVFPLAPSSKS |
| | VQWKVDNALQ | LGTQTYICNVNHKPS | PVTVSWNSGAL | TSGGTAALGC |
| | SGNSQESVTEQ | NTKVDKKVEPKSCD | TSGVHTFPAVLQ | LVKDYFPEPVT |
| | DSKDSTYSLSST | KTHTCPPCPAPEAAG | SSGLYSLSSVVT | VSWNSGALTS |
| | LTLSKADYEKH | APSVFLFPPKPKDTL | VPSSSLGTQTYIC | GVHTFPAVLQS |
| | KVYACEVTHQ | MISRTPEVTCVVVDV | NVNHKPSNTKV | SGLYSLSSVVT |
| | GLSSPVTKSFNR | SHEDPEVKFNWYVD | DKKVEPKSCDK | VPSSSLGTQTY |
| | GEC | GVEVHNAKTKPREE | THTCPPCPAPEA | ICNVNHKPSNT |
| | (SEQ ID NO: | QYNSTYRVVSVLTVL | AGAPSVFLFPPK | KVDKKVEPKS |
| | 314) | HQDWLNGKEYKCKV | PKDTLMISRTPE | CDKTHTCPPCP |
| | | SNKALPAPIEKTISKA | VTCVVVDVSHE | APEAAGAPSVF |
| | | KGQPREPQVYTLPPC | DPEVKFNWYVD | LFPPKPKDTLM |
| | | REEMTKNQVSLSCA | GVEVHNAKTKP | ISRTPEVTCVV |
| | | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSAPTSSSTKK | NQVSLWCLVKG | SNKALPAPIEK |
| | | TQLQLEHLLLDLQMI | FYPSDIAVEWES | TISKAKGQPRE |
| | | LNGINNYKNPKLTEM | NGQPENNYKTTP | PQVCTLPPSRE |
| | | LTAKFYMPKKATEL | PVLDSDGSFFLY | EMTKNQVSLW |
| | | KHLQCLEEELKPLEE | SKLTVDKSRWQ | CLVKGFYPSDI |
| | | VLNLAQSKNFHLRPR | QGNVFSCSVMH | AVEWESNGQP |
| | | DLISAINVIVLELKGS | EALHNHYTQKS | ENNYKTTPPVL |
| | | ETTFMCEYADETATI | LSLSPG | DSDGSFFLYSK |
| | | VEFLNRWITFAQSIIS | (SEQ ID | LTVDKSRWQQ |
| | | TLT | NO: 316) | GNVFSCSVMH |
| | | (SEQ ID NO: 315) | | EALHNHYTQK |
| | | | | SLSLSPGK |
| | | | | (SEQ ID |
| | | | | NO: 317) |
| xhCD8v14- | EIVLTQSPGTLS | EVQLVESGGGLVQPG | EVQLVESGGGL | EVQLVESGGG |
| IL-2m1 | LSPGERATLSCR | GSLRLSCAASGFTFSS | VQPGGSLRLSCA | LVQPGGSLRLS |
| | ASQSVSSNLAW | YAMSWVRQAPGKGL | ASGFTFSSYAMS | CAASGFTFSSY |
| | YQQKPGQAPRL | EWVSDISYAGGSTAY | WVRQAPGKGLE | AMSWVRQAPG |
| | LIYGASSRATGI | ADSVKGRFTISRDNA | WVSDISYAGGST | KGLEWVSDISY |
| | PDRFSGSGSGT | KNSLYLQMNSLRAE | AYADSVKGRFTI | AGGSTAYADS |
| | DFTLTISRLEPE | DTAVYYCARSNAYA | SRDNAKNSLYL | VKGRFTISRDN |
| | DFAVYYCQQY | WDDALDIWGQGTM | QMNSLRAEDTA | AKNSLYLQMN |
| | GSSPPVTFGQG | VTVSSASTKGPSVFPL | VYYCARSNAYA | SLRAEDTAVY |
| | TKVEIKRTVAA | APSSKSTSGGTAALG | WDDALDIWGQG | YCARSNAYAW |
| | PSVFIFPPSDEQ | CLVKDYFPEPVTVSW | TMVTVSSASTKG | DDALDIWGQG |
| | LKSGTASVVCL | NSGALTSGVHTFPAV | PSVFPLAPSSKST | TMVTVSSAST |
| | LNNFYPREAKV | LQSSGLYSLSSVVTV | SGGTAALGCLV | KGPSVFPLAPS |
| | QWKVDNALQS | PSSSLGTQTYICNVN | KDYFPEPVTVSW | SKSTSGGTAAL |
| | GNSQESVTEQD | HKPSNTKVDKKVEP | NSGALTSGVHTF | GCLVKDYFPEP |
| | SKDSTYSLSSTL | KSCDKTHTCPPCPAP | PAVLQSSGLYSL | VTVSWNSGAL |
| | TLSKADYEKHK | EAAGAPSVFLFPPKP | SSVVTVPSSSLG | TSGVHTFPAVL |
| | VYACEVTHQGL | KDTLMISRTPEVTCV | TQTYICNVNHKP | QSSGLYSLSSV |
| | SSPVTKSFNRGE | VVDVSHEDPEVKFN | SNTKVDKKVEP | VTVPSSSLGTQ |
| | C | WYVDGVEVHNAKT | KSCDKTHTCPPC | TYICNVNHKPS |
| | (SEQ ID | KPREEQYNSTYRVVS | PAPEAAGAPSVF | NTKVDKKVEP |
| | NO: 318) | VLTVLHQDWLNGKE | LFPPKPKDTLMIS | KSCDKTHTCPP |
| | | YKCKVSNKALPAPIE | RTPEVTCVVVD | CPAPEAAGAPS |
| | | KTISKAKGQPREPQV | VSHEDPEVKFN | VFLFPPKPKDT |
| | | YTLPPCREEMTKNQV | WYVDGVEVHN | LMISRTPEVTC |
| | | SLSCAVKGFYPSDIA | AKTKPREEQYNS | VVVDVSHEDP |
| | | VEWESNGQPENNYK | TYRVVSVLTVLH | EVKFNWYVDG |
| | | TTPPVLDSDGSFFLVS | QDWLNGKEYKC | VEVHNAKTKP |
| | | KLTVDKSRWQQGNV | KVSNKALPAPIE | REEQYNSTYR |
| | | FSCSVMHEALHNHY | KTISKAKGQPRE | VVSVLTVLHQ |
| | | TQKSLSLSPGSGGGG | PQVCTLPPSREE | DWLNGKEYKC |
| | | SGGGGSGGGGSAPTS | MTKNQVSLWCL | KVSNKALPAPI |
| | | SSTKKTQLQLEHLLL | VKGFYPSDIAVE | EKTISKAKGQP |
| | | DLQMILNGINNYKNP | WESNGQPENNY | REPQVCTLPPS |
| | | KLTEMLTAKFYMPK | KTTPPVLDSDGS | REEMTKNQVS |
| | | KATELKHLQCLEEEL | FFLYSKLTVDKS | LWCLVKGFYP |
| | | KPLEEVLNLAQSKNF | RWQQGNVFSCS | SDIAVEWESNG |
| | | HLRPRDLISAINVIVL | VMHEALHNHYT | QPENNYKTTPP |
| | | ELKGSETTFMCEYAD | QKSLSLSPG | VLDSDGSFFLY |
| | | ETATIVEFLNRWITFA | (SEQ ID | SKLTVDKSRW |
| | | QSIISTLT | NO: 320) | QQGNVFSCSV |
| | | (SEQ ID NO: 319) | | MHEALHNHYT |
| | | | | QKSLSLSPGK |
| | | | | (SEQ ID |
| | | | | NO: 321) |
| xhCD8v15- | EIVLTQSPGTLS | EVQLVESGGGLVQPG | EVQLVESGGGL | EVQLVESGGG |
| IL-2m1 | LSPGERATLSCR | GSLRLSCAASGFTFSS | VQPGGSLRLSCA | LVQPGGSLRLS |
| | ASQSVSSNLAW | YAMSWVRQAPGKGL | ASGFTFSSYAMS | CAASGFTFSSY |
| | YQQKPGQAPRL | EWVSDISYAGGSTAY | WVRQAPGKGLE | AMSWVRQAPG |
| | LIYGASSRATGI | ADSVKGRFTISRDNA | WVSDISYAGGST | KGLEWVSDISY |
| | PDRFSGSGSGT | KNSLYLQMNSLRAE | AYADSVKGRFTI | AGGSTAYADS |
| | DFTLTISRLEPE | DTAVYYCARSNAYA | SRDNAKNSLYL | VKGRFTISRDN |
| | DFAVYYCQQY | WDDALDIWGQGTLV | QMNSLRAEDTA | AKNSLYLQMN |
| | GSSPPVTFGQG | TVSSASTKGPSVFPLA | VYYCARSNAYA | SLRAEDTAVY |
| | TKVEIKRTVAA | PSSKSTSGGTAALGC | WDDALDIWGQG | YCARSNAYAW |
| | PSVFIFPPSDEQ | LVKDYFPEPVTVSWN | TLVTVSSASTKG | DDALDIWGQG |
| | LKSGTASVVCL | SGALTSGVHTFPAVL | PSVFPLAPSSKST | TLVTVSSASTK |
| | LNNFYPREAKV | QSSGLYSLSSVVTVPS | SGGTAALGCLV | GPSVFPLAPSS |
| | QWKVDNALQS | SSLGTQTYICNVNHK | KDYFPEPVTVSW | KSTSGGTAAL |
| | GNSQESVTEQD | PSNTKVDKKVEPKSC | NSGALTSGVHTF | GCLVKDYFPEP |
| | SKDSTYSLSSTL | DKTHTCPPCPAPEAA | PAVLQSSGLYSL | VTVSWNSGAL |
| | TLSKADYEKHK | GAPSVFLFPPKPKDTL | SSVVTVPSSSLG | TSGVHTFPAVL |
| | VYACEVTHQGL | MISRTPEVTCVVVDV | TQTYICNVNHKP | QSSGLYSLSSV |
| | SSPVTKSFNRGE | SHEDPEVKFNWYVD | SNTKVDKKVEP | VTVPSSSLGTQ |
| | C | GVEVHNAKTKPREE | KSCDKTHTCPPC | TYICNVNHKPS |
| | (SEQ ID | QYNSTYRVVSVLTVL | PAPEAAGAPSVF | NTKVDKKVEP |
| | NO: 322) | HQDWLNGKEYKCKV | LFPPKPKDTLMIS | KSCDKTHTCPP |
| | | SNKALPAPIEKTISKA | RTPEVTCVVVD | CPAPEAAGAPS |
| | | KGQPREPQVYTLPPC | VSHEDPEVKFN | VFLFPPKPKDT |
| | | REEMTKNQVSLSCA | WYVDGVEVHN | LMISRTPEVTC |
| | | VKGFYPSDIAVEWES | AKTKPREEQYNS | VVVDVSHEDP |
| | | NGQPENNYKTTPPVL | TYRVVSVLTVLH | EVKFNWYVDG |
| | | DSDGSFFLVSKLTVD | QDWLNGKEYKC | VEVHNAKTKP |
| | | KSRWQQGNVFSCSV | KVSNKALPAPIE | REEQYNSTYR |
| | | MHEALHNHYTQKSL | KTISKAKGQPRE | VVSVLTVLHQ |
| | | SLSPGSGGGGSGGGG | PQVCTLPPSREE | DWLNGKEYKC |
| | | SGGGGSAPTSSSTKK | MTKNQVSLWCL | KVSNKALPAPI |
| | | TQLQLEHLLLDLQMI | VKGFYPSDIAVE | EKTISKAKGQP |
| | | LNGINNYKNPKLTEM | WESNGQPENNY | REPQVCTLPPS |
| | | LTAKFYMPKKATEL | KTTPPVLDSDGS | REEMTKNQVS |
| | | KHLQCLEEELKPLEE | FFLYSKLTVDKS | LWCLVKGFYP |
| | | VLNLAQSKNFHLRPR | RWQQGNVFSCS | SDIAVEWESNG |
| | | DLISAINVIVLELKGS | VMHEALHNHYT | QPENNYKTTPP |
| | | ETTFMCEYADETATI | QKSLSLSPG | VLDSDGSFFLY |
| | | VEFLNRWITFAQSIIS | (SEQ ID | SKLTVDKSRW |
| | | TLT | NO: 324) | QQGNVFSCSV |
| | | (SEQ ID NO: 323) | | MHEALHNHYT |
| | | | | QKSLSLSPGK |
| | | | | (SEQ ID |
| | | | | NO: 325) |
| xhCD8v9- | DIQMTQSPSSLS | QVQLVQSGAEVKKP | QVQLVQSGAEV | QVQLVQSGAE |
| IL-2m4 | ASVGDRVTITC | GSSVKVSCKASGGTF | KKPGSSVKVSCK | VKKPGSSVKV |
| | RASQEIYGALN | SSYAISWVRQAPGQG | ASGGTFSSYAIS | SCKASGGTFSS |
| | WYQQKPGKAP | LEWMGGIIPGAATAN | WVRQAPGQGLE | YAISWVRQAP |
| | KLLIYGATNLQ | YAQKFQGRVTITADE | WMGGIIPGAATA | GQGLEWMGGI |
| | SGVPSRFSGSGS | STSTAYMELSSLRSE | NYAQKFQGRVTI | IPGAATANYA |
| | GTDFTLTISSLQ | DTAVYYCARDAAGI | TADESTSTAYME | QKFQGRVTITA |
| | PEDFATYYCQS | RLFADWGQGTLVTV | LSSLRSEDTAVY | DESTSTAYMEL |
| | TYDAPWTFGG | SSASTKGPSVFPLAPS | YCARDAAGIRLF | SSLRSEDTAVY |
| | GTKVEIKRTVA | SKSTSGGTAALGCLV | ADWGQGTLVTV | YCARDAAGIR |
| | APSVFIFPPSDE | KDYFPEPVTVSWNSG | SSASTKGPSVFPL | LFADWGQGTL |
| | QLKSGTASVVC | ALTSGVHTFPAVLQS | APSSKSTSGGTA | VTVSSASTKGP |
| | LLNNFYPREAK | SGLYSLSSVVTVPSSS | ALGCLVKDYFPE | SVFPLAPSSKS |
| | VQWKVDNALQ | LGTQTYICNVNHKPS | PVTVSWNSGAL | TSGGTAALGC |
| | SGNSQESVTEQ | NTKVDKKVEPKSCD | TSGVHTFPAVLQ | LVKDYFPEPVT |
| | DSKDSTYSLSST | KTHTCPPCPAPEAAG | SSGLYSLSSVVT | VSWNSGALTS |
| | LTLSKADYEKH | APSVFLFPPKPKDTL | VPSSSLGTQTYIC | GVHTFPAVLQS |
| | KVYACEVTHQ | MISRTPEVTCVVVDV | NVNHKPSNTKV | SGLYSLSSVVT |
| | GLSSPVTKSFNR | SHEDPEVKFNWYVD | DKKVEPKSCDK | VPSSSLGTQTY |
| | GEC | GVEVHNAKTKPREE | THTCPPCPAPEA | ICNVNHKPSNT |
| | (SEQ ID | QYNSTYRVVSVLTVL | AGAPSVFLFPPK | KVDKKVEPKS |
| | NO: 326) | HQDWLNGKEYKCKV | PKDTLMISRTPE | CDKTHTCPPCP |
| | | SNKALPAPIEKTISKA | VTCVVVDVSHE | APEAAGAPSVF |
| | | KGQPREPQVYTLPPC | DPEVKFNWYVD | LFPPKPKDTLM |
| | | REEMTKNQVSLSCA | GVEVHNAKTKP | ISRTPEVTCVV |
| | | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVSN | VHNAKTKPRE |
| | | KSRWQQGNVFSCSV | KALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSAPTSSSTKK | NQVSLWCLVKG | SNKALPAPIEK |
| | | TQLQLEELLLDLQMI | FYPSDIAVEWES | TISKAKGQPRE |
| | | LNGINNYKNPKLTEM | NGQPENNYKTTP | PQVCTLPPSRE |
| | | LTAKFYMPKKATEL | PVLDSDGSFFLY | EMTKNQVSLW |
| | | KHLQCLEEELKPLEE | SKLTVDKSRWQ | CLVKGFYPSDI |
| | | VLNLAQSKNFHLRPR | QGNVFSCSVMH | AVEWESNGQP |
| | | DLISNINVIVLELKGS | EALHNHYTQKS | ENNYKTTPPVL |
| | | ETTFMCEYADETATI | LSLSPG | DSDGSFFLYSK |
| | | VEFLNRWITFAQSIIS | (SEQ ID | LTVDKSRWQQ |
| | | TLT | NO: 328) | GNVFSCSVMH |
| | | (SEQ ID NO: 327) | | EALHNHYTQK |
| | | | | SLSLSPGK |
| | | | | (SEQ ID |
| | | | | NO: 329) |
| xhCD8v10- | EIVLTQSPGTLS | EVQLVESGGGLVQPG | EVQLVESGGGL | EVQLVESGGG |
| IL-2m4 | LSPGERATLSCR | GSLRLSCAASGFTFSS | VQPGGSLRLSCA | LVQPGGSLRLS |
| | ASQSVSSNLAW | YAMSWVRQAPGKGL | ASGFTFSSYAMS | CAASGFTFSSY |
| | YQQKPGQAPRL | EWVSDITYAGGSTAY | WVRQAPGKGLE | AMSWVRQAPG |
| | LIYGASSRATGI | ADSVKGRFTISRDNA | WVSDITYAGGST | KGLEWVSDIT |
| | PDRFSGSGSGT | KNSLYLQMNSLRAE | AYADSVKGRFTI | YAGGSTAYAD |
| | DFTLTISRLEPE | DTAVYYCARSNAYA | SRDNAKNSLYL | SVKGRFTISRD |
| | DFAVYYCQQY | WDDALDIWGQGTM | QMNSLRAEDTA | NAKNSLYLQM |
| | GSSPPVTFGQG | VTVSSASTKGPSVFPL | VYYCARSNAYA | NSLRAEDTAV |
| | TKVEIKRTVAA | APSSKSTSGGTAALG | WDDALDIWGQG | YYCARSNAYA |
| | PSVFIFPPSDEQ | CLVKDYFPEPVTVSW | TMVTVSSASTKG | WDDALDIWGQ |
| | LKSGTASVVCL | NSGALTSGVHTFPAV | PSVFPLAPSSKST | GTMVTVSSAS |
| | LNNFYPREAKV | LQSSGLYSLSSVVTV | SGGTAALGCLV | TKGPSVFPLAP |
| | QWKVDNALQS | PSSSLGTQTYICNVN | KDYFPEPVTVSW | SSKSTSGGTAA |
| | GNSQESVTEQD | HKPSNTKVDKKVEP | NSGALTSGVHTF | LGCLVKDYFPE |
| | SKDSTYSLSSTL | KSCDKTHTCPPCPAP | PAVLQSSGLYSL | PVTVSWNSGA |
| | TLSKADYEKHK | EAAGAPSVFLFPPKP | SSVVTVPSSSLG | LTSGVHTFPAV |
| | VYACEVTHQGL | KDTLMISRTPEVTCV | TQTYICNVNHKP | LOSSGLYSLSS |
| | SSPVTKSFNRGE | VVDVSHEDPEVKFN | SNTKVDKKVEP | VVTVPSSSLGT |
| | C | WYVDGVEVHNAKT | KSCDKTHTCPPC | QTYICNVNHKP |
| | (SEQ ID | KPREEQYNSTYRVVS | PAPEAAGAPSVF | SNTKVDKKVE |
| | NO: 330) | VLTVLHQDWLNGKE | LFPPKPKDTLMIS | PKSCDKTHTCP |
| | | YKCKVSNKALPAPIE | RTPEVTCVVVD | PCPAPEAAGAP |
| | | KTISKAKGQPREPQV | VSHEDPEVKFN | SVFLFPPKPKD |
| | | YTLPPCREEMTKNQV | WYVDGVEVHN | TLMISRTPEVT |
| | | SLSCAVKGFYPSDIA | AKTKPREEQYNS | CVVVDVSHED |
| | | VEWESNGQPENNYK | TYRVVSVLTVLH | PEVKFNWYVD |
| | | TTPPVLDSDGSFFLVS | QDWLNGKEYKC | GVEVHNAKTK |
| | | KLTVDKSRWQQGNV | KVSNKALPAPIE | PREEQYNSTYR |
| | | FSCSVMHEALHNHY | KTISKAKGQPRE | VVSVLTVLHQ |
| | | TQKSLSLSPGSGGGG | PQVCTLPPSREE | DWLNGKEYKC |
| | | SGGGGSGGGGSAPTS | MTKNQVSLWCL | KVSNKALPAPI |
| | | SSTKKTQLQLEELLL | VKGFYPSDIAVE | EKTISKAKGQP |
| | | DLQMILNGINNYKNP | WESNGQPENNY | REPQVCTLPPS |
| | | KLTEMLTAKFYMPK | KTTPPVLDSDGS | REEMTKNQVS |
| | | KATELKHLQCLEEEL | FFLYSKLTVDKS | LWCLVKGFYP |
| | | KPLEEVLNLAQSKNF | RWQQGNVFSCS | SDIAVEWESNG |
| | | HLRPRDLISNINVIVL | VMHEALHNHYT | QPENNYKTTPP |
| | | ELKGSETTFMCEYAD | QKSLSLSPG | VLDSDGSFFLY |
| | | ETATIVEFLNRWITFA | (SEQ ID | SKLTVDKSRW |
| | | QSIISTLT | NO: 332) | QQGNVFSCSV |
| | | (SEQ ID NO: 331) | | MHEALHNHYT |
| | | | | QKSLSLSPGK |
| | | | | (SEQ ID |
| | | | | NO: 333) |
| xhCD8v11- | EIVLTQSPGTLS | EVQLVESGGGLVQPG | EVQLVESGGGL | EVQLVESGGG |
| IL-2m4 | LSPGERATLSCR | GSLRLSCAASGFTFSS | VQPGGSLRLSCA | LVQPGGSLRLS |
| | ASQSVSSNLAW | YAMSWVRQAPGKGL | ASGFTFSSYAMS | CAASGFTFSSY |
| | YQQKPGQAPRL | EWVSDITYAGGSTAY | WVRQAPGKGLE | AMSWVRQAPG |
| | LIYGASSRATGI | ADSVKGRFTISRDNA | WVSDITYAGGST | KGLEWVSDIT |
| | PDRFSGSGSGT | KNSLYLQMNSLRAE | AYADSVKGRFTI | YAGGSTAYAD |
| | DFTLTISRLEPE | DTAVYYCARSNAYA | SRDNAKNSLYL | SVKGRFTISRD |
| | DFAVYYCQQY | WDDALDIWGQGTLV | QMNSLRAEDTA | NAKNSLYLQM |
| | GSSPPVTFGQG | TVSSASTKGPSVFPLA | VYYCARSNAYA | NSLRAEDTAV |
| | TKVEIKRTVAA | PSSKSTSGGTAALGC | WDDALDIWGQG | YYCARSNAYA |
| | PSVFIFPPSDEQ | LVKDYFPEPVTVSWN | TLVTVSSASTKG | WDDALDIWGQ |
| | LKSGTASVVCL | SGALTSGVHTFPAVL | PSVFPLAPSSKST | GTLVTVSSAST |
| | LNNFYPREAKV | QSSGLYSLSSVVTVPS | SGGTAALGCLV | KGPSVFPLAPS |
| | QWKVDNALQS | SSLGTQTYICNVNHK | KDYFPEPVTVSW | SKSTSGGTAAL |
| | GNSQESVTEQD | PSNTKVDKKVEPKSC | NSGALTSGVHTF | GCLVKDYFPEP |
| | SKDSTYSLSSTL | DKTHTCPPCPAPEAA | PAVLQSSGLYSL | VTVSWNSGAL |
| | TLSKADYEKHK | GAPSVFLFPPKPKDTL | SSVVTVPSSSLG | TSGVHTFPAVL |
| | VYACEVTHQGL | MISRTPEVTCVVVDV | TQTYICNVNHKP | QSSGLYSLSSV |
| | SSPVTKSFNRGE | SHEDPEVKFNWYVD | SNTKVDKKVEP | VTVPSSSLGTQ |
| | C | GVEVHNAKTKPREE | KSCDKTHTCPPC | TYICNVNHKPS |
| | (SEQ ID | QYNSTYRVVSVLTVL | PAPEAAGAPSVF | NTKVDKKVEP |
| | NO: 334) | HQDWLNGKEYKCKV | LFPPKPKDTLMIS | KSCDKTHTCPP |
| | | SNKALPAPIEKTISKA | RTPEVTCVVVD | CPAPEAAGAPS |
| | | KGQPREPQVYTLPPC | VSHEDPEVKFN | VFLFPPKPKDT |
| | | REEMTKNQVSLSCA | WYVDGVEVHN | LMISRTPEVTC |
| | | VKGFYPSDIAVEWES | AKTKPREEQYNS | VVVDVSHEDP |
| | | NGQPENNYKTTPPVL | TYRVVSVLTVLH | EVKFNWYVDG |
| | | DSDGSFFLVSKLTVD | QDWLNGKEYKC | VEVHNAKTKP |
| | | KSRWQQGNVFSCSV | KVSNKALPAPIE | REEQYNSTYR |
| | | MHEALHNHYTQKSL | KTISKAKGQPRE | VVSVLTVLHQ |
| | | SLSPGSGGGGSGGGG | PQVCTLPPSREE | DWLNGKEYKC |
| | | SGGGGSAPTSSSTKK | MTKNQVSLWCL | KVSNKALPAPI |
| | | TQLQLEELLLDLQMI | VKGFYPSDIAVE | EKTISKAKGQP |
| | | LNGINNYKNPKLTEM | WESNGQPENNY | REPQVCTLPPS |
| | | LTAKFYMPKKATEL | KTTPPVLDSDGS | REEMTKNQVS |
| | | KHLQCLEEELKPLEE | FFLYSKLTVDKS | LWCLVKGFYP |
| | | VLNLAQSKNFHLRPR | RWQQGNVFSCS | SDIAVEWESNG |
| | | DLISNINVIVLELKGS | VMHEALHNHYT | QPENNYKTTPP |
| | | ETTFMCEYADETATI | QKSLSLSPG | VLDSDGSFFLY |
| | | VEFLNRWITFAQSIIS | (SEQ ID | SKLTVDKSRW |
| | | TLT | NO: 336) | QQGNVFSCSV |
| | | (SEQ ID NO: 335) | | MHEALHNHYT |
| | | | | QKSLSLSPGK |
| | | | | (SEQ ID |
| | | | | NO: 337) |
| xhCD8v12- | DIQMTQSPSSLS | QVQLVQSGAEVKKP | QVQLVQSGAEV | QVQLVQSGAE |
| IL-2m4 | ASVGDRVTITC | GSSVKVSCKASGGTF | KKPGSSVKVSCK | VKKPGSSVKV |
| | RASQSIYGALN | SSYAISWVRQAPGQG | ASGGTFSSYAIS | SCKASGGTFSS |
| | WYQQKPGKAP | LEWMGGIIPGYATAN | WVRQAPGQGLE | YAISWVRQAP |
| | KLLIYGASNLQ | YAQKFQGRVTITADE | WMGGIIPGYATA | GQGLEWMGGI |
| | SGVPSRFSGSGS | STSTAYMELSSLRSE | NYAQKFQGRVTI | IPGYATANYA |
| | GTDFTLTISSLQ | DTAVYYCARDAAGI | TADESTSTAYME | QKFQGRVTITA |
| | PEDFATYYCQS | RLFADWGQGTLVTV | LSSLRSEDTAVY | DESTSTAYMEL |
| | TYTAPWTFGGG | SSASTKGPSVFPLAPS | YCARDAAGIRLF | SSLRSEDTAVY |
| | TKVEIKRTVAA | SKSTSGGTAALGCLV | ADWGQGTLVTV | YCARDAAGIR |
| | PSVFIFPPSDEQ | KDYFPEPVTVSWNSG | SSASTKGPSVFPL | LFADWGQGTL |
| | LKSGTASVVCL | ALTSGVHTFPAVLQS | APSSKSTSGGTA | VTVSSASTKGP |
| | LNNFYPREAKV | SGLYSLSSVVTVPSSS | ALGCLVKDYFPE | SVFPLAPSSKS |
| | QWKVDNALQS | LGTQTYICNVNHKPS | PVTVSWNSGAL | TSGGTAALGC |
| | GNSQESVTEQD | NTKVDKKVEPKSCD | TSGVHTFPAVLQ | LVKDYFPEPVT |
| | SKDSTYSLSSTL | KTHTCPPCPAPEAAG | SSGLYSLSSVVT | VSWNSGALTS |
| | TLSKADYEKHK | APSVFLFPPKPKDTL | VPSSSLGTQTYIC | GVHTFPAVLQS |
| | VYACEVTHQGL | MISRTPEVTCVVVDV | NVNHKPSNTKV | SGLYSLSSVVT |
| | SSPVTKSFNRGE | SHEDPEVKFNWYVD | DKKVEPKSCDK | VPSSSLGTQTY |
| | C | GVEVHNAKTKPREE | THTCPPCPAPEA | ICNVNHKPSNT |
| | (SEQ ID | QYNSTYRVVSVLTVL | AGAPSVFLFPPK | KVDKKVEPKS |
| | NO: 338) | HQDWLNGKEYKCKV | PKDTLMISRTPE | CDKTHTCPPCP |
| | | SNKALPAPIEKTISKA | VTCVVVDVSHE | APEAAGAPSVF |
| | | KGQPREPQVYTLPPC | DPEVKFNWYVD | LFPPKPKDTLM |
| | | REEMTKNQVSLSCA | GVEVHNAKTKP | ISRTPEVTCVV |
| | | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSAPTSSSTKK | NQVSLWCLVKG | SNKALPAPIEK |
| | | TQLQLEELLLDLQMI | FYPSDIAVEWES | TISKAKGQPRE |
| | | LNGINNYKNPKLTEM | NGQPENNYKTTP | PQVCTLPPSRE |
| | | LTAKFYMPKKATEL | PVLDSDGSFFLY | EMTKNQVSLW |
| | | KHLQCLEEELKPLEE | SKLTVDKSRWQ | CLVKGFYPSDI |
| | | VLNLAQSKNFHLRPR | QGNVFSCSVMH | AVEWESNGQP |
| | | DLISNINVIVLELKGS | EALHNHYTQKS | ENNYKTTPPVL |
| | | ETTFMCEYADETATI | LSLSPG | DSDGSFFLYSK |
| | | VEFLNRWITFAQSIIS | (SEQ ID | LTVDKSRWQQ |
| | | TLT | NO: 340) | GNVFSCSVMH |
| | | (SEQ ID NO: 339) | | EALHNHYTQK |
| | | | | SLSLSPGK |
| | | | | (SEQ ID |
| | | | | NO: 341) |
| xhCD8v13- | DIQMTQSPSSLS | QVQLVQSGAEVKKP | QVQLVQSGAEV | QVQLVQSGAE |
| IL-2m4 | ASVGDRVTITC | GSSVKVSCKASGGTF | KKPGSSVKVSCK | VKKPGSSVKV |
| | RASQEIYGALN | SSYAISWVRQAPGQG | ASGGTFSSYAIS | SCKASGGTFSS |
| | WYQQKPGKAP | LEWMGGIIPGYATAN | WVRQAPGQGLE | YAISWVRQAP |
| | KLLIYGATNLQ | YAQKFQGRVTITADE | WMGGIIPGYATA | GQGLEWMGGI |
| | SGVPSRFSGSGS | STSTAYMELSSLRSE | NYAQKFQGRVTI | IPGYATANYA |
| | GTDFTLTISSLQ | DTAVYYCARDAAGI | TADESTSTAYME | QKFQGRVTITA |
| | PEDFATYYCQS | RLFADWGQGTLVTV | LSSLRSEDTAVY | DESTSTAYMEL |
| | TYDAPWTFGG | SSASTKGPSVFPLAPS | YCARDAAGIRLF | SSLRSEDTAVY |
| | GTKVEIKRTVA | SKSTSGGTAALGCLV | ADWGQGTLVTV | YCARDAAGIR |
| | APSVFIFPPSDE | KDYFPEPVTVSWNSG | SSASTKGPSVFPL | LFADWGQGTL |
| | QLKSGTASVVC | ALTSGVHTFPAVLQS | APSSKSTSGGTA | VTVSSASTKGP |
| | LLNNFYPREAK | SGLYSLSSVVTVPSSS | ALGCLVKDYFPE | SVFPLAPSSKS |
| | VQWKVDNALQ | LGTQTYICNVNHKPS | PVTVSWNSGAL | TSGGTAALGC |
| | SGNSQESVTEQ | NTKVDKKVEPKSCD | TSGVHTFPAVLQ | LVKDYFPEPVT |
| | DSKDSTYSLSST | KTHTCPPCPAPEAAG | SSGLYSLSSVVT | VSWNSGALTS |
| | LTLSKADYEKH | APSVFLFPPKPKDTL | VPSSSLGTQTYIC | GVHTFPAVLQS |
| | KVYACEVTHQ | MISRTPEVTCVVVDV | NVNHKPSNTKV | SGLYSLSSVVT |
| | GLSSPVTKSFNR | SHEDPEVKFNWYVD | DKKVEPKSCDK | VPSSSLGTQTY |
| | GEC | GVEVHNAKTKPREE | THTCPPCPAPEA | ICNVNHKPSNT |
| | (SEQ ID | QYNSTYRVVSVLTVL | AGAPSVFLFPPK | KVDKKVEPKS |
| | NO: 342) | HQDWLNGKEYKCKV | PKDTLMISRTPE | CDKTHTCPPCP |
| | | SNKALPAPIEKTISKA | VTCVVVDVSHE | APEAAGAPSVF |
| | | KGQPREPQVYTLPPC | DPEVKFNWYVD | LFPPKPKDTLM |
| | | REEMTKNQVSLSCA | GVEVHNAKTKP | ISRTPEVTCVV |
| | | VKGFYPSDIAVEWES | REEQYNSTYRV | VDVSHEDPEV |
| | | NGQPENNYKTTPPVL | VSVLTVLHQDW | KFNWYVDGVE |
| | | DSDGSFFLVSKLTVD | LNGKEYKCKVS | VHNAKTKPRE |
| | | KSRWQQGNVFSCSV | NKALPAPIEKTIS | EQYNSTYRVV |
| | | MHEALHNHYTQKSL | KAKGQPREPQV | SVLTVLHQDW |
| | | SLSPGSGGGGSGGGG | CTLPPSREEMTK | LNGKEYKCKV |
| | | SGGGGSAPTSSSTKK | NQVSLWCLVKG | SNKALPAPIEK |
| | | TQLQLEELLLDLQMI | FYPSDIAVEWES | TISKAKGQPRE |
| | | LNGINNYKNPKLTEM | NGQPENNYKTTP | PQVCTLPPSRE |
| | | LTAKFYMPKKATEL | PVLDSDGSFFLY | EMTKNQVSLW |
| | | KHLQCLEEELKPLEE | SKLTVDKSRWQ | CLVKGFYPSDI |
| | | VLNLAQSKNFHLRPR | QGNVFSCSVMH | AVEWESNGQP |
| | | DLISNINVIVLELKGS | EALHNHYTQKS | ENNYKTTPPVL |
| | | ETTFMCEYADETATI | LSLSPG | DSDGSFFLYSK |
| | | VEFLNRWITFAQSIIS | (SEQ ID | LTVDKSRWQQ |
| | | TLT | NO: 344) | GNVFSCSVMH |
| | | (SEQ ID NO: 343) | | EALHNHYTQK |
| | | | | SLSLSPGK |
| | | | | (SEQ ID |
| | | | | NO: 345) |
| xhCD8v14- | EIVLTQSPGTLS | EVQLVESGGGLVQPG | EVQLVESGGGL | EVQLVESGGG |
| IL-2m4 | LSPGERATLSCR | GSLRLSCAASGFTFSS | VQPGGSLRLSCA | LVQPGGSLRLS |
| | ASQSVSSNLAW | YAMSWVRQAPGKGL | ASGFTFSSYAMS | CAASGFTFSSY |
| | YQQKPGQAPRL | EWVSDISYAGGSTAY | WVRQAPGKGLE | AMSWVRQAPG |
| | LIYGASSRATGI | ADSVKGRFTISRDNA | WVSDISYAGGST | KGLEWVSDISY |
| | PDRFSGSGSGT | KNSLYLQMNSLRAE | AYADSVKGRFTI | AGGSTAYADS |
| | DFTLTISRLEPE | DTAVYYCARSNAYA | SRDNAKNSLYL | VKGRFTISRDN |
| | DFAVYYCQQY | WDDALDIWGQGTM | QMNSLRAEDTA | AKNSLYLQMN |
| | GSSPPVTFGQG | VTVSSASTKGPSVFPL | VYYCARSNAYA | SLRAEDTAVY |
| | TKVEIKRTVAA | APSSKSTSGGTAALG | WDDALDIWGQG | YCARSNAYAW |
| | PSVFIFPPSDEQ | CLVKDYFPEPVTVSW | TMVTVSSASTKG | DDALDIWGQG |
| | LKSGTASVVCL | NSGALTSGVHTFPAV | PSVFPLAPSSKST | TMVTVSSAST |
| | LNNFYPREAKV | LQSSGLYSLSSVVTV | SGGTAALGCLV | KGPSVFPLAPS |
| | QWKVDNALQS | PSSSLGTQTYICNVN | KDYFPEPVTVSW | SKSTSGGTAAL |
| | GNSQESVTEQD | HKPSNTKVDKKVEP | NSGALTSGVHTF | GCLVKDYFPEP |
| | SKDSTYSLSSTL | KSCDKTHTCPPCPAP | PAVLQSSGLYSL | VTVSWNSGAL |
| | TLSKADYEKHK | EAAGAPSVFLFPPKP | SSVVTVPSSSLG | TSGVHTFPAVL |
| | VYACEVTHQGL | KDTLMISRTPEVTCV | TQTYICNVNHKP | QSSGLYSLSSV |
| | SSPVTKSFNRGE | VVDVSHEDPEVKFN | SNTKVDKKVEP | VTVPSSSLGTQ |
| | C | WYVDGVEVHNAKT | KSCDKTHTCPPC | TYICNVNHKPS |
| | (SEQ ID | KPREEQYNSTYRVVS | PAPEAAGAPSVF | NTKVDKKVEP |
| | NO: 346) | VLTVLHQDWLNGKE | LFPPKPKDTLMIS | KSCDKTHTCPP |
| | | YKCKVSNKALPAPIE | RTPEVTCVVVD | CPAPEAAGAPS |
| | | KTISKAKGQPREPQV | VSHEDPEVKFN | VFLFPPKPKDT |
| | | YTLPPCREEMTKNQV | WYVDGVEVHN | LMISRTPEVTC |
| | | SLSCAVKGFYPSDIA | AKTKPREEQYNS | VVVDVSHEDP |
| | | VEWESNGQPENNYK | TYRVVSVLTVLH | EVKFNWYVDG |
| | | TTPPVLDSDGSFFLVS | QDWLNGKEYKC | VEVHNAKTKP |
| | | KLTVDKSRWQQGNV | KVSNKALPAPIE | REEQYNSTYR |
| | | FSCSVMHEALHNHY | KTISKAKGQPRE | VVSVLTVLHQ |
| | | TQKSLSLSPGSGGGG | PQVCTLPPSREE | DWLNGKEYKC |
| | | SGGGGSGGGGSAPTS | MTKNQVSLWCL | KVSNKALPAPI |
| | | SSTKKTQLQLEELLL | VKGFYPSDIAVE | EKTISKAKGQP |
| | | DLQMILNGINNYKNP | WESNGQPENNY | REPQVCTLPPS |
| | | KLTEMLTAKFYMPK | KTTPPVLDSDGS | REEMTKNQVS |
| | | KATELKHLQCLEEEL | FFLYSKLTVDKS | LWCLVKGFYP |
| | | KPLEEVLNLAQSKNF | RWQQGNVFSCS | SDIAVEWESNG |
| | | HLRPRDLISNINVIVL | VMHEALHNHYT | QPENNYKTTPP |
| | | ELKGSETTFMCEYAD | QKSLSLSPG | VLDSDGSFFLY |
| | | ETATIVEFLNRWITFA | (SEQ ID | SKLTVDKSRW |
| | | QSIISTLT | NO: 348) | QQGNVFSCSV |
| | | (SEQ ID NO: 347) | | MHEALHNHYT |
| | | | | QKSLSLSPGK |
| | | | | (SEQ ID |
| | | | | NO: 349) |
| xhCD8v15- | EIVLTQSPGTLS | EVQLVESGGGLVQPG | EVQLVESGGGL | EVQLVESGGG |
| IL-2m4 | LSPGERATLSCR | GSLRLSCAASGFTFSS | VQPGGSLRLSCA | LVQPGGSLRLS |
| | ASQSVSSNLAW | YAMSWVRQAPGKGL | ASGFTFSSYAMS | CAASGFTFSSY |
| | YQQKPGQAPRL | EWVSDISYAGGSTAY | WVRQAPGKGLE | AMSWVRQAPG |
| | LIYGASSRATGI | ADSVKGRFTISRDNA | WVSDISYAGGST | KGLEWVSDISY |
| | PDRFSGSGSGT | KNSLYLQMNSLRAE | AYADSVKGRFTI | AGGSTAYADS |
| | DFTLTISRLEPE | DTAVYYCARSNAYA | SRDNAKNSLYL | VKGRFTISRDN |
| | DFAVYYCQQY | WDDALDIWGQGTLV | QMNSLRAEDTA | AKNSLYLQMN |
| | GSSPPVTFGQG | TVSSASTKGPSVFPLA | VYYCARSNAYA | SLRAEDTAVY |
| | TKVEIKRTVAA | PSSKSTSGGTAALGC | WDDALDIWGQG | YCARSNAYAW |
| | PSVFIFPPSDEQ | LVKDYFPEPVTVSWN | TLVTVSSASTKG | DDALDIWGQG |
| | LKSGTASVVCL | SGALTSGVHTFPAVL | PSVFPLAPSSKST | TLVTVSSASTK |
| | LNNFYPREAKV | QSSGLYSLSSVVTVPS | SGGTAALGCLV | GPSVFPLAPSS |
| | QWKVDNALQS | SSLGTQTYICNVNHK | KDYFPEPVTVSW | KSTSGGTAAL |
| | GNSQESVTEQD | PSNTKVDKKVEPKSC | NSGALTSGVHTF | GCLVKDYFPEP |
| | SKDSTYSLSSTL | DKTHTCPPCPAPEAA | PAVLQSSGLYSL | VTVSWNSGAL |
| | TLSKADYEKHK | GAPSVFLFPPKPKDTL | SSVVTVPSSSLG | TSGVHTFPAVL |
| | VYACEVTHQGL | MISRTPEVTCVVVDV | TQTYICNVNHKP | QSSGLYSLSSV |
| | SSPVTKSFNRGE | SHEDPEVKFNWYVD | SNTKVDKKVEP | VTVPSSSLGTQ |
| | C | GVEVHNAKTKPREE | KSCDKTHTCPPC | TYICNVNHKPS |
| | (SEQ ID | QYNSTYRVVSVLTVL | PAPEAAGAPSVF | NTKVDKKVEP |
| | NO: 350) | HQDWLNGKEYKCKV | LFPPKPKDTLMIS | KSCDKTHTCPP |
| | | SNKALPAPIEKTISKA | RTPEVTCVVVD | CPAPEAAGAPS |
| | | KGQPREPQVYTLPPC | VSHEDPEVKFN | VFLFPPKPKDT |
| | | REEMTKNQVSLSCA | WYVDGVEVHN | LMISRTPEVTC |
| | | VKGFYPSDIAVEWES | AKTKPREEQYNS | VVVDVSHEDP |
| | | NGQPENNYKTTPPVL | TYRVVSVLTVLH | EVKFNWYVDG |
| | | DSDGSFFLVSKLTVD | QDWLNGKEYKC | VEVHNAKTKP |
| | | KSRWQQGNVFSCSV | KVSNKALPAPIE | REEQYNSTYR |
| | | MHEALHNHYTQKSL | KTISKAKGQPRE | VVSVLTVLHQ |
| | | SLSPGSGGGGSGGGG | PQVCTLPPSREE | DWLNGKEYKC |
| | | SGGGGSAPTSSSTKK | MTKNQVSLWCL | KVSNKALPAPI |
| | | TQLQLEELLLDLQMI | VKGFYPSDIAVE | EKTISKAKGQP |
| | | LNGINNYKNPKLTEM | WESNGQPENNY | REPQVCTLPPS |
| | | LTAKFYMPKKATEL | KTTPPVLDSDGS | REEMTKNQVS |
| | | KHLQCLEEELKPLEE | FFLYSKLTVDKS | LWCLVKGFYP |
| | | VLNLAQSKNFHLRPR | RWQQGNVFSCS | SDIAVEWESNG |
| | | DLISNINVIVLELKGS | VMHEALHNHYT | QPENNYKTTPP |
| | | ETTFMCEYADETATI | QKSLSLSPG | VLDSDGSFFLY |
| | | VEFLNRWITFAQSIIS | (SEQ ID | SKLTVDKSRW |
| | | TLT | NO: 352) | QQGNVFSCSV |
| | | (SEQ ID NO: 351) | | MHEALHNHYT |
| | | | | QKSLSLSPGK |
| | | | | (SEQ ID |
| | | | | NO: 353) |

### Production of Antibodies and Fusion Proteins

Antibodies, antibody fragments, and fusion proteins may be produced using recombinant methods, e.g., as exemplified infra. In some embodiments, nucleic acid encoding the antibody/fusion protein can be isolated and inserted into a replicable vector for further cloning or for expression. DNA encoding the antibody/fusion protein may be readily isolated and sequenced using conventional procedures (e.g., via oligonucleotide probes capable of binding specifically to genes encoding the heavy and light chains of the antibody/fragment). Many vectors are known in the art; vector components generally include, but are not limited to, one or more of the following: a signal sequence, an origin of replication, one or more marker genes, an enhancer element, a promoter, and a transcription termination sequence. Suitable host cells for cloning or expressing the DNA in the vectors herein are the prokaryote, yeast, or higher eukaryote cells. When using recombinant techniques, the antibody/fusion protein can be produced intracellularly, in the periplasmic space, or directly secreted into the medium. If the antibody/fragment is produced intracellularly, the particulate debris, either host cells or lysed fragments, are removed, for example, by centrifugation or ultrafiltration. Where the antibody/fusion protein is secreted into the medium, supernatants from such expression systems are generally first concentrated using a commercially available protein concentration filter. Non-limiting descriptions of fusion protein production are provided in the Examples infra.

### Pharmaceutical Compositions

In some embodiments, a fusion protein of the present disclosure is administered to an individual as part of a pharmaceutical composition, e.g., including the fusion protein and one or more pharmaceutically acceptable carriers. Suitable routes of administration are known in the art and include, without limitation, subcutaneous injection and intravenous injection or infusion.

Pharmaceutical compositions and formulations as described herein can be prepared by mixing the active ingredients (such as a fusion protein) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives; low molecular weight (less than about 10 residues) polypeptides; proteins, such as serum albumin, gelatin, or immunoglobulins; hydrophilic polymers such as polyvinylpyrrolidone; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). In some embodiments, a fusion protein of the present disclosure is lyophilized. In some embodiments, the lyophilized fusion protein is reconstituted in a liquid prior to administration.

In some embodiments, the methods of the present disclosure further comprise administering to the individual a second antiviral or immunomodulatory agent. In some embodiments, the fusion protein of the present disclosure is administered to the individual (e.g., in need thereof) before administration of the second antiviral or immunomodulatory agent. In some embodiments, the fusion protein of the present disclosure is administered to the individual (e.g., in need thereof) concurrently with administration of the second antiviral or immunomodulatory agent. In some embodiments, the fusion protein of the present disclosure is administered to the individual (e.g., in need thereof) after administration of the second antiviral or immunomodulatory agent. In some embodiments, the fusion protein of the present disclosure is administered via the same route as the second antiviral or immunomodulatory agent. In some embodiments, the fusion protein of the present disclosure and the second antiviral or immunomodulatory agent are administered to the individual via different routes. In some embodiments, the fusion protein of the present disclosure is administered in the same composition or formulation as the second antiviral or immunomodulatory agent. In some embodiments, the fusion protein of the present disclosure and the second antiviral or immunomodulatory agent are administered in different compositions or formulations.

In some embodiments, the second antiviral or immunomodulatory agent comprises a nucleoside or nucleotide analog. Exemplary nucleoside/nucleotide analogs for treating HBV infection are known in the art and include, without limitation, adefovir dipivoxil, emtricitabine, entecavir, lamivudine, telbivudine, and tenofovir (e.g., tenofovir disoproxil fumarate or tenofovir alafenamide).

In some embodiments, the second antiviral or immunomodulatory agent comprises an antiretroviral therapy (ART). Exemplary ARTs for treating HIV infection are known in the art and include, without limitation, nucleoside reverse transcriptase inhibitors (NRTIs), non-nucleoside reverse transcriptase inhibitors (NNRTIs), protease inhibitors (PIS), fusion inhibitors, CCR5 antagonists, integrase strand transfer inhibitors (INSTIs), attachment inhibitors, post-attachment inhibitors, and pharmacokinetic enhancers. Approved NRTIs include abacavir (ZIAGEN®, GlaxoSmithKline; abacavir sulfate, ABC), didanosine (VIDEX®, Bristol-Myers Squibb; ddl), stavudine (ZERIT®, Bristol-Myers Squibb; d4T), emtricitabine (EMTRIVAR, Gilead; FTC), lamivudine (EPIVIR®, GlaxoSmithKline; 3TC), tenofovir disoproxil fumarate (VIREAD®, Gilead; tenofovir DF, TDF), tenofovir alafenamide (VEMLIDY®, Gilead; TAF), and zidovudine (RETROVIR®, GlaxoSmithKline; azidothymidine, AZT, ZDV). Approved NNRTIs include doravirine (PIFELTRO™, Merck; DOR), efavirenz (SUSTIVA®, Bristol-Myers Squibb; EFV), cabotegravir/ripivirine (CABENUVA, ViiV), delavirdine (RESCRIPOR, ViiV; DLV), etravirine (INTELENCE®, Janssen; ETR), nevirapine (VIRAMUNE®, Boehringer Ingelheim; VIRAMUNE XR®, Boehringer Ingelheim; extended-release nevirapine, NVP), and rilpivirine (EDURANT®, Janssen; rilpivirine hydrochloride, RPV). Approved PIs include atazanavir (REYATAZ®, Bristol-Myers Squibb; atazanavir sulfate, ATV), darunavir (PREZISTAR, Janssen; darunavir ethanolate, DRV), fosamprenavir (LEXIVAR, GlaxoSmithKline; fosamprenavir calcium, FOS-APV, FPV), idinavir (CRIXIVAN®, Merck; IDV), nelfinavir (VIRACEPT®, Pfizer; NFV), saquinavir (INVIRASE®, Genentech; FORTOVASER, Roche; SQV), ritonavir (NORVIR®, Abbvie; RTV), and tipranavir (APTIVUS®, Boehringer Ingelheim; TPV). Approved fusion inhibitors include enfuvirtide (FUZEON®, Genentech; T-20). Approved CCR5 antagonists include maraviroc (SELZENTRY®, ViiV; MVC). Approved INSTIs include bictegravir, elvitegravir (VITEKTA®, Gilead; EVG), cabotegravir (VOCABRIA®, GlaxoSmithKline; cabotegravir sodium, CAB), dolutegravir (TIVICAY®, ViiV; TIVICAY PD®, ViiV; dolutegravir sodium, DTG), and raltegravir (ISENTRESS®, Merck; INSENTRESS HD®, Merck; raltegravir potassium, RAL). Approved attachment inhibitors include fostemsavir (RUKOBIAR, GlaxoSmithKline; fostemsavir tromethamine, FTR). Approved post-attachment inhibitors include ibalizumab-uiyk (TROGARZO®, TaiMed; Hu5A8, IBA, ibalizumab, TMB-355, TNX-355). Approved pharmacokinetic enhancers include cobicistat (TYBOST®, Gilead; COBI, c) and ritonavir (RTV).

Exemplary ARTs for treating HIV infection also include combination HIV medicines, including without limitation abacavir and lamivudine (EPZICOM®, GlaxoSmithKline); abacavir, dolutegravir, and lamivudine (TRIUMEQ®, ViiV; TRIUMEQ PD®, ViiV); abacavir, lamivudine, and zidovudine (TRIZIVIR®, ViiV); atazanavir and cobicistat (EVOTAZ®, Bristol-Myers Squibb); bictegravir, emtricitabine, tenofovir alafenamide (BIKTARVY®, Gilead); cabotegravir and rilpivirine (CABENUVA®, ViiV); darunavir and cobicistat (PREZCOBIX®, Janssen); darunavir, cobicistat, emtricitabine, and tenofovir alafenamide (SYMTUZA®, Janssen); dolutegravir and lamivudine (DOVATO®, GlaxoSmithKline); dolutegravir and rilpivirine (JULUCA®, ViiV); doravirine, lamivudine, and tenofovir disoproxil fumarate (DELSTRIGO™, Merck); efavirenz, emtricitabine, and tenofovir disoproxil fumarate (ATRIPLA®, Bristol-Myers Squibb & Gilead); efavirenz, lamivudine, and tenofovir disoproxil fumarate (SYMFI™, Mylan); efavirenz, lamivudine, and tenofovir disoproxil fumarate (SYMFI LO™, Mylan); elvitegravir, cobicistat, emtricitabine, and tenofovir alafenamide (GENVOYA®, Gilead); elvitegravir, cobicistat, emtricitabine, and tenofovir disoproxil fumarate (STRIBILD®, Gilead); emtricitabine, rilpivirine, and tenofovir alafenamide (ODEFSEY®, Gilead); emtricitabine, rilpivirine, and tenofovir disoproxil fumarate (COMPLERAR, Gilead); emtricitabine and tenofovir alafenamide (DESCOVY®, Gilead); emtricitabine and tenofovir disoproxil fumarate (TRUVADA®, Gilead); lamivudine and tenofovir disoproxil fumarate (CIMDUO®, Mylan); lamivudine and zidovudine (COMBIVIR®, GlaxoSmithKline); and lopinavir and ritonavir (KALETRA®, Abbvie).

In some embodiments, the second antiviral or immunomodulatory agent comprises a capsid assembly modulator (CAM) or capsid assembly inhibitor. Exemplary capsid (e.g., nucleocapsid) assembly modulators/inhibitors for treating HBV infection are known in the art and include, without limitation, JNJ-56136379 (JNJ-6379), JNJ-632, BAY41-4109, NVR3-778, and ABI-H3733. See, e.g., Berke, J. M. et al. (2020) Antimicrob. Agents Chemother. 64 (5): e02439-19.

In some embodiments, the second antiviral or immunomodulatory agent comprises a TLR agonist, e.g., a TLR7, TLR8, or TLR9 agonist. Exemplary TLR agonists for treating HBV infection are known in the art and include, without limitation, RG7854, JNJ-64794964, GS-9688, and AIC649.

In some embodiments, the second antiviral or immunomodulatory agent comprises a vaccine, e.g., a nucleic acid-, peptide-, or inactivated/attenuated virus-based vaccine. Exemplary vaccines for treating HBV infection are known in the art and include, without limitation, BRII-179.

In some embodiments, the second antiviral or immunomodulatory agent comprises an RNAi-based agent, e.g., an siRNA-based agent. Exemplary RNAi-based agents for treating HBV infection are known in the art and include, without limitation, JNJ-3989, RG6346, VIR-2218, GSK3389404, and ARB-729.

In some embodiments, the second antiviral or immunomodulatory agent comprises interferon-α, including pegylated interferon-α.

In some embodiments, the second antiviral or immunomodulatory agent comprises an HBV entry inhibitor. Exemplary HBV entry inhibitors for treating HBV infection are known in the art and include, without limitation, bulevirtide (Myrcludex B). See, e.g., Leowattana, W. and Leowattana, T. (2022) World J. Virol. 11 (1): 57-72.

In some embodiments, the second antiviral or immunomodulatory agent comprises a covalently closed circular DNA (cccDNA) disruptor. For example, a gene editing approach such as CRISPR/Cas9 or zinc finger nucleases, ZFNs) can be used to disrupt cccDNA.

In some embodiments, the second antiviral or immunomodulatory agent comprises an HBV transcription inhibitor.

In some embodiments, the second antiviral or immunomodulatory agent comprises a CD3 bispecific T cell redirection agent. Exemplary CD3 bispecific T cell redirection agents for treating HBV infection are known in the art and include, without limitation, IMC-1109V.

In some embodiments, the second antiviral or immunomodulatory agent comprises an HBV antigen targeting agent. Exemplary HBV antigen targeting agents for treating HBV infection are known in the art and include, without limitation, VIR-3434.

In some embodiments, the second antiviral or immunomodulatory agent comprises a PD1/PDL1 blocking agent. Exemplary PD1/PDL1 blocking agents for treating HBV infection are known in the art and include, without limitation, ASC22.

In some embodiments, the second antiviral or immunomodulatory agent comprises a agent to reduce PDL1 expression in hepatocytes. Exemplary agents to reduce PDL1 expression in hepatocytes are known in the art and include, without limitation, RO7191863.

In some embodiments, the second antiviral or immunomodulatory agent comprises a RNA destabilizer agent. Exemplary PD1/PDL1 blocking agents for treating HBV infection are known in the art and include, without limitation, AB-161.

In some embodiments, the second antiviral or immunomodulatory agent comprises a Hepatitis B surface antigen (HBsAg) release inhibitor. Exemplary HBsAg release inhibitors for treating HBV infection are known in the art and include, without limitation, REP 2055, REP 2139-Ca, REP 2139-Mg, and REP 2165-Mg.

Certain aspects of the present disclosure relate to kits or articles of manufacture comprising any of the antibodies, antibody fragments, or fusion proteins disclosed herein. In some embodiments, the article of manufacture comprises a container and a label or package insert on or associated with the container. In some embodiments, the kit or article of manufacture further comprises instructions for using the antibody or fusion protein according to any of the methods disclosed herein, e.g., for treating chronic viral infection.

Suitable containers include, for example, bottles, vials, syringes, etc. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). At least one active agent in the composition is an antibody or fusion protein as described herein. The label or package insert indicates that the composition is used for treating the particular condition. The label or package insert will further comprise instructions for administering the antibody or fusion protein composition to the subject. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials considered from a commercial and user standpoint, including other buffers, diluents, filters, needles, and syringes.

### EXAMPLES

### Example 1: Generation of CD8-Targeted IL-2 and Untargeted IL-2 Fusion Proteins

CD8-targeted IL-2 fusion proteins were developed to selectively act on CD8+ T cells. These fusion proteins were used to test whether providing IL-2 signaling to CD8+ T cells alone is sufficient for anti-viral activity and whether this approach would enable a more optimal way of activating CD8+ T cells in the context of HBV infection, e.g., by avoiding cell types associated with toxicity and with counterproductive and immunosuppressive effects of IL-2, hence enabling optimal activation of CD8+ T cells in chronically infected HBV patients.

### Materials and Methods

### Cloning of Fusion Constructs

Techniques involving recombinant DNA manipulation were previously described in Sambrook et al., Molecular cloning: A laboratory manual; Cold Spring Harbor Laboratory Press, Cold Spring Harbor, N.Y., 1989. All reagents were used according to the manufacturer's instructions. DNA sequences were determined by double strand sequencing. Desired gene segments were either generated by PCR using appropriate templates or synthesized at Genewiz (South Plainfield, NJ), Integrated DNA Technologies (Coralville, IA) or GeneScript (Piscataway, NJ) from synthetic oligonucleotides. The gene segments were cloned into the expression vectors using either Gibson assembly® method or using restriction digest followed by ligation. DNA was purified from transformed bacteria and concentration was determined by UV visible spectroscopy. DNA sequencing was used to confirm the DNA sequences of the subcloned gene fragments.

To generate a representative CD8-targeted IL-2 fusion protein, mouse IL-2 mutein mIL2v1 with sequence depicted in SEQ ID: 384 was fused to an anti-mouse CD8 antibody, xmCD8, containing heterodimeric Fc mutations according to format A as shown in FIG. 2A. A (G4S)₃ 15-mer linker was inserted between the C-terminus of the IgG heavy chain and the N-terminus of IL-2 molecule. xmCD8 binds to the mCD8b region of the mCD8 antigen. Mouse IL-2 mutein mIL2v1 was representative of a mutein with abolished affinity to IL-2Ra (IL-2 receptor alpha) and reduced affinity to IL-2Rbg (IL-2 receptor beta gamma). mIL2v1 also contained a C140A mutation relative to SEQ ID NO:385 (WT mouse IL-2 sequence) to improve expression and purification.

mouse IL-2 mutein mIL2v1 sequence
(SEQ ID NO: 384)
APTSSSTSSSTAEAQQQQQQQQQQQQHLEQLLMDLQELLSRMENY
RNLKLPEMLTAKFALPKQATELKDLQCLEDELGPLRHVLDLTQSK
SFQLEDAENFISSIRVTVVKLKGSDNTFECQFDDESATVVDELRR
WIAFAQSIISTSPQ
Wild-type (WT) mouse IL-2 sequence
(SEQ ID NO: 385)
APTSSSTSSSTAEAQQQQQQQQQQQQHLEQLLMDLQELLSRMENY
RNLKLPRMLTFKFYLPKQATELKDLQCLEDELGPLRHVLDLTQSK
SFQLEDAENFISNIRVTVVKLKGSDNTFECQFDDESATVVDELRR
WIAFCQSIISTSPQ

A representative untargeted IL-2 fusion protein was similarly generated as the CD8-targeted IL-2 fusion. Specifically, mouse IL-2 mutein mIL2v2 with sequence depicted in SEQ ID: 390 was fused to a control antibody not binding to any mouse antigens, according to format A as shown in FIG. 2A. Mouse IL-2 mutein mIL2v2 was representative of a IL-2Rbg binding polypeptide with wild-type binding to IL-2Rbg and abolished binding to IL-2Ra (wild-type IL-2Rbg agonist or “not alpha” IL-2). mIL2v2 also contained a C140A mutation relative to SEQ ID NO:385 (WT mouse IL-2 sequence) to improve expression and purification.

(SEQ ID NO: 390)
mouse IL-2 mutein mIL2v2 sequence
APTSSSTSSSTAEAQQQQQQQQQQQQHLEQLLMDLQELLSRMENY
RNLKLPEMLTAKFALPKQATELKDLQCLEDELGPLRHVLDLTQSK
SFQLEDAENFISNIRVTVVKLKGSDNTFECQFDDESATVVDELRR
WIAFAQSIISTSPQ

Preparation of Fusion Proteins with IL-2 Polypeptides

Constructs encoding fusion proteins with IL-2 polypeptides as used in the examples were produced by co-transfecting exponentially growing Expi293 cells with the mammalian expression vectors using polyethylenimine (PEI). Supernatants were collected after 4-5 days of culture. IL-2 fusion constructs were first purified by affinity chromatography using a protein A matrix. The protein A column was equilibrated and washed in phosphate-buffered saline (PBS). The fusion constructs were eluted with 20 mM sodium citrate, 50 mM sodium chloride, pH 3.6. The eluted fractions were pooled and dialyzed into 10 mM MES, 25 mM sodium chloride pH 6. The proteins were further purified using ion-exchange chromatograph (Mono-S, GE Healthcare) to purify the heterodimers over the homodimers. After loading the protein, the column was washed with 10 mM MES 25 mM sodium chloride pH 6. The protein was then eluted with increasing gradient of sodium chloride from 25 mM up to 500 mM in 10 mM MES pH 6 buffer. The major eluent peak corresponding to the heterodimer was collected and concentrated. The purified protein was then polished by size exclusion chromatography (Superdex 200, GE Healthcare) in PBS.

The protein concentration of purified IL-2 fusion constructs was determined by measuring the optical density (OD) at 280 nm, using the molar extinction coefficient calculated on the basis of the amino acid sequence. Purity, integrity and monomeric state of the fusion constructs were analyzed by SDS-PAGE in the presence and absence of a reducing agent (5 mM 1,4-dithiothreitol) and stained with Coomassie blue (SimpleBlue™ SafeStain, Invitrogen). The NuPAGE® Pre-Cast gel system (Invitrogen) was used according to the manufacturer's instructions (4-20% Tris-glycine gels or 3-12% Bis-Tris). The aggregate content of immunoconjugate samples was analyzed using a Superdex 200 10/300 GL analytical size-exclusion column (GE Healthcare).

### Example 2: Selective STAT5 Activation in CD8 T Cells by mCD8-mIL2v1

Splenocytes were isolated from spleens of C57/BL6 mice by placing a spleen onto a 70 mM strainer and using a plunger to wash the cells with PBS through the strainer. Red blood cells were lysed with ACK lysis buffer according to the manufacturer's protocol, and cells were resuspended at 20×10⁶ cells per mL of RPMI media. Cells were then plated in U-bottom plates at 50 μL per well, and mCD8-mIL2v1 or CTRL-mIL2v2 were added to cells (50 μL as 2× stimulus). CD49b antibody (5 μl, DX5 clone, BioLegend) was added to each well prior to incubating the cells at 37° C. for 25 minutes. Cells were then fixed by adding 100 μL of 8% PFA solution (to provide a 4% PFA final concentration). Cells were washed 2× with PBS-2% FBS and resuspended in 75 μL Phosflow Perm buffer III buffer (BD Biosciences) and incubated for 1 hour at 4° C. Cells were then washed 3× with PBS-2% FBS and stained in 50 μL of FACS buffer (PBS with 0.5% BSA+2 mM EDTA) containing antibodies against CD3 (17A2, BD Biosciences), CD4 (GK1.5, BD Biosciences), CD8a (53-6.7, Biolegend), CD25 (7D4, BD Biosciences), and pSTAT5 (clone 47, BD Biosciences). Samples were washed 2× in FACS buffer and then analyzed on a flow cytometer.

Results are shown in FIG. 2B. Selective activation of CD8+ T cells was observed with mCD8-mIL2v1, with an EC50 on CD8+ T cells of 0.18 nM and EC50s on NK cells and Tregs exceeding 100 nM. This indicates a greater than 500-fold preference for CD8+ T cells over NK cells and Tregs for mCD8-mIL2v1. In contrast, the control untargeted “not alpha” IL-2 molecule, CTRL-mIL2v2, showed similar activity on NK cells, CD8+ T cells, and Treg cells. The highest potency was observed on NK cells (EC50 of 8.6 nM) followed by CD8+ T cells (EC50 of 26.0 nM), followed by Treg cells (EC50 of 42.5 nM) for CTRL-mIL2v2.

### Example 3: Effectiveness of CD8-Targeted IL-2 Fusion Protein in a Mouse Model of HBV

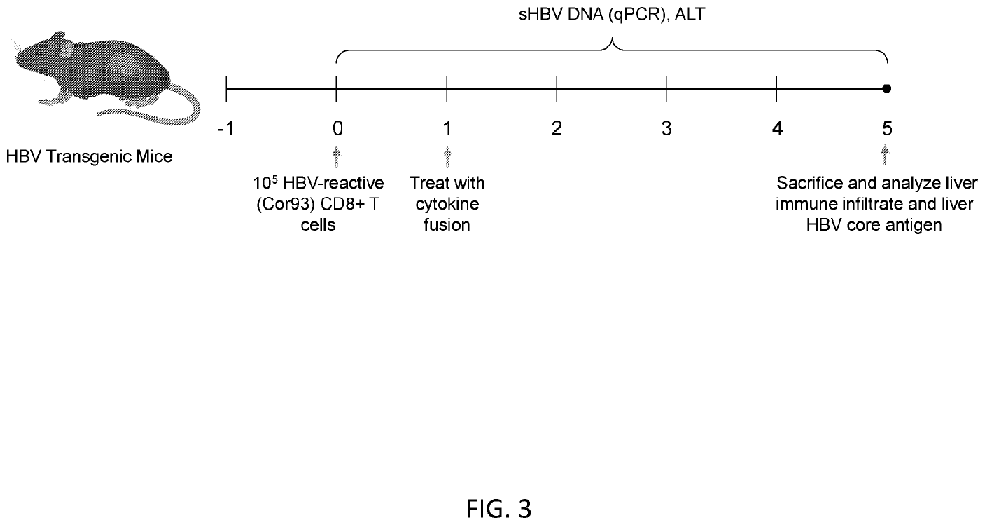

The effectiveness of CD8-targeted IL-2 fusion protein described in Example 1 and its ability to reverse CD8+ T cell dysfunction and enhance viral clearance were evaluated in a model similar to the previously published mouse model of HBV described in Bénéchet, A. P. et al. (2019) Nature 574 (7777): 200-205. General study design is outlined in FIG. 3. HBV-replication competent transgenic mice (lineage 1.3.32 on a C57BL/6 background) which constitutively express all HBV antigens in the liver were crossed to antibody-deficient mice (lineage DH-LMP2A on a C57BL/6 background), as previously described. Mice expressing HBeAg at >100 IU/mL were enrolled on study and randomized, then injected intravenously with 10⁵ HBV core antigen-specific naïve CD8+ T cells (Cor93T) intravenously. One day later, mice were treated with cytokine fusions of interest or control treatment (PBS). Serum HBV DNA and serum alanine aminotransferase (ALT) were tracked over time, and on Day 5, mice were sacrificed for characterization of the liver immune infiltrate and HBV viral level by HBV core antigen (HBcAg) staining by IHC. Serum HBV DNA levels indicate viral load. Serum ALT activity indicates killing of infected hepatocytes (liver cells) by HBV antigen-specific CD8+ T cells. Body weight was used to measure any overt systemic toxicity. This model mimics features of the CD8+ T cell dysfunction seen in HBV infected patients, as Cor93 T cells alone are unable to induce effective cytotoxicity against HBV-infected hepatocytes (as assessed by ALT), secrete IFNγ in response to HBV antigen, or effectively decrease viral load (Bénéchet, A. P. et al. (2019) Nature 574 (7777): 200-205).

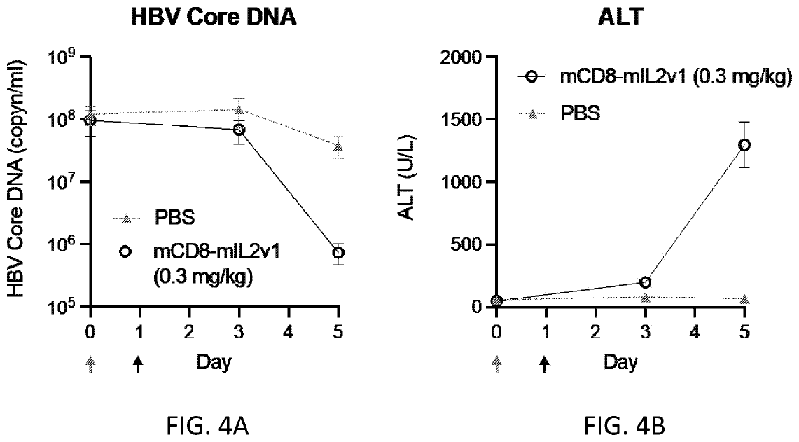

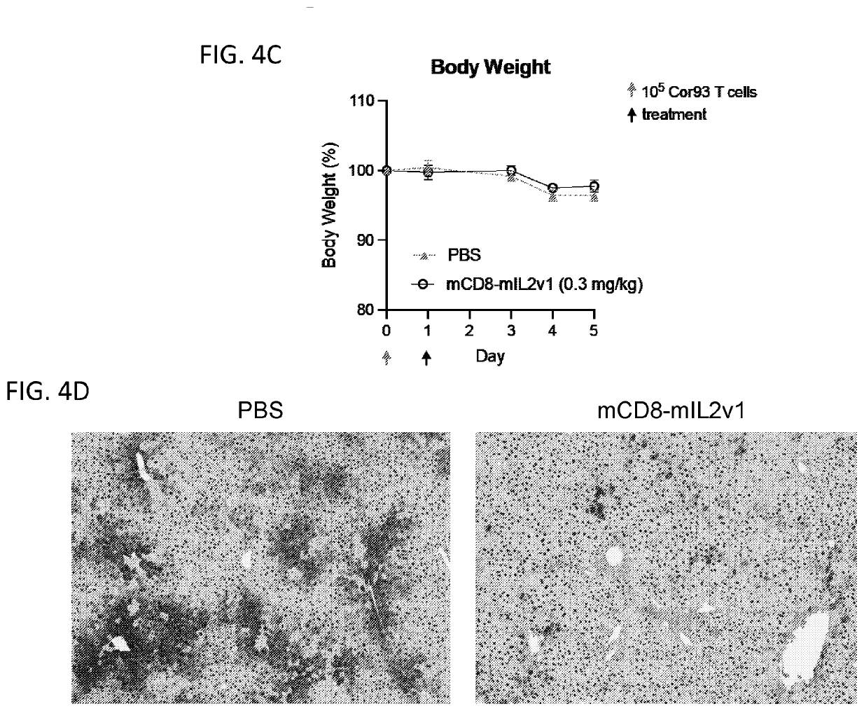

In one experiment, HBV transgenic mice received 10⁵ Cor93 T cells and were treated with CD8-targeted IL-2 fusion protein, mCD8-mIL2v1, at 0.3 mg/kg intravenously on day 1 and the following parameters were assessed: HBV DNA (FIG. 4A), serum ALT levels (FIG. 4B), and body weight (FIG. 4C). Serum HBV Core DNA was measured by qPCR. Serum ALT activity was measured with an International Federation of Clinical Chemistry and Laboratory Medicine optimized kinetic UV method in a SABA chemical analyzer (Seac-Radim). Body weight was measured throughout the study. Liver staining for HBV core antigen (HBcAg) was assessed on Day 5 by immunohistochemistry (IHC; FIG. 4D). For liver HBcAg IHC, livers were perfused with PBS, collected in Zn-formalin and then 24 hours later transferred into 70% ethanol. Tissue was embedded in paraffin and stained as previously described (Guidotti, L. G. et al. Immunosurveillance of the liver by intravascular effector CD8+ T cells. Cell 2015). Images were acquired using an Aperio Scanscope System CS2 microscope and utilized an ImageScope program following the manufacturer's (Leica Biosystem) instructions.

The results indicated that mice receiving 0.3 mg/kg of mCD8-mIL2v1 showed 130.8-fold reduced levels of HBV DNA (decreased viral load) compared to pre-treatment levels, whereas control (PBS) treated mice showed only 3.1-fold reduced levels (FIG. 4A). In addition, substantially increased levels of serum ALT were observed in mice that received 0.3 mg/kg of mCD8-mIL2v1, suggesting that the killing of HBV-infected liver cells was induced (FIG. 4B). At the same time, no overt systemic toxicity was induced, as mice that received 0.3 mg/kg of mCD8-mIL2v1 showed similar body weight as control treated mice (FIG. 4C). Furthermore, loss of HBV core antigen (HBcAg) was observed by IHC only in mice that received 0.3 mg/kg of mCD8-mIL2v1, with representative IHC staining shown in FIG. 4D.

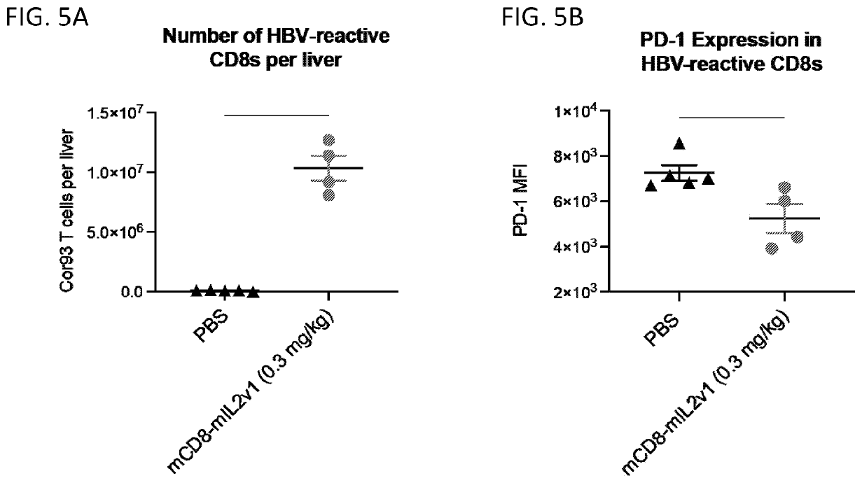

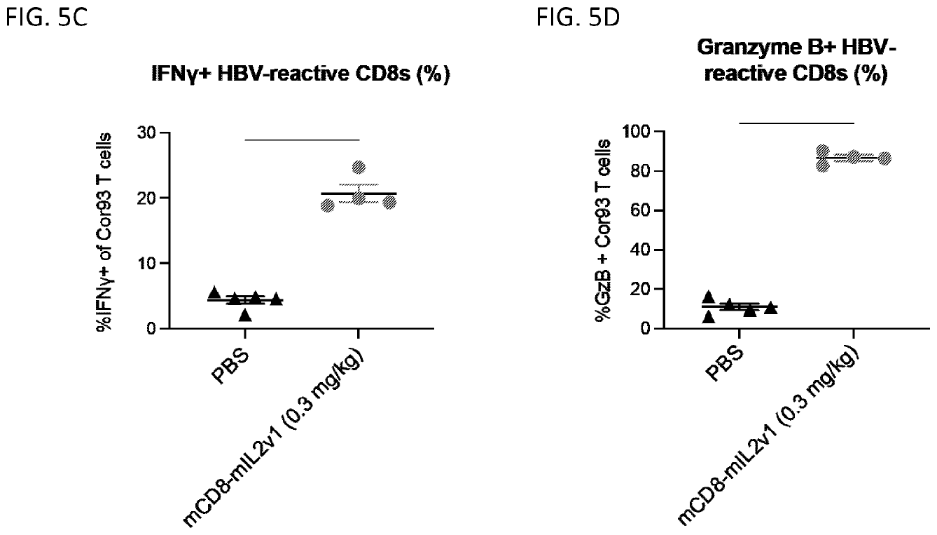

In the same experiment, additional parameters were analyzed as shown in FIGS. 5A-5D. On Day 5, livers were analyzed via flow cytometry, and HBV core antigen-reactive Cor93 CD8+ T cell numbers and phenotype were characterized. Cor93 T cells were identified via the Thy 1.1 congenic marker, and absolute counts in the liver and the mean fluorescence intensity of PD1 staining were quantified. In addition, ability of liver-infiltrating T cells to secrete IFNγ cytokine following stimulation with cognate peptide and expression of the cytotoxic mediator Granzyme B were analyzed. Specifically, a single cell suspension from the liver was pulsed with Cor93 cognate peptide MGLKFRQL (SEQ ID NO:391), residues 93-100 in the HBV core protein, for 5 hours at 37° C. in the presence of brefeldin A (BFA). Then cells were stained intracellularly and run on the flow cytometer. The fraction of Cor93 T cells expressing IFNγ and granzyme B were measured.

The results indicated that mice receiving 0.3 mg/kg of mCD8-mIL2v1 showed significantly increased numbers of HBV core antigen-reactive CD8+ T cells compared to control-treated mice (104-fold over PBS-treated mice) as depicted in FIG. 5A, and these cells expressed significantly lower levels of inhibitory marker PD-1 (FIG. 5B). Furthermore, increased ability to secrete IFNγ and express the cytotoxic mediator GzmB was observed in mCD8-mIL2v1-treated mice, with approximately 20% and 85% of HBV core antigen-reactive CD8+ T cells expressing IFNγ (FIG. 5C) and GzmB (FIG. 5D), respectively, compared to 5% and 10% in control mice, respectively.

### Example 4: Comparison of CD8-Targeted IL-2 and Untargeted “not α” IL-2 Fusion Proteins In the Mouse Model of HBV

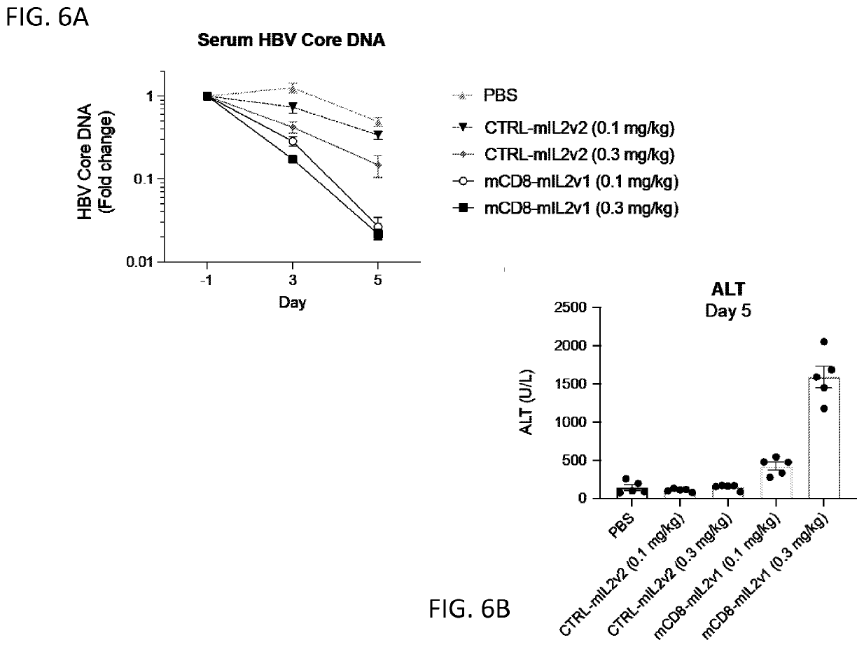

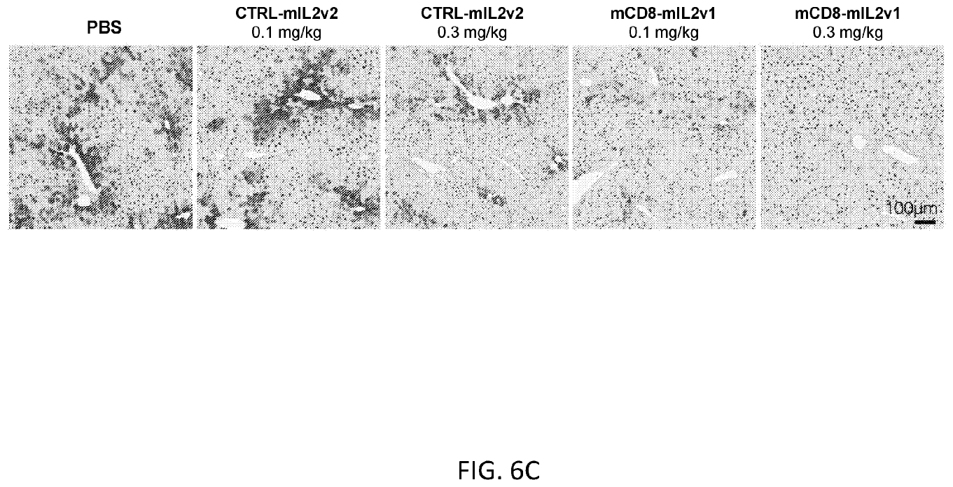

In a second experiment, the effectiveness of CD8-targeted IL-2 fusion protein in the same mouse model of HBV was analyzed in comparison to an untargeted “not α” IL-2 fusion protein. Proteins were generated as described in Example 1. HBV transgenic mice as described in Example 2 received 105 Cor93 T cells and were treated with mCD8-mIL2v1 or a control untargeted “not α” IL-2 (CTRL-mIL2v2) intravenously at 0.1 or 0.3 mg/kg on day 1. Serum HBV Core DNA, serum ALT activity, and liver staining for HBV core antigen by IHC were assessed as depicted in FIGS. 6A-6C.

As shown in FIG. 6A, mice that received mCD8-mIL2v1 showed strongly reduced levels of HBV DNA (decreased viral load) compared to pre-treatment levels at both dose levels tested: 37.6-fold at 0.1 mg/kg and 46.0-fold at 0.3 mg/kg. In contrast, mice that received control untargeted “not α” IL-2 (CTRL-mIL2v2) showed much more modest decreases in HBV DNA: 2.95-fold at 0.1 mg/kg and 6.76-fold at 0.3 mg/kg. Control (PBS) treated mice showed the smallest reduction in HBV DNA levels, only 2.03-fold. Furthermore, only mice that received mCD8-mIL2v1 showed strongly increased serum ALT activity including the mice treated with a lower dose of mCD8-mIL2v1 at 0.1 mg/kg (FIG. 6B). The strongest reduction in HBV core antigen staining of liver by IHC was also observed in mice treated with mCD8-mIL2v1 as depicted in FIG. 6C.

In the same experiment, livers were analyzed via flow cytometry, and HBV core antigen-reactive Cor93 CD8+ T cell numbers and phenotype were characterized as depicted in FIGS. 7A-7C. Results indicated that the largest increases in HBV-reactive CD8+ T cell counts were observed with mice treated with mCD8-mIL2v1 compared to those treated with CTRL-mIL2v2: 30.8-fold for 0.1 mg/kg mCD8-mIL2v1, 89.0 fold for 0.3 mg/kg mCD8-mIL2v1, 1.53-fold for 0.1 mg/kg CTRL-mIL2v2, and 4.61-fold for 0.3 mg/kg of CTRL-mIL2v2 over PBS-treated mice (FIG. 7A). With respect to phenotype of HBV-reactive CD8+ T cells in the liver, the highest IFNγ and granzyme B levels were detected in mice treated with mCD8-mIL2v1. Approximately 23% and 14% of CD8+ T cells expressed IFNγ in mice treated with 0.3 and 0.1 mg/kg of mCD8-mIL2v1, respectively whereas 11% and 7% of CD8+ T cells expressed IFNγ in mice treated with 0.3 and 0.1 mg/kg of CTRL-mIL2v2, respectively (FIG. 7B). Furthermore, approximately 93% and 61% of CD8+ T cells expressed GzmB in mice treated with 0.3 and 0.1 mg/kg of mCD8-mIL2v1, respectively whereas 29% and 15% of CD8+ T cells expressed GzmB in mice treated with 0.3 and 0.1 mg/kg of CTRL-mIL2v2, respectively (FIG. 7C).

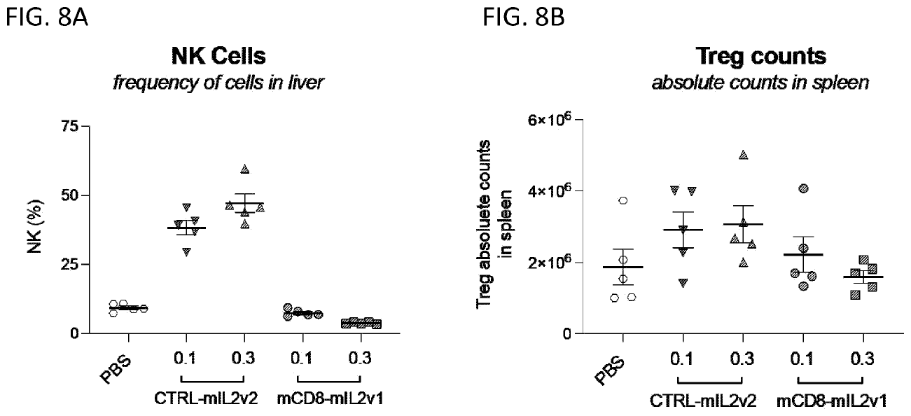

In addition, in the same experiment, the number of NK cells was quantified in the liver and regulatory T cells in the spleen as depicted in FIGS. 8A & 8B. In addition to CD8+ T cells, wild-type IL-2 and untargeted IL-2 proteins also activate NK cells and Treg cells, whereas the CD8-targeted IL-2 fusion protein preferentially activates CD8+ T cells over other cell types (see, e.g., International Patent Pub. No. WO2020/247843). Accordingly, CTRL-mIL2v2 induced a strong expansion of NK cells in the liver at both 0.1 and 0.3 mg/kg levels (increased up to 47.2% of intrahepatic leukocytes in the liver compared to 9.56% in the PBS group; FIG. 8A) as well as a minor expansion of Tregs in the periphery (1.64×; FIG. 8B). These results demonstrated that CTRL-mIL2v2 was active on its target cell types (CD8 T cells, NK cells and Tregs) but did not result in effective activation of CD8+ T cells like the CD8-targeted IL-2 fusion protein.

In summary, these results indicate that the fusion protein comprising a CD8 targeting antibody and an IL-2 mutein with attenuated IL2Rα and IL2Rβγ binding was highly selective for human CD8+ T cells over NK and Treg cells, and selectively enhanced IFN-γ production in CD8+ T cells in conjunction with TCR signaling. In the murine HBV model, the CD8-targeted IL-2 mutein fusion protein increased the number of HBV-reactive CD8+ T cells in the liver by >100-fold without substantial changes to Treg and NK cell numbers. The expanded CD8+ T cells showed increased expression of IFN-γ and granzyme B and decreased expression of PD-1 compared to controls. An increase in ALT, indicative of effective cytolysis of HBV-expressing hepatocytes, and decreased HBV DNA and antigens were observed with the CD8-targeted IL-2 mutein fusion protein. Despite profound changes to the CD8+ T cell compartment, the CD8-targeted IL-2 mutein fusion protein was well tolerated as indicated by a lack of body weight loss, unlike untargeted IL-2-based therapy. Thus, selectively providing an IL-2 signal to CD8+ T cells showed considerable anti-viral activity in a preclinical model of HBV. These data support the development of CD8-targeted IL-2 mutein fusion proteins as a potentially more tolerable and more active IL-2-based therapy for the treatment of HBV, such as chronic HBV.

What is claimed is:

1. A method of treating hepatitis B virus (HBV) and/or human immunodeficiency virus (HIV) infection, comprising administering to an individual in need thereof an effective amount of a fusion protein that comprises: (a) a first moiety comprising an antibody or antigen-binding fragment thereof that specifically binds human CD8b and/or human CD8ab with at least 10-fold higher affinity than its binding to human CD8a and/or human CD8aa; and (b) a second moiety comprising a cytokine, chemokine, or growth factor, wherein the first moiety is fused to the second moiety directly or via a linker.

2. The method of claim 1, wherein the HBV infection is chronic HBV infection.

3. The method of claim 1 or claim 2, wherein the administration results in an increase in HBV-reactive CD8+ T cells in the liver of the individual.

4. The method of any one of claims 1-3, wherein the administration results in reduced levels of serum HBV DNA of the individual.

5. The method of claim 1, wherein the HIV infection is HIV-1 or HIV-2 infection.

6. The method of claim 1 or claim 5, wherein the administration results in an increase in HIV-reactive CD8+ T cells in the individual.

7. The method of any one of claims 1-6, wherein the infection is HBV/HIV co-infection.

8. The method of any one of claims 1-7, wherein the individual is a human.

9. The method of any one of claims 1-8, wherein the second moiety induces activation of CD8+ T cells.

10. The method of claim 9, wherein the fusion protein induces activation of cells expressing a human CD8ab heterodimer with at least 10-fold higher potency than activation of cells expressing a human CD8aa homodimer.

11. The method of claim 9, wherein the fusion protein induces activation of CD8+ T cells with at least 10-fold higher potency than activation of NK cells.

12. The method of claim 10 or claim 11, wherein potency of activation is measured by EC50, as assessed by cell proliferation.

13. The method of claim 10 or claim 11, wherein potency of activation is measured by EC50, as assessed by STAT5 phosphorylation.

14. The method of claim 10 or claim 11, wherein potency of activation is measured by EC50, as assessed by cellular cytotoxic function.

15. The method of claim 14, wherein cellular cytotoxic function comprises expression of IFNγ and/or granzyme B.

16. The method of any one of claims 1-15, wherein the second moiety comprises an IL-2 polypeptide.

17. The method of claim 16, wherein the IL-2 polypeptide is a mutant IL-2 polypeptide comprising one or more mutations relative to a human IL-2 polypeptide comprising the sequence of
(SEQ ID NO: 81)
APTSSSTKKTQLQLEHLLLDLQMILNGINNYKNPKLTRMLTFKFY
MPKKATELKHLQCLEEELKPLEEVLNLAQSKNFHLRPRDLISNIN
VIVLELKGSETTFMCEYADETATIVEFLNRWITFCQSIISTLT.

18. The method of claim 17, wherein the mutant IL-2 polypeptide has a binding affinity to IL-2Rα that is reduced by 50% or more, compared to binding affinity of a wild-type IL-2 polypeptide comprising the sequence of SEQ ID: 81 for IL-2Rα.

19. The method of claim 18, wherein the mutant IL-2 polypeptide has a binding affinity to IL-2Rβ that is reduced by 50% or more, compared to binding affinity of a wild-type IL-2 polypeptide comprising the sequence of SEQ ID: 81 for IL-2Rβ; and/or
wherein the mutant IL-2 polypeptide has a binding affinity to IL-2Rγ that is reduced by 50% or more, compared to binding affinity of a wild-type IL-2 polypeptide comprising the sequence of SEQ ID: 81 for IL-2Rγ.

20. The method of any one of claims 17-19, wherein the IL-2 polypeptide comprises the sequence of SEQ ID NO:81 with one, two, three, four, or five amino acid substitutions relative to SEQ ID NO:81, and wherein the one, two, three, four, or five substitution(s) comprise substitution(s) at positions of SEQ ID NO:81 selected from the group consisting of: Q11, H16, L18, L19, D20, Q22, R38, F42, K43, Y45, E62, P65, E68, V69, L72, D84, S87, N88, V91, 192, T123, Q126, S127, 1129, and S130.

21. The method of claim 20, wherein the IL-2 polypeptide comprises the sequence of SEQ ID NO:81 with one of the following sets of amino acid substitutions (relative to the sequence of SEQ ID NO:81): R38E and F42A; R38D and F42A; F42A and E62Q; R38A and F42K; R38E, F42A, and N88S; R38E, F42A, and N88A; R38E, F42A, and N88G; R38E, F42A, and N88R; R38E, F42A, and N88T; R38E, F42A, and N88D; R38E, F42A, and V91E; R38E, F42A, and D84H; R38E, F42A, and D84K; R38E, F42A, and D84R; H16D, R38E and F42A; H16E, R38E and F42A; R38E, F42A and Q126S; R38D, F42A and N88S; R38D, F42A and N88A; R38D, F42A and N88G; R38D, F42A and N88R; R38D, F42A and N88T; R38D, F42A and N88D; R38D, F42A and V91E; R38D, F42A, and D84H; R38D, F42A, and D84K; R38D, F42A, and D84R; H16D, R38D and F42A; H16E, R38D and F42A; R38D, F42A and Q126S; R38A, F42K, and N88S; R38A, F42K, and N88A; R38A, F42K, and N88G; R38A, F42K, and N88R; R38A, F42K, and N88T; R38A, F42K, and N88D; R38A, F42K, and V91E; R38A, F42K, and D84H; R38A, F42K, and D84K; R38A, F42K, and D84R; H16D, R38A, and F42K; H16E, R38A, and F42K; R38A, F42K, and Q126S; F42A, E62Q, and N88S; F42A, E62Q, and N88A; F42A, E62Q, and N88G; F42A, E62Q, and N88R; F42A, E62Q, and N88T; F42A, E62Q, and N88D; F42A, E62Q, and V91E; F42A, E62Q, and D84H; F42A, E62Q, and D84K; and F42A, E62Q, and D84R.

22. The method of claim 20, wherein the IL-2 polypeptide comprises the sequence of SEQ ID NO:81 with a further amino acid substitution relative to SEQ ID NO:81 at position C125.

23. The method of claim 22, wherein the IL-2 polypeptide comprises the sequence of SEQ ID NO:81 with one of the following sets of amino acid substitutions (relative to the sequence of SEQ ID NO:81): R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, N88G, and C125A; R38E, F42A, N88R, and C125A; R38E, F42A, N88D, and C125A; R38E, F42A, N88T, and C125A; R38E, F42A, V91E, and C125A; R38E, F42A, D84H, and C125A; R38E, F42A, D84K, and C125A; R38E, F42A, D84R, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, N88G, and C125A; R38D, F42A, N88R, and C125A; R38D, F42A, N88T, and C125A; R38D, F42A, N88D, and C125A; R38D, F42A, V91E, and C125A; R38D, F42A, D84H, and C125A; R38D, F42A, D84K, and C125A; R38D, F42A, D84R, and C125A; H16D, R38D, F42A, and C125A; H16E, R38D, F42A, and C125A; R38D, F42A, C125A, and Q126S; R38A, F42K, N88S, and C125A; R38A, F42K, N88G, and C125A; R38A, F42K, N88R, and C125A; R38A, F42K, N88T, and C125A; R38A, F42K, N88D, and C125A; R38A, F42K, N88A, and C125A; R38A, F42K, V91E, and C125A; R38A, F42K, D84H, and C125A; R38A, F42K, D84K, and C125A; R38A, F42K, D84R, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, N88G, and C125A; F42A, E62Q, N88R, and C125A; F42A, E62Q, N88T, and C125A; F42A, E62Q, N88D, and C125A; F42A, E62Q, V91E, and C125A; F42A, E62Q, and D84H, and C125A; F42A, E62Q, and D84K, and C125A; F42A, E62Q, and D84R, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, N88G, and C125A; F42A, N88R, and C125A; F42A, N88T, and C125A; F42A, N88D, and C125A; F42A, V91E, and C125A; F42A, D84H, and C125A; F42A, D84K, and C125A; F42A, D84R, and C125A; H16D, F42A, and C125A; H16E, F42A, and C125A; and F42A, C125A and Q126S.

24. The method of any one of claims 17-19, wherein the IL-2 polypeptide comprises the sequence
(SEQ ID NO: 297)
APTSSSTKKTQLQLEHLLLDLQMILNGINNYKNPKLTEMLTAKFY
MPKKATELKHLQCLEEELKPLEEVLNLAQSKNFHLRPRDLISAIN
VIVLELKGSETTFMCEYADETATIVEFLNRWITFAQSIISTLT
or the sequence
(SEQ ID NO: 80)
APTSSSTKKTQLQLEELLLDLQMILNGINNYKNPKLTEMLTAKFY
MPKKATELKHLQCLEEELKPLEEVLNLAQSKNFHLRPRDLISNIN
VIVLELKGSETTFMCEYADETATIVEFLNRWITFAQSIISTLT

25. The method of any one of claims 17-19, wherein the IL-2 polypeptide comprises a sequence selected from the group consisting of SEQ ID NOs: 80, 85-155, 190-216, 297, and 354-383.

26. The method of any one of claims 1-25, wherein the first moiety binds to a cell expressing a human CD8ab heterodimer on its surface with an EC50 that is less than 1000 nM.

27. The method of any one of claims 1-26, wherein the first moiety binds human CD8+ T cells.

28. The method of any one of claims 1-27, wherein the antibody or antigen-binding fragment thereof is humanized or human.

29. The method of any one of claims 1-28, wherein the antibody or fragment comprises a heavy chain variable (VH) domain and a light chain variable (VL) domain, and wherein:
(a) the VH domain comprises a CDR-H1 comprising the amino acid sequence of SEQ ID NO: 13, a CDR-H2 comprising the amino acid sequence of SEQ ID NO: 14, and a CDR-H3 comprising the amino acid sequence of SEQ ID NO:15, and the VL domain comprises a CDR-L1 comprising the amino acid sequence of SEQ ID NO:16, a CDR-L2 comprising the amino acid sequence of SEQ ID NO: 17, and a CDR-L3 comprising the amino acid sequence of SEQ ID NO:18;
(b) the VH domain comprises a CDR-H1 comprising the amino acid sequence of SEQ ID NO: 19, a CDR-H2 comprising the amino acid sequence of SEQ ID NO:20, and a CDR-H3 comprising the amino acid sequence of SEQ ID NO:21, and the VL domain comprises a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24;
(c) the VH domain comprises a CDR-H1 comprising the amino acid sequence of SEQ ID NO: 25, a CDR-H2 comprising the amino acid sequence of SEQ ID NO:26, and a CDR-H3 comprising the amino acid sequence of SEQ ID NO:27, and the VL domain comprises a CDR-L1 comprising the amino acid sequence of SEQ ID NO:28, a CDR-L2 comprising the amino acid sequence of SEQ ID NO:29, and a CDR-L3 comprising the amino acid sequence of SEQ ID NO:30;
(d) the VH domain comprises a CDR-H1 comprising the amino acid sequence of SEQ ID NO: 31, a CDR-H2 comprising the amino acid sequence of SEQ ID NO:32, and a CDR-H3 comprising the amino acid sequence of SEQ ID NO:33, and the VL domain comprises a CDR-L1 comprising the amino acid sequence of SEQ ID NO:34, a CDR-L2 comprising the amino acid sequence of SEQ ID NO:35, and a CDR-L3 comprising the amino acid sequence of SEQ ID NO:36;
(e) the VH domain comprises a CDR-H1 comprising the amino acid sequence of SEQ ID NO: 37, a CDR-H2 comprising the amino acid sequence of SEQ ID NO:38, and a CDR-H3 comprising the amino acid sequence of SEQ ID NO:39, and the VL domain comprises a CDR-L1 comprising the amino acid sequence of SEQ ID NO:40, a CDR-L2 comprising the amino acid sequence of SEQ ID NO:41, and a CDR-L3 comprising the amino acid sequence of SEQ ID NO:42;
(f) the VH domain comprises a CDR-H1 comprising the amino acid sequence of SEQ ID NO: 43, a CDR-H2 comprising the amino acid sequence of SEQ ID NO:44, and a CDR-H3 comprising the amino acid sequence of SEQ ID NO:45, and the VL domain comprises a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48;
(g) the VH domain comprises a CDR-H1 comprising the amino acid sequence of SEQ ID NO: 1, a CDR-H2 comprising the amino acid sequence of SEQ ID NO:2, and a CDR-H3 comprising the amino acid sequence of SEQ ID NO:3, and the VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO: 6;
(h) the VH domain comprises a CDR-H1 comprising the amino acid sequence of SEQ ID NO: 177, a CDR-H2 comprising the amino acid sequence of SEQ ID NO:178, and a CDR-H3 comprising the amino acid sequence of SEQ ID NO: 179, and the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO:182;
(i) the VH domain comprises a CDR-H1 comprising the amino acid sequence of SEQ ID NO: 225, a CDR-H2 comprising the amino acid sequence of SEQ ID NO:226, and a CDR-H3 comprising the amino acid sequence of SEQ ID NO:227, and the VL domain comprises a CDR-L1 comprising the amino acid sequence of SEQ ID NO: 16, a CDR-L2 comprising the amino acid sequence of SEQ ID NO:17, and a CDR-L3 comprising the amino acid sequence of SEQ ID NO:228;
(j) the VH domain comprises a CDR-H1 comprising the amino acid sequence of SEQ ID NO: 225, a CDR-H2 comprising the amino acid sequence of SEQ ID NO:232, and a CDR-H3 comprising the amino acid sequence of SEQ ID NO:233, and the VL domain comprises a CDR-L1 comprising the amino acid sequence of SEQ ID NO:234, a CDR-L2 comprising the amino acid sequence of SEQ ID NO:235, and a CDR-L3 comprising the amino acid sequence of SEQ ID NO:236;
(k) the VH domain comprises a CDR-H1 comprising the amino acid sequence of SEQ ID NO: 225, a CDR-H2 comprising the amino acid sequence of SEQ ID NO:232, and a CDR-H3 comprising the amino acid sequence of SEQ ID NO:233, and the VL domain comprises a CDR-L1 comprising the amino acid sequence of SEQ ID NO:16, a CDR-L2 comprising the amino acid sequence of SEQ ID NO:17, and a CDR-L3 comprising the amino acid sequence of SEQ ID NO:228;
(l) the VH domain comprises a CDR-H1 comprising the amino acid sequence of SEQ ID NO: 229, a CDR-H2 comprising the amino acid sequence of SEQ ID NO:230, and a CDR-H3 comprising the amino acid sequence of SEQ ID NO:231, and the VL domain comprises a CDR-L1 comprising the amino acid sequence of RASQSVSSNLA (SEQ ID NO:40), a CDR-L2 comprising the amino acid sequence of GASSRAT (SEQ ID NO:41), and a CDR-L3 comprising the amino acid sequence of QQYGSSPPVT (SEQ ID NO:42); or
(m) the VH domain comprises a CDR-H1 comprising the amino acid sequence of SEQ ID NO:229, a CDR-H2 comprising the amino acid sequence of SEQ ID NO:237, and a CDR-H3 comprising the amino acid sequence of SEQ ID NO:231, and the VL domain comprises a CDR-L1 comprising the amino acid sequence of RASQSVSSNLA (SEQ ID NO: 40), a CDR-L2 comprising the amino acid sequence of GASSRAT (SEQ ID NO:41), and a CDR-L3 comprising the amino acid sequence of QQYGSSPPVT (SEQ ID NO:42).

30. The method of any one of claims 1-28, wherein the antibody or fragment comprises a heavy chain variable (VH) domain and a light chain variable (VL) domain, and wherein:
(a) the VH domain comprises:
a CDR-H1 comprising the amino acid sequence of X₁X₂AIS, wherein X₁ is S, K, G, N, R, D, T, or G, and wherein X₂ is Y, L, H, or F (SEQ ID NO:259),
a CDR-H2 comprising the amino acid sequence of X₁X₂X₃PX₄X₅X₆X₇X₈X₉YX₁₀QKFX₁G, wherein X₁ is G or H, X₂ is I or F, X₃ is I, N, or M, X₄ is G, N, H, S, R, I, or A, X₅ is A, N, H, S, T, F, or Y, X₆ is A, D, or G, X₇ is T, E, K, V, Q, or A, X₈ is A or T, X₉ is N or K, X₁₀ is A or N, and X₁₁ is Q or T (SEQ ID NO:260), and
a CDR-H3 comprising the amino acid sequence of X₁X₂X₃GX₄X₅LFX₆X₇, wherein X₁ is D or A, X₂ is A, G, E, R, Y, K, N, Q, L, or F, X₃ is A, L, P, or Y, X₄ is I or L, X₅ is R, A, Q, or S, X₆ is A or D, and X₇ is D, E, A, or S (SEQ ID NO:261); and
wherein the VL domain comprises:
a CDR-L1 comprising the amino acid sequence of X₁X₂SX₃X₄IX₅GX₆LN, wherein X₁ is R or G, X₂ is A or T, X₃ is Q or E, X₄ is E, N, T, S, A, K, D, G, R, or Q, X₅ is Y or S, and X₆ is A or V (SEQ ID NO:262),
a CDR-L2 comprising the amino acid sequence of GX₁X₂X₃LX₄X₅, wherein X₁ is A or S, X₂ is T, S, E, Q, or D, X₃ is N, R, A, E, or H, X₄ is Q or A, and X₅ is S or D (SEQ ID NO:263), and
a CDR-L3 comprising the amino acid sequence of QX₁X₂X₃X₄X₅PWT, wherein X₁ is S, N, D, Q, A, or E, X₂ is T, I, or S, X₃ is Y, L, or F, X₄ is D, G, T, E, Q, A, or Y, and X₅ is A, T, R, S, K, or Y (SEQ ID NO:264); or
(b) the VH domain comprises:
a CDR-H1 comprising the amino acid sequence of X₁YX₂MS, wherein X₁ is S, D, E, A, or Q and X₂ is A, G, or T (SEQ ID NO:268),
a CDR-H2 comprising the amino acid sequence of DIX₁X₂X₃GX₄X₅TX₆YADSVKG, wherein X₁ is T, N, S, Q, E, H, R, or A, X₂ is Y, W, F, or H, X₃ is A, S, Q, E, or T, X₄ is G or E, X₅ is S or I, and X₆ is A or G (SEQ ID NO: 269), and
a CDR-H3 comprising the amino acid sequence of X₁X₂X₃YXAWX₅X₆AX₇DX₈, wherein X₁ is S or A, X₂ is N, H, A, D, L, Q, Y, or R, X₃ is A, N, S, or G, X₄ is A, V, R, E, or S, X₅ is D or S, X₆ is D, N, Q, E, S, T, or L, X₇ is L, F, or M, and X₈ is I, Y, or V (SEQ ID NO:270); and
wherein the VL domain comprises:
a CDR-L1 comprising the amino acid sequence of RASQSVSSNLA (SEQ ID NO: 40),
a CDR-L2 comprising the amino acid sequence of GASSRAT (SEQ ID NO: 41), and
a CDR-L3 comprising the amino acid sequence of QQYGSSPPVT (SEQ ID NO: 42).

31. The method of claim 29 or claim 30, wherein:
(a) the VH domain comprises an amino acid sequence that is at least 90%, at least 95%, at least 99%, or 100% identical to the sequence of SEQ ID NO:62, and the VL domain comprises an amino acid sequence that is at least 90%, at least 95%, at least 99%, or 100% identical to the sequence of SEQ ID NO:63;
(b) the VH domain comprises an amino acid sequence that is at least 90%, at least 95%, at least 99%, or 100% identical to the sequence of SEQ ID NO:64, and the VL domain comprises an amino acid sequence that is at least 90%, at least 95%, at least 99%, or 100% identical to the sequence of SEQ ID NO:65;
(c) the VH domain comprises an amino acid sequence that is at least 90%, at least 95%, at least 99%, or 100% identical to the sequence of SEQ ID NO:66, and the VL domain comprises an amino acid sequence that is at least 90%, at least 95%, at least 99%, or 100% identical to the sequence of SEQ ID NO:67;
(d) the VH domain comprises an amino acid sequence that is at least 90%, at least 95%, at least 99%, or 100% identical to the sequence of SEQ ID NO:68, and the VL domain comprises an amino acid sequence that is at least 90%, at least 95%, at least 99%, or 100% identical to the sequence of SEQ ID NO:69;
(e) the VH domain comprises an amino acid sequence that is at least 90%, at least 95%, at least 99%, or 100% identical to the sequence of SEQ ID NO:70, and the VL domain comprises an amino acid sequence that is at least 90%, at least 95%, at least 99%, or 100% identical to the sequence of SEQ ID NO:71;
(f) the VH domain comprises an amino acid sequence that is at least 90%, at least 95%, at least 99%, or 100% identical to the sequence of SEQ ID NO:72, and the VL domain comprises an amino acid sequence that is at least 90%, at least 95%, at least 99%, or 100% identical to the sequence of SEQ ID NO:73;
(g) the VH domain comprises an amino acid sequence that is at least 90%, at least 95%, at least 99%, or 100% identical to the sequence of SEQ ID NO:185, and the VL domain comprises an amino acid sequence that is at least 90%, at least 95%, at least 99%, or 100% identical to the sequence of SEQ ID NO:186;
(h) the VH domain comprises an amino acid sequence that is at least 90%, at least 95%, at least 99%, or 100% identical to the sequence of SEQ ID NO:245, and the VL domain comprises an amino acid sequence that is at least 90%, at least 95%, at least 99%, or 100% identical to the sequence of SEQ ID NO:246;
(i) the VH domain comprises an amino acid sequence that is at least 90%, at least 95%, at least 99%, or 100% identical to the sequence of SEQ ID NO:251, and the VL domain comprises an amino acid sequence that is at least 90%, at least 95%, at least 99%, or 100% identical to the sequence of SEQ ID NO:252;
(j) the VH domain comprises an amino acid sequence that is at least 90%, at least 95%, at least 99%, or 100% identical to the sequence of SEQ ID NO:253, and the VL domain comprises an amino acid sequence that is at least 90%, at least 95%, at least 99%, or 100% identical to the sequence of SEQ ID NO:254;
(k) the VH domain comprises an amino acid sequence that is at least 90%, at least 95%, at least 99%, or 100% identical to the sequence of SEQ ID NO:247, and the VL domain comprises an amino acid sequence that is at least 90%, at least 95%, at least 99%, or 100% identical to the sequence of SEQ ID NO:248;
(l) the VH domain comprises an amino acid sequence that is at least 90%, at least 95%, at least 99%, or 100% identical to the sequence of SEQ ID NO:249, and the VL domain comprises an amino acid sequence that is at least 90%, at least 95%, at least 99%, or 100% identical to the sequence of SEQ ID NO:250;
(m) the VH domain comprises an amino acid sequence that is at least 90%, at least 95%, at least 99%, or 100% identical to the sequence of SEQ ID NO:255, and the VL domain comprises an amino acid sequence that is at least 90%, at least 95%, at least 99%, or 100% identical to the sequence of SEQ ID NO:256; or
(n) the VH domain comprises an amino acid sequence that is at least 90%, at least 95%, at least 99%, or 100% identical to the sequence of SEQ ID NO:257, and the VL domain comprises an amino acid sequence that is at least 90%, at least 95%, at least 99%, or 100% identical to the sequence of SEQ ID NO:258.

32. The method of any one of claims 1-31, wherein the antibody or antigen-binding fragment thereof is multispecific.

33. The method of claim 32, wherein the antibody or antigen-binding fragment thereof is bispecific.

34. The method of any one of claims 1-33, wherein the first moiety comprises two antibody heavy chain polypeptides comprising a structure according to formula [I], from N-terminus to C-terminus: 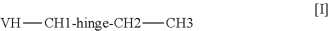
and two antibody light chain polypeptides comprising a structure according to formula [II], from N-terminus to C-terminus: 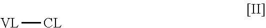
wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain; and wherein the N-terminus of the second moiety is fused to the C-terminus of one of the two CH3 domains.

35. The method of any one of claims 1-33, wherein the first moiety comprises a first antibody heavy chain polypeptide comprising a structure according to formula [I], from N-terminus to C-terminus: 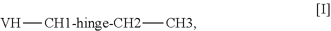
an antibody light chain polypeptide comprising a structure according to formula [II], from N-terminus to C-terminus: 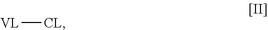
and a second antibody heavy chain polypeptide comprising a structure according to formula [III], from N-terminus to C-terminus: 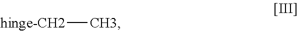
wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain; and
wherein the N-terminus of the second moiety is fused to the C-terminus of the CH3 domain of the second antibody heavy chain polypeptide.

36. The method of any one of claims 1-33, wherein the first moiety comprises a first antibody heavy chain polypeptide comprising a structure according to formula [I], from N-terminus to C-terminus: 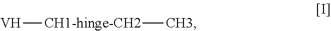
an antibody light chain polypeptide comprising a structure according to formula [II], from N-terminus to C-terminus: 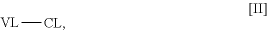
and a second antibody heavy chain polypeptide comprising a structure according to formula [III], from N-terminus to C-terminus: 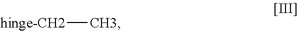
wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain; and
wherein the C-terminus of the second moiety is fused to the N-terminus of the hinge domain of the second antibody heavy chain polypeptide.

37. The method of any one of claims 1-33, wherein the first moiety comprises a first antibody heavy chain polypeptide comprising a structure according to formula [I], from N-terminus to C-terminus: 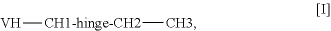
an antibody light chain polypeptide comprising a structure according to formula [II], from N-terminus to C-terminus: 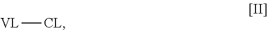
and a second antibody heavy chain polypeptide comprising a structure according to formula [III], from N-terminus to C-terminus: 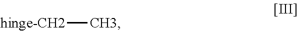
wherein VH is the VH domain, wherein CH1 is an antibody CH1 domain, wherein hinge is an antibody hinge domain, wherein CH2-CH3 is an antibody Fc domain, wherein VL is the VL domain, and wherein CL is an antibody constant light chain domain; and
wherein the N-terminus of the second moiety is fused to the C-terminus of the CH3 domain of the first antibody heavy chain polypeptide.

38. The method of claim 34, wherein:
the VH domain of both antibody heavy chain polypeptides comprises a CDR-H1 comprising the amino acid sequence of SEQ ID NO:13, a CDR-H2 comprising the amino acid sequence of SEQ ID NO:14, and a CDR-H3 comprising the amino acid sequence of SEQ ID NO: 15; and wherein the VL domain of both antibody light chain polypeptides comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:18;
the VH domain of both antibody heavy chain polypeptides comprises a CDR-H1 comprising the amino acid sequence of SEQ ID NO: 19, a CDR-H2 comprising the amino acid sequence of SEQ ID NO:20, and a CDR-H3 comprising the amino acid sequence of SEQ ID NO:21; and wherein the VL domain of both antibody light chain polypeptides comprises a CDR-L1 comprising the amino acid sequence of SEQ ID NO: 22, a CDR-L2 comprising the amino acid sequence of SEQ ID NO:23, and a CDR-L3 comprising the amino acid sequence of SEQ ID NO:24;
the VH domain of both antibody heavy chain polypeptides comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; and wherein the VL domain of both antibody light chain polypeptides comprises a CDR-L1 comprising the amino acid sequence of SEQ ID NO: 28, a CDR-L2 comprising the amino acid sequence of SEQ ID NO:29, and a CDR-L3 comprising the amino acid sequence of SEQ ID NO:30;
the VH domain of both antibody heavy chain polypeptides comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; and wherein the VL domain of both antibody light chain polypeptides comprises a CDR-L1 comprising the amino acid sequence of SEQ ID NO: 34, a CDR-L2 comprising the amino acid sequence of SEQ ID NO:35, and a CDR-L3 comprising the amino acid sequence of SEQ ID NO:36;
the VH domain of both antibody heavy chain polypeptides comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39; and wherein the VL domain of both antibody light chain polypeptides comprises a CDR-L1 comprising the amino acid sequence of SEQ ID NO: 40, a CDR-L2 comprising the amino acid sequence of SEQ ID NO:41, and a CDR-L3 comprising the amino acid sequence of SEQ ID NO:42;
the VH domain of both antibody heavy chain polypeptides comprises a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45; and wherein the VL domain of both antibody light chain polypeptides comprises a CDR-L1 comprising the amino acid sequence of SEQ ID NO: 46, a CDR-L2 comprising the amino acid sequence of SEQ ID NO:47, and a CDR-L3 comprising the amino acid sequence of SEQ ID NO:48;
the VH domain of both antibody heavy chain polypeptides comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3; and wherein the VL domain of both antibody light chain polypeptides comprises a CDR-L1 comprising the amino acid sequence of SEQ ID NO: 4, a CDR-L2 comprising the amino acid sequence of SEQ ID NO:5, and a CDR-L3 comprising the amino acid sequence of SEQ ID NO:6;
the VH domain of both antibody heavy chain polypeptides comprises a CDR-H1 comprising the amino acid sequence of SEQ ID NO: 177, a CDR-H2 comprising the amino acid sequence of SEQ ID NO: 178, and a CDR-H3 comprising the amino acid sequence of SEQ ID NO: 179; and wherein the VL domain of both antibody light chain polypeptides comprises a CDR-L1 comprising the amino acid sequence of SEQ ID NO: 180, a CDR-L2 comprising the amino acid sequence of SEQ ID NO:181, and a CDR-L3 comprising the amino acid sequence of SEQ ID NO: 182;
the VH domain of both antibody heavy chain polypeptides comprises a CDR-H1 comprising the amino acid sequence of SEQ ID NO:225, a CDR-H2 comprising the amino acid sequence of SEQ ID NO:226, and a CDR-H3 comprising the amino acid sequence of SEQ ID NO:227; and wherein the VL domain of both antibody light chain polypeptides comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:228;
the VH domain of both antibody heavy chain polypeptides comprises a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231; and wherein the VL domain of both antibody light chain polypeptides comprises a CDR-L1 comprising the amino acid sequence of SEQ ID NO: 40, a CDR-L2 comprising the amino acid sequence of SEQ ID NO:41, and a CDR-L3 comprising the amino acid sequence of SEQ ID NO:42;
the VH domain of both antibody heavy chain polypeptides comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233; and wherein the VL domain of both antibody light chain polypeptides comprises a CDR-L1 comprising the amino acid sequence of SEQ ID NO: 234, a CDR-L2 comprising the amino acid sequence of SEQ ID NO:235, and a CDR-L3 comprising the amino acid sequence of SEQ ID NO:236;
the VH domain of both antibody heavy chain polypeptides comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233; and wherein the VL domain of both antibody light chain polypeptides comprises a CDR-L1 comprising the amino acid sequence of SEQ ID NO: 16, a CDR-L2 comprising the amino acid sequence of SEQ ID NO:17, and a CDR-L3 comprising the amino acid sequence of SEQ ID NO:228; or
the VH domain of both antibody heavy chain polypeptides comprises a CDR-H1 comprising the amino acid sequence of SEQ ID NO:229, a CDR-H2 comprising the amino acid sequence of SEQ ID NO:237, and a CDR-H3 comprising the amino acid sequence of SEQ ID NO:231; and wherein the VL domain of both antibody light chain polypeptides comprises a CDR-L1 comprising the amino acid sequence of SEQ ID NO: 40, a CDR-L2 comprising the amino acid sequence of SEQ ID NO:41, and a CDR-L3 comprising the amino acid sequence of SEQ ID NO:42.

39. The method of claim 34 or claim 38, wherein:
the VH domain of both antibody heavy chain polypeptides comprises an amino acid sequence that is at least 90%, at least 95%, at least 99%, or 100% identical to the sequence of SEQ ID NO:62, and wherein the VL domain of both antibody light chain polypeptides comprises an amino acid sequence that is at least 90%, at least 95%, at least 99%, or 100% identical to the sequence of SEQ ID NO:63; optionally the VH domain of both antibody heavy chain polypeptides comprises the amino acid sequence of SEQ ID NO:62, and the VL domain of both antibody light chain polypeptides comprises the amino acid sequence of SEQ ID NO:63;
the VH domain of both antibody heavy chain polypeptides comprises an amino acid sequence that is at least 90%, at least 95%, at least 99%, or 100% identical to the sequence of SEQ ID NO:64, and wherein the VL domain of both antibody light chain polypeptides comprises an amino acid sequence that is at least 90%, at least 95%, at least 99%, or 100% identical to the sequence of SEQ ID NO:65; optionally the VH domain of both antibody heavy chain polypeptides comprises the amino acid sequence of SEQ ID NO:64, and the VL domain of both antibody light chain polypeptides comprises the amino acid sequence of SEQ ID NO:65;
the VH domain of both antibody heavy chain polypeptides comprises an amino acid sequence that is at least 90%, at least 95%, at least 99%, or 100% identical to the sequence of SEQ ID NO:66, and wherein the VL domain of both antibody light chain polypeptides comprises an amino acid sequence that is at least 90%, at least 95%, at least 99%, or 100% identical to the sequence of SEQ ID NO:67; optionally the VH domain of both antibody heavy chain polypeptides comprises the amino acid sequence of SEQ ID NO:66, and the VL domain of both antibody light chain polypeptides comprises the amino acid sequence of SEQ ID NO:67;
the VH domain of both antibody heavy chain polypeptides comprises an amino acid sequence that is at least 90%, at least 95%, at least 99%, or 100% identical to the sequence of SEQ ID NO:68, and wherein the VL domain of both antibody light chain polypeptides comprises an amino acid sequence that is at least 90%, at least 95%, at least 99%, or 100% identical to the sequence of SEQ ID NO:69; optionally the VH domain of both antibody heavy chain polypeptides comprises the amino acid sequence of SEQ ID NO:68, and the VL domain of both antibody light chain polypeptides comprises the amino acid sequence of SEQ ID NO:69;
the VH domain of both antibody heavy chain polypeptides comprises an amino acid sequence that is at least 90%, at least 95%, at least 99%, or 100% identical to the sequence of SEQ ID NO:70, and wherein the VL domain of both antibody light chain polypeptides comprises an amino acid sequence that is at least 90%, at least 95%, at least 99%, or 100% identical to the sequence of SEQ ID NO:71; optionally the VH domain of both antibody heavy chain polypeptides comprises the amino acid sequence of SEQ ID NO:70, and the VL domain of both antibody light chain polypeptides comprises the amino acid sequence of SEQ ID NO:71;
the VH domain of both antibody heavy chain polypeptides comprises an amino acid sequence that is at least 90%, at least 95%, at least 99%, or 100% identical to the sequence of SEQ ID NO: 72, and wherein the VL domain of both antibody light chain polypeptides comprises an amino acid sequence that is at least 90%, at least 95%, at least 99%, or 100% identical to the sequence of SEQ ID NO:73; optionally the VH domain of both antibody heavy chain polypeptides comprises the amino acid sequence of SEQ ID NO:72, and the VL domain of both antibody light chain polypeptides comprises the amino acid sequence of SEQ ID NO:73;
the VH domain of both antibody heavy chain polypeptides comprises an amino acid sequence that is at least 90%, at least 95%, at least 99%, or 100% identical to the sequence of SEQ ID NO:185, and wherein the VL domain of both antibody light chain polypeptides comprises an amino acid sequence that is at least 90%, at least 95%, at least 99%, or 100% identical to the sequence of SEQ ID NO:186; optionally the VH domain of both antibody heavy chain polypeptides comprises the amino acid sequence of SEQ ID NO:185, and the VL domain of both antibody light chain polypeptides comprises the amino acid sequence of SEQ ID NO:186;
the VH domain of both antibody heavy chain polypeptides comprises an amino acid sequence that is at least 90%, at least 95%, at least 99%, or 100% identical to the sequence of SEQ ID NO:245, and wherein the VL domain of both antibody light chain polypeptides comprises an amino acid sequence that is at least 90%, at least 95%, at least 99%, or 100% identical to the sequence of SEQ ID NO:246; optionally the VH domain of both antibody heavy chain polypeptides comprises the amino acid sequence of SEQ ID NO:245, and the VL domain of both antibody light chain polypeptides comprises the amino acid sequence of SEQ ID NO:246;
the VH domain of both antibody heavy chain polypeptides comprises an amino acid sequence that is at least 90%, at least 95%, at least 99%, or 100% identical to the sequence of SEQ ID NO:251, and wherein the VL domain of both antibody light chain polypeptides comprises an amino acid sequence that is at least 90%, at least 95%, at least 99%, or 100% identical to the sequence of SEQ ID NO:252; optionally the VH domain of both antibody heavy chain polypeptides comprises the amino acid sequence of SEQ ID NO:251, and the VL domain of both antibody light chain polypeptides comprises the amino acid sequence of SEQ ID NO:252;
the VH domain of both antibody heavy chain polypeptides comprises an amino acid sequence that is at least 90%, at least 95%, at least 99%, or 100% identical to the sequence of SEQ ID NO:253, and wherein the VL domain of both antibody light chain polypeptides comprises an amino acid sequence that is at least 90%, at least 95%, at least 99%, or 100% identical to the sequence of SEQ ID NO:254; optionally the VH domain of both antibody heavy chain polypeptides comprises the amino acid sequence of SEQ ID NO:253, and the VL domain of both antibody light chain polypeptides comprises the amino acid sequence of SEQ ID NO:254;
the VH domain of both antibody heavy chain polypeptides comprises an amino acid sequence that is at least 90%, at least 95%, at least 99%, or 100% identical to the sequence of SEQ ID NO:247, and wherein the VL domain of both antibody light chain polypeptides comprises an amino acid sequence that is at least 90%, at least 95%, at least 99%, or 100% identical to the sequence of SEQ ID NO:248; optionally the VH domain of both antibody heavy chain polypeptides comprises the amino acid sequence of SEQ ID NO:247, and the VL domain of both antibody light chain polypeptides comprises the amino acid sequence of SEQ ID NO:248;
the VH domain of both antibody heavy chain polypeptides comprises an amino acid sequence that is at least 90%, at least 95%, at least 99%, or 100% identical to the sequence of SEQ ID NO:249, and wherein the VL domain of both antibody light chain polypeptides comprises an amino acid sequence that is at least 90%, at least 95%, at least 99%, or 100% identical to the sequence of SEQ ID NO:250; optionally the VH domain of both antibody heavy chain polypeptides comprises the amino acid sequence of SEQ ID NO:249, and the VL domain of both antibody light chain polypeptides comprises the amino acid sequence of SEQ ID NO:250;
the VH domain of both antibody heavy chain polypeptides comprises an amino acid sequence that is at least 90%, at least 95%, at least 99%, or 100% identical to the sequence of SEQ ID NO:255, and wherein the VL domain of both antibody light chain polypeptides comprises an amino acid sequence that is at least 90%, at least 95%, at least 99%, or 100% identical to the sequence of SEQ ID NO:256; optionally the VH domain of both antibody heavy chain polypeptides comprises the amino acid sequence of SEQ ID NO:255, and the VL domain of both antibody light chain polypeptides comprises the amino acid sequence of SEQ ID NO:256; or
the VH domain of both antibody heavy chain polypeptides comprises an amino acid sequence that is at least 90%, at least 95%, at least 99%, or 100% identical to the sequence of SEQ ID NO:257, and wherein the VL domain of both antibody light chain polypeptides comprises an amino acid sequence that is at least 90%, at least 95%, at least 99%, or 100% identical to the sequence of SEQ ID NO:258; optionally the VH domain of both antibody heavy chain polypeptides comprises the amino acid sequence of SEQ ID NO:257, and the VL domain of both antibody light chain polypeptides comprises the amino acid sequence of SEQ ID NO:258.

40. The method of any one of claims 35-37, wherein:
the VH domain comprises a CDR-H1 comprising the amino acid sequence of SEQ ID NO:13, a CDR-H2 comprising the amino acid sequence of SEQ ID NO:14, and a CDR-H3 comprising the amino acid sequence of SEQ ID NO:15; and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:18;
the VH domain comprises a CDR-H1 comprising the amino acid sequence of SEQ ID NO:19, a CDR-H2 comprising the amino acid sequence of SEQ ID NO:20, and a CDR-H3 comprising the amino acid sequence of SEQ ID NO:21; and wherein the VL domain comprises a CDR-L1 comprising the amino acid sequence of SEQ ID NO: 22, a CDR-L2 comprising the amino acid sequence of SEQ ID NO:23, and a CDR-L3 comprising the amino acid sequence of SEQ ID NO:24;
the VH domain comprises a CDR-H1 comprising the amino acid sequence of SEQ ID NO:25, a CDR-H2 comprising the amino acid sequence of SEQ ID NO:26, and a CDR-H3 comprising the amino acid sequence of SEQ ID NO:27; and wherein the VL domain comprises a CDR-L1 comprising the amino acid sequence of SEQ ID NO: 28, a CDR-L2 comprising the amino acid sequence of SEQ ID NO:29, and a CDR-L3 comprising the amino acid sequence of SEQ ID NO:30;
the VH domain comprises a CDR-H1 comprising the amino acid sequence of SEQ ID NO:31, a CDR-H2 comprising the amino acid sequence of SEQ ID NO:32, and a CDR-H3 comprising the amino acid sequence of SEQ ID NO:33; and wherein the VL domain comprises a CDR-L1 comprising the amino acid sequence of SEQ ID NO: 34, a CDR-L2 comprising the amino acid sequence of SEQ ID NO:35, and a CDR-L3 comprising the amino acid sequence of SEQ ID NO:36;
the VH domain comprises a CDR-H1 comprising the amino acid sequence of SEQ ID NO:37, a CDR-H2 comprising the amino acid sequence of SEQ ID NO:38, and a CDR-H3 comprising the amino acid sequence of SEQ ID NO:39; and wherein the VL domain comprises a CDR-L1 comprising the amino acid sequence of SEQ ID NO: 40, a CDR-L2 comprising the amino acid sequence of SEQ ID NO:41, and a CDR-L3 comprising the amino acid sequence of SEQ ID NO:42;
the VH domain comprises a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45; and wherein the VL domain comprises a CDR-L1 comprising the amino acid sequence of SEQ ID NO: 46, a CDR-L2 comprising the amino acid sequence of SEQ ID NO:47, and a CDR-L3 comprising the amino acid sequence of SEQ ID NO:48;
the VH domain comprises a CDR-H1 comprising the amino acid sequence of SEQ ID NO:1, a CDR-H2 comprising the amino acid sequence of SEQ ID NO:2, and a CDR-H3 comprising the amino acid sequence of SEQ ID NO:3; and wherein the VL domain comprises a CDR-L1 comprising the amino acid sequence of SEQ ID NO:4, a CDR-L2 comprising the amino acid sequence of SEQ ID NO:5, and a CDR-L3 comprising the amino acid sequence of SEQ ID NO:6;
the VH domain comprises a CDR-H1 comprising the amino acid sequence of SEQ ID NO:177, a CDR-H2 comprising the amino acid sequence of SEQ ID NO:178, and a CDR-H3 comprising the amino acid sequence of SEQ ID NO:179; and wherein the VL domain comprises a CDR-L1 comprising the amino acid sequence of SEQ ID NO: 180, a CDR-L2 comprising the amino acid sequence of SEQ ID NO: 181, and a CDR-L3 comprising the amino acid sequence of SEQ ID NO:182;
the VH domain comprises a CDR-H1 comprising the amino acid sequence of SEQ ID NO:225, a CDR-H2 comprising the amino acid sequence of SEQ ID NO:226, and a CDR-H3 comprising the amino acid sequence of SEQ ID NO:227; and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:228;
the VH domain comprises a CDR-H1 comprising the amino acid sequence of SEQ ID NO:229, a CDR-H2 comprising the amino acid sequence of SEQ ID NO:230, and a CDR-H3 comprising the amino acid sequence of SEQ ID NO:231; and wherein the VL domain comprises a CDR-L1 comprising the amino acid sequence of SEQ ID NO: 40, a CDR-L2 comprising the amino acid sequence of SEQ ID NO:41, and a CDR-L3 comprising the amino acid sequence of SEQ ID NO:42;
the VH domain comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233; and wherein the VL domain comprises a CDR-L1 comprising the amino acid sequence of SEQ ID NO: 234, a CDR-L2 comprising the amino acid sequence of SEQ ID NO:235, and a CDR-L3 comprising the amino acid sequence of SEQ ID NO:236;
the VH domain comprises a CDR-H1 comprising the amino acid sequence of SEQ ID NO:225, a CDR-H2 comprising the amino acid sequence of SEQ ID NO:232, and a CDR-H3 comprising the amino acid sequence of SEQ ID NO:233; and wherein the VL domain comprises a CDR-L1 comprising the amino acid sequence of SEQ ID NO: 16, a CDR-L2 comprising the amino acid sequence of SEQ ID NO: 17, and a CDR-L3 comprising the amino acid sequence of SEQ ID NO:228; or
the VH domain comprises a CDR-H1 comprising the amino acid sequence of SEQ ID NO:229, a CDR-H2 comprising the amino acid sequence of SEQ ID NO:237, and a CDR-H3 comprising the amino acid sequence of SEQ ID NO:231; and wherein the VL domain comprises a CDR-L1 comprising the amino acid sequence of SEQ ID NO: 40, a CDR-L2 comprising the amino acid sequence of SEQ ID NO:41, and a CDR-L3 comprising the amino acid sequence of SEQ ID NO:42.

41. The method of any one of claims 35-37 and 40, wherein:
the VH domain comprises an amino acid sequence that is at least 90%, at least 95%, at least 99%, or 100% identical to the sequence of SEQ ID NO:62, and wherein the VL domain comprises an amino acid sequence that is at least 90%, at least 95%, at least 99%, or 100% identical to the sequence of SEQ ID NO:63; optionally, the VH domain comprises the amino acid sequence of SEQ ID NO:62, and the VL domain comprises the amino acid sequence of SEQ ID NO:63;
the VH domain comprises an amino acid sequence that is at least 90%, at least 95%, at least 99%, or 100% identical to the sequence of SEQ ID NO:64, and wherein the VL domain comprises an amino acid sequence that is at least 90%, at least 95%, at least 99%, or 100% identical to the sequence of SEQ ID NO:65; optionally, the VH domain comprises the amino acid sequence of SEQ ID NO:64, and the VL domain comprises the amino acid sequence of SEQ ID NO:65;
the VH domain comprises an amino acid sequence that is at least 90%, at least 95%, at least 99%, or 100% identical to the sequence of SEQ ID NO:66, and wherein the VL domain comprises an amino acid sequence that is at least 90%, at least 95%, at least 99%, or 100% identical to the sequence of SEQ ID NO:67; optionally, the VH domain comprises the amino acid sequence of SEQ ID NO:66, and the VL domain comprises the amino acid sequence of SEQ ID NO:67;
the VH domain comprises an amino acid sequence that is at least 90%, at least 95%, at least 99%, or 100% identical to the sequence of SEQ ID NO:68, and wherein the VL domain comprises an amino acid sequence that is at least 90%, at least 95%, at least 99%, or 100% identical to the sequence of SEQ ID NO:69; optionally, the VH domain comprises the amino acid sequence of SEQ ID NO:68, and the VL domain comprises the amino acid sequence of SEQ ID NO:69;
the VH domain comprises an amino acid sequence that is at least 90%, at least 95%, at least 99%, or 100% identical to the sequence of SEQ ID NO:70, and wherein the VL domain comprises an amino acid sequence that is at least 90%, at least 95%, at least 99%, or 100% identical to the sequence of SEQ ID NO:71; optionally, the VH domain comprises the amino acid sequence of SEQ ID NO:70, and the VL domain comprises the amino acid sequence of SEQ ID NO:71;
the VH domain comprises an amino acid sequence that is at least 90%, at least 95%, at least 99%, or 100% identical to the sequence of SEQ ID NO:72, and wherein the VL domain comprises an amino acid sequence that is at least 90%, at least 95%, at least 99%, or 100% identical to the sequence of SEQ ID NO:73; optionally, the VH domain comprises the amino acid sequence of SEQ ID NO:72, and the VL domain comprises the amino acid sequence of SEQ ID NO:73;
the VH domain comprises an amino acid sequence that is at least 90%, at least 95%, at least 99%, or 100% identical to the sequence of SEQ ID NO: 185, and wherein the VL domain comprises an amino acid sequence that is at least 90%, at least 95%, at least 99%, or 100% identical to the sequence of SEQ ID NO:186; optionally, the VH domain comprises the amino acid sequence of SEQ ID NO:185, and the VL domain comprises the amino acid sequence of SEQ ID NO:186;
the VH domain comprises an amino acid sequence that is at least 90%, at least 95%, at least 99%, or 100% identical to the sequence of SEQ ID NO:245, and wherein the VL domain comprises an amino acid sequence that is at least 90%, at least 95%, at least 99%, or 100% identical to the sequence of SEQ ID NO:246; optionally, the VH domain comprises the amino acid sequence of SEQ ID NO:245, and the VL domain comprises the amino acid sequence of SEQ ID NO:246;
the VH domain comprises an amino acid sequence that is at least 90%, at least 95%, at least 99%, or 100% identical to the sequence of SEQ ID NO:251, and wherein the VL domain comprises an amino acid sequence that is at least 90%, at least 95%, at least 99%, or 100% identical to the sequence of SEQ ID NO:252; optionally, the VH domain comprises the amino acid sequence of SEQ ID NO:251, and the VL domain comprises the amino acid sequence of SEQ ID NO:252;
the VH domain comprises an amino acid sequence that is at least 90%, at least 95%, at least 99%, or 100% identical to the sequence of SEQ ID NO:253, and wherein the VL domain comprises an amino acid sequence that is at least 90%, at least 95%, at least 99%, or 100% identical to the sequence of SEQ ID NO:254; optionally, the VH domain comprises the amino acid sequence of SEQ ID NO:253, and the VL domain comprises the amino acid sequence of SEQ ID NO:254;
the VH domain comprises an amino acid sequence that is at least 90%, at least 95%, at least 99%, or 100% identical to the sequence of SEQ ID NO:247, and wherein the VL domain comprises an amino acid sequence that is at least 90%, at least 95%, at least 99%, or 100% identical to the sequence of SEQ ID NO:248; optionally, the VH domain comprises the amino acid sequence of SEQ ID NO:247, and the VL domain comprises the amino acid sequence of SEQ ID NO:248;
the VH domain comprises an amino acid sequence that is at least 90%, at least 95%, at least 99%, or 100% identical to the sequence of SEQ ID NO:249, and wherein the VL domain comprises an amino acid sequence that is at least 90%, at least 95%, at least 99%, or 100% identical to the sequence of SEQ ID NO:250; optionally the VH domain comprises the amino acid sequence of SEQ ID NO:249, and the VL domain comprises the amino acid sequence of SEQ ID NO:250;
the VH domain comprises an amino acid sequence that is at least 90%, at least 95%, at least 99%, or 100% identical to the sequence of SEQ ID NO:255, and wherein the VL domain comprises an amino acid sequence that is at least 90%, at least 95%, at least 99%, or 100% identical to the sequence of SEQ ID NO:256; or optionally, the VH domain comprises the amino acid sequence of SEQ ID NO:255, and the VL domain comprises the amino acid sequence of SEQ ID NO:256; or
the VH domain comprises an amino acid sequence that is at least 90%, at least 95%, at least 99%, or 100% identical to the sequence of SEQ ID NO:257, and wherein the VL domain comprises an amino acid sequence that is at least 90%, at least 95%, at least 99%, or 100% identical to the sequence of SEQ ID NO:258; optionally, the VH domain comprises the amino acid sequence of SEQ ID NO:257, and the VL domain comprises the amino acid sequence of SEQ ID NO:258.

42. The method of any one of claims 34-41, wherein one or both of the antibody heavy chain polypeptides comprise(s) the following amino acid substitutions: L234A, L235A, and G237A, numbering according to EU index.

43. The method of any one of claims 34-42, wherein a first of the antibody heavy chain polypeptides comprises amino acid substitutions Y349C and T366W, and a second of the antibody heavy chain polypeptides comprises amino acid substitutions S354C, T366S, L368A and Y407V, numbering according to EU index.

44. The method of any one of claims 1-33, wherein the first moiety comprises one or two antibody heavy chain polypeptides and one or two antibody light chain polypeptides.

45. The method of any one of claims 1-33, wherein the first moiety comprises a single chain antibody or single chain variable fragment (scFv).

46. The method of any one of claims 1-33, wherein the first moiety comprises a VHH antibody.

47. The method of any one of claims 1-46, wherein the linker comprises the sequence (GGGS)xGn (SEQ ID NO:74), (GGGGS)xGn (SEQ ID NO:75), or (GGGGGS)xGn (SEQ ID NO: 76), S(GGGS)xGn (SEQ ID NO:386), S(GGGGS)xGn (SEQ ID NO:387), or S(GGGGGS)xGn (SEQ ID NO:388), wherein x=1, 2, 3, 4, 5, 6, 7, 8, 9, 10, 11, or 12, and wherein n=0, 1, 2 or 3.

48. The method of any one of claims 1-46, wherein the linker comprises the sequence GGGGSGGGGSGGGGS (SEQ ID NO:79) or SGGGGSGGGGSGGGGS (SEQ ID NO:389).

49. The method of any one of claims 1-33, wherein the fusion protein comprises:
one or two light chains comprising the amino acid sequence of SEQ ID NO: 156, a heavy chain comprising the amino acid sequence of SEQ ID NO: 157, and a heavy chain comprising the amino acid sequence of SEQ ID NO:158;
one or two light chains comprising the amino acid sequence of SEQ ID NO:159, a heavy chain comprising the amino acid sequence of SEQ ID NO:160, and a heavy chain comprising the amino acid sequence of SEQ ID NO:161;
one or two light chains comprising the amino acid sequence of SEQ ID NO: 162, a heavy chain comprising the amino acid sequence of SEQ ID NO: 163, and a heavy chain comprising the amino acid sequence of SEQ ID NO: 164;
one or two light chains comprising the amino acid sequence of SEQ ID NO: 165, a heavy chain comprising the amino acid sequence of SEQ ID NO:166, and a heavy chain comprising the amino acid sequence of SEQ ID NO: 167;
one or two light chains comprising the amino acid sequence of SEQ ID NO:168, a heavy chain comprising the amino acid sequence of SEQ ID NO:169, and a heavy chain comprising the amino acid sequence of SEQ ID NO:170;
one or two light chains comprising the amino acid sequence of SEQ ID NO:171, a heavy chain comprising the amino acid sequence of SEQ ID NO:172, and a heavy chain comprising the amino acid sequence of SEQ ID NO:173;
one or two light chains comprising the amino acid sequence of SEQ ID NO:174, a heavy chain comprising the amino acid sequence of SEQ ID NO: 175, and a heavy chain comprising the amino acid sequence of SEQ ID NO:176;
one or two light chains comprising the amino acid sequence of SEQ ID NO:187, a heavy chain comprising the amino acid sequence of SEQ ID NO: 188, and a heavy chain comprising the amino acid sequence of SEQ ID NO:189;
one or two light chains comprising the amino acid sequence of SEQ ID NO:298, a heavy chain comprising the amino acid sequence of SEQ ID NO:299, and a heavy chain comprising the amino acid sequence of SEQ ID NO:300;
one or two light chains comprising the amino acid sequence of SEQ ID NO:302, a heavy chain comprising the amino acid sequence of SEQ ID NO:303, and a heavy chain comprising the amino acid sequence of SEQ ID NO:304;
one or two light chains comprising the amino acid sequence of SEQ ID NO:306, a heavy chain comprising the amino acid sequence of SEQ ID NO:307, and a heavy chain comprising the amino acid sequence of SEQ ID NO:308;
one or two light chains comprising the amino acid sequence of SEQ ID NO:310, a heavy chain comprising the amino acid sequence of SEQ ID NO:311, and a heavy chain comprising the amino acid sequence of SEQ ID NO:312;
one or two light chains comprising the amino acid sequence of SEQ ID NO:314, a heavy chain comprising the amino acid sequence of SEQ ID NO:315, and a heavy chain comprising the amino acid sequence of SEQ ID NO:316;
one or two light chains comprising the amino acid sequence of SEQ ID NO:318, a heavy chain comprising the amino acid sequence of SEQ ID NO:319, and a heavy chain comprising the amino acid sequence of SEQ ID NO:320;
one or two light chains comprising the amino acid sequence of SEQ ID NO:322, a heavy chain comprising the amino acid sequence of SEQ ID NO:323, and a heavy chain comprising the amino acid sequence of SEQ ID NO:324;
one or two light chains comprising the amino acid sequence of SEQ ID NO:326, a heavy chain comprising the amino acid sequence of SEQ ID NO:327, and a heavy chain comprising the amino acid sequence of SEQ ID NO:328;
one or two light chains comprising the amino acid sequence of SEQ ID NO:330, a heavy chain comprising the amino acid sequence of SEQ ID NO:331, and a heavy chain comprising the amino acid sequence of SEQ ID NO:332;
one or two light chains comprising the amino acid sequence of SEQ ID NO:334, a heavy chain comprising the amino acid sequence of SEQ ID NO:335, and a heavy chain comprising the amino acid sequence of SEQ ID NO:336;
one or two light chains comprising the amino acid sequence of SEQ ID NO:338, a heavy chain comprising the amino acid sequence of SEQ ID NO:339, and a heavy chain comprising the amino acid sequence of SEQ ID NO:340;
one or two light chains comprising the amino acid sequence of SEQ ID NO:342, a heavy chain comprising the amino acid sequence of SEQ ID NO:343, and a heavy chain comprising the amino acid sequence of SEQ ID NO:344;
one or two light chains comprising the amino acid sequence of SEQ ID NO:346, a heavy chain comprising the amino acid sequence of SEQ ID NO:347, and a heavy chain comprising the amino acid sequence of SEQ ID NO:348;
one or two light chains comprising the amino acid sequence of SEQ ID NO:350, a heavy chain comprising the amino acid sequence of SEQ ID NO:351, and a heavy chain comprising the amino acid sequence of SEQ ID NO:352;
one or two light chains comprising the amino acid sequence of SEQ ID NO:156, a heavy chain comprising the amino acid sequence of SEQ ID NO: 157, and a heavy chain comprising the amino acid sequence of SEQ ID NO:217;
one or two light chains comprising the amino acid sequence of SEQ ID NO:159, a heavy chain comprising the amino acid sequence of SEQ ID NO: 160, and a heavy chain comprising the amino acid sequence of SEQ ID NO:218;
one or two light chains comprising the amino acid sequence of SEQ ID NO:162, a heavy chain comprising the amino acid sequence of SEQ ID NO: 163, and a heavy chain comprising the amino acid sequence of SEQ ID NO:219;
one or two light chains comprising the amino acid sequence of SEQ ID NO:165, a heavy chain comprising the amino acid sequence of SEQ ID NO:166, and a heavy chain comprising the amino acid sequence of SEQ ID NO:220;
one or two light chains comprising the amino acid sequence of SEQ ID NO:168, a heavy chain comprising the amino acid sequence of SEQ ID NO:169, and a heavy chain comprising the amino acid sequence of SEQ ID NO:221;
one or two light chains comprising the amino acid sequence of SEQ ID NO:171, a heavy chain comprising the amino acid sequence of SEQ ID NO: 172, and a heavy chain comprising the amino acid sequence of SEQ ID NO:222;
one or two light chains comprising the amino acid sequence of SEQ ID NO:174, a heavy chain comprising the amino acid sequence of SEQ ID NO: 175, and a heavy chain comprising the amino acid sequence of SEQ ID NO:223;
one or two light chains comprising the amino acid sequence of SEQ ID NO:187, a heavy chain comprising the amino acid sequence of SEQ ID NO:188, and a heavy chain comprising the amino acid sequence of SEQ ID NO:224;
one or two light chains comprising the amino acid sequence of SEQ ID NO:298, a heavy chain comprising the amino acid sequence of SEQ ID NO:299, and a heavy chain comprising the amino acid sequence of SEQ ID NO:301;
one or two light chains comprising the amino acid sequence of SEQ ID NO:302, a heavy chain comprising the amino acid sequence of SEQ ID NO:303, and a heavy chain comprising the amino acid sequence of SEQ ID NO:305;
one or two light chains comprising the amino acid sequence of SEQ ID NO:306, a heavy chain comprising the amino acid sequence of SEQ ID NO:307, and a heavy chain comprising the amino acid sequence of SEQ ID NO:309;
one or two light chains comprising the amino acid sequence of SEQ ID NO:310, a heavy chain comprising the amino acid sequence of SEQ ID NO:311, and a heavy chain comprising the amino acid sequence of SEQ ID NO:313;
one or two light chains comprising the amino acid sequence of SEQ ID NO:314, a heavy chain comprising the amino acid sequence of SEQ ID NO:315, and a heavy chain comprising the amino acid sequence of SEQ ID NO:317;
one or two light chains comprising the amino acid sequence of SEQ ID NO:318, a heavy chain comprising the amino acid sequence of SEQ ID NO:319, and a heavy chain comprising the amino acid sequence of SEQ ID NO:321;
one or two light chains comprising the amino acid sequence of SEQ ID NO:322, a heavy chain comprising the amino acid sequence of SEQ ID NO:323, and a heavy chain comprising the amino acid sequence of SEQ ID NO:325;
one or two light chains comprising the amino acid sequence of SEQ ID NO:326, a heavy chain comprising the amino acid sequence of SEQ ID NO:327, and a heavy chain comprising the amino acid sequence of SEQ ID NO:329;
one or two light chains comprising the amino acid sequence of SEQ ID NO:330, a heavy chain comprising the amino acid sequence of SEQ ID NO:331, and a heavy chain comprising the amino acid sequence of SEQ ID NO:333;
one or two light chains comprising the amino acid sequence of SEQ ID NO:334, a heavy chain comprising the amino acid sequence of SEQ ID NO:335, and a heavy chain comprising the amino acid sequence of SEQ ID NO:337;
one or two light chains comprising the amino acid sequence of SEQ ID NO:338, a heavy chain comprising the amino acid sequence of SEQ ID NO:339, and a heavy chain comprising the amino acid sequence of SEQ ID NO:341;
one or two light chains comprising the amino acid sequence of SEQ ID NO:342, a heavy chain comprising the amino acid sequence of SEQ ID NO:343, and a heavy chain comprising the amino acid sequence of SEQ ID NO:345;
one or two light chains comprising the amino acid sequence of SEQ ID NO:346, a heavy chain comprising the amino acid sequence of SEQ ID NO:347, and a heavy chain comprising the amino acid sequence of SEQ ID NO:349; or
one or two light chains comprising the amino acid sequence of SEQ ID NO:350, a heavy chain comprising the amino acid sequence of SEQ ID NO:351, and a heavy chain comprising the amino acid sequence of SEQ ID NO:353.

50. The method of any one of claims 1-33, wherein the fusion protein comprises:
(1) a first polypeptide chain comprising the amino acid sequence of SEQ ID NO:334, a second polypeptide chain comprising the amino acid sequence of SEQ ID NO:335, a third polypeptide chain comprising the amino acid sequence of SEQ ID NO:336, and a fourth polypeptide chain comprising the amino acid sequence of SEQ ID NO:334;
(2) a first polypeptide chain comprising the amino acid sequence of SEQ ID NO:334, a second polypeptide chain comprising the amino acid sequence of SEQ ID NO:335, a third polypeptide chain comprising the amino acid sequence of SEQ ID NO:337, and a fourth polypeptide chain comprising the amino acid sequence of SEQ ID NO:334;
(3) a first polypeptide chain comprising the amino acid sequence of SEQ ID NO:338, a second polypeptide chain comprising the amino acid sequence of SEQ ID NO:339, a third polypeptide chain comprising the amino acid sequence of SEQ ID NO:340, and a fourth polypeptide chain comprising the amino acid sequence of SEQ ID NO:338; or
(4) a first polypeptide chain comprising the amino acid sequence of SEQ ID NO:338, a second polypeptide chain comprising the amino acid sequence of SEQ ID NO:339, a third polypeptide chain comprising the amino acid sequence of SEQ ID NO:341, and a fourth polypeptide chain comprising the amino acid sequence of SEQ ID NO:338.

51. The method of any one of claims 1-50, wherein the fusion protein is administered in a pharmaceutical composition comprising the fusion protein and a pharmaceutically acceptable carrier.

52. The method of any one of claims 1-51, further comprising administering to the individual a second antiviral or immunomodulatory agent.

53. The method of claim 52, wherein the second antiviral or immunomodulatory agent is a nucleoside or nucleotide analog, capsid assembly modulator or inhibitor, TLR agonist, vaccine, RNAi-based agent, interferon-α, HBV entry inhibitor, covalently closed circular DNA (cccDNA) disruptor, HBV transcription inhibitor, CD3 bispecific T-cell redirection agent, HBV antigen targeting agent, PD1 and/or PDL1 blocking agent, agent that reduces PDL1 expression in hepatocytes, RNA destabilizer, or Hepatitis B surface antigen (HBsAg) release inhibitor.

54. The method of claim 52, wherein the second antiviral or immunomodulatory agent is an antiretroviral therapy (ART), PD1 and/or PDL1 blocking agent, or agent that reduces PDL1 expression.

55. The method of claim 54, wherein the ART comprises a nucleoside reverse transcriptase inhibitor (NRTI), non-nucleoside reverse transcriptase inhibitor (NNRTI), protease inhibitor (PI), fusion inhibitor, CCR5 antagonist, integrase strand transfer inhibitor (INSTI), attachment inhibitor, post-attachment inhibitor, pharmacokinetic enhancer, or combination thereof.

56. A kit comprising a fusion protein as described in any one of claims 1-50 for use in the method according to any one of claims 1-55.
